(19) United States
(12) Patent Application Publication
(10) Pub. No.: US 2025/0154112 A1
(43) Pub. Date: May 15, 2025

(54) A PREPARATION OF QUINAZOLINEDIONES AND USE THEREOF

(71) Applicant: COUNCIL OF SCIENTIFIC AND INDUSTRIAL RESEARCH AN INDIAN REGISTERED BODY INCORPORATED UNDER THE REGN., New Delhi (IN)

(72) Inventors: Arindam TALUKDAR, Kolkata (IN); Partha CHAKRABARTI, Kolkata (IN); Dipayan SARKAR, Kolkata (IN); Saheli Kumar CHOWDHURY, Kolkata (IN); Sunny GOON, Kolkata (IN); Abhishek SEN, Kolkata (IN); Uddipta GHOSH DASTIDAR, Kolkata (IN); Binita PATRA, Kolkata (IN); Israful HOQUE, Kolkata (IN)

(73) Assignee: COUNCIL OF SCIENTIFIC AND INDUSTRIAL RESEARCH AN INDIAN REGISTERED BODY INCORPORATED UNDER THE REGN., New Delhi (IN)

(21) Appl. No.: 18/723,847

(22) PCT Filed: Dec. 19, 2022
(86) PCT No.: PCT/IN2022/051099

§ 371 Date: Jun. 24, 2024

(30) Foreign Application Priority Data
Dec. 25, 2021 (IN) 202111061087

(51) Int. Cl. C07D 239/96; A61K 31/517; A61K 31/5377; C07D 401/04; C07D 401/06; C07D 403/06; C07D 403/12

(52) U.S. Cl. CPC C07D 239/96; A61K 31/517; A61K 31/5377; C07D 401/04; C07D 401/06; C07D 403/06; C07D 403/12

(57) ABSTRACT

The present invention described herein relates to a compound having Structure I for treating diseases and disorders for which inhibition or modulation of the Ubiquitin Ligase COP1 enzyme produces a physiologically beneficial response, in particular for the treatment of Non-Alcoholic Fatty Liver Disease (NAFLD). These compounds having Structure I are capable of increasing the level of adipose triglyceride lipase (ATGL). Also provided is the process of preparing compounds having Structure I.

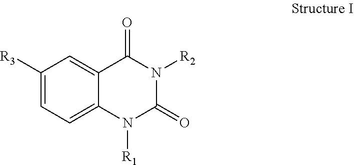

### FIELD OF THE INVENTION

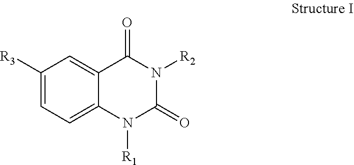

The present invention relates to the preparation of new compounds having structure I in free form or in an acceptable salt form for modulation of Ubiquitin Ligase COP1 through its stabilization as a potential therapeutic target for Non-Alcoholic Fatty

### Liver Disease (NAFLD).

The invention relates to small molecules where R₁, R₂, R₃ are as defined in the description, capable of increasing the level of adipose triglyceride lipase (ATGL) through modulation of Ubiquitin Ligase COP1 through its stabilization as a potential therapeutic target for treatment of Non-Alcoholic Fatty Liver Disease (NAFLD)

### BACKGROUND OF THE INVENTION

NonAlcoholic Fatty Liver Disease (NAFLD) has garnered considerable attention due to the increasing worldwide prevalence of this disease spectrum. NAFLD is an umbrella term encompassing simple steatosis progressing to steatohepatitis, fibrosis, cirrhosis, and HCC. Steatosis is mostly a reversible condition whereby fat droplets, mostly in the form of triglycerides, accumulate in the liver without pronounced hepatocyte injury. Steatohepatitis (NASH) denotes the stage wherein hepatocytes are significantly injured and is histologically characterized by the presence of ballooned hepatocytes, Mallory-Denk bodies, glycogenated nuclei and other distinguishing features. NASH may, in some cases, progress to fibrosis and cirrhosis which are more critical stages whereby extracellular matrix proteins, notably collagen fibres, accumulate in the liver encircling hepatocytes and forming scar tissue resulting in irreversible damage to the normal physiology of the liver. The prevalence of NAFLD is reported to be 20%-30% in Western countries and 5%-18% in Asia. While the incidence of NAFLD is rising at an alarming rate, with it being considered now as the second most common reason for liver transplantation, no robust therapies are available to reverse the advanced stages of this condition.

NAFLD is a complex multifactorial disorder involving the interplay of several molecules and their associated signalling pathways. A multitude of risk factors have been attributed to the development of NAFLD with type 2 diabetes and metabolic syndrome considered as the most important ones. As is evident, the most prominent feature of NAFLD is the deposition of excessive TAG in hepatocytes and, therefore, deregulation of enzymes responsible for controlling intracellular lipid turnover and homeostasis may play an important role in NAFLD (Ong et al. Hepatology. 2011, 53, 116-126). A pivotal enzyme associated with the intracellular degradation of TAG is Adipose triglyceride lipase (ATGL) also known as patatin-like phospholipase domain-containing protein 2 (PNPLA2). It catalyzes the initial and rate limiting step in the TAG lipolysis cascade. Indeed studies have shown that ATGL levels are decreased in NAFLD patients and liver injury is aggravated in mice with liver specific ATGL depletion (Jha et al. Hepatology, 2014, 59, 858-869).

Ubiquitin-proteasome system is a pivotal pathway for regulation of protein turnover in cells. Ubiquitination of a protein requires the stepwise involvement of 3 enzymes: E1-ubiquitin-activating enzymes, E2-ubiquitin-conjugating enzymes, and E3 ubiquitin ligases.COP1 is one such evolutionary conserved ubiquitin ligase which plays a central role in a myriad of important cellular pathways like insulin secretion from pancreatic B cells, regulating the stability of p53, etc.

Our previous study has identified a novel interaction between COP1 and the VP motif of ATGL. This interaction targets ATGL for proteasomal degradation by K-48 linked polyubiquitination, predominantly at the lysine 100 residue. In NAFLD, increased degradation of ATGL by COP1 would cause more TAG to accumulate in the liver manifesting a more severe form of the disease (Ghosh et al. Diabetes, 2016, 65, 3561-3572). Therefore, curtailing the ubiquitin mediated degradation of ATGL by inhibiting COP1 can be a potential area for therapeutics. Indeed in the same study it was validated that steatosis in mice liver could be ameliorated with adenovirus mediated depletion of COP1 in mice. In this context, if small molecules with the potential to target the interacting site of COP1 and ATGL can be developed to target COP1 and hinder its capability to ubiquitinate ATGL, ATGL would be able to hydrolyse the accumulated TAG in the liver and abort the progression of NAFLD. Therefore, if we can achieve this increased lipolysis in liver in the context of NAFLD, we would have a robust therapy at hand to combat the progression of steatosis to steatohepatitis ultimately restricting NAFLD at its very onset (Niyogi et al., Biochemical and Biophysical Research Communications, 2019, 512, 806-811).

At present, treatment strategies are mainly directed towards various targets that mediate hepatocyte dysregulation, inflammation, apoptosis and oxidative stress. Extrahepatic targets whose role are implicated in NASH like microbiome, gut liver axis, organs like muscle and adipose tissue are also being considered for designing therapeutic targets. Certain drugs are in clinical trials at various phases. Notably, elafibranor (PPAR-α/δ ligand), selonsertib (ASK-1 inhibitor), obeticholic acid (FXR agonist), cenicriviroc (CCR 2/5 inhibitor) are in Phase 3 trial. All these drugs aim at a much-advanced stage of fibrosis in NASH. Few drugs like Aramchol (SCD-1 inhibitor), IMM-124E (Anti-LPS), MGL-3196 (THR-β agonist), NGM282 (FGF19 analog), PF-05221304 (ACC inhibitor), etc. which are in Phase 2 clinical trials aim at an improvement in liver fat and therefore, target mainly the steatotic stage. Targeting the fibrotic stage in NASH may not always prove to be beneficial since mostly the stage is irreversible and much damage has already been inflicted in liver with deposition of collagen fibres and beginning of scar tissue formation. Hence, if we can curb the progression of NASH at the reversible stage of steatosis by curtailing the deposition of fat, a much effective therapy can be established.

Thus, keeping in view the drawbacks of the hitherto reported prior arts, there is a need to solve the problem of providing an innovative process for quinazolindione derivatives for treating diseases and disorders for which inhibition or modulation of the Ubiquitin Ligase COP1 enzyme produces a physiologically beneficial response, in particular for the treatment of Non-Alcoholic Fatty Liver Disease (NAFLD).

### OBJECTIVES OF THE INVENTION

The main objective of the present invention is to provide a compound having structure I.

Another objective of the present invention is to provide a process for the preparation of compound having structure I.

Still another objective of the present invention is to evaluate the efficacy of active compounds using screening methods including fluorescence microscopy and measurement of levels of ATGL protein.

Yet another objective of the present invention is to provide a method for testing the specificity of the compounds for targeting the interaction of ATGL-COP1.

Still another objective of the present invention is to increase the level of ATGL in hepatocytes that can decrease the level of cellular lipids.

Yet another objective of the present invention is to decrease the ubiquitination and proteasomal degradation of ATGL.

Still another objective of the present invention is to identify the specific E1 and E2 enzyme in ubiquitination process.

Yet another objective of the present invention is to decrease the level of triglycerides in hepatocytes.

Still another objective of the present invention is to test the efficacy of the compounds in vivo in preclinical models.

Yet another objective of the present invention is to provide a composition comprising compounds of structure I for use in a number of clinical applications, including pharmaceutical agents and methods for treating conditions like Non-Alcoholic Fatty Liver Disease (NAFLD).

Still another objective of the present invention is to provide a composition and methods of using the compounds having general structure I without considerable cytotoxicity in hepatocytes.

### SUMMARY OF THE INVENTION

In an embodiment of the present invention relates to a compound having structure I or a pharmaceutically acceptable salt thereof:

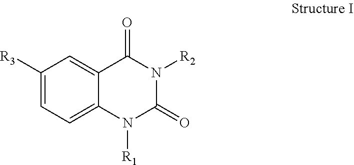

wherein
R₁ is independently selected from the group consisting of:

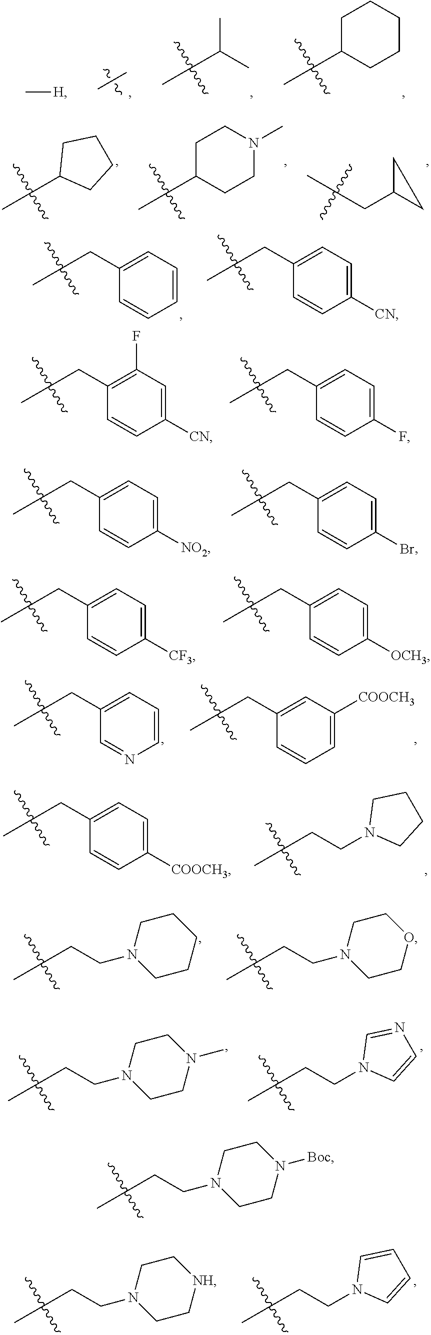

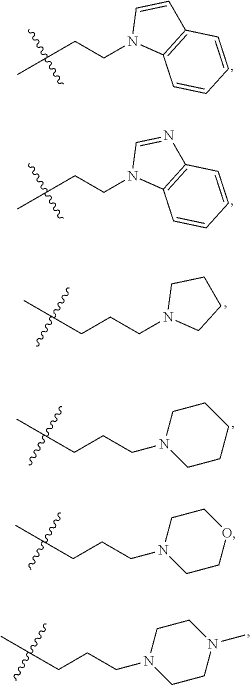

R₂ is independently selected from the group consisting of:

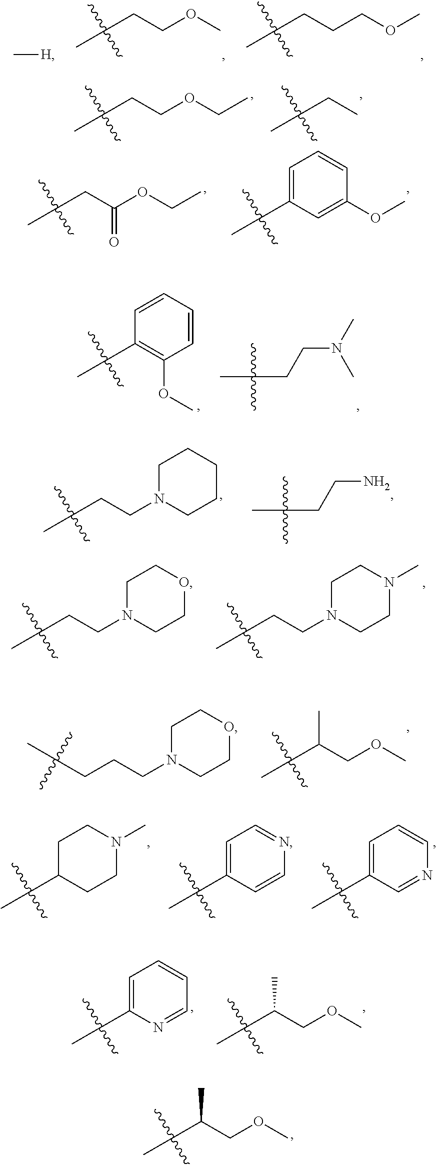

R₃ is independently selected from the group consisting of:

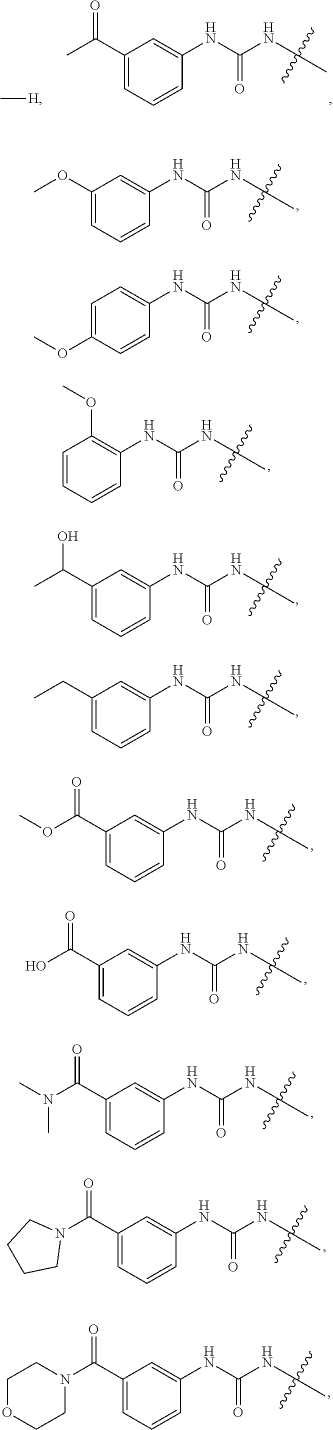

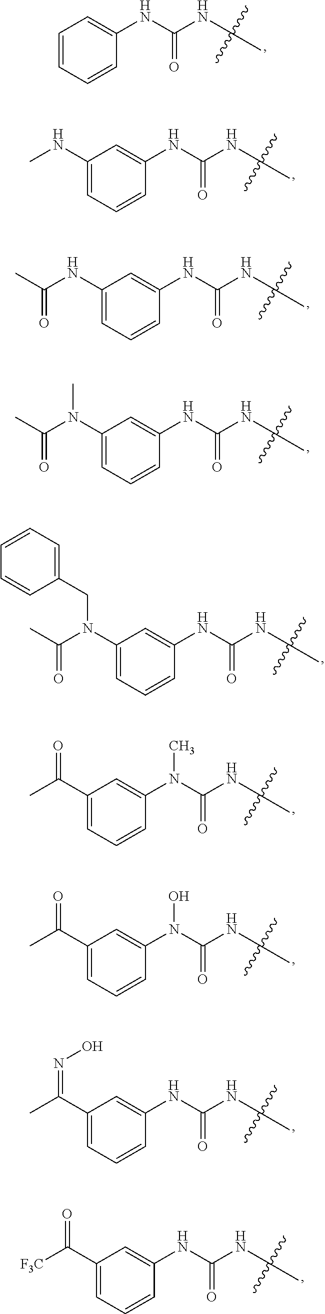

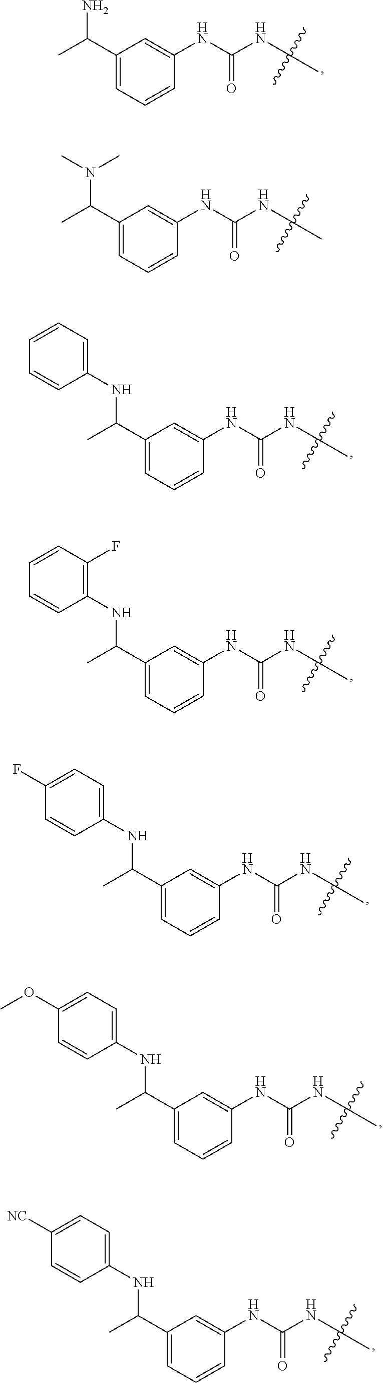

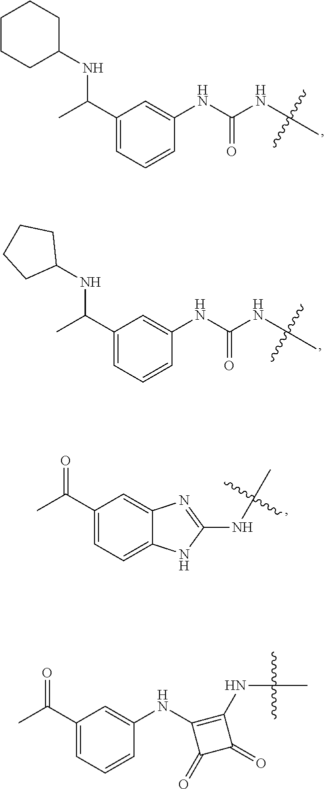 [SMILES: *NC(=O)Nc1cccc(C(C)NC2CCCCC2)c1 ; *NC(=O)Nc1cccc(C(C)NC2CCCC2)c1 ; *Nc1nc2cc(C(C)=O)ccc2[nH]1 ; *Nc1c(Nc2cccc(C(C)=O)c2)c(=O)c1=O]

In an embodiment of the present invention, the compound having structure I is selected from the group consisting of:
1-(3-acetylphenyl)-3-(3-(2-methoxyethyl)-2,4-dioxo-1,2,3,4-tetrahydroquinazolin-6-yl)urea (5),
1-(3-acetylphenyl)-3-(3-(2-methoxyethyl)-1-methyl-2,4-dioxo-1,2,3,4-tetrahydroquinazolin-6-yl)urea (8),
1-(3-acetylphenyl)-3-(1-isopropyl-3-(2-methoxyethyl)-2,4-dioxo-1,2,3,4-tetrahydroquinazolin-6-yl)urea (11),
1-(3-acetylphenyl)-3-(1-cyclohexyl-3-(2-methoxyethyl)-2,4-dioxo-1,2,3,4-tetrahydroquinazolin-6-yl)urea (14),
1-(3-acetylphenyl)-3-(1-cyclopentyl-3-(2-methoxyethyl)-2,4-dioxo-1,2,3,4-tetrahydroquinazolin-6-yl)urea (17),
1-(3-acetylphenyl)-3-(3-(2-methoxyethyl)-1-(1-methylpiperidin-4-yl)-2,4-dioxo-1,2,3,4-tetrahydroquinazolin-6-yl)urea (20),
1-(3-acetylphenyl)-3-(1-(cyclopropylmethyl)-3-(2-methoxyethyl)-2,4-dioxo-1,2,3,4-tetrahydroquinazolin-6-yl)urea (23),
1-(3-acetylphenyl)-3-(1-benzyl-3-(2-methoxyethyl)-2,4-dioxo-1,2,3,4-tetrahydroquinazolin-6-yl)urea (26),
1-(3-acetylphenyl)-3-(1-(4-cyanobenzyl)-3-(2-methoxyethyl)-2,4-dioxo-1,2,3,4-tetrahydroquinazolin-6-yl)urea (29),
1-(3-acetylphenyl)-3-(1-(4-cyano-2-fluorobenzyl)-3-(2-methoxyethyl)-2,4-dioxo-1,2,3,4-tetrahydroquinazolin-6-yl)urea (32),
1-(3-acetylphenyl)-3-(1-(4-fluorobenzyl)-3-(2-methoxyethyl)-2,4-dioxo-1,2,3,4-tetrahydroquinazolin-6-yl)urea (35),
1-(3-acetylphenyl)-3-(3-(2-methoxyethyl)-1-(4-nitrobenzyl)-2,4-dioxo-1,2,3,4-tetrahydroquinazolin-6-yl)urea (38),
1-(3-acetylphenyl)-3-(1-(4-bromobenzyl)-3-(2-methoxyethyl)-2,4-dioxo-1,2,3,4-tetrahydroquinazolin-6-yl)urea (41),
1-(3-acetylphenyl)-3-(3-(2-methoxyethyl)-2,4-dioxo-1-(4-(trifluoromethyl)benzyl)-1,2,3,4-tetrahydroquinazolin-6-yl)urea (44),
1-(3-acetylphenyl)-3-(1-(4-methoxybenzyl)-3-(2-methoxyethyl)-2,4-dioxo-1,2,3,4-tetrahydroquinazolin-6-yl)urea (47),
1-(3-acetylphenyl)-3-(3-(2-methoxyethyl)-2,4-dioxo-1-(pyridin-3-ylmethyl)-1,2,3,4-tetrahydroquinazolin-6-yl)urea (50),
Methyl 3-((6-(3-(3-acetylphenyl)ureido)-3-(2-methoxyethyl)-2,4-dioxo-3,4-dihydroquinazolin-1 (2H)-yl)methyl)benzoate (53),
methyl 4-((6-(3-(3-acetylphenyl)ureido)-3-(2-methoxyethyl)-2,4-dioxo-3,4-dihydroquinazolin-1 (2H)-yl)methyl)benzoate (56),
1-(3-acetylphenyl)-3-(3-(2-methoxyethyl)-2,4-dioxo-1-(2-(pyrrolidin-1-yl)ethyl)-1,2,3,4-tetrahydroquinazolin-6-yl)urea (59),
1-(3-acetylphenyl)-3-(3-(2-methoxyethyl)-2,4-dioxo-1-(2-(piperidin-1-yl)ethyl)-1,2,3,4-tetrahydroquinazolin-6-yl)urea (62),
1-(3-acetylphenyl)-3-(3-(2-methoxyethyl)-1-(2-morpholinoethyl)-2,4-dioxo-1,2,3,4-tetrahydroquinazolin-6-yl)urea (65),
1-(3-acetylphenyl)-3-(3-(2-methoxyethyl)-1-(2-(4-methylpiperazin-1-yl)ethyl)-2,4-dioxo-1,2,3,4-tetrahydroquinazolin-6-yl)urea (68),
1-(1-(2-(1H-imidazol-1-yl)ethyl)-3-(2-methoxyethyl)-2,4-dioxo-1,2,3,4-tetrahydroquinazolin-6-yl)-3-(3-acetylphenyl)urea (72),
tert-butyl 4-(2-(6-(3-(3-acetylphenyl)ureido)-3-(2-methoxyethyl)-2,4-dioxo-3,4-dihydroquinazolin-1 (2H)-yl)ethyl) piperazine-1-carboxylate (75),
1-(3-acetylphenyl)-3-(3-(2-methoxyethyl)-2,4-dioxo-1-(2-(piperazin-1-yl)ethyl)-1,2,3,4-tetrahydroquinazolin-6-yl)urea (76),
1-(1-(2-(1H-pyrrol-1-yl)ethyl)-3-(2-methoxyethyl)-2,4-dioxo-1,2,3,4-tetrahydroquinazolin-6-yl)-3-(3-acetylphenyl)urea (79),
1-(1-(2-(1H-indol-1-yl)ethyl)-3-(2-methoxyethyl)-2,4-dioxo-1,2,3,4-tetrahydroquinazolin-6-yl)-3-(3-acetylphenyl)urea (82),
1-(1-(2-(1H-benzo[d]imidazol-1-yl)ethyl)-3-(2-methoxyethyl)-2,4-dioxo-1,2,3,4-tetrahydroquinazolin-6-yl)-3-(3-acetylphenyl)urea (85),
1-(3-acetylphenyl)-3-(3-(2-methoxyethyl)-2,4-dioxo-1-(3-(pyrrolidin-1-yl) propyl)-1,2,3,4-tetrahydroquinazolin-6-yl)urea (88),
1-(3-acetylphenyl)-3-(3-(2-methoxyethyl)-2,4-dioxo-1-(3-(piperidin-1-yl)propyl)-1,2,3,4-tetrahydroquinazolin-6-yl)urea (91),
1-(3-acetylphenyl)-3-(3-(2-methoxyethyl)-1-(3-morpholinopropyl)-2,4-dioxo-1,2,3,4-tetrahydroquinazolin-6-yl)urea (94),
1-(3-acetylphenyl)-3-(3-(2-methoxyethyl)-1-(3-(4-methylpiperazin-1-yl)propyl)-2,4-dioxo-1,2,3,4-tetrahydroquinazolin-6-yl)urea (97),
1-(3-acetylphenyl)-3-(3-(3-methoxypropyl)-2,4-dioxo-1-(2-(piperidin-1-yl)ethyl)-1,2,3,4-tetrahydroquinazolin-6-yl)urea (102),
1-(3-acetylphenyl)-3-(3-(2-ethoxyethyl)-2,4-dioxo-1-(2-(piperidin-1-yl)ethyl)-1,2,3,4-tetrahydroquinazolin-6-yl)urea (107),
1-(3-acetylphenyl)-3-(3-ethyl-2,4-dioxo-1-(2-(piperidin-1-yl)ethyl)-1,2,3,4-tetrahydroquinazolin-6-yl)urea (112),
ethyl 2-(6-(3-(3-acetylphenyl)ureido)-2,4-dioxo-1-(2-(piperidin-1-yl)ethyl)-1,2-dihydroquinazolin-3 (4H)-yl)acetate (117),
1-(3-acetylphenyl)-3-(3-(3-methoxyphenyl)-2,4-dioxo-1-(2-(piperidin-1-yl)ethyl)-1,2,3,4-tetrahydroquinazolin-6-yl)urea (122),
1-(3-acetylphenyl)-3-(3-(2-methoxyphenyl)-2,4-dioxo-1-(2-(piperidin-1-yl)ethyl)-1,2,3,4-tetrahydroquinazolin-6-yl)urea (127),
1-(3-acetylphenyl)-3-(3-(2-(dimethylamino)ethyl)-2,4-dioxo-1-(2-(piperidin-1-yl)ethyl)-1,2,3,4-tetrahydroquinazolin-6-yl)urea (132),
1-(3-acetylphenyl)-3-(2,4-dioxo-1,3-bis(2-(piperidin-1-yl)ethyl)-1,2,3,4-tetrahydroquinazolin-6-yl)urea (137),
1-(3-acetylphenyl)-3-(3-(2-aminoethyl)-2,4-dioxo-1-(2-(piperidin-1-yl)ethyl)-1,2,3,4-tetrahydroquinazolin-6-yl)urea (142),
1-(3-acetylphenyl)-3-(3-(2-(4-methylpiperazin-1-yl)ethyl)-2,4-dioxo-1-(2-(piperidin-1-yl)ethyl)-1,2,3,4-tetrahydroquinazolin-6-yl)urea (147),
1-(3-acetylphenyl)-3-(3-(3-morpholinopropyl)-2,4-dioxo-1-(2-(piperidin-1-yl)ethyl)-1,2,3,4-tetrahydroquinazolin-6-yl)urea (152),
1-(3-acetylphenyl)-3-(3-(1-methoxypropan-2-yl)-2,4-dioxo-1-(2-(piperidin-1-yl)ethyl)-1,2,3,4-tetrahydroquinazolin-6-yl)urea (157),
1-(3-acetylphenyl)-3-(3-(1-methylpiperidin-4-yl)-2,4-dioxo-1-(2-(piperidin-1-yl)ethyl)-1,2,3,4-tetrahydroquinazolin-6-yl)urea (162),
1-(3-acetylphenyl)-3-(2,4-dioxo-1-(2-(piperidin-1-yl)ethyl)-3-(pyridin-4-yl)-1,2,3,4-tetrahydroquinazolin-6-yl)urea (167),
1-(3-acetylphenyl)-3-(2,4-dioxo-1-(2-(piperidin-1-yl)ethyl)-3-(pyridin-3-yl)-1,2,3,4-tetrahydroquinazolin-6-yl)urea (172),
1-(3-acetylphenyl)-3-(2,4-dioxo-1-(2-(piperidin-1-yl)ethyl)-3-(pyridin-2-yl)-1,2,3,4-tetrahydroquinazolin-6-yl)urea (177),
1-(3-(2-methoxyethyl)-2,4-dioxo-1-(2-(piperidin-1-yl)ethyl)-1,2,3,4-tetrahydroquinazolin-6-yl)-3-(3-methoxyphenyl)urea (178),
1-(3-(2-methoxyethyl)-2,4-dioxo-1-(2-(piperidin-1-yl)ethyl)-1,2,3,4-tetrahydroquinazolin-6-yl)-3-(2-methoxyphenyl)urea (179),
1-(3-(1-hydroxyethyl)phenyl)-3-(3-(2-methoxyethyl)-2,4-dioxo-1-(2-(piperidin-1-yl)ethyl)-1,2,3,4-tetrahydroquinazolin-6-yl)urea (180),
1-(3-ethylphenyl)-3-(3-(2-methoxyethyl)-2,4-dioxo-1-(2-(piperidin-1-yl)ethyl)-1,2,3,4-tetrahydroquinazolin-6-yl)urea (181),
Methyl3-(3-(3-(2-methoxyethyl)-2,4-dioxo-1-(2-(piperidin-1-yl)ethyl)-1,2,3,4-tetrahydroquinazolin-6-yl) ureido)benzoate (182),
3-(3-(3-(2-methoxyethyl)-2,4-dioxo-1-(2-(piperidin-1-yl)ethyl)-1,2,3,4-tetrahydroquinazolin-6-yl) ureido)benzoic acid (183),
3-(3-(3-(2-methoxyethyl)-2,4-dioxo-1-(2-(piperidin-1-yl)ethyl)-1,2,3,4-tetrahydroquinazolin-6-yl) ureido)-N,N-dimethylbenzamide (184),
1-(3-(2-methoxyethyl)-2,4-dioxo-1-(2-(piperidin-1-yl)ethyl)-1,2,3,4-tetrahydroquinazolin-6-yl)-3-(3-(pyrrolidine-1-carbonyl)phenyl)urea (185),
1-(3-(2-methoxyethyl)-2,4-dioxo-1-(2-(piperidin-1-yl)ethyl)-1,2,3,4-tetrahydroquinazolin-6-yl)-3-(3-(morpholine-4-carbonyl)phenyl)urea (186),
1-(3-(2-methoxyethyl)-2,4-dioxo-1-(2-(piperidin-1-yl)ethyl)-1,2,3,4-tetrahydroquinazolin-6-yl)-3-phenylurea (187),
1-(3-(2-methoxyethyl)-2,4-dioxo-1-(2-(piperidin-1-yl)ethyl)-1,2,3,4-tetrahydroquinazolin-6-yl)-3-(3-(methylamino)phenyl)urea (188),
N-(3-(3-(3-(2-methoxyethyl)-2,4-dioxo-1-(2-(piperidin-1-yl)ethyl)-1,2,3,4-tetrahydroquinazolin-6-yl)ureido)phenyl)acetamide (189),
N-(3-(3-(3-(2-methoxyethyl)-2,4-dioxo-1-(2-(piperidin-1-yl)ethyl)-1,2,3,4-tetrahydroquinazolin-6-yl) ureido)phenyl)-N-methylacetamide (190),
N-benzyl-N-(3-(3-(3-(2-methoxyethyl)-2,4-dioxo-1-(2-(piperidin-1-yl)ethyl)-1,2,3,4-tetrahydroquinazolin-6-yl)ureido)phenyl)acetamide (191),
1-(3-acetylphenyl)-3-(3-(2-methoxyethyl)-2,4-dioxo-1-(2-(piperidin-1-yl)ethyl)-1,2,3,4-tetrahydroquinazolin-6-yl)-1-methylurea (192),
1-(3-acetylphenyl)-1-hydroxy-3-(3-(2-methoxyethyl)-2,4-dioxo-1-(2-(piperidin-1-yl)ethyl)-1,2,3,4-tetrahydroquinazolin-6-yl)urea (193),
(Z)-1-(3-(1-(hydroxyimino)ethyl)phenyl)-3-(3-(2-methoxyethyl)-2,4-dioxo-1-(2-(piperidin-1-yl)ethyl)-1,2,3,4-tetrahydroquinazolin-6-yl)urea (194),
1-(3-(2-methoxyethyl)-2,4-dioxo-1-(2-(piperidin-1-yl)ethyl)-1,2,3,4-tetrahydroquinazolin-6-yl)-3-(3-(2,2,2-trifluoroacetyl)phenyl)urea (195),
1-(3-(1-aminoethyl)phenyl)-3-(3-(2-methoxyethyl)-2,4-dioxo-1-(2-(piperidin-1-yl)ethyl)-1,2,3,4-tetrahydroquinazolin-6-yl)urea (196),
1-(3-(1-(dimethylamino)ethyl)phenyl)-3-(3-(2-methoxyethyl)-2,4-dioxo-1-(2-(piperidin-1-yl)ethyl)-1,2,3,4-tetrahydroquinazolin-6-yl)urea (197),
1-(3-(2-methoxyethyl)-2,4-dioxo-1-(2-(piperidin-1-yl)ethyl)-1,2,3,4-tetrahydroquinazolin-6-yl)-3-(3-(1-(phenylamino)ethyl)phenyl)urea (199),
1-(3-(1-((2-fluorophenyl)amino)ethyl)phenyl)-3-(3-(2-methoxyethyl)-2,4-dioxo-1-(2-(piperidin-1-yl)ethyl)-1,2,3,4-tetrahydroquinazolin-6-yl)urea (201),
1-(3-(1-((4-fluorophenyl)amino)ethyl)phenyl)-3-(3-(2-methoxyethyl)-2,4-dioxo-1-(2-(piperidin-1-yl)ethyl)-1,2,3,4-tetrahydroquinazolin-6-yl)urea (203),
1-(3-(2-methoxyethyl)-2,4-dioxo-1-(2-(piperidin-1-yl)ethyl)-1,2,3,4-tetrahydroquinazolin-6-yl)-3-(3-(1-((4-methoxyphenyl)amino)ethyl)phenyl)urea
(205),
1-(3-(1-((4-cyanophenyl)amino)ethyl)phenyl)-3-(3-(2-methoxyethyl)-2,4-dioxo-1-(2-(piperidin-1-yl)ethyl)-1,2,3,4-tetrahydroquinazolin-6-yl)urea (207),
1-(3-(1-(cyclohexylamino)ethyl)phenyl)-3-(3-(2-methoxyethyl)-2,4-dioxo-1-(2-(piperidin-1-yl)ethyl)-1,2,3,4-tetrahydroquinazolin-6-yl)urea (209),
1-(3-(1-(cyclopentylamino)ethyl)phenyl)-3-(3-(2-methoxyethyl)-2,4-dioxo-1-(2-(piperidin-1-yl)ethyl)-1,2,3,4-tetrahydroquinazolin-6-yl)urea (211),
6-((5-acetyl-1H-benzo[d]imidazol-2-yl)amino)-3-(2-methoxyethyl)-1-(2-(piperidin-1-yl)ethyl) quinazoline-2,4 (1H,3H)-dione (216),
6-((2-((3-acetylphenyl)amino)-3,4-dioxocyclobut-1-en-1-yl)amino)-3-(2-methoxyethyl)-1-(2-(piperidin-1-yl)ethyl) quinazoline-2,4 (1H,3H)-dione (217),
(R)-1-(3-acetylphenyl)-3-(3-(1-methoxypropan-2-yl)-2,4-dioxo-1-(2-(piperidin-1-yl)ethyl)-1,2,3,4-tetrahydroquinazolin-6-yl)urea (222),
(S)-1-(3-acetylphenyl)-3-(3-(1-methoxypropan-2-yl)-2,4-dioxo-1-(2-(piperidin-1-yl)ethyl)-1,2,3,4-tetrahydroquinazolin-6-yl)urea (226),
(R)-3-(3-(3-(1-methoxypropan-2-yl)-2,4-dioxo-1-(2-(piperidin-1-yl)ethyl)-1,2,3,4-tetrahydroquinazolin-6-yl) ureido)-N,N-dimethylbenzamide (228),
(S)-3-(3-(3-(1-methoxypropan-2-yl)-2,4-dioxo-1-(2-(piperidin-1-yl)ethyl)-1,2,3,4-tetrahydroquinazolin-6-yl) ureido)-N,N-dimethylbenzamide (229),
(R)-1-(3-(1-methoxypropan-2-yl)-2,4-dioxo-1-(2-(piperidin-1-yl)ethyl)-1,2,3,4-tetrahydroquinazolin-6-yl)-3-(3-(2,2,2-trifluoroacetyl)phenyl)urea (230),
(S)-1-(3-(1-methoxypropan-2-yl)-2,4-dioxo-1-(2-(piperidin-1-yl)ethyl)-1,2,3,4-tetrahydroquinazolin-6-yl)-3-(3-(2,2,2-trifluoroacetyl)phenyl)urea (231).

Yet another embodiment of the present invention provides a process for the preparation of compounds having structure I, the process comprising:
(i) reacting 2-amino-5-nitrobenzoic acid (compound 1) with an aliphatic or an aromatic amine selected from the group consisting of 2-methoxyethylamine, methoxypropylamine, 2-ethoxyethylamine, ethylamine 2M in THF, glycine ethylester hydrochloride, m-anisidine, o-anisidine, N,N-dimethylethylenediamine, 1-(2-aminoethyl) piperidine, 4-(2-aminoethyl) morpholine, 1-(2-Aminoethyl)-4-methylpiperazine, 3-(4-morpholinyl) propylamine, rac-1-methoxy-2-propylamine, 4-amino-1-methylpiperidine, 4-aminopyridine, 3-aminopyridine, 2-aminopyridine, (R)-1-methoxy-2-propylamine, (S)-1-methoxy-2-propylamine in presence of HATU/DMF followed by TEA as a base at room temperature for 1-3 hours to obtain an amide compound selected from the group consisting of 2, 98, 103, 108, 113, 118, 123, 128, 133, 138, 143, 148, 153, 158, 163, 168, 173, 218, and 223;
(ii) cyclizing the compound selected from the group consisting of 2, 98, 103, 108, 113, 118, 123, 128, 133, 138, 143, 148, 153, 158, 163, 168, 173, 218, and 223 obtained in step (i) using a cyclizing agent CDI in DMF as solvent at 100° C. for 12-16 hours to obtain a compound selected from the group consisting of 3, 99, 104, 109, 114, 119, 124, 129, 134, 139, 144, 149, 154, 159, 164, 169, 174, 219, and 224;
(iii) reacting the compound 3 obtained in step (ii) with methyliodide and dry DMF at 0° C. for 12 hours to obtain the compound 6;
(iv) reacting the compound 3, 99, 104, 109, 114, 119, 124, 129, 134, 139, 144, 149, 154, 159, 164, 169, 174, 219, and 224 obtained in step (ii) with suitable alkyl chloride selected from the group consisting of2-iodopropane, bromocyclohexane, bromocyclopentane, 4-bromo 1-methyl piperidine, (bromomethyl)cyclopropane, benzyl bromide, 4-(bromomethyl)benzonitrile, 4-(bromomethyl)-3-fluorobenzonitrile, 4-fluorobenzyl bromide, 4-nitrobenzyl bromide, 4-bromobenzyl bromide, 4-Methoxybenzyl bromide, 3-(bromomethyl)pyridine hydrobromide, methyl 3-(bromomethyl)benzoate, methyl 4-(bromomethyl)benzoate, 1-(2-chloroethyl) pyrrolidine, 1-(2-chloroethyl) piperidine, 4-(2-chloroethyl) morpholine, 1-(2-chloroethyl)-4-methylpiperazine, 1-(3-chloropropyl) pyrrolidine, 1-(3-chloropropyl) piperidine, 4-(3-chloropropyl) morpholine, 1-(3-chloropropyl)-4-methylpiperazine with K₂CO₃ and dry DMF at 120° C. for 12 hours to obtain the compound selected from the group consisting of 9, 12, 18, 21, 24, 27, 30, 33, 36, 39, 42, 45, 48, 51, 54, 57, 60, 63, 66, 86, 89, 92, 95, 100, 105, 110, 115, 120, 125, 130, 135, 140, 145, 150, 155, 160, 165, 170, 175, 220, 225;
(v) reacting the compound 3 obtained in step (ii) with chlorobromoethane K₂CO₃ and dry DMF at 120° C. for 12 hours to obtain the compound 69;
(vi) reacting the compound 69 obtained in step (v) with suitable amine from the group consisting of imidazole, N-Boc piperazine, pyrrole, indole, benzimidazole K₂CO₃ and dry DMF at 120° C. for 12 hours to obtain the compound selected from the group consisting of 70, 73, 77, 80, and 83;
(vii) reducing the compound selected from the group consisting of 3, 6, 9, 12, 18, 21, 24, 27, 30, 33, 36, 39, 42, 45, 48, 51, 54, 57, 60, 63, 66, 70, 73, 77, 80, 83, 86, 89, 92, 225 obtained in steps (ii), (iii) and (iv) using Palladium-Charcoal (5% or 10% wet) at room temperature for 2-5 hours in presence of H₂ to obtain an amine compound selected from the group consisting of 4, 7, 10, 13, 19, 22, 25, 28, 31, 34, 37, 40, 43, 46, 49, 52, 55, 58, 61, 64, 67, 71, 74, 78, 81, 84, 87, 90, 93, 96, 101, 106, 111, 116, 121, 126, 131, 136, 141, 146, 151, 156, 161, 166, 171, 176, 221, 226;
(viii) treating the compound 4, 7, 10, 13, 19, 22, 25, 28, 31, 34, 37, 40, 43, 46, 49, 52, 55, 58, 61, 64, 67, 71, 74, 78, 81, 84, 87, 90, 93, 96, 101, 106, 111, 116, 121, 126, 131, 136, 141, 146, 151, 156, 161, 166, 171, 176, 221, 226 obtained in step (vii) with 4-nitrophenylchloroformate in presence of TEA as a base followed by reaction with an amine selected from the group consisting of 3′-aminoacetophenone in dry THF at room temperature for 3-8 hours to obtain the compound having structure I selected from the group consisting of 5, 8, 11, 14, 20, 23, 26, 29, 32, 35, 38, 41, 44, 47, 50, 53, 56, 59, 62, 65, 68, 72, 75, 79, 82, 85, 88, 91, 94, 97, 102, 107, 112, 117, 122, 127, 132, 137, 142, 147, 152, 157, 162, 167, 172, 177, 222, 227;
(ix) treating the compound 61, 221, 226 obtained in step (vii) with 4-nitrophenylchloroformate in presence of TEA as a base followed by reaction with an amine selected from the group consisting of m-anisidine, o-anisidine, 1-(3-aminophenyl) ethanol, 3-ethylaniline, methyl 3-aminobenzoate, 3-amino-N,N-dimethylbenzamide, (3-aminophenyl) (pyrrolidin-1-yl) methanone, (3-aminophenyl) (morpholino) methanone, aniline, N1-methylbenzene-1,3-diamine, N-(3-aminophenyl) acetamide, N-(3-aminophenyl)-N-methylacetamide, N-(3-aminophenyl)-N-benzylacetamide, 1-(3-(methylamino)phenyl) ethanone, 1-(3-(hydroxyamino)phenyl) ethanone, 1-(3-aminophenyl)-2,2,2-trifluoroethanone in dry THF at room temperature for 3-8 hours to obtain the compound having structure I selected from the group consisting of 178, 179, 180, 181, 182, 184, 185, 186, 187, 188, 189, 190, 191, 192, 193, 195, 228, 229, 230, 231;
(x) treating the compound 182 obtained in step (ix) with LiOH. H₂O in THE, Methanol and H₂O in proportion of (3:2:1) at room temperature for 12 hours to obtain the compound 183 having structure I;
(xi) treating the compound 62 obtained in step (viii) with hydroxylamine hydrochloride in EtOH at room temperature for 12 hours to obtain the compound 194 having structure I;
(xii) treating the compound 75 obtained in step (viii) with trifluoroacetic acid in DCM at room temperature for 8 hours to obtain the compound 76 having structure I;
(xiii) treating the compound 62 obtained in step (viii) with NH₃/Methanol and followed by NaBH₄ in Methanol at room temperature for 12 hours to obtain the compound 196 having structure I;
(xiv) treating the compound 196 obtained in step (xiii) with HCOOH and formaldehyde in presence of conc. HCl for 8 hours at 100° C. to obtain the compound 197 having structure I;
(xv) treating the compound 62 obtained in step (viii) with various aromatic amines (Aniline, 2-Fluoroaniline, 4-Fluoroaniline, 4-Methoxyaniline, 4-Aminobenzonitrile, cyclohexylamine, cyclopentylamine) in presence of p-toluenesulfonic acid (PTSA) in EtOH at 100° C. to obtain the compounds having structure I selected from the group consisting of 196, 200, 202, 204, 206, 208, 210;
(xvi) treating the compounds196, 200, 202, 204, 206, 208, 210 obtained in step (xv) with Sodium cyanoborohydride (NaCNBH₃) in dry methanol at room temperature for 8 hours to obtain the compounds having structure I selected from the group consisting of197, 201, 203, 205, 207, 209, 211;
(xvii) treating the compound which is commercially available 212 with HATU and 2-methoxyethylamine in DMF at room temperature for 2 hours to obtain the compound213;
(xviii) treating the compound obtained in step (xvii) with CDI in DMF for 12 hours at 100° C. to obtain the compound 214;
(xix) reacting the compound 214 obtained step (xviii) with 1-(2-chloroethyl) piperidine and K₂CO₃ and dry DMF at 120° C. for 12 hours to obtain the compound 215;
(xx) reacting the compound 215 obtained in step (xix) with 1-(2-amino-1H-benzo[d]imidazol-5-yl) ethanone and Pd₂(dba)₃ as catalyst and X-Phos as ligand and base K₂CO₃ at 100° C. for 12 hours to obtain the compound 216;
(xxi) reacting the compound 61 obtained in step (vii) with 3,4-Diethoxy-3-cyclobutene-1,2-dione and p-toluenesulfonic acid (PTSA) in EtOH and 3-aminoacetophenone at 80° C. for 12 hours to obtain the compound 217.

Still another embodiment of the present application provides a compound having structure I or salts thereof for use in treating diseases and disorders related to modulation of COP1 enzyme through its stabilization or modulation of ATGL.

Another embodiment of the present invention provides a compound having structure I or salts thereof for use in decreasing the level of triglycerides in hepatocytes.

Yet another embodiment of the present invention provides a compound having structure I or salts thereof for use in treatment of disease selected from Non-Alcoholic Fatty Liver Disease (NAFLD) or Non-Alcoholic Steatohepatitis (NASH).

Another embodiment of the present invention provides a compound having structure I or salts thereof along with pharmaceutically acceptable excipients.

Still another embodiment of the present invention provides a method of modulation COP1 enzyme through its stabilization by compound having structure I.

Yet another aspect of the present invention provides a method of increasing the level of ATGL by compound having structure I.

### BRIEF DESCRIPTION OF DRAWING

The invention has other advantages and features which will be more readily apparent from the following detailed description of the invention and the appended claims, when taken in conjunction with the accompanying drawings, in which:

FIG. 1 (A to G) illustrates results of Western Blot Analysis in HepG2 cells after treatment with compounds 5, 11, 62, 94, 8, 59 and 91, in accordance with an implementation of the present disclosure.

FIG. 2 illustrates ATGL protein status in mouse primary hepatocytes after compound treatment, in accordance with an implementation of the present disclosure.

FIG. 3 (A to C) illustrates results of immunoprecipitation assay to check ubiquitination status of ATGL after treatment with compounds, in accordance with an implementation of the present disclosure.

FIG. 4 illustrates images of HepG2 cells upon compound treatment using confocal microscopy, in accordance with an implementation of the present disclosure.

FIG. 5 illustrates the results of Western Blot Analysis in HepG2 cells after treatment with compounds 5, 8, 11, 59, 62, 65, 91, 94 in a single panel at 5 μM of dose, in accordance with an implementation of the present disclosure.

FIG. 6 illustrates the status of induction of ATGL and stabilization of COP1 in mice liver and adipose tissue by the treatment of compound by oral administration for 9 days at 80 mg/Kg dose, in accordance with an implementation of the present disclosure.

FIG. 7 (A,B,C,D,E) illustrates histological data (H&E, Liver weight, Body weight, Serum Cholesterol, Liver triglyceride) of randomly distributed C57BL/6 mice which were fed with HFD for 12 weeks and orally gavaged with vehicle (red) and compound 62 (blue) for the last 4 weeks.

### DETAILED DESCRIPTION OF THE INVENTION

While the invention has been disclosed with reference to certain embodiments, it will be understood by those skilled in the art that various changes may be made, and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise.

The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

A complex multifactorial disorder like NAFLD leads to the deposition of excessive TAG in hepatocytes and, therefore, deregulation of enzymes responsible for controlling intracellular lipid turnover and homeostasis may play an important role in NAFLD. Adipose triglyceride lipase (ATGL) also known as patatin-like phospholipase domain-containing protein 2 (PNPLA2) is a pivotal enzyme associated with the intracellular degradation of TAG which catalyses the initial rate limiting step in the TAG lipolysis cascade.

In line with the above objectives, the present invention relates to a compound having general structure I or salts thereof:

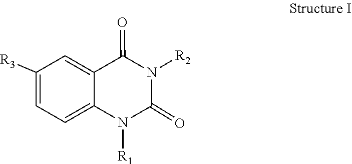

wherein
R₁ is independently selected from the group consisting of:

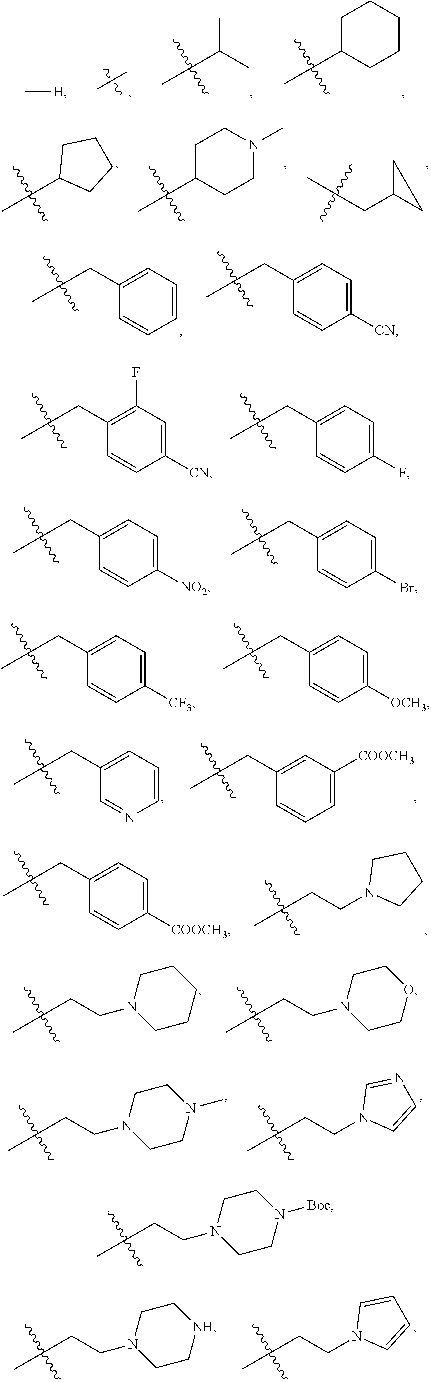

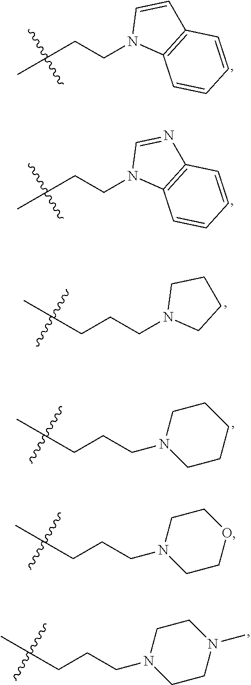

R₂ is independently selected from the group consisting of:

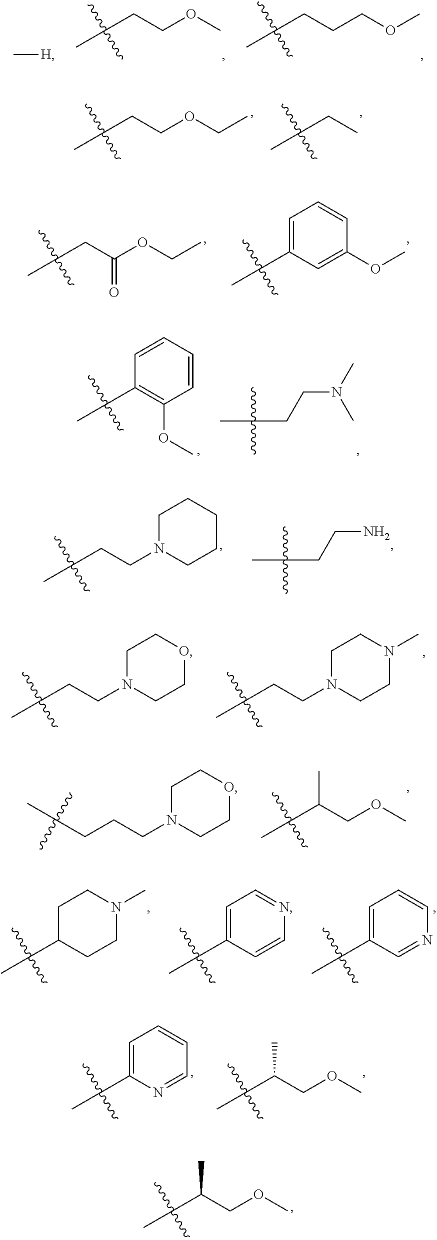

R₃ is independently selected from the group consisting of:

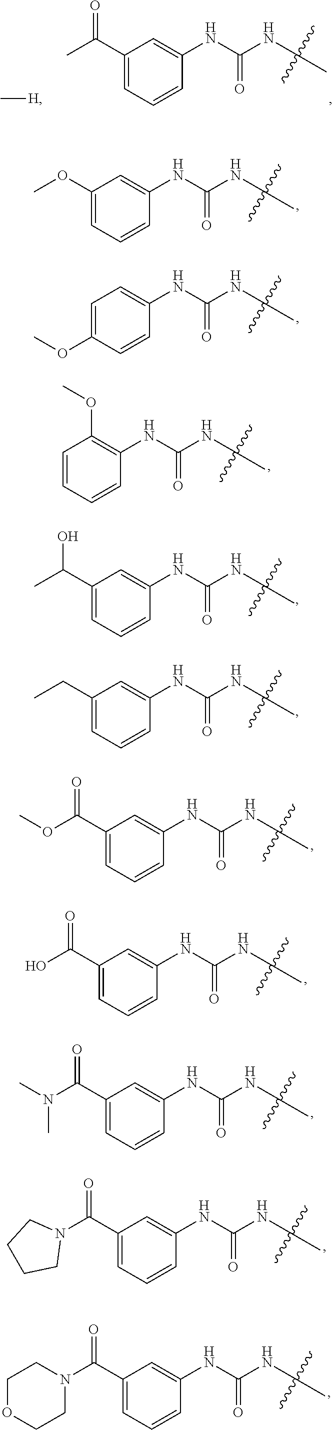

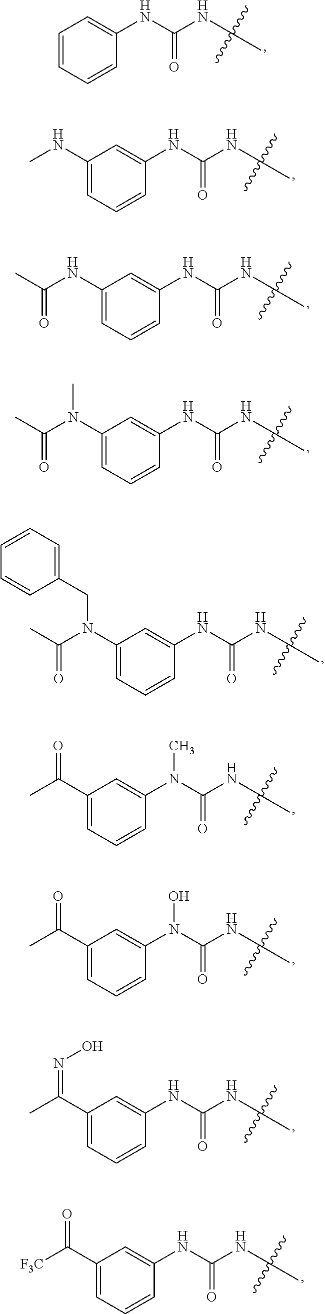

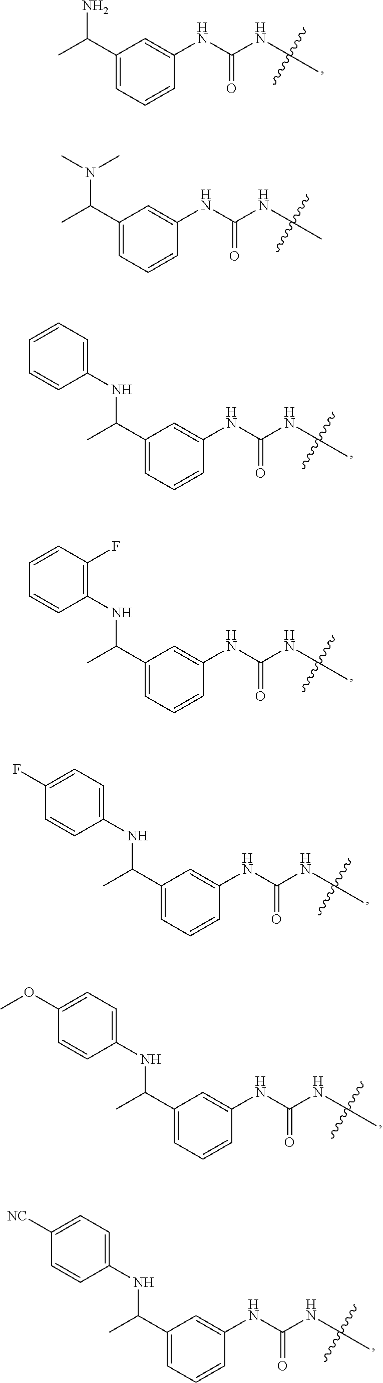

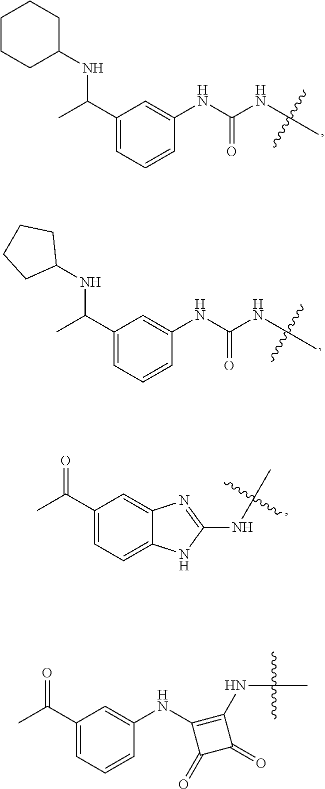 [SMILES: *NC(=O)Nc1cccc(C(C)NC2CCCCC2)c1 ; *NC(=O)Nc1cccc(C(C)NC2CCCC2)c1 ; *Nc1nc2cc(C(C)=O)ccc2[nH]1 ; *Nc1c(Nc2cccc(C(C)=O)c2)c(=O)c1=O]

All compounds disclosed in present invention having Structure I are depicted in the table I:

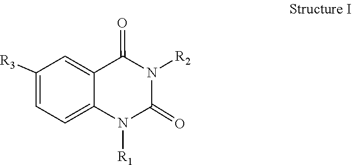

TABLE 1
Structure of the compounds disclosed

| SI. | Compound | | |
|---|---|---|---|
| No. | No. | Structure | IUPAC Name |
| 1 | 5 | 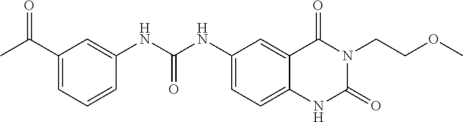 [SMILES: COCCn1c(=O)[nH]c2ccc(NC(=O)Nc3cccc(C(C)=O)c3)cc2c1=O] | 1-(3-acetylphenyl)-3-(3-(2- methoxyethyl)-2,4-dioxo- 1,2,3,4-tetrahydroquinazolin-6- yl)urea |
| 2 | 8 | 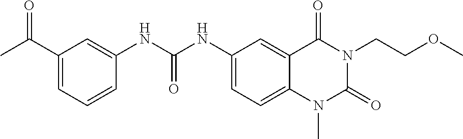 [SMILES: COCCn1c(=O)c2cc(NC(=O)Nc3cccc(C(C)=O)c3)ccc2n(C)c1=O] | 1-(3-acetylphenyl)-3-(3-(2- methoxyethyl)-1-methyl-2,4- dioxo-1,2,3,4- tetrahydroquinazolin-6-yl)urea |
| 3 | 11 | 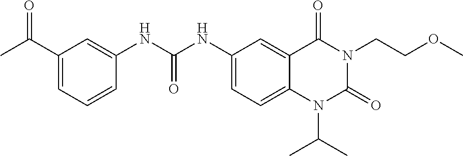 [SMILES: COCCn1c(=O)c2cc(NC(=O)Nc3cccc(C(C)=O)c3)ccc2n(C(C)C)c1=O] | 1-(3-acetylphenyl)-3-(1- isopropyl-3-(2-methoxyethyl)- 2,4-dioxo-1,2,3,4- tetrahydroquinazolin-6-yl)urea |
| 4 | 14 | 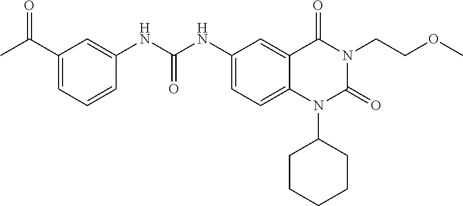 [SMILES: COCCn1c(=O)c2cc(NC(=O)Nc3cccc(C(C)=O)c3)ccc2n(C2CCCCC2)c1=O] | 1-(3-acetylphenyl)-3-(1- cyclohexyl-3-(2-methoxyethyl)- 2,4-dioxo-1,2,3,4- tetrahydroquinazolin-6-yl)urea |
| 5 | 17 | 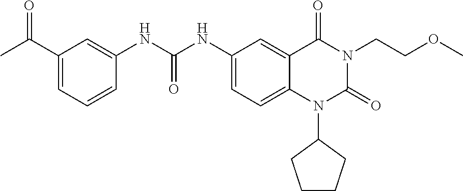 [SMILES: COCCn1c(=O)c2cc(NC(=O)Nc3cccc(C(C)=O)c3)ccc2n(C2CCCC2)c1=O] | 1-(3-acetylphenyl)-3-(1- cyclopentyl-3-(2-methoxyethyl)- 2,4-dioxo-1,2,3,4- tetrahydroquinazolin-6-yl)urea |
| 6 | 20 | 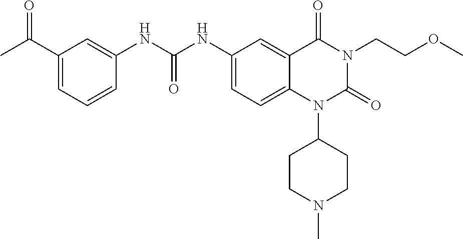 [SMILES: COCCn1c(=O)c2cc(NC(=O)Nc3cccc(C(C)=O)c3)ccc2n(C2CCN(C)CC2)c1=O] | 1-(3-acetylphenyl)-3-(3-(2- methoxyethyl)-1-(1- methylpiperidin-4-yl)-2,4- dioxo-1,2,3,4- tetrahydroquinazolin-6-yl)urea |
| 7 | 23 | 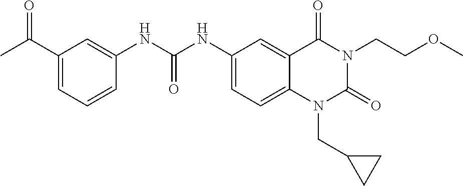 [SMILES: COCCn1c(=O)c2cc(NC(=O)Nc3cccc(C(C)=O)c3)ccc2n(CC2CC2)c1=O] | 1-(3-acetylphenyl)-3-(1- (cyclopropylmethyl)-3-(2- methoxyethyl)-2,4-dioxo- 1,2,3,4-tetrahydroquinazolin-6- yl)urea |
| 8 | 26 | 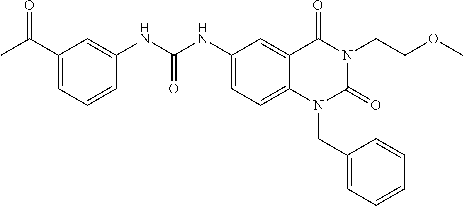 [SMILES: COCCn1c(=O)c2cc(NC(=O)Nc3cccc(C(C)=O)c3)ccc2n(Cc2ccccc2)c1=O] | 1-(3-acetylphenyl)-3-(1-benzyl- 3-(2-methoxyethyl)-2,4-dioxo- 1,2,3,4-tetrahydroquinazolin-6- yl)urea |
| 9 | 29 | 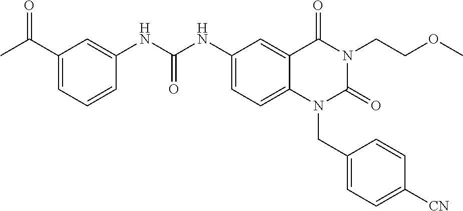 [SMILES: COCCn1c(=O)c2cc(NC(=O)Nc3cccc(C(C)=O)c3)ccc2n(Cc2ccc(C#N)cc2)c1=O] | 1-(3-acetylphenyl)-3-(1-(4- cyanobenzyl)-3-(2- methoxyethyl)-2,4-dioxo- 1,2,3,4-tetrahydroquinazolin-6- yl)urea |
| 10 | 32 | 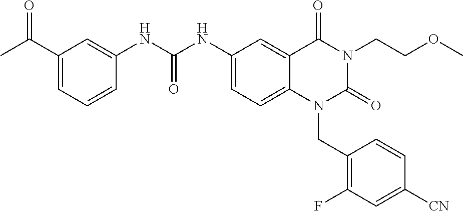 [SMILES: COCCn1c(=O)c2cc(NC(=O)Nc3cccc(C(C)=O)c3)ccc2n(Cc2ccc(C#N)cc2F)c1=O] | 1-(3-acetylphenyl)-3-(1-(4- cyano-2-fluorobenzyl)-3-(2- methoxyethyl)-2,4-dioxo- 1,2,3,4-tetrahydroquinazolin-6- yl)urea |
| 11 | 35 | 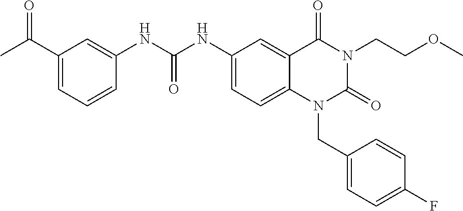 [SMILES: COCCn1c(=O)c2cc(NC(=O)Nc3cccc(C(C)=O)c3)ccc2n(Cc2ccc(F)cc2)c1=O] | 1-(3-acetylphenyl)-3-(1-(4- fluorobenzyl)-3-(2- methoxyethyl)-2,4-dioxo- 1,2,3,4-tetrahydroquinazolin-6- yl)urea |
| 12 | 38 | 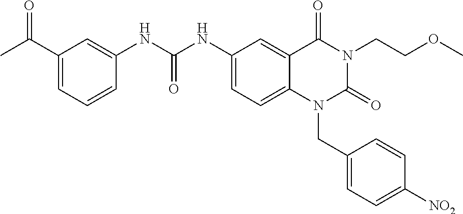 [SMILES: COCCn1c(=O)c2cc(NC(=O)Nc3cccc(C(C)=O)c3)ccc2n(Cc2ccc([N+](=O)[O-])cc2)c1=O] | 1-(3-acetylphenyl)-3-(3-(2- methoxyethyl)-1-(4- nitrobenzyl)-2,4-dioxo-1,2,3,4- tetrahydroquinazolin-6-yl)urea |
| 13 | 41 | 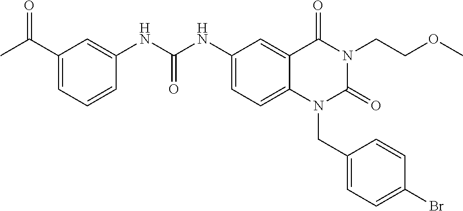 [SMILES: COCCn1c(=O)c2cc(NC(=O)Nc3cccc(C(C)=O)c3)ccc2n(Cc2ccc(Br)cc2)c1=O] | 1-(3-acetylphenyl)-3-(1-(4- bromobenzyl)-3-(2- methoxyethyl)-2,4-dioxo- 1,2,3,4-tetrahydroquinazolin-6- yl)urea |
| 14 | 44 | 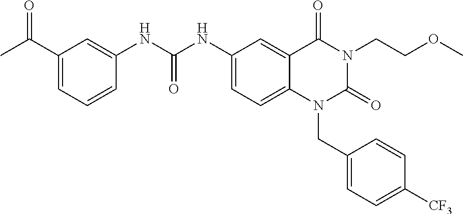 [SMILES: COCCn1c(=O)c2cc(NC(=O)Nc3cccc(C(C)=O)c3)ccc2n(Cc2ccc(C(F)(F)F)cc2)c1=O] | 1-(3-acetylphenyl)-3-(3-(2- methoxyethyl)-2,4-dioxo-1-(4- (trifluoromethyl)benzyl)- 1,2,3,4-tetrahydroquinazolin-6- yl)urea |
| 15 | 47 | 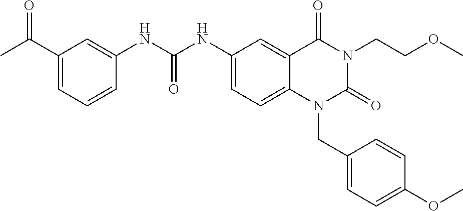 [SMILES: COCCn1c(=O)c2cc(NC(=O)Nc3cccc(C(C)=O)c3)ccc2n(Cc2ccc(OC)cc2)c1=O] | 1-(3-acetylphenyl)-3-(1-(4- methoxybenzyl)-3-(2- methoxyethyl)-2,4-dioxo- 1,2,3,4-tetrahydroquinazolin-6- yl)urea |
| 16 | 50 | 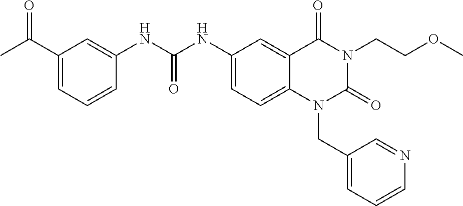 [SMILES: COCCn1c(=O)c2cc(NC(=O)Nc3cccc(C(C)=O)c3)ccc2n(Cc2cccnc2)c1=O] | 1-(3-acetylphenyl)-3-(3-(2- methoxyethyl)-2,4-dioxo-1- (pyridin-3-ylmethyl)-1,2,3,4- tetrahydroquinazolin-6-yl)urea |
| 17 | 53 | 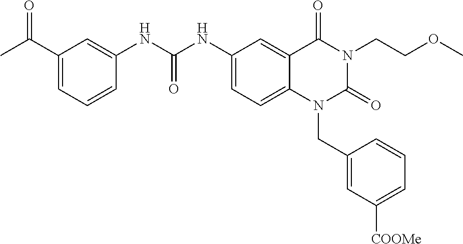 [SMILES: COCCn1c(=O)c2cc(NC(=O)Nc3cccc(C(C)=O)c3)ccc2n(Cc2cccc(C(=O)OC)c2)c1=O] | methyl 3-((6-(3-(3- acetylphenyl)ureido)-3-(2- methoxyethyl)-2,4-dioxo-3,4- dihydroquinazolin-1(2H)- yl)methyl)benzoate |
| 18 | 56 | 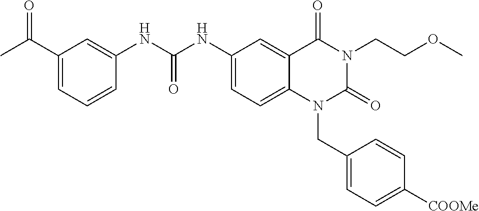 [SMILES: COCCn1c(=O)c2cc(NC(=O)Nc3cccc(C(C)=O)c3)ccc2n(Cc2ccc(C(=O)OC)cc2)c1=O] | methyl 4-((6-(3-(3- acetylphenyl)ureido)-3-(2- methoxyethyl)-2,4-dioxo-3,4- dihydroquinazolin-1(2H)- yl)methyl)benzoate |
| 19 | 59 | 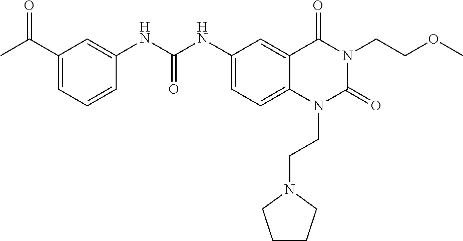 [SMILES: COCCn1c(=O)c2cc(NC(=O)Nc3cccc(C(C)=O)c3)ccc2n(CCN2CCCC2)c1=O] | 1-(3-acetylphenyl)-3-(3-(2- methoxyethyl)-2,4-dioxo-1-(2- (pyrrolidin-1-yl)ethyl)-1,2,3,4- tetrahydroquinazolin-6-yl)urea |
| 20 | 62 | 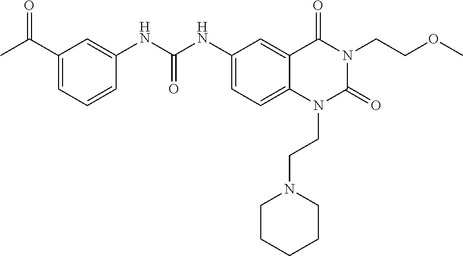 [SMILES: COCCn1c(=O)c2cc(NC(=O)Nc3cccc(C(C)=O)c3)ccc2n(CCN2CCCCC2)c1=O] | 1-(3-acetylphenyl)-3-(3-(2- methoxyethyl)-2,4-dioxo-1-(2- (piperidin-1-yl)ethyl)-1,2,3,4- tetrahydroquinazolin-6-yl)urea |
| 21 | 65 | 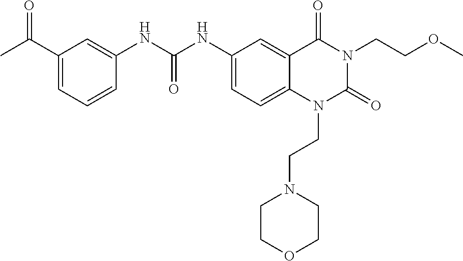 [SMILES: COCCn1c(=O)c2cc(NC(=O)Nc3cccc(C(C)=O)c3)ccc2n(CCN2CCOCC2)c1=O] | 1-(3-acetylphenyl)-3-(3-(2- methoxyethyl)-1-(2- morpholinoethyl)-2,4-dioxo- 1,2,3,4-tetrahydroquinazolin-6- yl)urea |
| 22 | 68 | 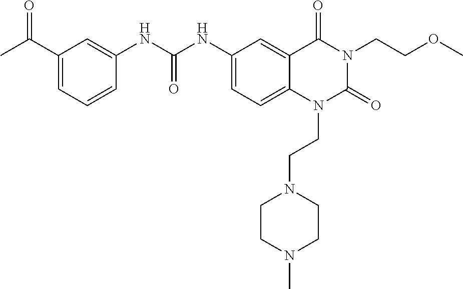 [SMILES: COCCn1c(=O)c2cc(NC(=O)Nc3cccc(C(C)=O)c3)ccc2n(CCN2CCN(C)CC2)c1=O] | 1-(3-acetylphenyl)-3-(3-(2- methoxyethyl)-1-(2-(4- methylpiperazin-1-yl)ethyl)-2,4- dioxo-1,2,3,4- tetrahydroquinazolin-6-yl)urea |
| 23 | 72 | 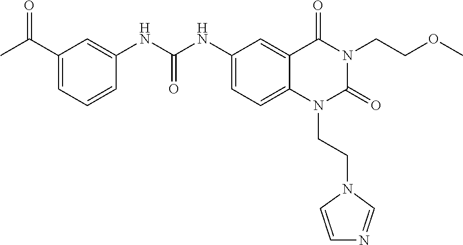 [SMILES: COCCn1c(=O)c2cc(NC(=O)Nc3cccc(C(C)=O)c3)ccc2n(CCn2ccnc2)c1=O] | 1-(1-(2-(1H-imidazol-1- yl)ethyl)-3-(2-methoxyethyl)- 2,4-dioxo-1,2,3,4- tetrahydroquinazolin-6-yl)-3-(3- acetylphenyl)urea |
| 24 | 75 | 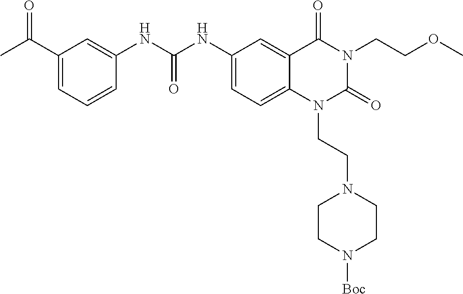 [SMILES: COCCn1c(=O)c2cc(NC(=O)Nc3cccc(C(C)=O)c3)ccc2n(CCN2CCN(C(=O)OC(C)(C)C)CC2)c1=O] | tert-butyl 4-(2-(6-(3-(3- acetylphenyl)ureido)-3-(2- methoxyethyl)-2,4-dioxo-3,4- dihydroquinazolin-1(2H)- yl)ethyl)piperazine-1- carboxylate |
| 25 | 76 | 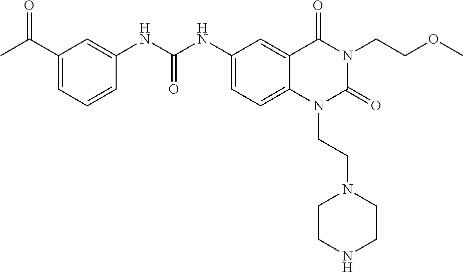 [SMILES: COCCn1c(=O)c2cc(NC(=O)Nc3cccc(C(C)=O)c3)ccc2n(CCN2CCNCC2)c1=O] | 1-(3-acetylphenyl)-3-(3-(2- methoxyethyl)-2,4-dioxo-1-(2- (piperazin-1-yl)ethyl)-1,2,3,4- tetrahydroquinazolin-6-yl)urea |
| 26 | 79 | 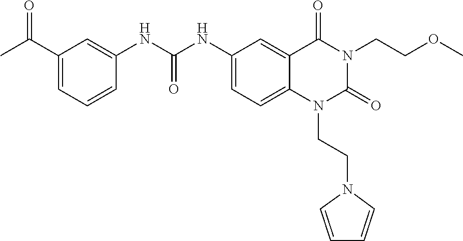 [SMILES: COCCn1c(=O)c2cc(NC(=O)Nc3cccc(C(C)=O)c3)ccc2n(CCn2cccc2)c1=O] | 1-(1-(2-(1H-pyrrol-1-yl)ethyl)- 3-(2-methoxyethyl)-2,4-dioxo- 1,2,3,4-tetrahydroquinazolin-6- yl)-3-(3-acetylphenyl)urea |
| 27 | 82 | 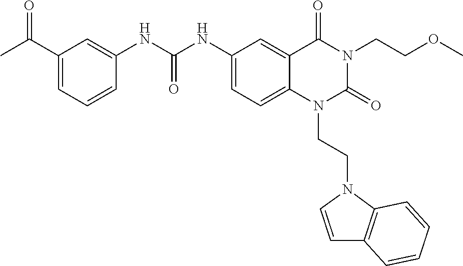 [SMILES: COCCn1c(=O)c2cc(NC(=O)Nc3cccc(C(C)=O)c3)ccc2n(CCn2ccc3ccccc32)c1=O] | 1-(1-(2-(1H-indol-1-yl)ethyl)-3- (2-methoxyethyl)-2,4-dioxo- 1,2,3,4-tetrahydroquinazolin-6- yl)-3-(3-acetylphenyl)urea |
| 28 | 85 | 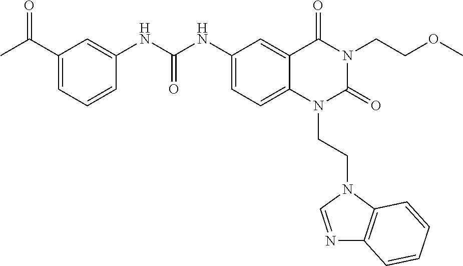 [SMILES: COCCn1c(=O)c2cc(NC(=O)Nc3cccc(C(C)=O)c3)ccc2n(CCn2cnc3ccccc32)c1=O] | 1-(1-(2-(1H-benzo[d]imidazol- 1-yl)ethyl)-3-(2-methoxyethyl)- 2,4-dioxo-1,2,3,4- tetrahydroquinazolin-6-yl)-3-(3- acetylphenyl)urea |
| 29 | 88 | 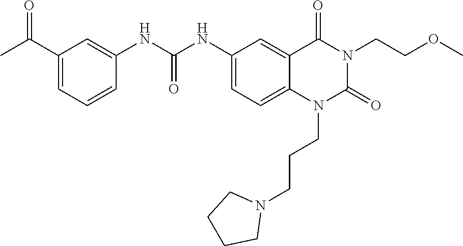 [SMILES: COCCn1c(=O)c2cc(NC(=O)Nc3cccc(C(C)=O)c3)ccc2n(CCCN2CCCC2)c1=O] | 1-(3-acetylphenyl)-3-(3-(2- methoxyethyl)-2,4-dioxo-1-(3- (pyrrolidin-1-yl)propyl)-1,2,3,4- tetrahydroquinazolin-6-yl)urea |
| 30 | 91 | 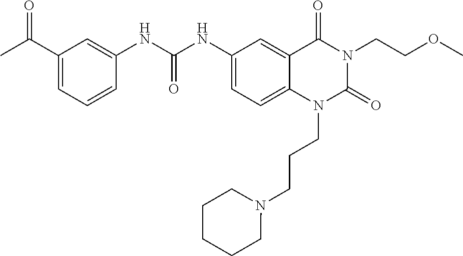 [SMILES: COCCn1c(=O)c2cc(NC(=O)Nc3cccc(C(C)=O)c3)ccc2n(CCCN2CCCCC2)c1=O] | 1-(3-acetylphenyl)-3-(3-(2- methoxyethyl)-2,4-dioxo-1-(3- (piperidin-1-yl)propyl)-1,2,3,4- tetrahydroquinazolin-6-yl)urea |
| 31 | 94 | 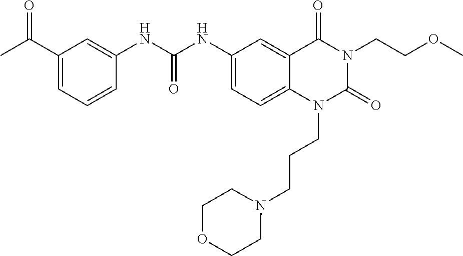 [SMILES: COCCn1c(=O)c2cc(NC(=O)Nc3cccc(C(C)=O)c3)ccc2n(CCCN2CCOCC2)c1=O] | 1-(3-acetylphenyl)-3-(3-(2- methoxyethyl)-1-(3- morpholinopropyl)-2,4-dioxo- 1,2,3,4-tetrahydroquinazolin-6- yl)urea |
| 32 | 97 | 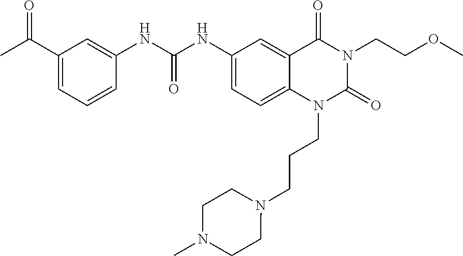 [SMILES: COCCn1c(=O)c2cc(NC(=O)Nc3cccc(C(C)=O)c3)ccc2n(CCCN2CCN(C)CC2)c1=O] | 1-(3-acetylphenyl)-3-(3-(2- methoxyethyl)-1-(3-(4- methylpiperazin-1-yl)propyl)- 2,4-dioxo-1,2,3,4- tetrahydroquinazolin-6-yl)urea |
| 33 | 102 | 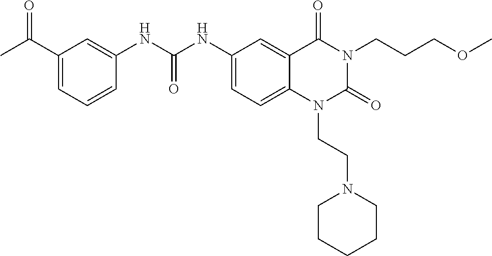 [SMILES: COCCCn1c(=O)c2cc(NC(=O)Nc3cccc(C(C)=O)c3)ccc2n(CCN2CCCCC2)c1=O] | 1-(3-acetylphenyl)-3-(3-(3- methoxypropyl)-2,4-dioxo-1-(2- (piperidin-1-yl)ethyl)-1,2,3,4- tetrahydroquinazolin-6-yl)urea |
| 34 | 107 | 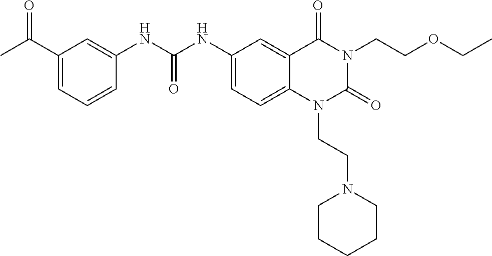 [SMILES: CCOCCn1c(=O)c2cc(NC(=O)Nc3cccc(C(C)=O)c3)ccc2n(CCN2CCCCC2)c1=O] | 1-(3-acetylphenyl)-3-(3-(2- ethoxyethyl)-2,4-dioxo-1-(2- (piperidin-1-yl)ethyl)-1,2,3,4- tetrahydroquinazolin-6-yl)urea |
| 35 | 112 | 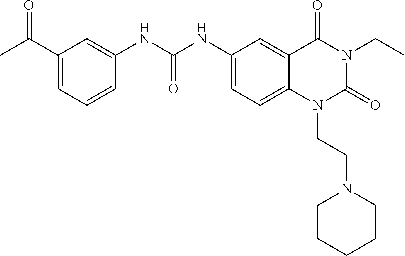 [SMILES: CCn1c(=O)c2cc(NC(=O)Nc3cccc(C(C)=O)c3)ccc2n(CCN2CCCCC2)c1=O] | 1-(3-acetylphenyl)-3-(3-ethyl- 2,4-dioxo-1-(2-(piperidin-1- yl)ethyl)-1,2,3,4- tetrahydroquinazolin-6-yl)urea |
| 36 | 117 | 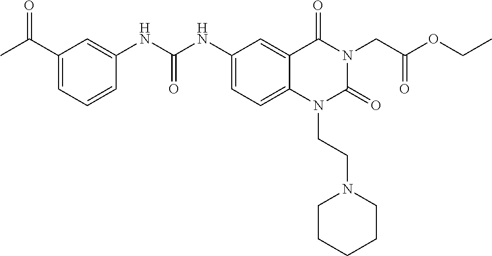 [SMILES: CCOC(=O)Cn1c(=O)c2cc(NC(=O)Nc3cccc(C(C)=O)c3)ccc2n(CCN2CCCCC2)c1=O] | ethyl 2-(6-(3-(3- acetylphenyl)ureido)-2,4-dioxo- 1-(2-(piperidin-1-yl)ethyl)-1,2- dihydroquinazolin-3(4H)- yl)acetate |
| 37 | 122 | 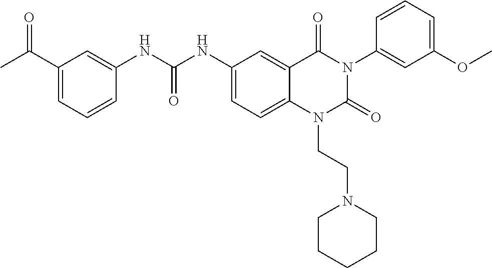 [SMILES: COc1cccc(-n2c(=O)c3cc(NC(=O)Nc4cccc(C(C)=O)c4)ccc3n(CCN3CCCCC3)c2=O)c1] | 1-(3-acetylphenyl)-3-(3-(3- methoxyphenyl)-2,4-dioxo-1-(2- (piperidin-1-yl)ethyl)-1,2,3,4- tetrahydroquinazolin-6-yl)urea |
| 38 | 127 | 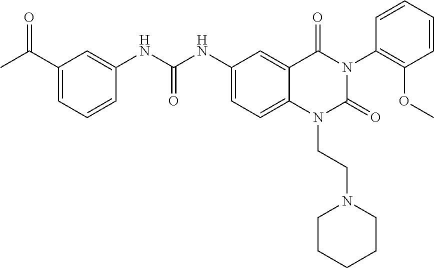 [SMILES: COc1ccccc1-n1c(=O)c2cc(NC(=O)Nc3cccc(C(C)=O)c3)ccc2n(CCN2CCCCC2)c1=O] | 1-(3-acetylphenyl)-3-(3-(2- methoxyphenyl)-2,4-dioxo-1-(2- (piperidin-1-yl)ethyl)-1,2,3,4- tetrahydroquinazolin-6-yl)urea |
| 39 | 132 | 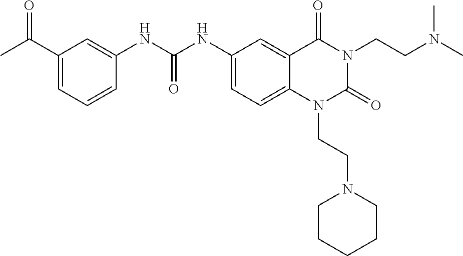 [SMILES: CC(=O)c1cccc(NC(=O)Nc2ccc3c(c2)c(=O)n(CCN(C)C)c(=O)n3CCN2CCCCC2)c1] | 1-(3-acetylphenyl)-3-(3-(2- (dimethylamino)ethyl)-2,4- dioxo-1-(2-(piperidin-1- yl)ethyl)-1,2,3,4- tetrahydroquinazolin-6-yl)urea |
| 40 | 137 | 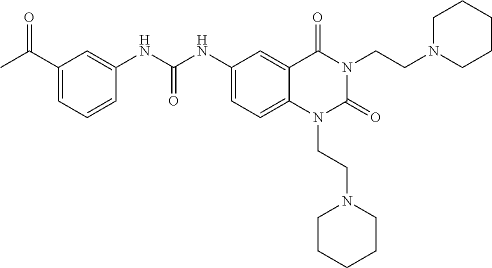 [SMILES: CC(=O)c1cccc(NC(=O)Nc2ccc3c(c2)c(=O)n(CCN2CCCCC2)c(=O)n3CCN2CCCCC2)c1] | 1-(3-acetylphenyl)-3-(2,4-dioxo- 1,3-bis(2-(piperidin-1-yl)ethyl)- 1,2,3,4-tetrahydroquinazolin-6- yl)urea |
| 41 | 142 | 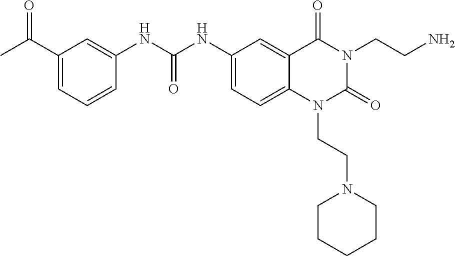 [SMILES: CC(=O)c1cccc(NC(=O)Nc2ccc3c(c2)c(=O)n(CCN)c(=O)n3CCN2CCCCC2)c1] | 1-(3-acetylphenyl)-3-(3-(2- aminoethyl)-2,4-dioxo-1-(2- (piperidin-1-yl)ethyl)-1,2,3,4- tetrahydroquinazolin-6-yl)urea |
| 42 | 147 | 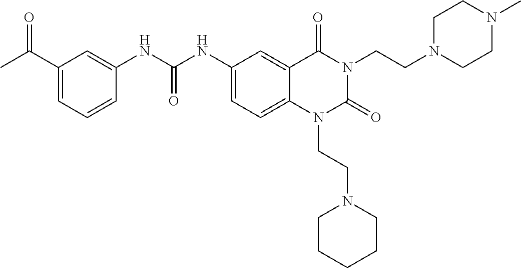 [SMILES: CC(=O)c1cccc(NC(=O)Nc2ccc3c(c2)c(=O)n(CCN2CCN(C)CC2)c(=O)n3CCN2CCCCC2)c1] | 1-(3-acetylphenyl)-3-(3-(2-(4- methylpiperazin-1-yl)ethyl)-2,4- dioxo-1-(2-(piperidin-1- yl)ethyl)-1,2,3,4- tetrahydroquinazolin-6-yl)urea |
| 43 | 152 | 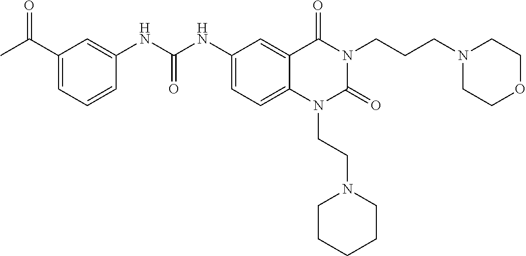 [SMILES: CC(=O)c1cccc(NC(=O)Nc2ccc3c(c2)c(=O)n(CCCN2CCOCC2)c(=O)n3CCN2CCCCC2)c1] | 1-(3-acetylphenyl)-3-(3-(3- morpholinopropyl)-2,4-dioxo-1- (2-(piperidin-1-yl)ethyl)-1,2,3,4- tetrahydroquinazolin-6-yl)urea |
| 44 | 157 | 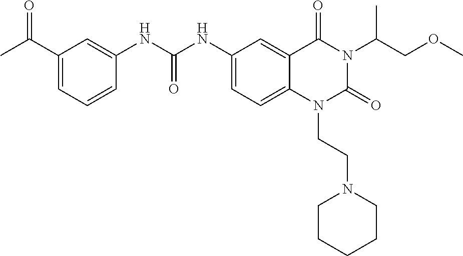 [SMILES: COCC(C)n1c(=O)c2cc(NC(=O)Nc3cccc(C(C)=O)c3)ccc2n(CCN2CCCCC2)c1=O] | 1-(3-acetylphenyl)-3-(3-(1- methoxypropan-2-yl)-2,4-dioxo- 1-(2-(piperidin-1-yl)ethyl)- 1,2,3,4-tetrahydroquinazolin-6- yl)urea |
| 45 | 162 | 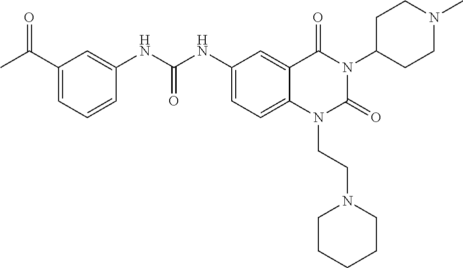 [SMILES: CC(=O)c1cccc(NC(=O)Nc2ccc3c(c2)c(=O)n(C2CCN(C)CC2)c(=O)n3CCN2CCCCC2)c1] | 1-(3-acetylphenyl)-3-(3-(1- methylpiperidin-4-yl)-2,4- dioxo-1-(2-(piperidin-1- yl)ethyl)-1,2,3,4- tetrahydroquinazolin-6-yl)urea |
| 46 | 167 | 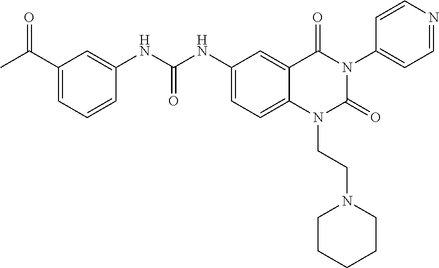 [SMILES: CC(=O)c1cccc(NC(=O)Nc2ccc3c(c2)c(=O)n(-c2ccncc2)c(=O)n3CCN2CCCCC2)c1] | 1-(3-acetylphenyl)-3-(2,4-dioxo- 1-(2-(piperidin-1-yl)ethyl)-3- (pyridin-4-yl)-1,2,3,4- tetrahydroquinazolin-6-yl)urea |
| 47 | 172 | 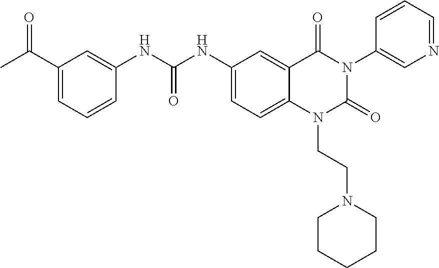 [SMILES: CC(=O)c1cccc(NC(=O)Nc2ccc3c(c2)c(=O)n(-c2cccnc2)c(=O)n3CCN2CCCCC2)c1] | 1-(3-acetylphenyl)-3-(2,4-dioxo- 1-(2-(piperidin-1-yl)ethyl)-3- (pyridin-3-yl)-1,2,3,4- tetrahydroquinazolin-6-yl)urea |
| 48 | 177 | 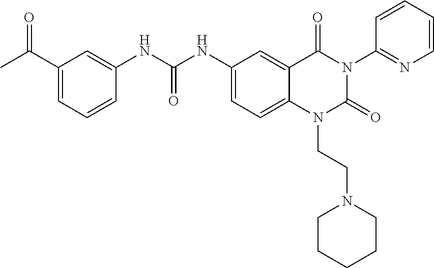 [SMILES: CC(=O)c1cccc(NC(=O)Nc2ccc3c(c2)c(=O)n(-c2ccccn2)c(=O)n3CCN2CCCCC2)c1] | 1-(3-acetylphenyl)-3-(2,4-dioxo- 1-(2-(piperidin-1-yl)ethyl)-3- (pyridin-2-yl)-1,2,3,4- tetrahydroquinazolin-6-yl)urea |
| 49 | 178 | 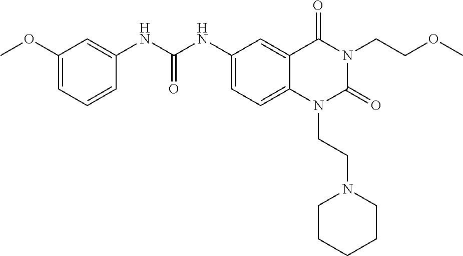 | 1-(3-(2-methoxyethyl)-2,4- dioxo-1-(2-(piperidin-1- yl)ethyl)-1,2,3,4- tetrahydroquinazolin-6-yl)-3-(3- methoxyphenyl)urea |
| 50 | 179 | 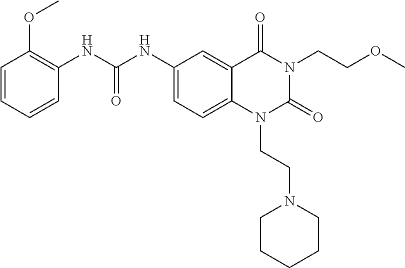 [SMILES: COCCn1c(=O)c2cc(NC(=O)Nc3ccccc3OC)ccc2n(CCN2CCCCC2)c1=O] | 1-(3-(2-methoxyethyl)-2,4- dioxo-1-(2-(piperidin-1- yl)ethyl)-1,2,3,4- tetrahydroquinazolin-6-yl)-3-(2- methoxyphenyl)urea |
| 51 | 180 | 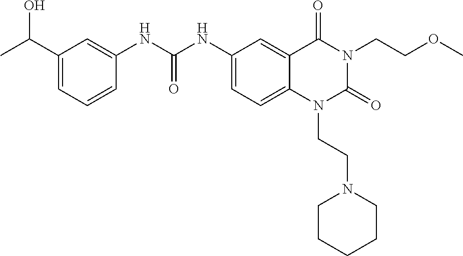 [SMILES: COCCn1c(=O)c2cc(NC(=O)Nc3cccc(C(C)O)c3)ccc2n(CCN2CCCCC2)c1=O] | 1-(3-(1-hydroxyethyl)phenyl)-3- (3-(2-methoxyethyl)-2,4-dioxo- 1-(2-(piperidin-1-yl)ethyl)- 1,2,3,4-tetrahydroquinazolin-6- yl)urea |
| 52 | 181 | 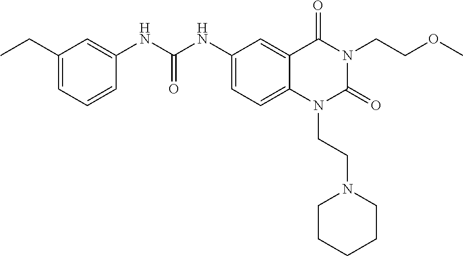 [SMILES: CCc1cccc(NC(=O)Nc2ccc3c(c2)c(=O)n(CCOC)c(=O)n3CCN2CCCCC2)c1] | 1-(3-ethylphenyl)-3-(3-(2- methoxyethyl)-2,4-dioxo-1-(2- (piperidin-1-yl)ethyl)-1,2,3,4- tetrahydroquinazolin-6-yl)urea |
| 53 | 182 | 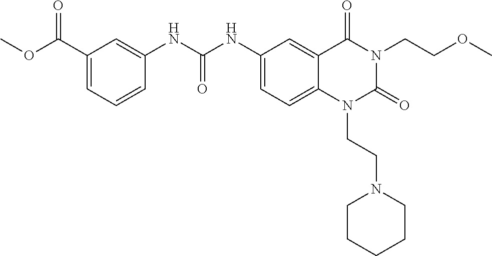 | methyl 3-(3-(3-(2- methoxyethyl)-2,4-dioxo-1-(2- (piperidin-1-yl)ethyl)-1,2,3,4- tetrahydroquinazolin-6- yl)ureido)benzoate |
| 54 | 183 | 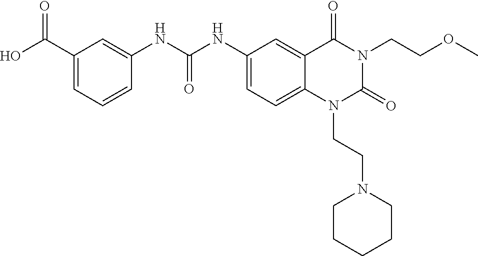 [SMILES: COCCn1c(=O)c2cc(NC(=O)Nc3cccc(C(=O)O)c3)ccc2n(CCN2CCCCC2)c1=O] | 3-(3-(3-(2-methoxyethyl)-2,4- dioxo-1-(2-(piperidin-1- yl)ethyl)-1,2,3,4- tetrahydroquinazolin-6- yl)ureido)benzoic acid |
| 55 | 184 | 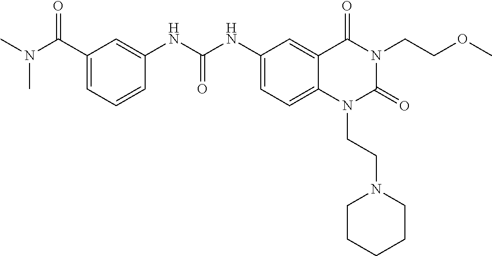 | 3-(3-(3-(2-methoxyethyl)-2,4- dioxo-1-(2-(piperidin-1- yl)ethyl)-1,2,3,4- tetrahydroquinazolin-6- yl)ureido)-N,N- dimethylbenzamide |
| 56 | 185 | 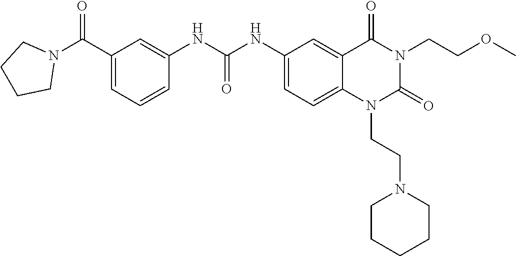 [SMILES: COCCn1c(=O)c2cc(NC(=O)Nc3cccc(C(=O)N4CCCC4)c3)ccc2n(CCN2CCCCC2)c1=O] | 1-(3-(2-methoxyethyl)-2,4- dioxo-1-(2-(piperidin-1- yl)ethyl)-1,2,3,4- tetrahydroquinazolin-6-yl)-3-(3- (pyrrolidine-1- carbonyl)phenyl)urea |
| 57 | 186 | 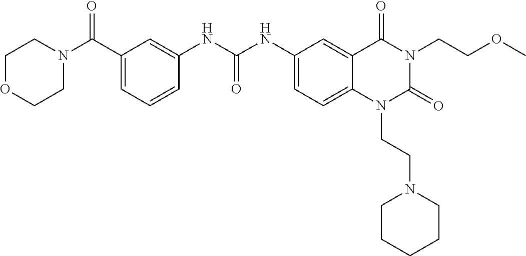 [SMILES: COCCn1c(=O)c2cc(NC(=O)Nc3cccc(C(=O)N4CCOCC4)c3)ccc2n(CCN2CCCCC2)c1=O] | 1-(3-(2-methoxyethyl)-2,4- dioxo-1-(2-(piperidin-1- yl)ethyl)-1,2,3,4- tetrahydroquinazolin-6-yl)-3-(3- (morpholine-4- carbonyl)phenyl)urea |
| 58 | 187 | 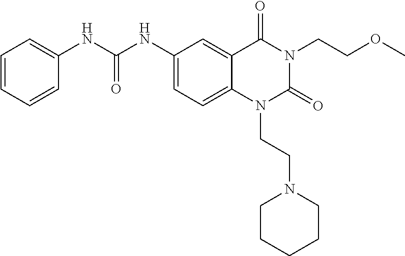 [SMILES: COCCn1c(=O)c2cc(NC(=O)Nc3ccccc3)ccc2n(CCN2CCCCC2)c1=O] | 1-(3-(2-methoxyethyl)-2,4- dioxo-1-(2-(piperidin-1- yl)ethyl)-1,2,3,4- tetrahydroquinazolin-6-yl)-3- phenylurea |
| 59 | 188 | 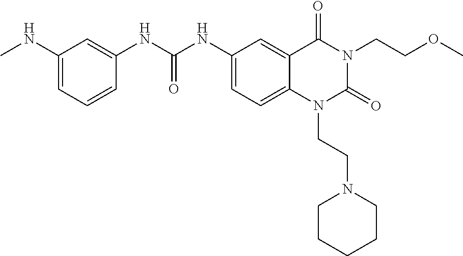 | 1-(3-(2-methoxyethyl)-2,4- dioxo-1-(2-(piperidin-1- yl)ethyl)-1,2,3,4- tetrahydroquinazolin-6-yl)-3-(3- (methylamino)phenyl)urea |
| 60 | 189 | 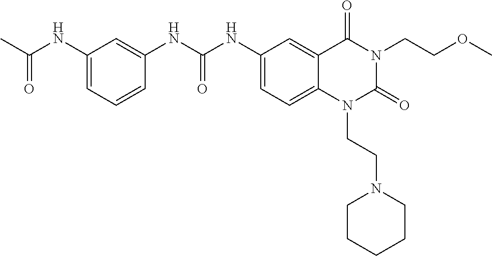 [SMILES: COCCn1c(=O)c2cc(NC(=O)Nc3cccc(NC(C)=O)c3)ccc2n(CCN2CCCCC2)c1=O] | N-(3-(3-(3-(2-methoxyethyl)- 2,4-dioxo-1-(2-(piperidin-1- yl)ethyl)-1,2,3,4- tetrahydroquinazolin-6- yl)ureido)phenyl)acetamide |
| 61 | 190 | 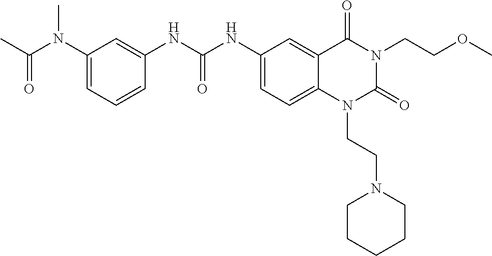 [SMILES: COCCn1c(=O)c2cc(NC(=O)Nc3cccc(N(C)C(C)=O)c3)ccc2n(CCN2CCCCC2)c1=O] | N-(3-(3-(3-(2-methoxyethyl)- 2,4-dioxo-1-(2-(piperidin-1- yl)ethyl)-1,2,3,4- tetrahydroquinazolin-6- yl)ureido)phenyl)-N- methylacetamide |
| 62 | 191 | 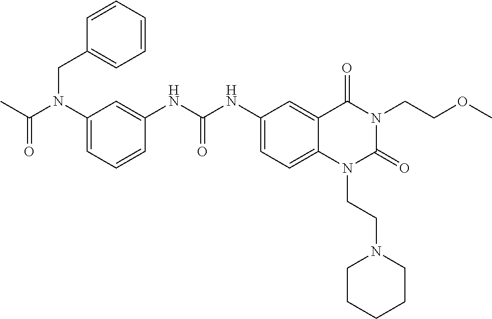 [SMILES: COCCn1c(=O)c2cc(NC(=O)Nc3cccc(N(Cc4ccccc4)C(C)=O)c3)ccc2n(CCN2CCCCC2)c1=O] | N-benzyl-N-(3-(3-(3-(2- methoxyethyl)-2,4-dioxo-1-(2- (piperidin-1-yl)ethyl)-1,2,3,4- tetrahydroquinazolin-6- yl)ureido)phenyl)acetamide |
| 63 | 192 | 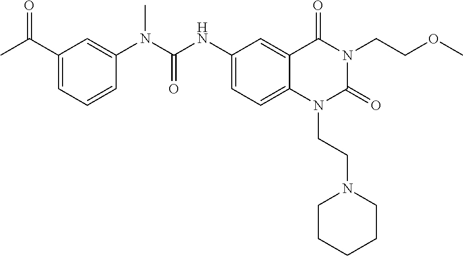 [SMILES: COCCn1c(=O)c2cc(NC(=O)N(C)c3cccc(C(C)=O)c3)ccc2n(CCN2CCCCC2)c1=O] | 1-(3-acetylphenyl)-3-(3-(2- methoxyethyl)-2,4-dioxo-1-(2- (piperidin-1-yl)ethyl)-1,2,3,4- tetrahydroquinazolin-6-yl)-1- methylurea |
| 64 | 193 | 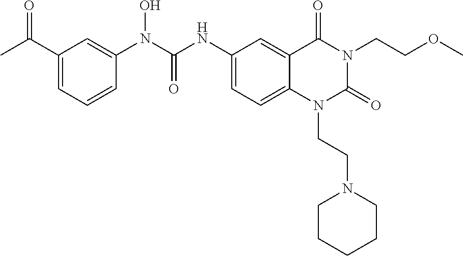 [SMILES: COCCn1c(=O)c2cc(NC(=O)N(O)c3cccc(C(C)=O)c3)ccc2n(CCN2CCCCC2)c1=O] | 1-(3-acetylphenyl)-1-hydroxy-3- (3-(2-methoxyethyl)-2,4-dioxo- 1-(2-(piperidin-1-yl)ethyl)- 1,2,3,4-tetrahydroquinazolin-6- yl)urea |
| 65 | 194 | 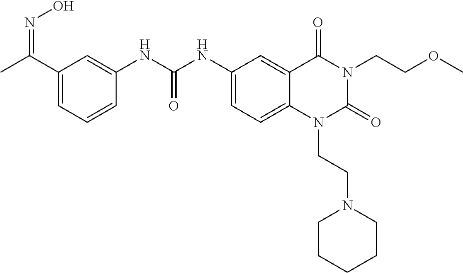 [SMILES: COCCn1c(=O)c2cc(NC(=O)Nc3cccc(/C(C)=N\O)c3)ccc2n(CCN2CCCCC2)c1=O] | (Z)-1-(3-(1- (hydroxyimino)ethyl)phenyl)-3- (3-(2-methoxyethyl)-2,4-dioxo- 1-(2-(piperidin-1-yl)ethyl)- 1,2,3,4-tetrahydroquinazolin-6- yl)urea |
| 66 | 195 | 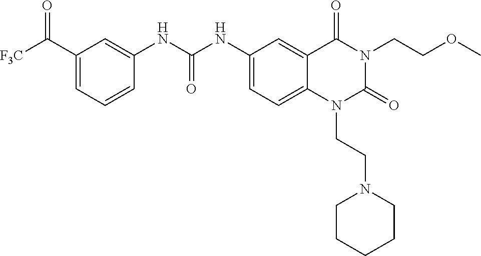 [SMILES: COCCn1c(=O)c2cc(NC(=O)Nc3cccc(C(=O)C(F)(F)F)c3)ccc2n(CCN2CCCCC2)c1=O] | 1-(3-(2-methoxyethyl)-2,4- dioxo-1-(2-(piperidin-1- yl)ethyl)-1,2,3,4- tetrahydroquinazolin-6-yl)-3-(3- (2,2,2- trifluoroacetyl)phenyl)urea |
| 67 | 196 | 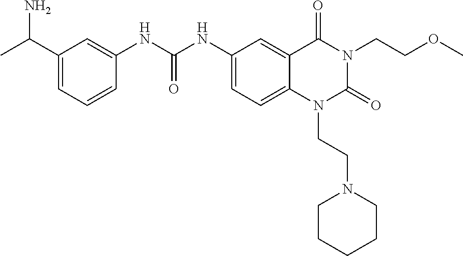 [SMILES: COCCn1c(=O)c2cc(NC(=O)Nc3cccc(C(C)N)c3)ccc2n(CCN2CCCCC2)c1=O] | 1-(3-(1-aminoethyl)phenyl)-3- (3-(2-methoxyethyl)-2,4-dioxo- 1-(2-(piperidin-1-yl)ethyl)- 1,2,3,4-tetrahydroquinazolin-6- yl)urea |
| 68 | 197 | 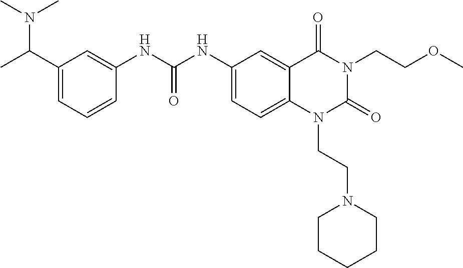 [SMILES: COCCn1c(=O)c2cc(NC(=O)Nc3cccc(C(C)N(C)C)c3)ccc2n(CCN2CCCCC2)c1=O] | 1-(3-(1- (dimethylamino)ethyl)phenyl)- 3-(3-(2-methoxyethyl)-2,4- dioxo-1-(2-(piperidin-1- yl)ethyl)-1,2,3,4- tetrahydroquinazolin-6-yl)urea |
| 69 | 199 | 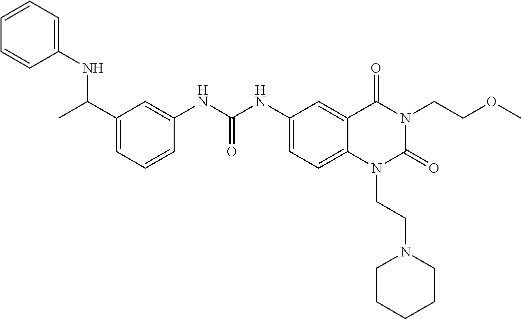 [SMILES: COCCn1c(=O)c2cc(NC(=O)Nc3cccc(C(C)Nc4ccccc4)c3)ccc2n(CCN2CCCCC2)c1=O] | 1-(3-(2-methoxyethyl)-2,4- dioxo-1-(2-(piperidin-1- yl)ethyl)-1,2,3,4- tetrahydroquinazolin-6-yl)-3-(3- (1- (phenylamino)ethyl)phenyl)urea |
| 70 | 201 | 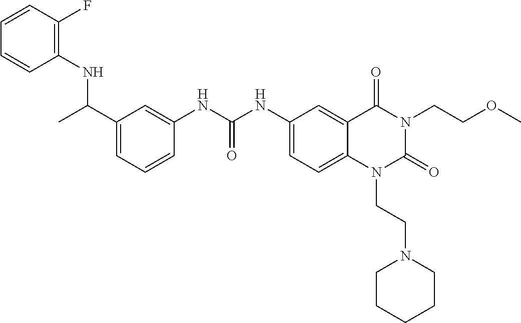 [SMILES: COCCn1c(=O)c2cc(NC(=O)Nc3cccc(C(C)Nc4ccccc4F)c3)ccc2n(CCN2CCCCC2)c1=O] | 1-(3-(1-((2- fluorophenyl)amino)ethyl)phenyl)- 3-(3-(2-methoxyethyl)-2,4- dioxo-1-(2-(piperidin-1- yl)ethyl)-1,2,3,4- tetrahydroquinazolin-6-yl)urea |
| 71 | 203 | 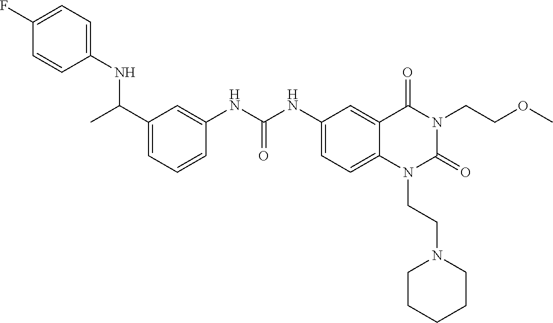 [SMILES: COCCn1c(=O)c2cc(NC(=O)Nc3cccc(C(C)Nc4ccc(F)cc4)c3)ccc2n(CCN2CCCCC2)c1=O] | 1-(3-(1-((4- fluorophenyl)amino)ethyl)phenyl)- 3-(3-(2-methoxyethyl)-2,4- dioxo-1-(2-(piperidin-1- yl)ethyl)-1,2,3,4- tetrahydroquinazolin-6-yl)urea |
| 72 | 205 | 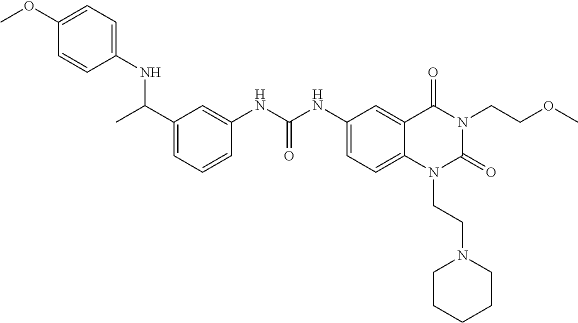 | 1-(3-(2-methoxyethyl)-2,4- dioxo-1-(2-(piperidin-1- yl)ethyl)-1,2,3,4- tetrahydroquinazolin-6-yl)-3-(3- (1-((4- methoxyphenyl)amino)ethyl) phenyl)urea |
| 73 | 207 | 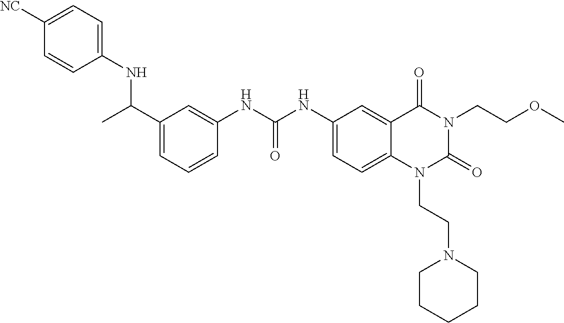 [SMILES: COCCn1c(=O)c2cc(NC(=O)Nc3cccc(C(C)Nc4ccc(C#N)cc4)c3)ccc2n(CCN2CCCCC2)c1=O] | 1-(3-(1-((4- cyanophenyl)amino)ethyl)phenyl)- 3-(3-(2-methoxyethyl)-2,4- dioxo-1-(2-(piperidin-1- yl)ethyl)-1,2,3,4- tetrahydroquinazolin-6-yl)urea |
| 74 | 209 | 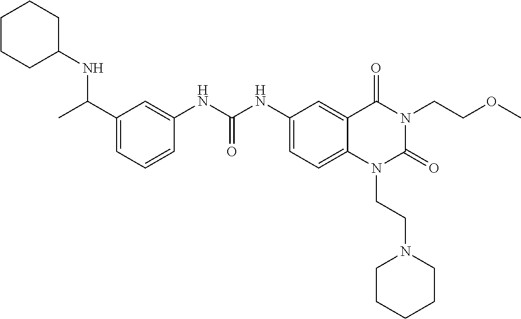 [SMILES: COCCn1c(=O)c2cc(NC(=O)Nc3cccc(C(C)NC4CCCCC4)c3)ccc2n(CCN2CCCCC2)c1=O] | 1-(3-(1- (cyclohexylamino)ethyl)phenyl)- -3-(3-(2-methoxyethyl)-2,4- dioxo-1-(2-(piperidin-1- yl)ethyl)-1,2,3,4- tetrahydroquinazolin-6-yl)urea (209) |
| 75 | 211 | 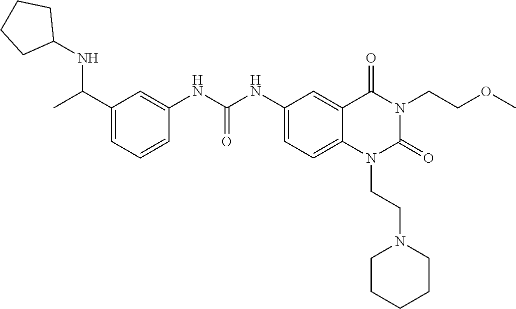 [SMILES: COCCn1c(=O)c2cc(NC(=O)Nc3cccc(C(C)NC4CCCC4)c3)ccc2n(CCN2CCCCC2)c1=O] | 1-(3-(1- (cyclopentylamino)ethyl)phenyl )-3-(3-(2-methoxyethyl)-2,4- dioxo-1-(2-(piperidin-1- yl)ethyl)-1,2,3,4- tetrahydroquinazolin-6-yl)urea (211) |
| 76 | 216 | 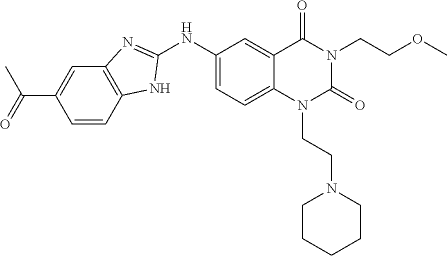 [SMILES: COCCn1c(=O)c2cc(Nc3nc4cc(C(C)=O)ccc4[nH]3)ccc2n(CCN2CCCCC2)c1=O] | 6-((5-acetyl-1H- benzo[d]imidazol-2-yl)amino)- 3-(2-methoxyethyl)-1-(2- (piperidin-1- yl)ethyl)quinazoline- 2,4(1H,3H)-dione |
| 77 | 217 | 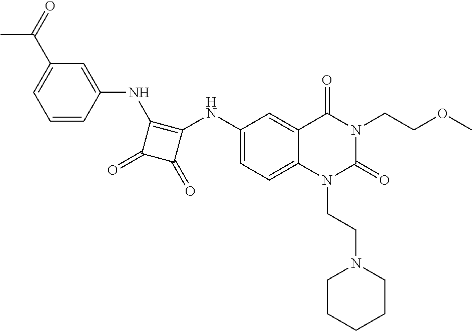 [SMILES: COCCn1c(=O)c2cc(Nc3c(Nc4cccc(C(C)=O)c4)c(=O)c3=O)ccc2n(CCN2CCCCC2)c1=O] | 6-((2-((3-acetylphenyl)amino)- 3,4-dioxocyclobut-1-en-1- yl)amino)-3-(2-methoxyethyl)- 1-(2-(piperidin-1- yl)ethyl)quinazoline- 2,4(1H,3H)-dione |
| 78 | 222 | 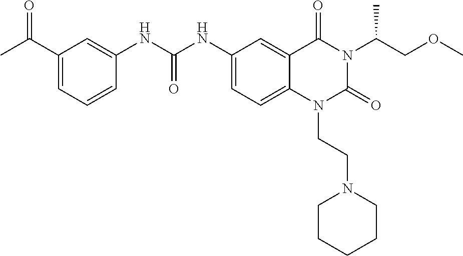 [SMILES: COC[C@@H](C)n1c(=O)c2cc(NC(=O)Nc3cccc(C(C)=O)c3)ccc2n(CCN2CCCCC2)c1=O] | (R)-1-(3-acetylphenyl)-3-(3-(1- methoxypropan-2-yl)-2,4-dioxo- 1 -(2-(piperidin-1-yl)ethyl)- 1,2,3,4-tetrahydroquinazolin-6- yl)urea |
| 79 | 227 | 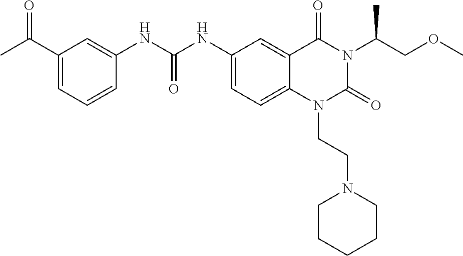 [SMILES: COC[C@H](C)n1c(=O)c2cc(NC(=O)Nc3cccc(C(C)=O)c3)ccc2n(CCN2CCCCC2)c1=O] | (S)-1-(3-acetylphenyl)-3-(3-(1- methoxypropan-2-yl)-2,4-dioxo- 1-(2-(piperidin-1-yl)ethyl)- 1,2,3,4-tetrahydroquinazolin-6- yl)urea |
| 80 | 228 | 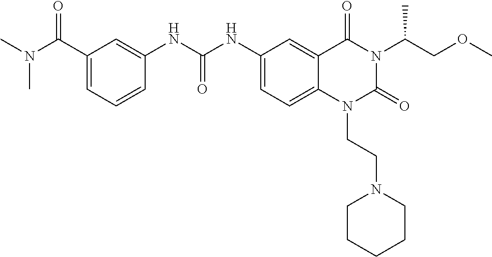 | (R)-3-(3-(3-(1-methoxypropan- 2-yl)-2,4-dioxo-1-(2-(piperidin- 1-yl)ethyl)-1,2,3,4- tetrahydroquinazolin-6- yl)ureido)-N,N- dimethylbenzamide |
| 81 | 229 | 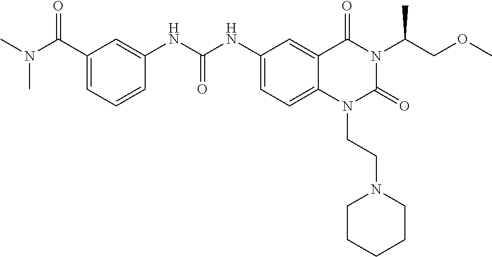 | (S)-3-(3-(3-(1-methoxypropan- 2-yl)-2,4-dioxo-1-(2-(piperidin- 1-yl)ethyl)-1,2,3,4- tetrahydroquinazolin-6- yl)ureido)-N,N- dimethylbenzamide |
| 82 | 230 | 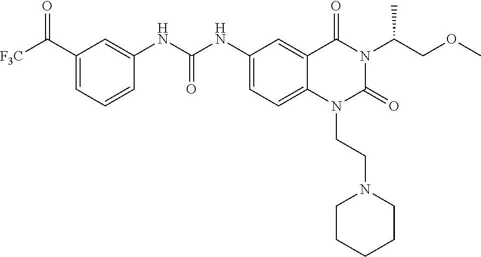 [SMILES: COC[C@@H](C)n1c(=O)c2cc(NC(=O)Nc3cccc(C(=O)C(F)(F)F)c3)ccc2n(CCN2CCCCC2)c1=O] | (R)-1-(3-(1-methoxypropan-2- yl)-2,4-dioxo-1-(2-(piperidin-1- yl)ethyl)-1,2,3,4- tetrahydroquinazolin-6-yl)-3-(3- (2,2,2- trifluoroacetyl)phenyl)urea |
| 83 | 231 | 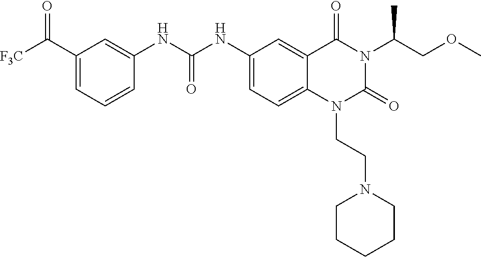 [SMILES: COC[C@H](C)n1c(=O)c2cc(NC(=O)Nc3cccc(C(=O)C(F)(F)F)c3)ccc2n(CCN2CCCCC2)c1=O] | (S)-1-(3-(1-methoxypropan-2- yl)-2,4-dioxo-1-(2-(piperidin-1- yl)ethyl)-1,2,3,4- tetrahydroquinazolin-6-yl)-3-(3- (2,2,2- trifluoroacetyl)phenyl)urea |

In an embodiment of the present invention provides a process for the preparation of compounds having structure I, the process comprising:
(i) reacting 2-amino-5-nitrobenzoic acid (compound 1) with an aliphatic or an aromatic amine selected from the group consisting of 2-methoxyethylamine, methoxypropylamine, 2-ethoxyethylamine, ethylamine 2M in THF, glycine ethylester hydrochloride, m-anisidine, o-anisidine, N,N-dimethylethylenediamine, 1-(2-aminoethyl) piperidine, 4-(2-aminoethyl) morpholine, 1-(2-Aminoethyl)-4-methylpiperazine, 3-(4-morpholinyl) propylamine, rac-1-methoxy-2-propylamine, 4-amino-1-methylpiperidine, 4-aminopyridine, 3-aminopyridine, 2-aminopyridine, (R)-1-methoxy-2-propylamine, (S)-1-methoxy-2-propylamine in presence of HATU/DMF followed by TEA as a base at room temperature for 1-3 hours to obtain an amide compound selected from the group consisting of 2, 98, 103, 108, 113, 118, 123, 128, 133, 138, 143, 148, 153, 158, 163, 168, 173, 218, 223;
(ii) cyclizing the compound selected from the group consisting of 2, 98, 103, 108, 113, 118, 123, 128, 133, 138, 143, 148, 153, 158, 163, 168, 173, 218, 223 obtained in step (i) using a cyclizing agent CDI in DMF as solvent at 100° C. for 12-16 hours to obtain a compound selected from the group consisting of 3, 99, 104, 109, 114, 119, 124, 129, 134, 139, 144, 149, 154, 159, 164, 169, 174, 219, 224;
(iii) reacting the compound 3 obtained in step (ii) with methyliodide and dry DMF at 0° C. for 12 hours to obtain the compound selected from the group consisting of 6;
(iv) Reacting the compound 3, 99, 104, 109, 114, 119, 124, 129, 134, 139, 144, 149, 154, 159, 164, 169, 174, 219, 224 obtained in step (ii) with suitable alkyl chloride selected from the group consisting of 2-iodopropane, bromocyclohexane, bromocyclopentane,4-bromo 1-methyl piperidine, (bromomethyl)cyclopropane, benzyl bromide, 4-(bromomethyl)benzonitrile, 4-(bromomethyl)-3-fluorobenzonitrile, 4-fluorobenzyl bromide, 4-nitrobenzyl bromide, 4-bromobenzyl bromide, 4-Methoxybenzyl bromide, 3-(bromomethyl)pyridine hydrobromide, methyl 3-(bromomethyl)benzoate, methyl 4-(bromomethyl)benzoate, 1-(2-chloroethyl) pyrrolidine, 1-(2-chloroethyl) piperidine, 4-(2-chloroethyl) morpholine, 1-(2-chloroethyl)-4-methylpiperazine, 1-(3-chloropropyl) pyrrolidine, 1-(3-chloropropyl) piperidine, 4-(3-chloropropyl) morpholine, 1-(3-chloropropyl)-4-methylpiperazine with K₂CO₃ and dry DMF at 120° C. for 12 hours to obtain the compound selected from the group consisting of 9, 12, 18, 21, 24, 27, 30, 33, 36, 39, 42, 45, 48, 51, 54, 57, 60, 63, 66, 86, 89, 92, 95, 100, 105, 110, 115, 120, 125, 130, 135, 140, 145, 150, 155, 160, 165, 170, 175, 220, 225;
(v) Reacting the compound 3 obtained in step (ii) with chlorobromoethane K₂CO₃ and dry DMF at 120° C. for 12 hours to obtain the compound selected from the group consisting of 69;
(vi) Reacting the compound 69 obtained in step (v) with suitable amine from the group consisting of Imidazole, N-Boc piperazine, pyrrole, indole, benzimidazole K₂CO₃ and dry DMF at 120° C. for 12 hours to obtain the compound selected from the group consisting of 70, 73, 77, 80, 83;
(vii) Reducing the compound selected from the group consisting of 3, 6, 9, 12, 18, 21, 24, 27, 30, 33, 36, 39, 42, 45, 48, 51, 54, 57, 60, 63, 66, 70, 73, 77, 80, 83, 86, 89, 92, 225 obtained in steps (ii), (iii) and (iv) using Palladium-Charcoal (5% or 10% wet) at room temperature for 2-5 hours in presence of H₂ to obtain an amine compound selected from the group consisting of 4, 7, 10, 13, 19, 22, 25, 28, 31, 34, 37, 40, 43, 46, 49, 52, 55, 58, 61, 64, 67, 71, 74, 78, 81, 84, 87, 90, 93, 96, 101, 106, 111, 116, 121, 126, 131, 136, 141, 146, 151, 156, 161, 166, 171, 176, 221, 226;
(viii) Treating the compound 4, 7, 10, 13, 19, 22, 25, 28, 31, 34, 37, 40, 43, 46, 49, 52, 55, 58, 61, 64, 67, 71, 74, 78, 81, 84, 87, 90, 93, 96, 101, 106, 111, 116, 121, 126, 131, 136, 141, 146, 151, 156, 161, 166, 171, 176, 221, 226 obtained in step (vii) with 4-nitrophenylchloroformate in presence of TEA as a base followed by reaction with an amine selected from the group consisting of 3′-aminoacetophenone in dry THF at room temperature for 3-8 hours to obtain the compound having structure I selected from the group consisting of 5, 8, 11, 14, 20, 23, 26, 29, 32, 35, 38, 41, 44, 47, 50, 53, 56, 59, 62, 65, 68, 72, 75, 79, 82, 85, 88, 91, 94, 97, 102, 107, 112, 117, 122, 127, 132, 137, 142, 147, 152, 157, 162, 167, 172, 177, 222, 227;
(ix) Treating the compound 61, 221, 226 obtained in step (vii) with 4-nitrophenylchloroformate in presence of TEA as a base followed by reaction with an amine selected from the group consisting of m-anisidine, o-anisidine, 1-(3-aminophenyl) ethanol, 3-ethylaniline, methyl 3-aminobenzoate, 3-amino-N,N-dimethylbenzamide, (3-aminophenyl) (pyrrolidin-1-yl) methanone, (3-aminophenyl) (morpholino) methanone, aniline, N1-methylbenzene-1,3-diamine, N-(3-aminophenyl) acetamide, N-(3-aminophenyl)-N-methylacetamide, N-(3-aminophenyl)-N-benzylacetamide, 1-(3-(methylamino)phenyl) ethanone, 1-(3-(hydroxyamino)phenyl) ethanone, 1-(3-aminophenyl)-2,2,2-trifluoroethanone in dry THF at room temperature for 3-8 hours to obtain the compound having structure I selected from the group consisting of 178, 179, 180, 181, 182, 184, 185, 186, 187, 188, 189, 190, 191, 192, 193, 195, 228, 229, 230, 231;
(x) Treating the compound 182 obtained in step (ix) with LiOH. H₂O in THF, Methanol and H₂O in proportion of (3:2:1) at room temperature for 12 hours to obtain the compound having structure I selected from the group consisting of 183;
(xi) Treating the compound 62 obtained in step (viii) with hydroxylamine hydrochloride in EtOH at room temperature for 12 hours to obtain the compound having structure I selected from the group consisting of 194;
(xii) Treating the compound 75 obtained in step (viii) with trifluoroacetic acid in DCM at room temperature for 8 hours to obtain the compound having structure I selected from the group consisting of 76;
(xiii) Treating the compound 62 obtained in step (viii) with NH₃/Methanol and followed by NaBH₄ in Methanol at room temperature for 12 hours to obtain the compound having structure I selected from the group consisting of 196;
(xiv) Treating the compound 196 obtained in step (xiii) with HCOOH and formaldehyde in presence of conc. HCl for 8 hours at 100° C. to obtain the compound having structure I selected from the group consisting of 197;
(xv) Treating the compound 62 obtained in step (viii) with various aromatic amines (Aniline, 2-Fluoroaniline, 4-Fluoroaniline, 4-Methoxyaniline, 4-Aminobenzonitrile, cyclohexylamine, cyclopentylamine) in presence of p-Toluene sulfonic acid (PTSA) in EtOH at 100° C. to obtain the compounds having structure I selected from the group consisting of 196, 200, 202, 204, 206, 208, 210;
(xvi) Treating the compounds 196, 200, 202, 204, 206, 208, 210 obtained in step (xv) with Sodium cyanoborohydride (NaCNBH₃) in dry methanol at room temperature for 8 hours to obtain the compounds having structure I selected from the group consisting of 197, 201, 203, 205, 207, 209, 211;
(xvii) Treating the compound which is commercially available 212 with HATU and 2-methoxyethylamine in DMF at room temperature for 2 hours to obtain the compounds having structure I selected from the group consisting of 213;
(xviii) Treating the compound obtained in step (xvii) with CDI in DMF for 12 hours at 100° C. to obtain the compounds having structure I selected from the group consisting of 214;
(xix) Reacting the compound 214 obtained in step (xviii) with 1-(2-chloroethyl) piperidine and K₂CO₃ and dry DMF at 120° C. for 12 hours to obtain the compound selected from the group consisting of 215;
(xx) Reacting the compound 215 obtained in step (xix) with 1-(2-amino-1H-benzo[d]imidazol-5-yl) ethenone and Pd₂ (dba) 3 as catalyst and X-Phos as ligand and base K₂CO₃ at 100° C. for 12 hours to obtain the compound selected from the group consisting of 216;
(xxi) Reacting the compound 61 obtained in step (vii) with 3,4-Diethoxy-3-cyclobutene-1,2-dione and p-toluene sulfonic acid (PTSA) in EtOH and 3-aminoacetophenone at 80° C. for 12 hours to obtain the compound selected from the group consisting of 217.

### General Procedure of Urea Formation Via Chloroformate Intermediate

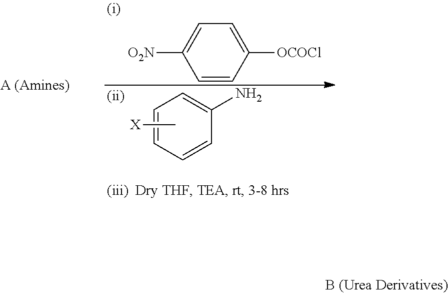

TABLE 2

| Serial No | Amine (A) | 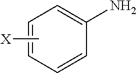 | B (Urea derivatives) |
|---|---|---|---|
| 1 | 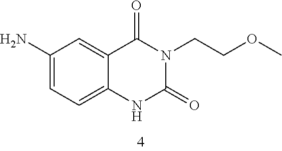 [SMILES: COCCn1c(=O)[nH]c2ccc(N)cc2c1=O] | 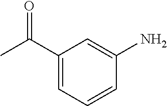 | 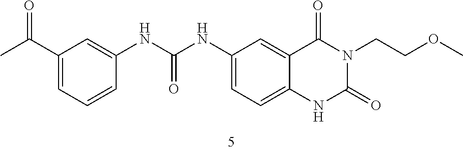 [SMILES: COCCn1c(=O)[nH]c2ccc(NC(=O)Nc3cccc(C(C)=O)c3)cc2c1=O] |
| 2 | 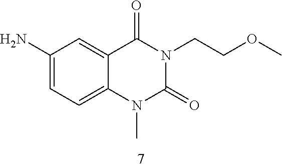 [SMILES: COCCn1c(=O)c2cc(N)ccc2n(C)c1=O] | 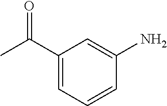 | 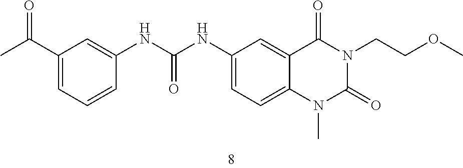 [SMILES: COCCn1c(=O)c2cc(NC(=O)Nc3cccc(C(C)=O)c3)ccc2n(C)c1=O] |
| 3 | 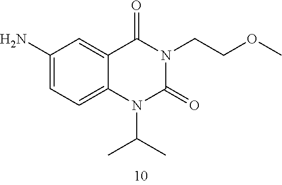 [SMILES: COCCn1c(=O)c2cc(N)ccc2n(C(C)C)c1=O] | 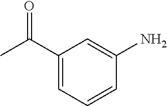 | 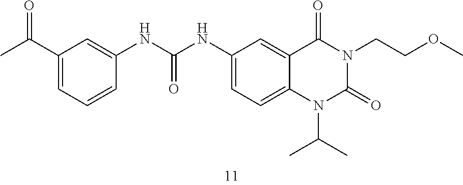 [SMILES: COCCn1c(=O)c2cc(NC(=O)Nc3cccc(C(C)=O)c3)ccc2n(C(C)C)c1=O] |
| 4 | 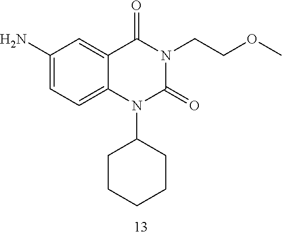 [SMILES: COCCn1c(=O)c2cc(N)ccc2n(C2CCCCC2)c1=O] | 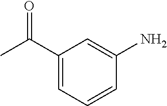 | 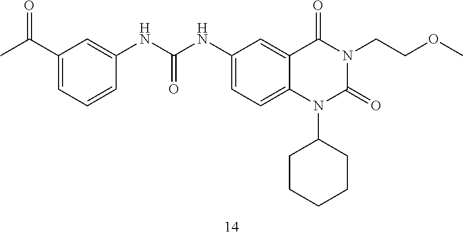 [SMILES: COCCn1c(=O)c2cc(NC(=O)Nc3cccc(C(C)=O)c3)ccc2n(C2CCCCC2)c1=O] |
| 5 | 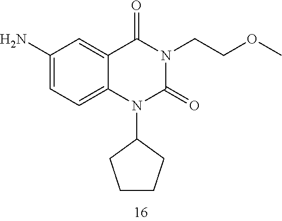 [SMILES: COCCn1c(=O)c2cc(N)ccc2n(C2CCCC2)c1=O] | 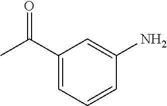 | 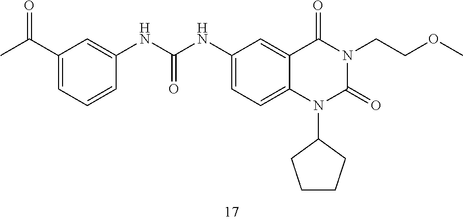 [SMILES: COCCn1c(=O)c2cc(NC(=O)Nc3cccc(C(C)=O)c3)ccc2n(C2CCCC2)c1=O] |
| 6 | 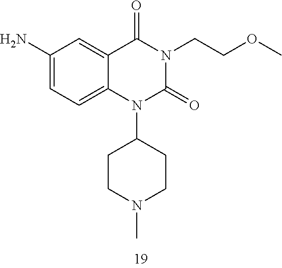 [SMILES: COCCn1c(=O)c2cc(N)ccc2n(C2CCN(C)CC2)c1=O] | 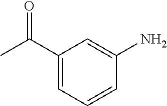 | 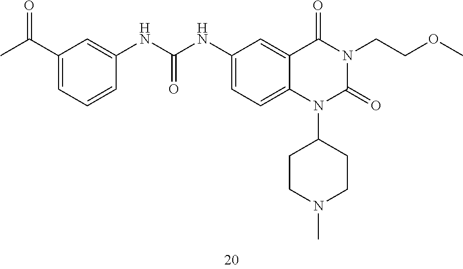 [SMILES: COCCn1c(=O)c2cc(NC(=O)Nc3cccc(C(C)=O)c3)ccc2n(C2CCN(C)CC2)c1=O] |
| 7 | 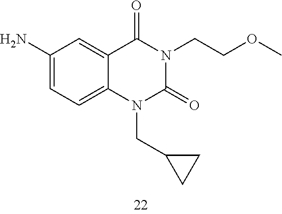 [SMILES: COCCn1c(=O)c2cc(N)ccc2n(CC2CC2)c1=O] | 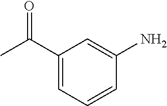 | 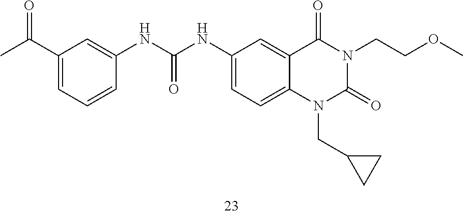 [SMILES: COCCn1c(=O)c2cc(NC(=O)Nc3cccc(C(C)=O)c3)ccc2n(CC2CC2)c1=O] |
| 8 | 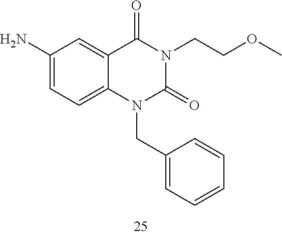 [SMILES: COCCn1c(=O)c2cc(N)ccc2n(Cc2ccccc2)c1=O] | 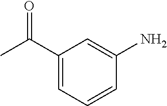 | 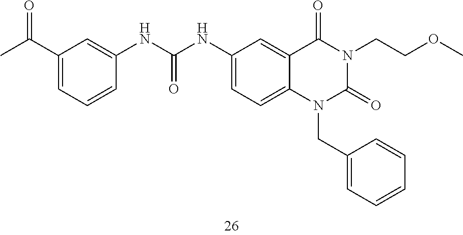 [SMILES: COCCn1c(=O)c2cc(NC(=O)Nc3cccc(C(C)=O)c3)ccc2n(Cc2ccccc2)c1=O] |
| 9 | 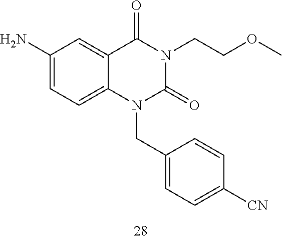 [SMILES: COCCn1c(=O)c2cc(N)ccc2n(Cc2ccc(C#N)cc2)c1=O] | 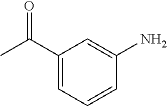 | 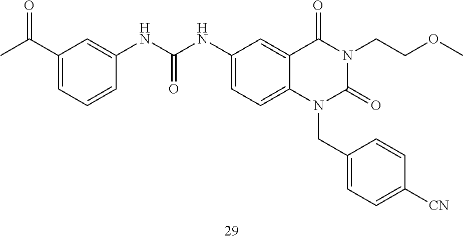 [SMILES: COCCn1c(=O)c2cc(NC(=O)Nc3cccc(C(C)=O)c3)ccc2n(Cc2ccc(C#N)cc2)c1=O] |
| 10 | 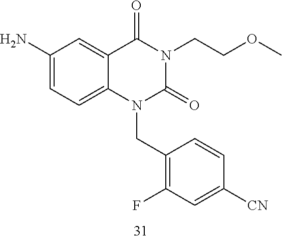 [SMILES: COCCn1c(=O)c2cc(N)ccc2n(Cc2ccc(C#N)cc2F)c1=O] | 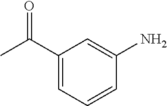 | 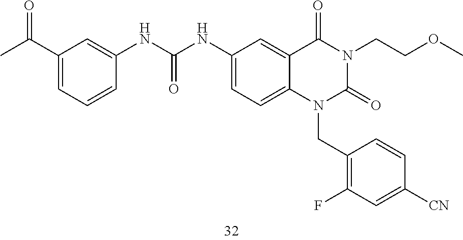 [SMILES: COCCn1c(=O)c2cc(NC(=O)Nc3cccc(C(C)=O)c3)ccc2n(Cc2ccc(C#N)cc2F)c1=O] |
| 11 | 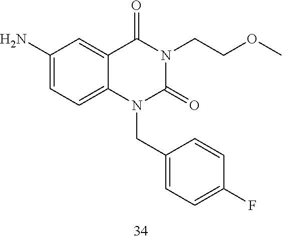 [SMILES: COCCn1c(=O)c2cc(N)ccc2n(Cc2ccc(F)cc2)c1=O] | 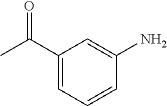 | 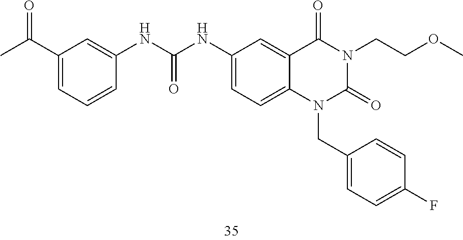 [SMILES: COCCn1c(=O)c2cc(NC(=O)Nc3cccc(C(C)=O)c3)ccc2n(Cc2ccc(F)cc2)c1=O] |
| 12 | 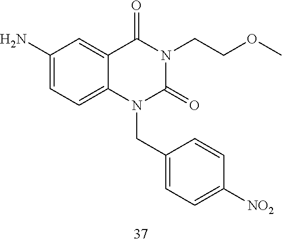 [SMILES: COCCn1c(=O)c2cc(N)ccc2n(Cc2ccc([N+](=O)[O-])cc2)c1=O] | 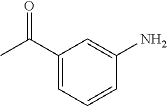 | 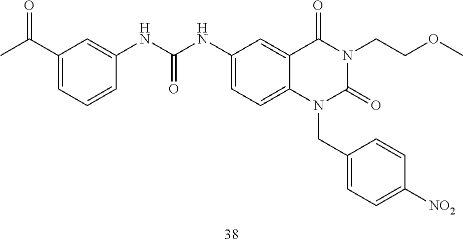 [SMILES: COCCn1c(=O)c2cc(NC(=O)Nc3cccc(C(C)=O)c3)ccc2n(Cc2ccc([N+](=O)[O-])cc2)c1=O] |
| 13 | 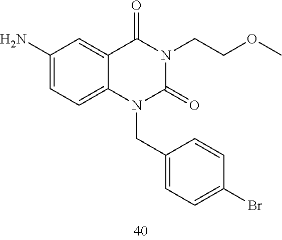 [SMILES: COCCn1c(=O)c2cc(N)ccc2n(Cc2ccc(Br)cc2)c1=O] | 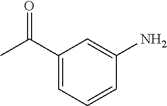 | 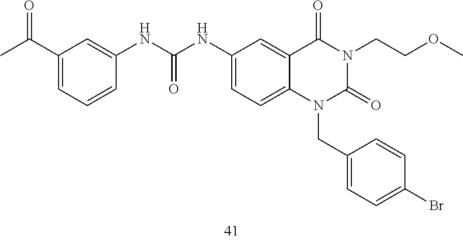 [SMILES: COCCn1c(=O)c2cc(NC(=O)Nc3cccc(C(C)=O)c3)ccc2n(Cc2ccc(Br)cc2)c1=O] |
| 14 | 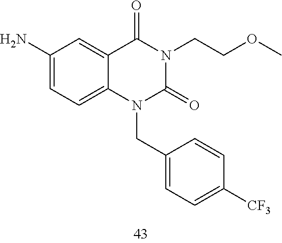 [SMILES: COCCn1c(=O)c2cc(N)ccc2n(Cc2ccc(C(F)(F)F)cc2)c1=O] | 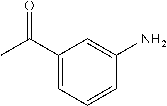 | 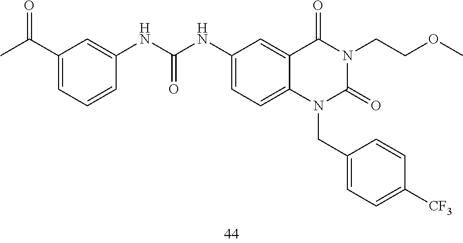 [SMILES: COCCn1c(=O)c2cc(NC(=O)Nc3cccc(C(C)=O)c3)ccc2n(Cc2ccc(C(F)(F)F)cc2)c1=O] |
| 15 | 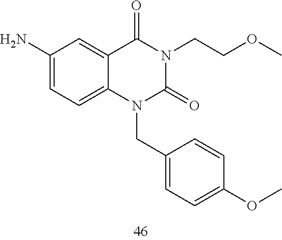 [SMILES: COCCn1c(=O)c2cc(N)ccc2n(Cc2ccc(OC)cc2)c1=O] | 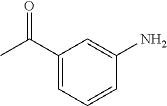 | 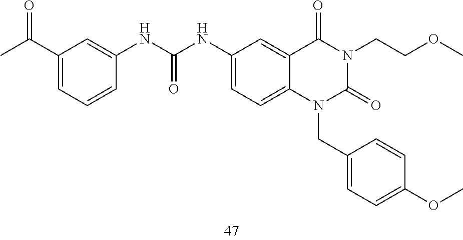 [SMILES: COCCn1c(=O)c2cc(NC(=O)Nc3cccc(C(C)=O)c3)ccc2n(Cc2ccc(OC)cc2)c1=O] |
| 16 | 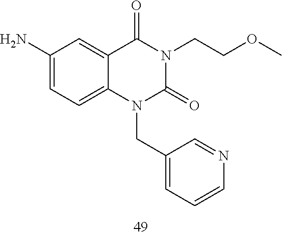 [SMILES: COCCn1c(=O)c2cc(N)ccc2n(Cc2cccnc2)c1=O] | 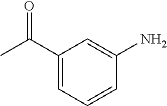 | 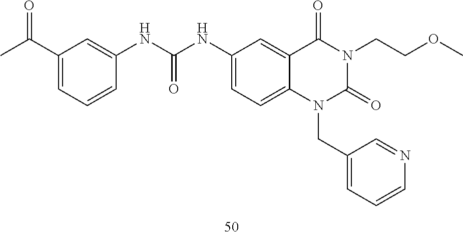 [SMILES: COCCn1c(=O)c2cc(NC(=O)Nc3cccc(C(C)=O)c3)ccc2n(Cc2cccnc2)c1=O] |
| 17 | 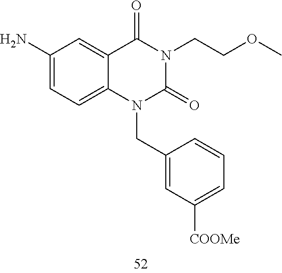 [SMILES: COCCn1c(=O)c2cc(N)ccc2n(Cc2cccc(C(=O)OC)c2)c1=O] | 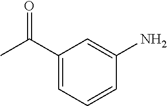 | 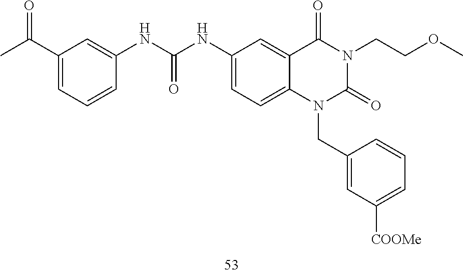 [SMILES: COCCn1c(=O)c2cc(NC(=O)Nc3cccc(C(C)=O)c3)ccc2n(Cc2cccc(C(=O)OC)c2)c1=O] |
| 18 | 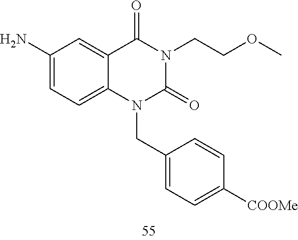 [SMILES: COCCn1c(=O)c2cc(N)ccc2n(Cc2ccc(C(=O)OC)cc2)c1=O] | 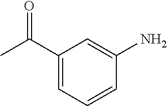 | 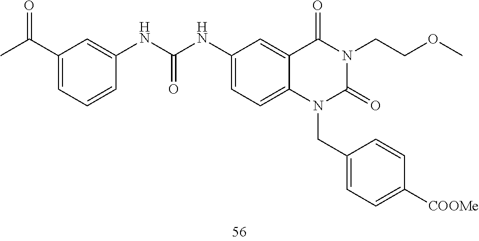 [SMILES: COCCn1c(=O)c2cc(NC(=O)Nc3cccc(C(C)=O)c3)ccc2n(Cc2ccc(C(=O)OC)cc2)c1=O] |
| 19 | 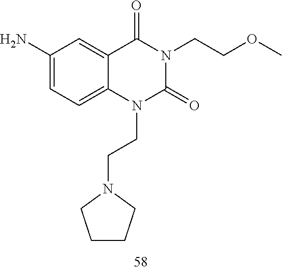 [SMILES: COCCn1c(=O)c2cc(N)ccc2n(CCN2CCCC2)c1=O] | 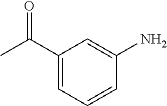 | 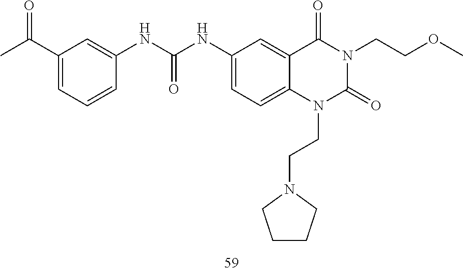 [SMILES: COCCn1c(=O)c2cc(NC(=O)Nc3cccc(C(C)=O)c3)ccc2n(CCN2CCCC2)c1=O] |
| 20 | 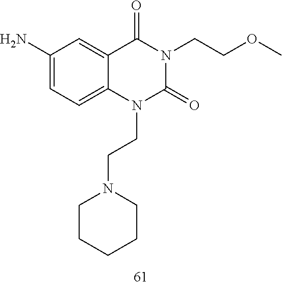 [SMILES: COCCn1c(=O)c2cc(N)ccc2n(CCN2CCCCC2)c1=O] | 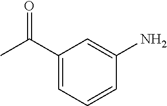 | 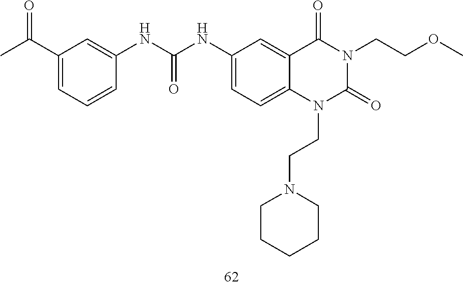 [SMILES: COCCn1c(=O)c2cc(NC(=O)Nc3cccc(C(C)=O)c3)ccc2n(CCN2CCCCC2)c1=O] |
| 21 | 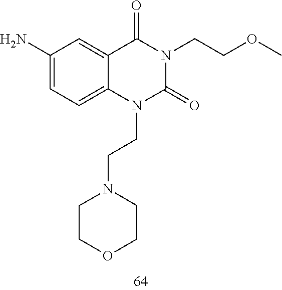 [SMILES: COCCn1c(=O)c2cc(N)ccc2n(CCN2CCOCC2)c1=O] | 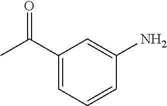 | 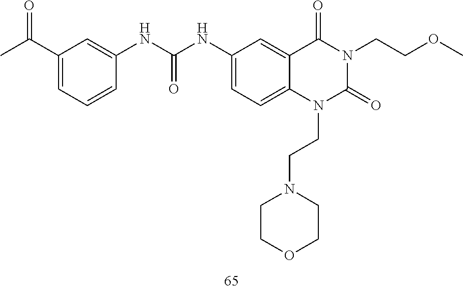 [SMILES: COCCn1c(=O)c2cc(NC(=O)Nc3cccc(C(C)=O)c3)ccc2n(CCN2CCOCC2)c1=O] |
| 22 | 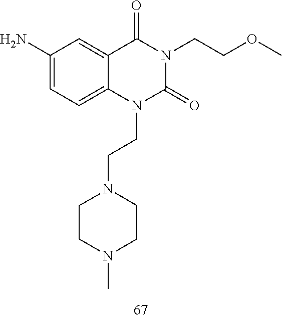 [SMILES: COCCn1c(=O)c2cc(N)ccc2n(CCN2CCN(C)CC2)c1=O] | 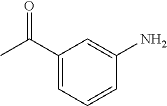 | 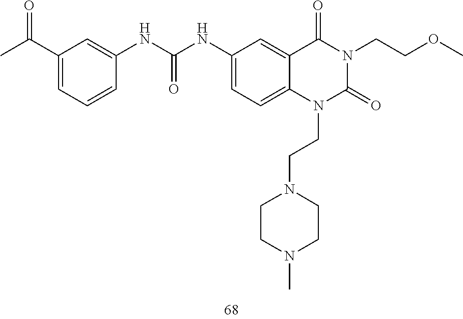 [SMILES: COCCn1c(=O)c2cc(NC(=O)Nc3cccc(C(C)=O)c3)ccc2n(CCN2CCN(C)CC2)c1=O] |
| 23 | 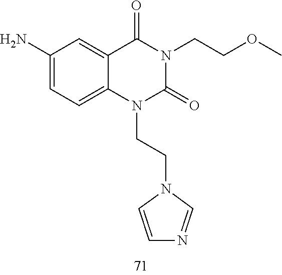 [SMILES: COCCn1c(=O)c2cc(N)ccc2n(CCn2ccnc2)c1=O] | 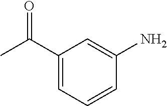 | 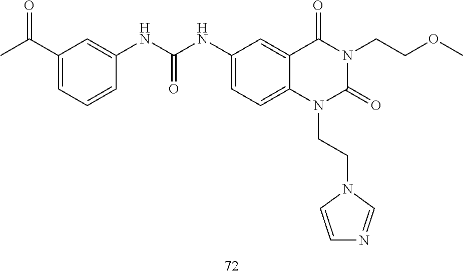 [SMILES: COCCn1c(=O)c2cc(NC(=O)Nc3cccc(C(C)=O)c3)ccc2n(CCn2ccnc2)c1=O] |
| 24 | 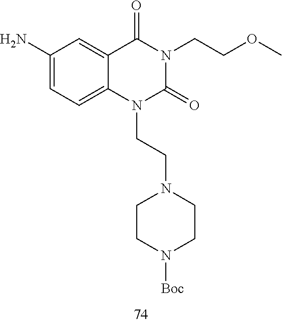 [SMILES: COCCn1c(=O)c2cc(N)ccc2n(CCN2CCN(C(=O)OC(C)(C)C)CC2)c1=O] | 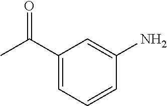 | 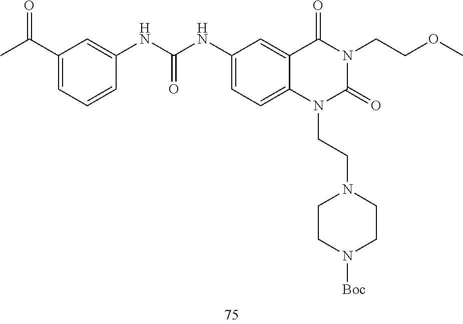 [SMILES: COCCn1c(=O)c2cc(NC(=O)Nc3cccc(C(C)=O)c3)ccc2n(CCN2CCN(C(=O)OC(C)(C)C)CC2)c1=O] |
| 25 | 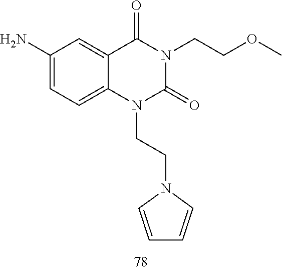 [SMILES: COCCn1c(=O)c2cc(N)ccc2n(CCn2cccc2)c1=O] | 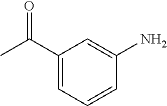 | 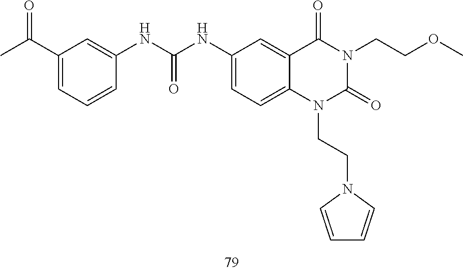 [SMILES: COCCn1c(=O)c2cc(NC(=O)Nc3cccc(C(C)=O)c3)ccc2n(CCn2cccc2)c1=O] |
| 26 | 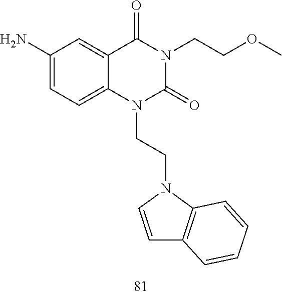 [SMILES: COCCn1c(=O)c2cc(N)ccc2n(CCn2ccc3ccccc32)c1=O] | 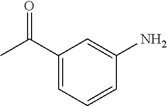 | 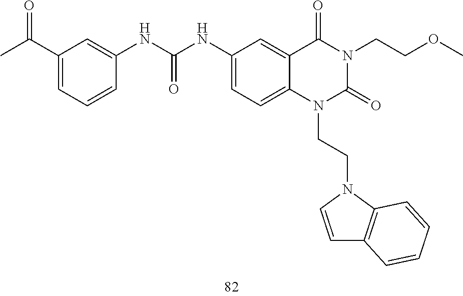 [SMILES: COCCn1c(=O)c2cc(NC(=O)Nc3cccc(C(C)=O)c3)ccc2n(CCn2ccc3ccccc32)c1=O] |
| 27 | 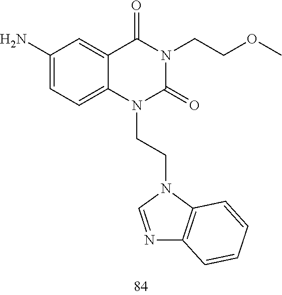 [SMILES: COCCn1c(=O)c2cc(N)ccc2n(CCn2cnc3ccccc32)c1=O] | 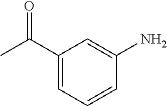 | 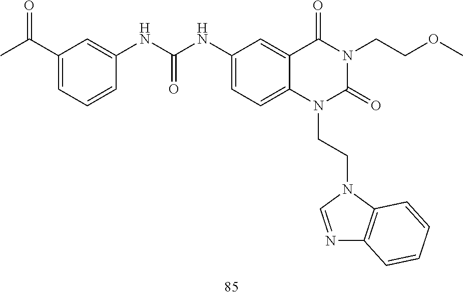 [SMILES: COCCn1c(=O)c2cc(NC(=O)Nc3cccc(C(C)=O)c3)ccc2n(CCn2cnc3ccccc32)c1=O] |
| 28 | 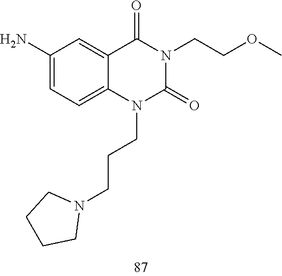 [SMILES: COCCn1c(=O)c2cc(N)ccc2n(CCCN2CCCC2)c1=O] | 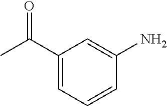 | 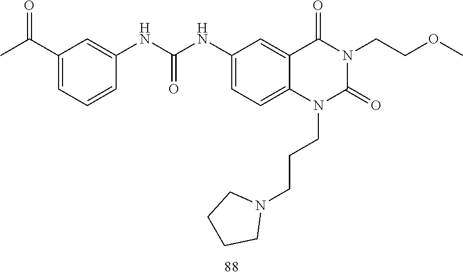 [SMILES: COCCn1c(=O)c2cc(NC(=O)Nc3cccc(C(C)=O)c3)ccc2n(CCCN2CCCC2)c1=O] |
| 29 | 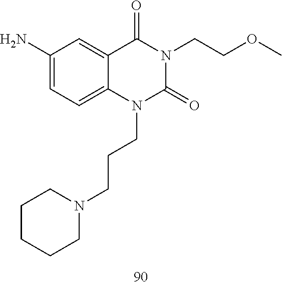 [SMILES: COCCn1c(=O)c2cc(N)ccc2n(CCCN2CCCCC2)c1=O] | 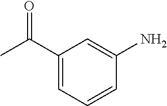 | 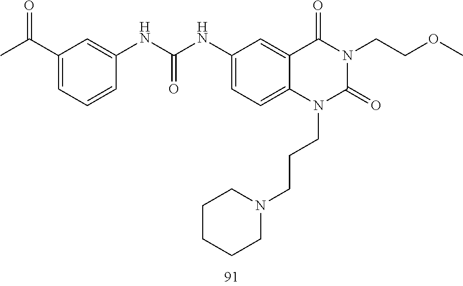 [SMILES: COCCn1c(=O)c2cc(NC(=O)Nc3cccc(C(C)=O)c3)ccc2n(CCCN2CCCCC2)c1=O] |
| 30 | 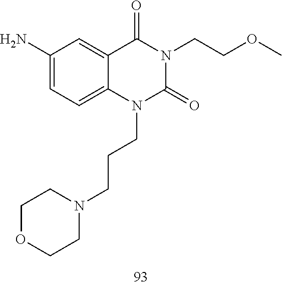 [SMILES: COCCn1c(=O)c2cc(N)ccc2n(CCCN2CCOCC2)c1=O] | 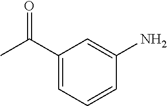 | 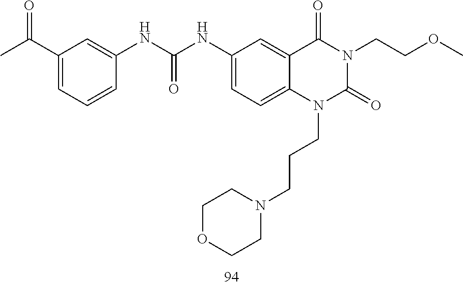 [SMILES: COCCn1c(=O)c2cc(NC(=O)Nc3cccc(C(C)=O)c3)ccc2n(CCCN2CCOCC2)c1=O] |
| 31 | 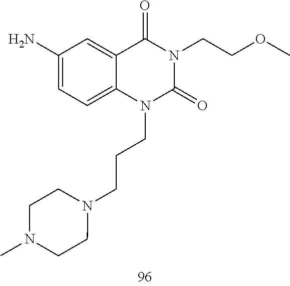 | 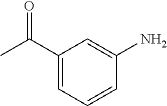 | 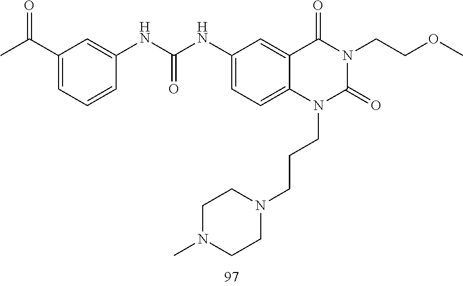 [SMILES: COCCn1c(=O)c2cc(NC(=O)Nc3cccc(C(C)=O)c3)ccc2n(CCCN2CCN(C)CC2)c1=O] |
| 32 | 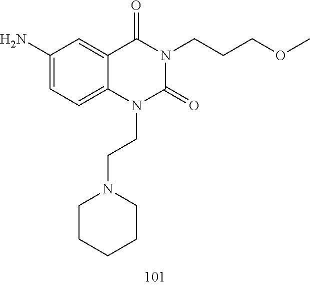 [SMILES: COCCCn1c(=O)c2cc(N)ccc2n(CCN2CCCCC2)c1=O] | 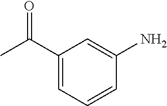 | 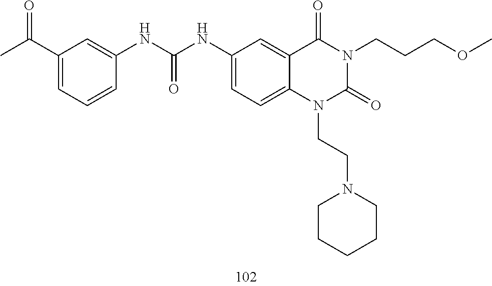 [SMILES: COCCCn1c(=O)c2cc(NC(=O)Nc3cccc(C(C)=O)c3)ccc2n(CCN2CCCCC2)c1=O] |
| 33 | 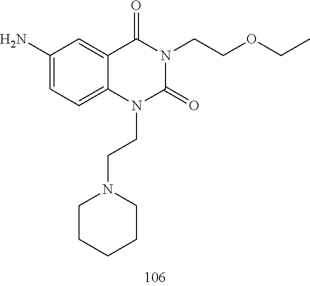 [SMILES: CCOCCn1c(=O)c2cc(N)ccc2n(CCN2CCCCC2)c1=O] | 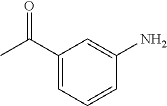 | 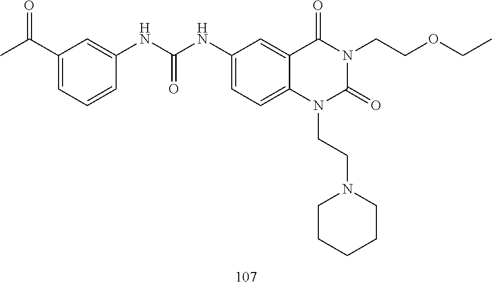 [SMILES: CCOCCn1c(=O)c2cc(NC(=O)Nc3cccc(C(C)=O)c3)ccc2n(CCN2CCCCC2)c1=O] |
| 34 | 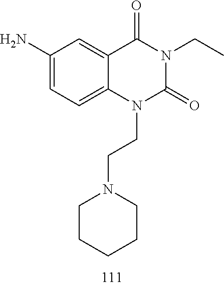 [SMILES: CCn1c(=O)c2cc(N)ccc2n(CCN2CCCCC2)c1=O] | 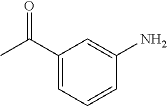 | 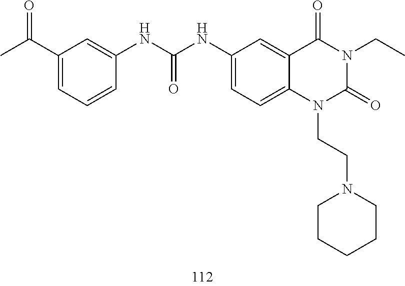 [SMILES: CCn1c(=O)c2cc(NC(=O)Nc3cccc(C(C)=O)c3)ccc2n(CCN2CCCCC2)c1=O] |
| 35 | 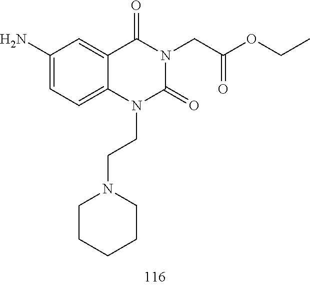 [SMILES: CCOC(=O)Cn1c(=O)c2cc(N)ccc2n(CCN2CCCCC2)c1=O] | 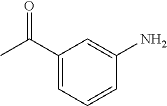 | 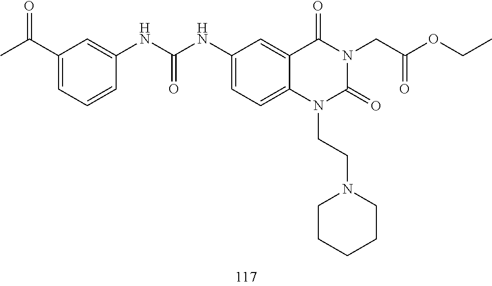 [SMILES: CCOC(=O)Cn1c(=O)c2cc(NC(=O)Nc3cccc(C(C)=O)c3)ccc2n(CCN2CCCCC2)c1=O] |
| 36 | 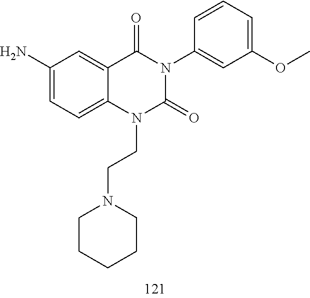 [SMILES: COc1cccc(-n2c(=O)c3cc(N)ccc3n(CCN3CCCCC3)c2=O)c1] | 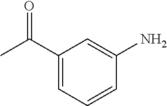 | 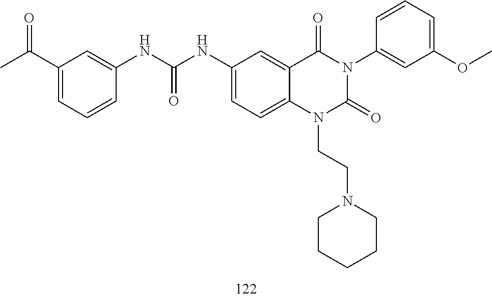 [SMILES: COc1cccc(-n2c(=O)c3cc(NC(=O)Nc4cccc(C(C)=O)c4)ccc3n(CCN3CCCCC3)c2=O)c1] |
| 37 | 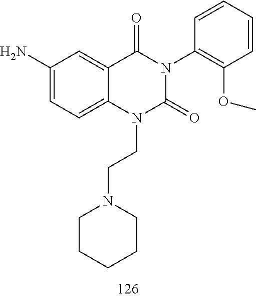 [SMILES: COc1ccccc1-n1c(=O)c2cc(N)ccc2n(CCN2CCCCC2)c1=O] | 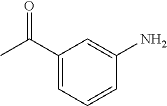 | 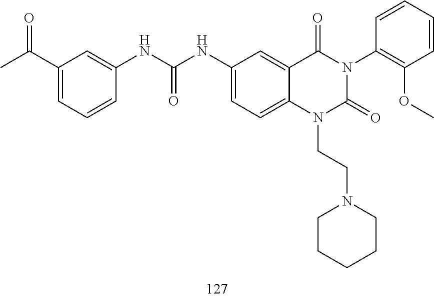 [SMILES: COc1ccccc1-n1c(=O)c2cc(NC(=O)Nc3cccc(C(C)=O)c3)ccc2n(CCN2CCCCC2)c1=O] |
| 38 | 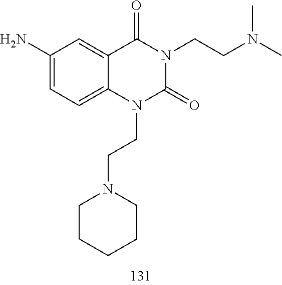 [SMILES: CN(C)CCn1c(=O)c2cc(N)ccc2n(CCN2CCCCC2)c1=O] | 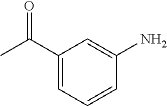 | 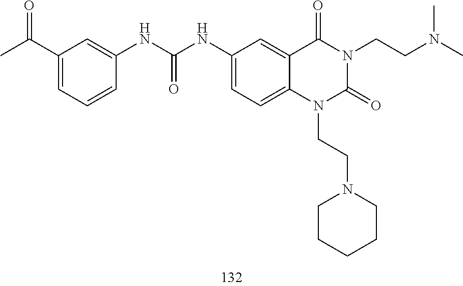 [SMILES: CC(=O)c1cccc(NC(=O)Nc2ccc3c(c2)c(=O)n(CCN(C)C)c(=O)n3CCN2CCCCC2)c1] |
| 39 | 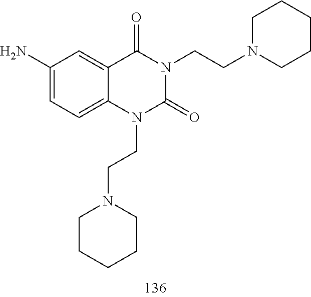 [SMILES: Nc1ccc2c(c1)c(=O)n(CCN1CCCCC1)c(=O)n2CCN1CCCCC1] | 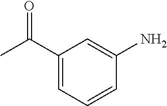 | 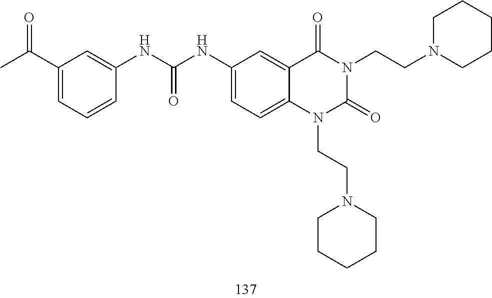 [SMILES: CC(=O)c1cccc(NC(=O)Nc2ccc3c(c2)c(=O)n(CCN2CCCCC2)c(=O)n3CCN2CCCCC2)c1] |
| 40 | 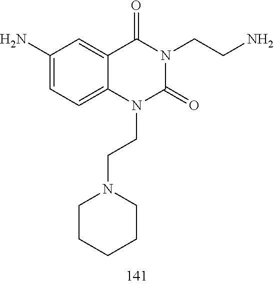 [SMILES: NCCn1c(=O)c2cc(N)ccc2n(CCN2CCCCC2)c1=O] | 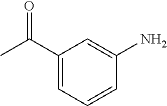 | 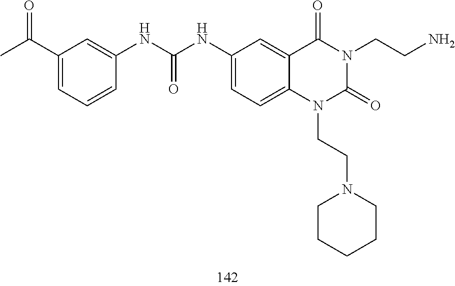 [SMILES: CC(=O)c1cccc(NC(=O)Nc2ccc3c(c2)c(=O)n(CCN)c(=O)n3CCN2CCCCC2)c1] |
| 41 | 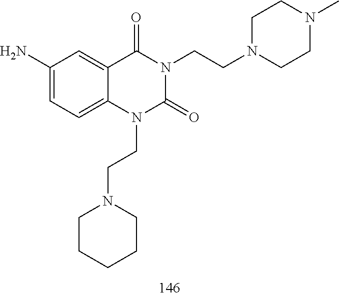 [SMILES: CN1CCN(CCn2c(=O)c3cc(N)ccc3n(CCN3CCCCC3)c2=O)CC1] | 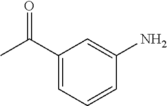 | 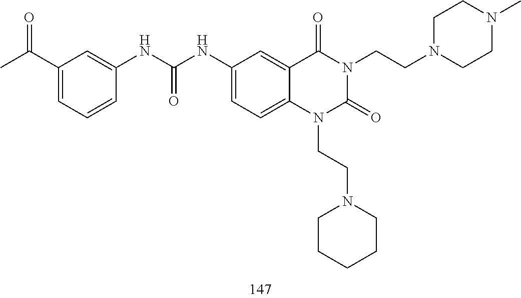 [SMILES: CC(=O)c1cccc(NC(=O)Nc2ccc3c(c2)c(=O)n(CCN2CCN(C)CC2)c(=O)n3CCN2CCCCC2)c1] |
| 42 | 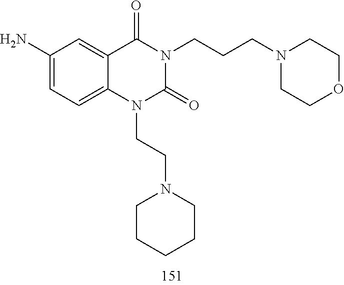 [SMILES: Nc1ccc2c(c1)c(=O)n(CCCN1CCOCC1)c(=O)n2CCN1CCCCC1] | 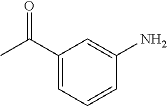 | 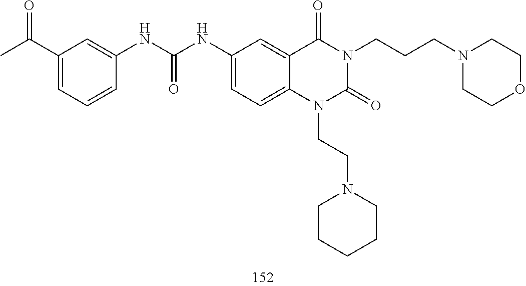 [SMILES: CC(=O)c1cccc(NC(=O)Nc2ccc3c(c2)c(=O)n(CCCN2CCOCC2)c(=O)n3CCN2CCCCC2)c1] |
| 43 | 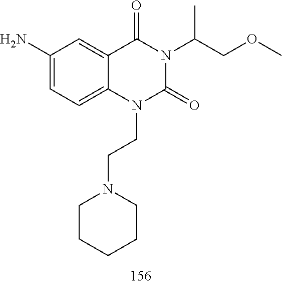 [SMILES: COCC(C)n1c(=O)c2cc(N)ccc2n(CCN2CCCCC2)c1=O] | 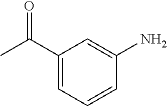 | 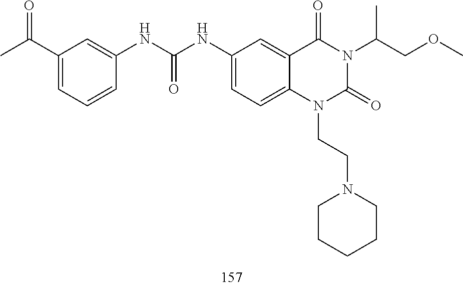 [SMILES: COCC(C)n1c(=O)c2cc(NC(=O)Nc3cccc(C(C)=O)c3)ccc2n(CCN2CCCCC2)c1=O] |
| 44 | 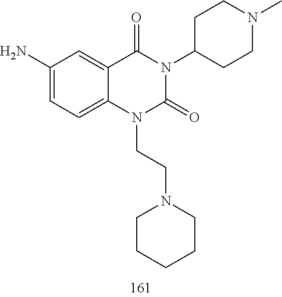 [SMILES: CN1CCC(n2c(=O)c3cc(N)ccc3n(CCN3CCCCC3)c2=O)CC1] | 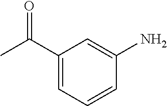 | 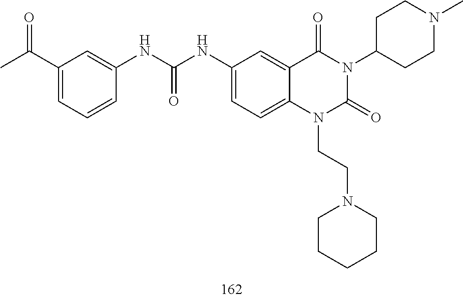 [SMILES: CC(=O)c1cccc(NC(=O)Nc2ccc3c(c2)c(=O)n(C2CCN(C)CC2)c(=O)n3CCN2CCCCC2)c1] |
| 45 | 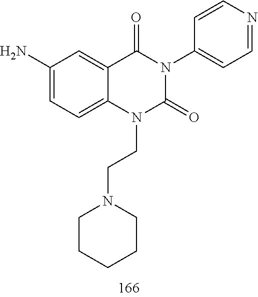 [SMILES: Nc1ccc2c(c1)c(=O)n(-c1ccncc1)c(=O)n2CCN1CCCCC1] | 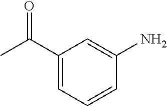 | 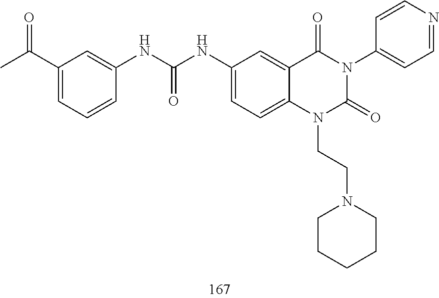 [SMILES: CC(=O)c1cccc(NC(=O)Nc2ccc3c(c2)c(=O)n(-c2ccncc2)c(=O)n3CCN2CCCCC2)c1] |
| 46 | 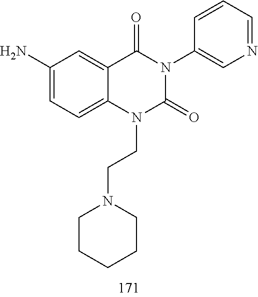 [SMILES: Nc1ccc2c(c1)c(=O)n(-c1cccnc1)c(=O)n2CCN1CCCCC1] | 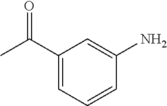 | 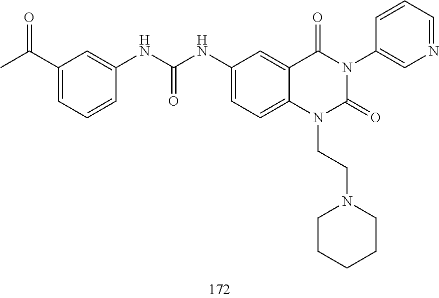 [SMILES: CC(=O)c1cccc(NC(=O)Nc2ccc3c(c2)c(=O)n(-c2cccnc2)c(=O)n3CCN2CCCCC2)c1] |
| 47 | 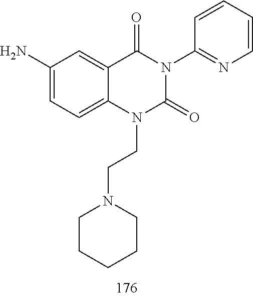 [SMILES: Nc1ccc2c(c1)c(=O)n(-c1ccccn1)c(=O)n2CCN1CCCCC1] | 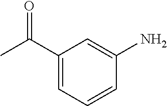 | 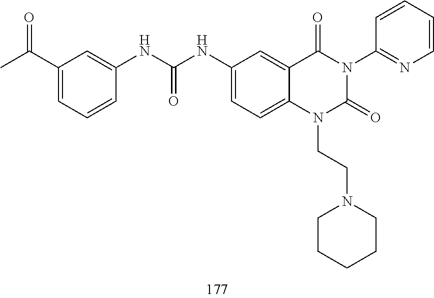 [SMILES: CC(=O)c1cccc(NC(=O)Nc2ccc3c(c2)c(=O)n(-c2ccccn2)c(=O)n3CCN2CCCCC2)c1] |
| 48 | 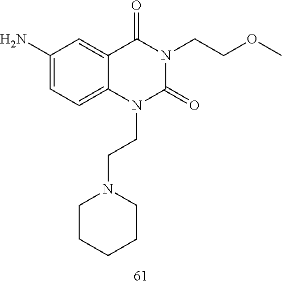 [SMILES: COCCn1c(=O)c2cc(N)ccc2n(CCN2CCCCC2)c1=O] | 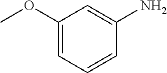 | 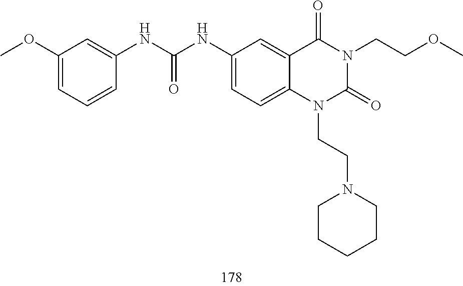 |
| 49 | 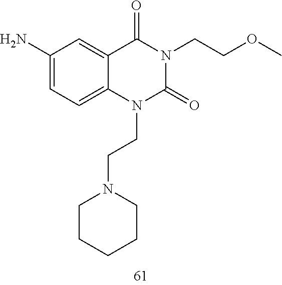 [SMILES: COCCn1c(=O)c2cc(N)ccc2n(CCN2CCCCC2)c1=O] | 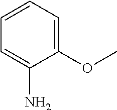 | 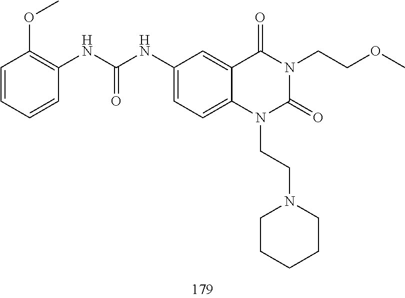 [SMILES: COCCn1c(=O)c2cc(NC(=O)Nc3ccccc3OC)ccc2n(CCN2CCCCC2)c1=O] |
| 50 | 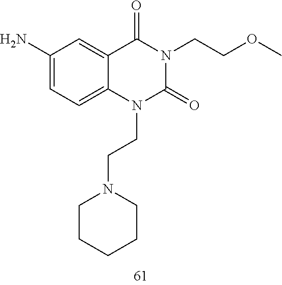 [SMILES: COCCn1c(=O)c2cc(N)ccc2n(CCN2CCCCC2)c1=O] | 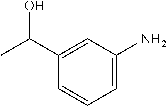 | 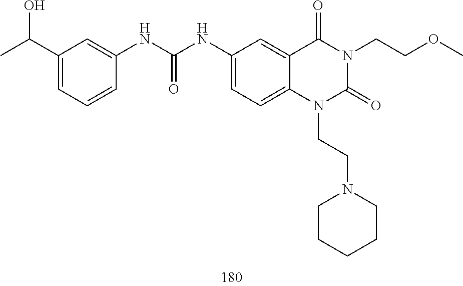 [SMILES: COCCn1c(=O)c2cc(NC(=O)Nc3cccc(C(C)O)c3)ccc2n(CCN2CCCCC2)c1=O] |
| 51 | 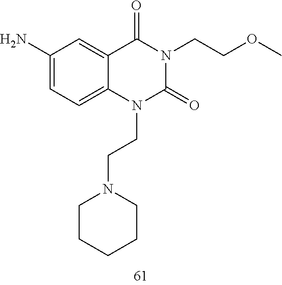 [SMILES: COCCn1c(=O)c2cc(N)ccc2n(CCN2CCCCC2)c1=O] | 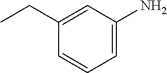 | 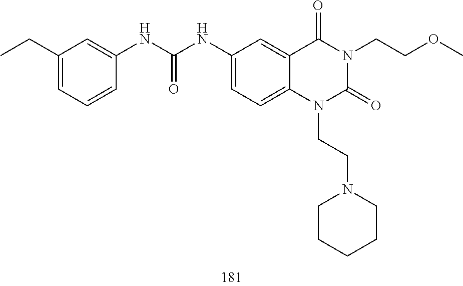 [SMILES: CCc1cccc(NC(=O)Nc2ccc3c(c2)c(=O)n(CCOC)c(=O)n3CCN2CCCCC2)c1] |
| 52 | 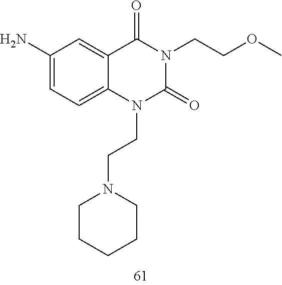 [SMILES: COCCn1c(=O)c2cc(N)ccc2n(CCN2CCCCC2)c1=O] | 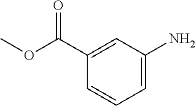 | 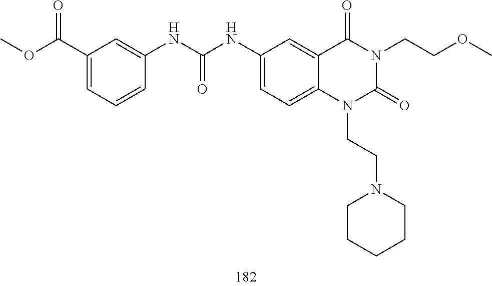 |
| 53 | 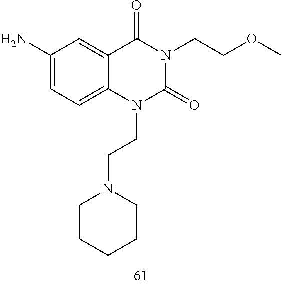 [SMILES: COCCn1c(=O)c2cc(N)ccc2n(CCN2CCCCC2)c1=O] | 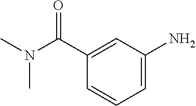 | 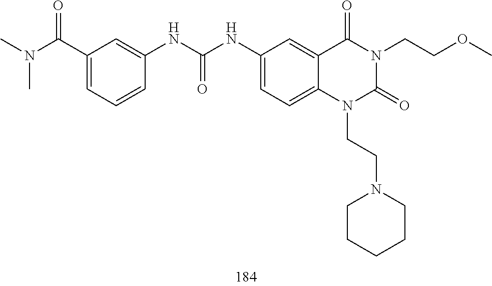 |
| 54 | 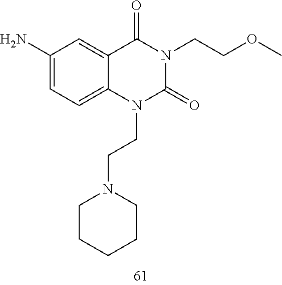 [SMILES: COCCn1c(=O)c2cc(N)ccc2n(CCN2CCCCC2)c1=O] | 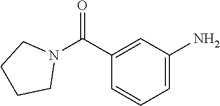 [SMILES: Nc1cccc(C(=O)N2CCCC2)c1] | 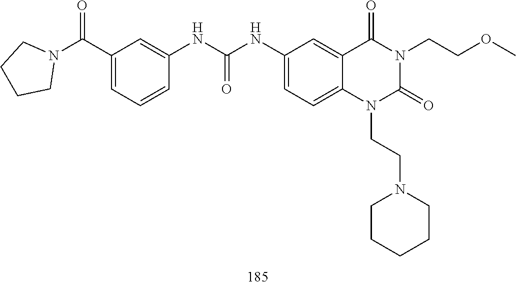 [SMILES: COCCn1c(=O)c2cc(NC(=O)Nc3cccc(C(=O)N4CCCC4)c3)ccc2n(CCN2CCCCC2)c1=O] |
| 55 | 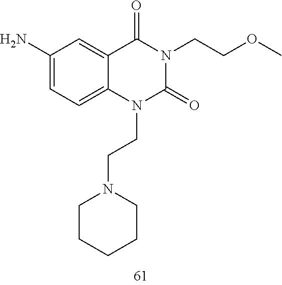 [SMILES: COCCn1c(=O)c2cc(N)ccc2n(CCN2CCCCC2)c1=O] | 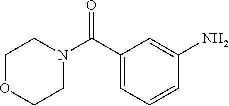 [SMILES: Nc1cccc(C(=O)N2CCOCC2)c1] | 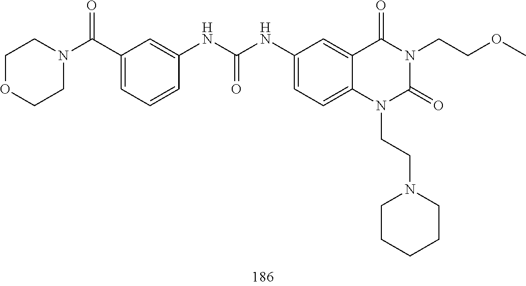 [SMILES: COCCn1c(=O)c2cc(NC(=O)Nc3cccc(C(=O)N4CCOCC4)c3)ccc2n(CCN2CCCCC2)c1=O] |
| 56 | 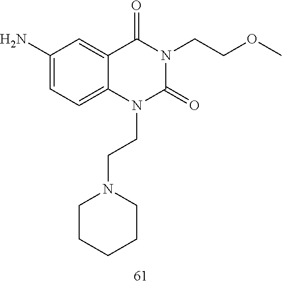 [SMILES: COCCn1c(=O)c2cc(N)ccc2n(CCN2CCCCC2)c1=O] | 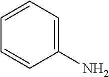 | 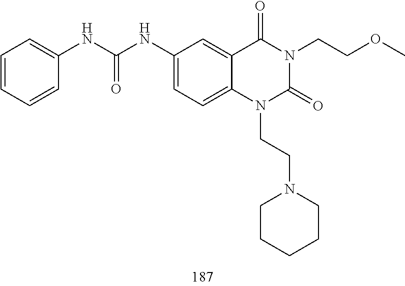 [SMILES: COCCn1c(=O)c2cc(NC(=O)Nc3ccccc3)ccc2n(CCN2CCCCC2)c1=O] |
| 57 | 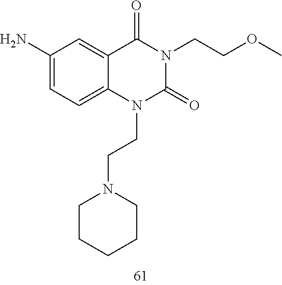 [SMILES: COCCn1c(=O)c2cc(N)ccc2n(CCN2CCCCC2)c1=O] | 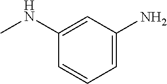 | 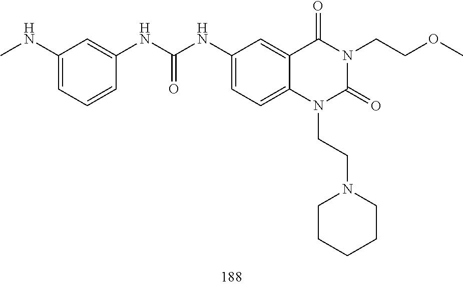 |
| 58 | 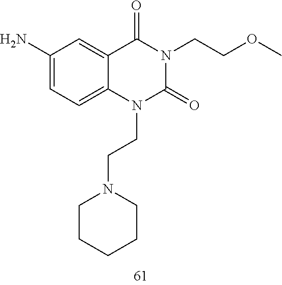 [SMILES: COCCn1c(=O)c2cc(N)ccc2n(CCN2CCCCC2)c1=O] | 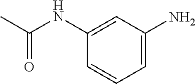 | 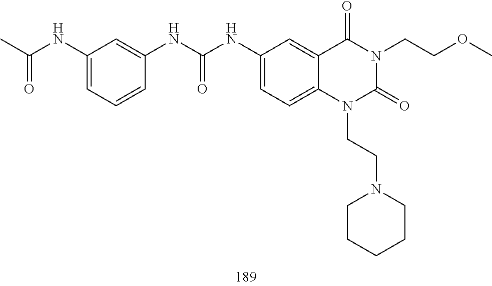 [SMILES: COCCn1c(=O)c2cc(NC(=O)Nc3cccc(NC(C)=O)c3)ccc2n(CCN2CCCCC2)c1=O] |
| 59 | 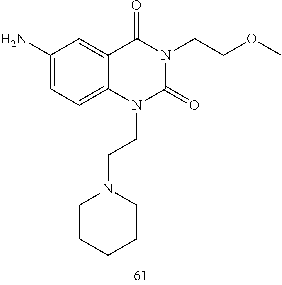 [SMILES: COCCn1c(=O)c2cc(N)ccc2n(CCN2CCCCC2)c1=O] | 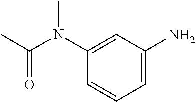 | 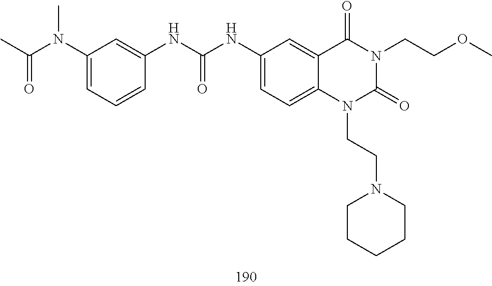 [SMILES: COCCn1c(=O)c2cc(NC(=O)Nc3cccc(N(C)C(C)=O)c3)ccc2n(CCN2CCCCC2)c1=O] |
| 60 | 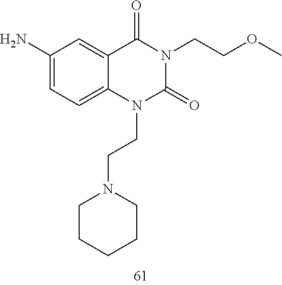 [SMILES: COCCn1c(=O)c2cc(N)ccc2n(CCN2CCCCC2)c1=O] | 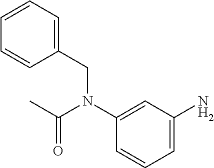 [SMILES: CC(=O)N(Cc1ccccc1)c1cccc(N)c1] | 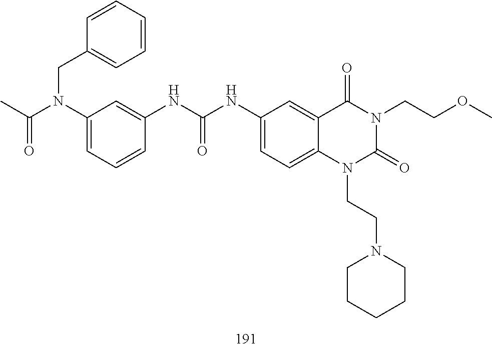 [SMILES: COCCn1c(=O)c2cc(NC(=O)Nc3cccc(N(Cc4ccccc4)C(C)=O)c3)ccc2n(CCN2CCCCC2)c1=O] |
| 61 | 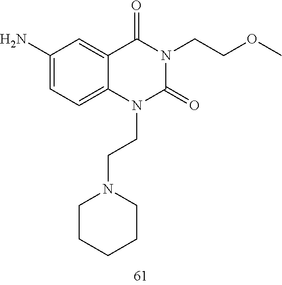 [SMILES: COCCn1c(=O)c2cc(N)ccc2n(CCN2CCCCC2)c1=O] | 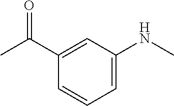 | 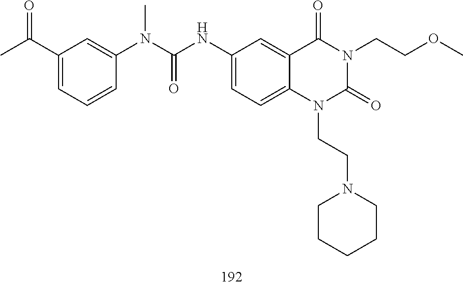 [SMILES: COCCn1c(=O)c2cc(NC(=O)N(C)c3cccc(C(C)=O)c3)ccc2n(CCN2CCCCC2)c1=O] |
| 62 | 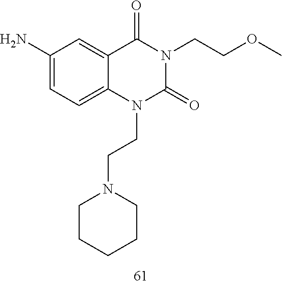 [SMILES: COCCn1c(=O)c2cc(N)ccc2n(CCN2CCCCC2)c1=O] | 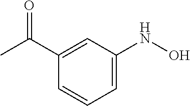 | 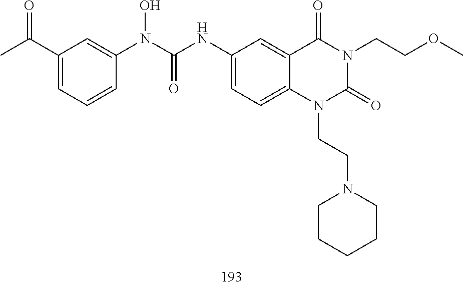 [SMILES: COCCn1c(=O)c2cc(NC(=O)N(O)c3cccc(C(C)=O)c3)ccc2n(CCN2CCCCC2)c1=O] |
| 63 | 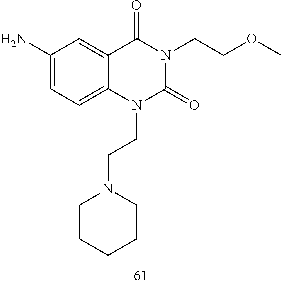 [SMILES: COCCn1c(=O)c2cc(N)ccc2n(CCN2CCCCC2)c1=O] | 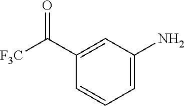 [SMILES: Nc1cccc(C(=O)C(F)(F)F)c1] | 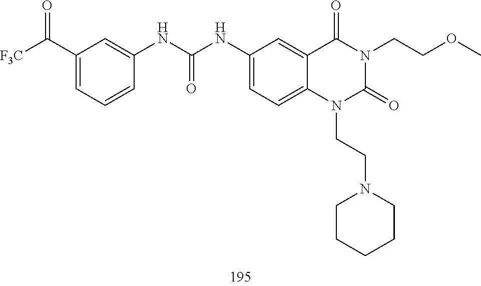 [SMILES: COCCn1c(=O)c2cc(NC(=O)Nc3cccc(C(=O)C(F)(F)F)c3)ccc2n(CCN2CCCCC2)c1=O] |
| 64 | 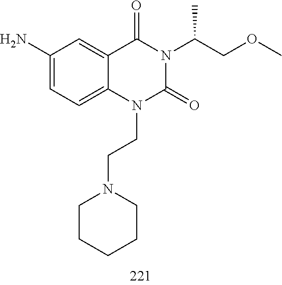 [SMILES: COC[C@@H](C)n1c(=O)c2cc(N)ccc2n(CCN2CCCCC2)c1=O] | 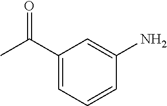 | 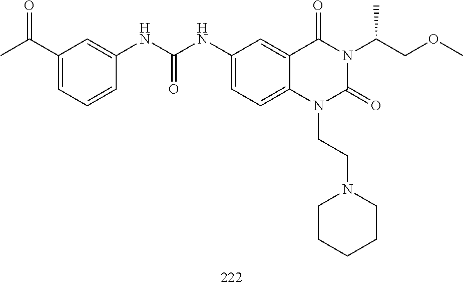 [SMILES: COC[C@@H](C)n1c(=O)c2cc(NC(=O)Nc3cccc(C(C)=O)c3)ccc2n(CCN2CCCCC2)c1=O] |
| 65 | 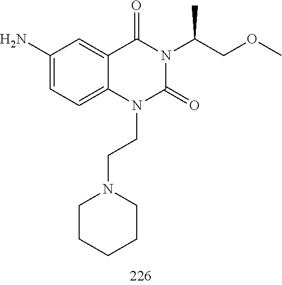 [SMILES: COC[C@H](C)n1c(=O)c2cc(N)ccc2n(CCN2CCCCC2)c1=O] | 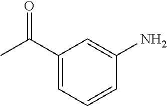 | 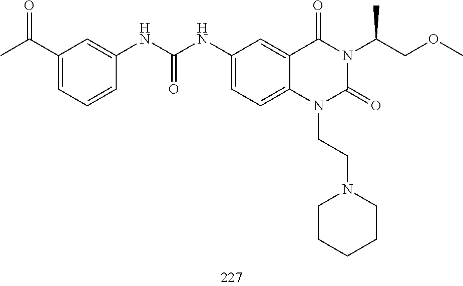 [SMILES: COC[C@H](C)n1c(=O)c2cc(NC(=O)Nc3cccc(C(C)=O)c3)ccc2n(CCN2CCCCC2)c1=O] |
| 66 | 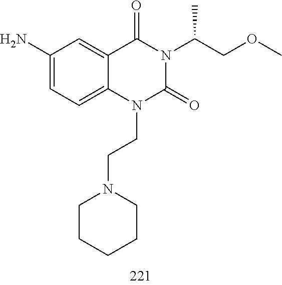 [SMILES: COC[C@@H](C)n1c(=O)c2cc(N)ccc2n(CCN2CCCCC2)c1=O] | 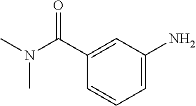 | 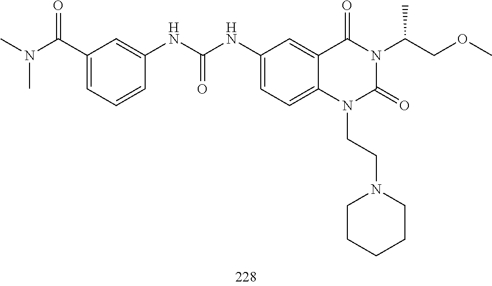 |
| 67 | 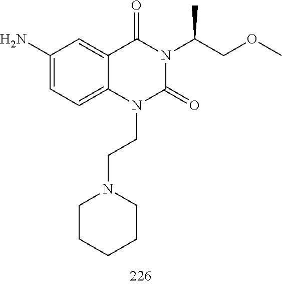 [SMILES: COC[C@H](C)n1c(=O)c2cc(N)ccc2n(CCN2CCCCC2)c1=O] | 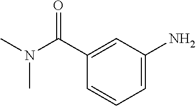 | 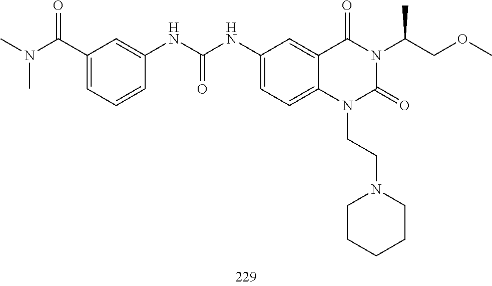 |
| 68 | 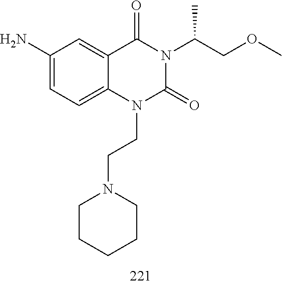 [SMILES: COC[C@@H](C)n1c(=O)c2cc(N)ccc2n(CCN2CCCCC2)c1=O] | 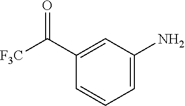 [SMILES: Nc1cccc(C(=O)C(F)(F)F)c1] | 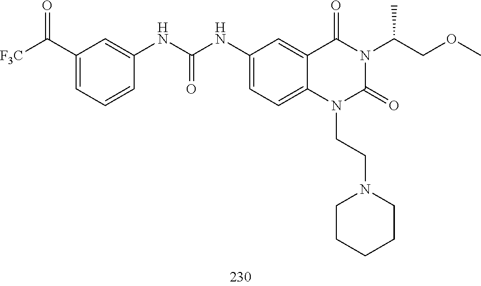 [SMILES: COC[C@@H](C)n1c(=O)c2cc(NC(=O)Nc3cccc(C(=O)C(F)(F)F)c3)ccc2n(CCN2CCCCC2)c1=O] |
| 69 | 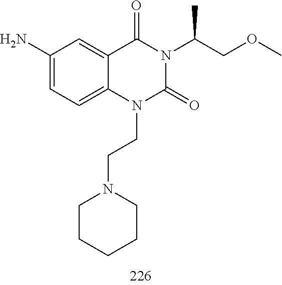 [SMILES: COC[C@H](C)n1c(=O)c2cc(N)ccc2n(CCN2CCCCC2)c1=O] | 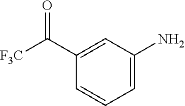 [SMILES: Nc1cccc(C(=O)C(F)(F)F)c1] | 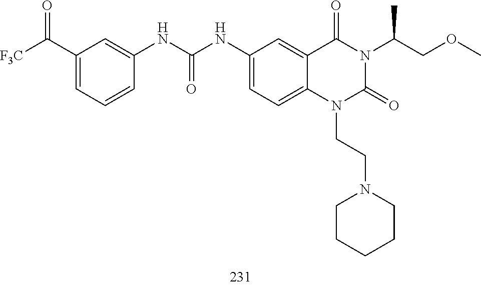 [SMILES: COC[C@H](C)n1c(=O)c2cc(NC(=O)Nc3cccc(C(=O)C(F)(F)F)c3)ccc2n(CCN2CCCCC2)c1=O] |

### Abbreviations

DMF N,N-dimethylformamide
TEA Triethylamine
MeOH Methanol
CHCl₃ Chloroform
Cs₂CO₃ Cesium carbonate
THE Tetrahydrofuran
Ar Argon
CDI Carbonyl diimidazole
HATU 1-[Bis(dimethylamino)methylene]-1H-1,2,3-triazolo[4,5-b]pyridinium 3-oxidchexafluorophosphate
LiOH·H₂O Lithium hydroxide monohydrate
PTSA p-Toluenesulfonic acid
NaCNBH₃ Sodium cyanoborohydride

In an embodiment of the present invention, there is provided a compound having structure I for use in treating diseases and disorders related to modulation of COP1 enzyme through its stabilization or modulation of ATGL.

In another embodiment of the present invention, there is provided a compound having structure I for use in decreasing the level of triglycerides in hepatocytes.

In yet another embodiment of the present invention, there is provided a compound having structure I for use in treatment of disease selected from Non-Alcoholic Fatty Liver Disease (NAFLD) or Non-Alcoholic Steatohepatitis (NASH).

In still another embodiment of the present invention, there is provided a composition comprising the compound having structure I along with pharmaceutically acceptable excipients.

Another embodiment of the present invention provides a method of modulation COP1 enzyme through its stabilization by the compound having structure I.

Yet another embodiment of the present invention provides a method of increasing the level of ATGL by the compound having structure I.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The following examples, which include preferred embodiments, will serve to illustrate the practice of this invention, it being understood that the particulars shown are by way of example and for purpose of illustrative discussion of preferred embodiments of the invention.

Temperatures are given in degree Celsius. The structures of final products, intermediates and starting materials are confirmed by standard analytical methods, spectroscopic characterization e.g., MS, NMR. Abbreviations used are those conventional in the art.

All starting materials, reagents, catalysts, building blocks, acids, bases, dehydrating agents and solvents utilized to synthesize the compounds of the present invention are either commercially available or can be produced by known organic synthesis methods in the art.

### Example 1

### General Procedure A: Amide Formation Reaction

5-Nitroanthranilic acid (1 mmol) was taken in DMF (1-2 mL) and HATU (1-1.2 equivalent) was added, and reaction mixture was allowed to stir at room temperature for 15 min-1 hour. Suitable substituted aliphatic or aromatic amine was added drop wise (1-1.5 equivalent) to the reaction mixture followed by TEA (2.5-3 equivalent) and the contents of the reaction mixture were stirred for another 45 min. Reaction was monitored by checking TLC. Upon completion, the reaction mixture was washed thoroughly with ice cold water to remove DMF and extracted with EtOAc. Column chromatography was performed to get the pure product.

### Example 2

### General Procedure B: Cyclization Using CDI

An amide compound (1 mmol) prepared by general procedure A provided in Example 1 was taken in DMF (1-2 mL) and CDI (1-1.5 equivalent) was added and heated at 110° C. for 8-12 hrs. Reaction was monitored by checking TLC. Upon completion, the reaction mixture was washed with water followed by extraction with EtOAc. Column chromatography was performed to get the pure product.

### Example 3

### General Procedure C: Substitution Reaction

The compound (quinazolinedione) prepared by general procedure B provided in Example 2 was taken in DMF (1-2 mL) and K₂CO₃ (1-1.5 equivalent) and suitable alkyl chloride (aromatic or aliphatic) was added and heated at 120° C. for 12 hrs.

Reaction was monitored by checking TLC. Upon completion, the reaction mixture was washed with water followed by extraction with EtOAc or chloroform. Column chromatography was performed to get the pure product.

### Example 4

### General Procedure D: Reduction

A compound prepared by general procedure B and C (1 mmol) provided in example 2 and 3 was dissolved in methanol (2-5 mL) and a pinch of 10% wet Pd-C was added. The reaction mixture was degassed by passing nitrogen and H₂ gas for 2-5 hours to get fully reduced compound. Reaction was thoroughly monitored by checking TLC. Upon completion of the reaction, Pd-C was filtered through celite bed and methanol was evaporated in vacuum to get the desired compound. Column chromatography was performed to get the pure product.

### Example 5

### General Procedure E: Urea Derivative Formation Via Chloroformate Intermediate

A compound prepared by general procedure C (1 mmol) provided in example 4 was dissolved in dry THF (5-10 mL). 4-nitrophenylchloroformate (1-1.5 equivalent) was added portion wise and reaction mixture was stirred for 15 min-3 hour till the amine got consumed. Reaction was monitored by checking TLC. Further, suitable amine (1-1.5 equivalent) was added to the reaction mixture followed by TEA (2-4 equivalent) and reaction mixture was stirred for another 2-8 hours. Upon completion of the reaction, reaction mass was evaporated in vacuum to remove THF and washed with satd. NaHCO₃ solution and extracted with EtOAc. Column chromatography was performed to get the pure product.

Preparation of compound 5, 8, and 11 (Scheme 1, Scheme 2, Scheme 3)

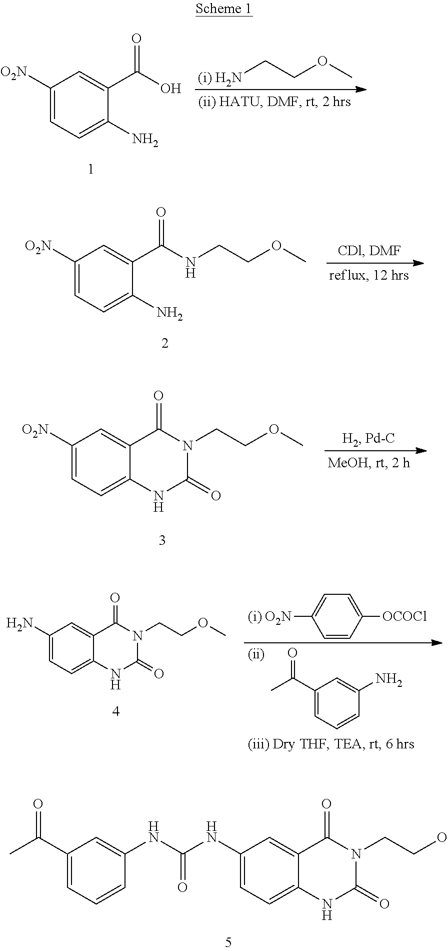

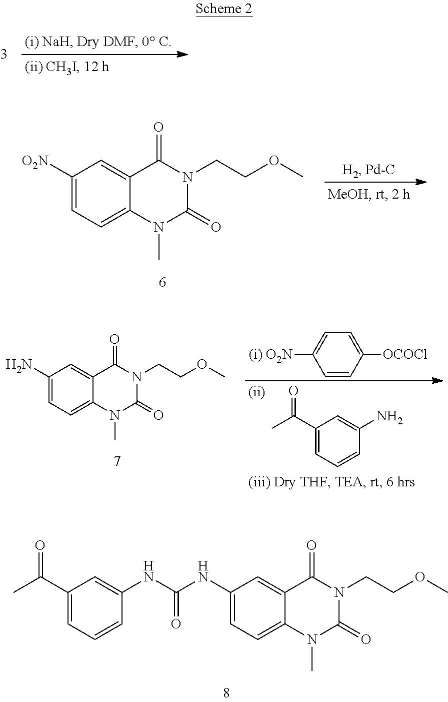

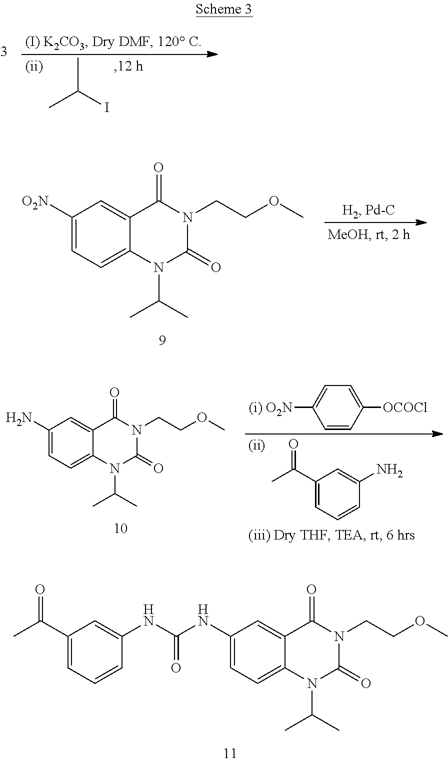

### Example 6

### Synthesis of 2-amino-N-(2-methoxyethyl)-5-nitrobenzamide (2) (DSR-01-27)

The following was made by general procedure A using 2-amino-5-nitrobenzoic acid 1 (4 g, 21.97 mmol), DMF (12 mL), HATU (9.1 g, 24.17 mmol), 2-methoxyethylamine (2.1 mL, 24.17 mmol) and TEA (7.6 mL, 54.93 mmol). After evaporation, the crude mass was diluted with chloroform and pet ether was added to obtain the precipitation. The precipitate was washed with pet ether to affordcompound 2 (4.2 g, 80%) as yellow solid.

¹H NMR (400 MHZ, d₆-DMSO) δ in ppm 8.71 (br.s, —NH), 8.46 (d, J=2.8 Hz, 1H), 7.96 (dd, J=9.4 Hz, 2.4 Hz, 1H), 7.71 (br.s, 2H), 6.75 (d, J=9.2 Hz, 1H), 3.43-3.40 (m, 2H), 3.37-3.33 (m, 2H), 3.23 (s, 3H).ESI-HRMS m/z 240.0995 (M+H⁺). Melting Point: 168° C.

### Example 7

### Synthesis of 2-hydroxy-3-(2-methoxyethyl)-6-nitroquinazolin-4 (3H)-one (3) (TSG-03-19)

Compound 2 was prepared by general procedure B (0.3 g, 1.25 mmol) and CDI (0.304 g, 1.88 mmol) were dissolved in DMF and stirred at 140° C. for 8 hours. After completion of the reaction, reaction mass was washed with water and extracted with ethyl acetate and purified by column chromatography (Silica gel, mesh size 100-200) eluting (50% Ethyl acetate-Pet Ether) to obtain compound 3 (0.286 g, 86%) as off white solid as shown in scheme 1.

¹H NMR (400 MHZ, DMSO-d6): δ=12.08 (s, 1H), 8.64-8.60 (m, 1H), 8.49-8.45 (m, 1H), 7.32 (d, J=12.0 Hz, 1H), 4.09 (t, J=8.0 Hz, 2H), 3.54 (t, J=8.0 Hz, 2H), 3.25 (s, 3H).ESI-HRMS m/z478.2463 (M+H⁺).

### Example 8

### Synthesis of 6-amino-2-hydroxy-3-(2-methoxyethyl) quinazolin-4 (3H)-one (4) (TSG-03-20)

The following compound was made by general procedure D using 3 (0.220 g, 0.83 mmol), methanol (5 mL) and pinch of 10% wet Pd-C to obtain compound 4 (0.121 g, 62%) as light brown solid shown in scheme 1.

¹H NMR (400 MHZ, DMSO-d6): δ=11.03 (s, 1H), 7.11-7.08 (m, 1H), 6.94-6.90 (m, 1H), 5.18 (s, 2H), 4.06 (t, J=8.4 Hz, 2H), 3.50 (t, J=8.0 Hz, 2H), 3.23 (s, 3H). ESI-HRMS m/z 236.1034 (M+H⁺).

### Example 9

### Synthesis of 1-(3-acetylphenyl)-3-(2-hydroxy-3-(2-methoxyethyl)-4-oxo-3,4-dihydroquinazolin-6-yl)urea (5) (TSG-03-23)

The following compound was made by general procedure E using 4 (0.080 g, 0.34 mmol), dry THF (3 mL), 4-nitrophenylchloroformate (0.082 g, 0.40 mmol), 3′-aminoacetophenone (0.054 g, 0.40 mmol), TEA (0.094 mL, 0.68 mmol) to obtain compound 5 (0.056 g, 45%) as light brown solid, depicted in scheme 1.

¹H NMR (400 MHZ, DMSO-d6), δ=11.29 (s, 1H), 8.85 (d, J=10.8 Hz, 1H), 8.07 (d, J=2.4 Hz, 1H), 8.04-8.02 (m, 1H), 7.64 (dd, J=9.2 Hz, 2.8 Hz, 2H), 7.55-7.53 (m, 1H), 7.41-7.37 (m, 1H), 7.09 (d, J=8.8 Hz, 1H), 4.05 (t, J=6.0 Hz, 2H), 3.49 (t, J=6.0 Hz, 2H), 3.20 (s, 3H), 2.52 (s, 3H).ESI-HRMS m/z397.1515 (M+H⁺).

### Example 10

### Synthesis of 3-(2-methoxyethyl)-1-methyl-6-nitroquinazoline-2,4 (1H,3H)-dione (6) (TSG-03-103)

Compound 3 (0.5 g, 2.00 mmol) was dissolved in dry DMF (5 mL) and then NaH (0.057 g, 2.4 mmol) was added at 0° C. keeping N2 atmosphere and the reaction mixture was allowed to stir at room temperature for 1 hr. Then CH₃I (0.36 ml, 2.4 mmol) was added drop wise at 0° C. and RM was allowed to stir at room temperature for overnight. Upon completion of the reaction, reaction mass was worked up with EtOAc and water, then purified by column chromatography (Silica gel, mesh size 100-200) eluting (50% EtOAc/Pet ether) to get compound 6 (0.442 g, 84%) as off-white solid, depicted in scheme 2.

### Example 11

### Synthesis of 6-amino-3-(2-methoxyethyl)-1-methylquinazoline-2,4 (1H,3H)-dione (7) (TSG-03-104)

Compound 6 (0.4 g, 1.5 mmol) was dissolved in methanol (8 mL) and pinch of 10% wet Pd-C was added. Reaction mixture was degassed by passing nitrogen and H₂ gas was passed for 2 hours to get fully reduced compound. Reaction was thoroughly monitored by checking TLC. Upon completion of the reaction, Pd-C was filtered through celite bed and methanol was evaporated in vacuum to obtain compound 7 (0.25 g, 72%) as light brown solid, shown in scheme 2.

### Example 12

### Synthesis of 1-(3-acetylphenyl)-3-(3-(2-methoxyethyl)-1-methyl-2,4-dioxo-1,2,3,4-tetrahydroquinazolin-6-yl)urea (8) (TSG-03-105)

Compound 7 (0.1 g, 0.4 mmol) was dissolved in dry THF (3 mL) and 4-Nitrophenylchloroformate (0.097 g, 0.48 mmol) was added portion wise and reaction mixture was stirred for 2 hours till consume of the amine. Reaction was monitored by checking TLC. 3′-aminoacetophenone (0.065 g, 0.48 mmol) was added followed by TEA (0.12 mL, 0.8 mmol) and reaction mixture was stirred for another 3 hours. Upon completion of the reaction, reaction mass was evaporated in vacuum to remove THE and washed with saturated NaHCO₃ solution and extracted with EtOAc to give yellow coloured crude mass which was then purified by column chromatography (Silica gel, mesh size 100-200) eluting (80% EtOAc/Pet ether) to get compound 8 (0.085 g, 52%) as off-white solid depicted in scheme 2.

¹H NMR (400 MHZ, d6-DMSO) δ in ppm 8.92 (s, 1H), 8.90 (s, 1H), 8.17 (d, 2.4 Hz, 1H), 8.04-8.02 (m, 1H), 7.77 (dd, J=9.2 Hz, 2.8 Hz, 1H), 7.66-7.63 (m, 1H), 7.56-7.53 (m, 1H), 7.42-7.34 (m, 2H), 4.10 (t, J=6.0 Hz, 2H), 3.49 (t, J=6.0 Hz, 2H), 3.45 (s, 3H), 3.20 (s, 3H), 2.52 (s, 3H). ESI-HRMS m/z 411.1659 (M+H⁺).

### Example 13

### Synthesis of 1-isopropyl-3-(2-methoxyethyl)-6-nitroquinazoline-2,4 (1H,3H)-dione (9) (TSG-03-106)

Compound 1 (0.3 g, 1.13 mmol), K₂CO₃ (0.314 g, 2.26 mmol), 2-iodopropane (0.288 g, 1.69 mmol) were taken in dry DMF in a pressure tube and stirred at 120° C. for overnight. Upon completion of the reaction, reaction mass was worked up with EtOAc and water, then purified by column chromatography (Silica gel, mesh size 100-200) eluting (50% EtOAc/Pet ether) to get compound 8 (0.225 g, 65%) as off white solid as shown in scheme 3.

¹H NMR (400 MHZ, CDCl3) δ in ppm 9.06 (d, J=2.8 Hz, 1H), 8.42 (dd, J=9.2 Hz, 2.58 Hz, 1H), 7.46 (d, J=9.2 Hz, 1H), 5.17-4.99 (m, 1H), 4.30 (t, J=6.0 Hz, 2H), 3.67 (t, J=6.0 Hz, 2H), 3.34 (s, 3H), 1.62 (d, J=6.8 Hz, 6H). ESI-HRMS m/z 308.1241 (M+H⁺).

### Example 14

### Synthesis of 6-amino-1-isopropyl-3-(2-methoxyethyl) quinazoline-2,4 (1H,3H)-dione (10) (TSG-03-112)

Compound 9 (0.180 g, 0.58 mmol) was dissolved in methanol (5 mL) and pinch of 10% wet Pd-C was added. Reaction mixture was degassed by passing nitrogen and H₂ gas was passed for 2 hours to get fully reduced compound. Reaction was thoroughly monitored by checking TLC. Upon completion of the reaction, Pd-C was filtered through celite bed and methanol was evaporated in vacuum to obtain compound 10(0.12 g, 75%) as light brown solid, depicted in scheme 3.

¹H NMR (400 MHZ, CDCl₃) δ in ppm 7.49 (d, J=2.8 Hz, 1H), 7.17 (d, J=8.8 Hz, 1H), 6.97 (dd, J=8.8 Hz, 2.8 Hz, 1H), 5.05-4.92 (m, 1H), 4.28 (t, J=6.0 Hz, 2H), 3.74 (s, 2H), 3.64 (t, J=6.0HZ, 2H), 3.35 (s, 3H), 1.56 (d, 6H). ESI-HRMS m/z 278.1503 (M+H⁺).

### Example 15

### Synthesis of 1-(3-acetylphenyl)-3-(1-isopropyl-3-(2-methoxyethyl)-2,4-dioxo-1,2,3,4-tetrahydroquinazolin-6-yl)urea(11) (TSG-03-114)

Compound 10 (0.08 g, 0.39 mmol) was dissolved in dry THF (3 mL) and 4-Nitrophenylchloroformate (0.095 g, 0.48 mmol) was added portion wise and reaction mixture was stirred for 2 hour till consume of the amine. Reaction was monitored by checking TLC. 3′-aminoacetophenone (0.065 g, 0.48 mmol) was added followed by TEA (0.12 mL, 0.8 mmol) and reaction mixture was stirred for another 3 hours. Upon completion of the reaction, reaction mass was evaporated in vacuum to remove THF and washed with saturated. NaHCO₃ solution and extracted with EtOAc to give yellow coloured crude mass which was then purified by column chromatography (Silica gel, mesh size 100-200) eluting (70% EtOAc/Pet ether) to get compound 11 (0.057 g, 35%) as off-white solid, depicted in scheme 3.

¹H NMR (400 MHZ, CDCl₃) δ in ppm 8.36 (s, 1H), 8.26 (dd, J=9.2 Hz, 2.8 Hz, 1H), 8.17 (s, 1H), 7.92 (d, J=2.8 Hz, 1H), 7.89-7.87 (m, 1H), 7.86-7.83 (m, 1H), 7.61-7.58 (m, 1H), 7.40-7.38 (m, 1H), 7.33 (d, J=9.2 Hz, 1H), 5.08-4.93 (m, 1H), 4.32 (t, J=5.6 Hz, 2H), 3.71 (t, J=5.6 Hz, 2H), 3.29 (s, 3H), 2.60 (s, 3H), 1.56 (d, J=6.8 Hz, 6H). ESI-HRMS m/z 439.1971 (M+H⁺).

### Preparation of Compound 14, 17, 20, 23, 26, 29, 32, 35, 38, 41, 44, 47, 50, 53 and 56 (Scheme 4-Scheme 18)

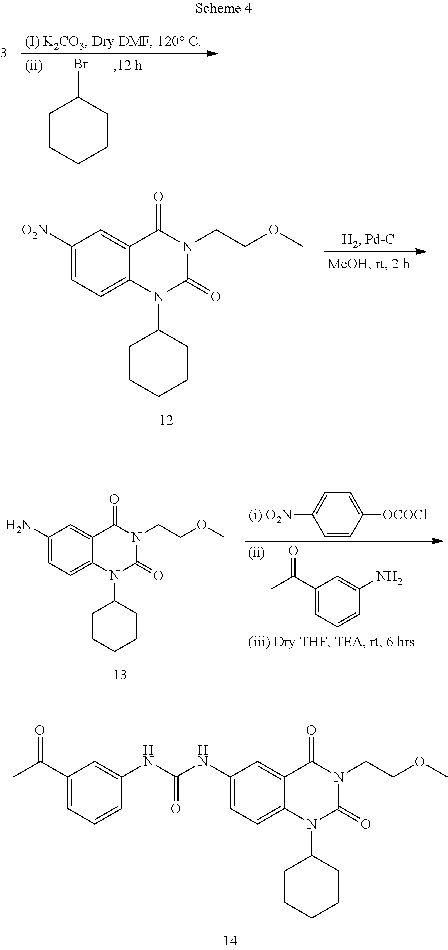

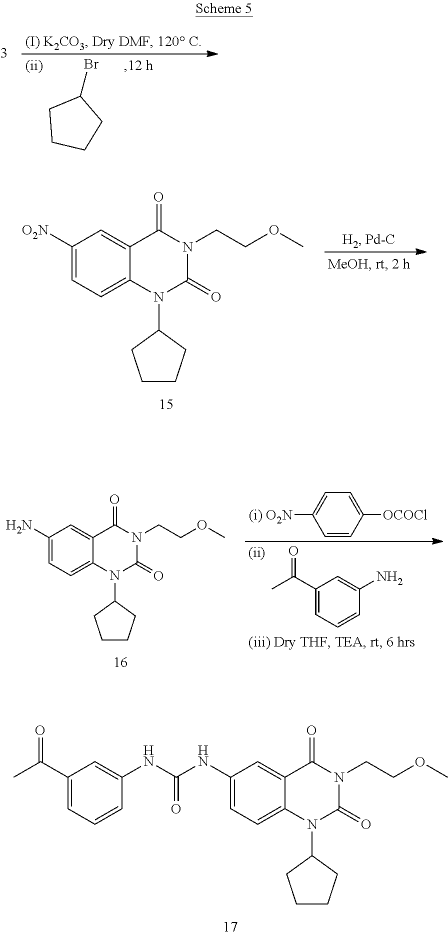

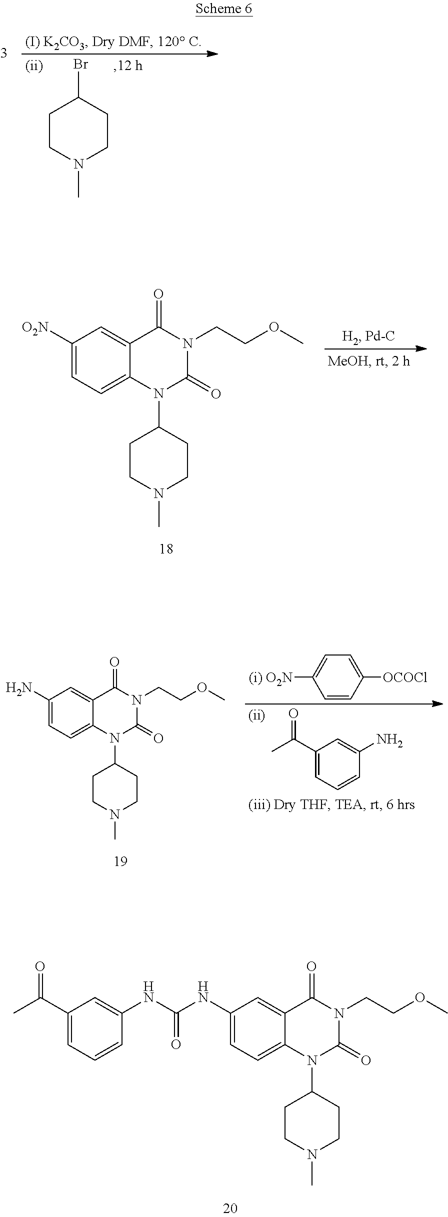

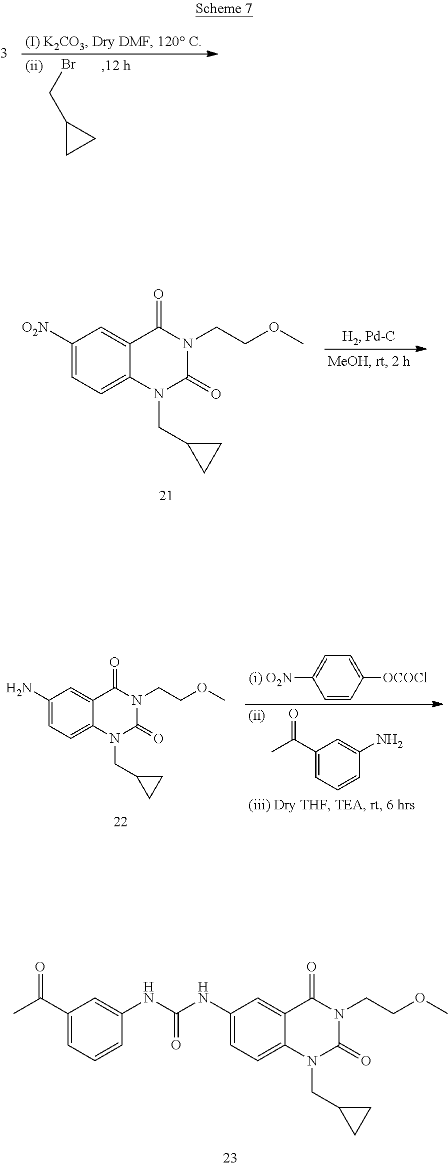

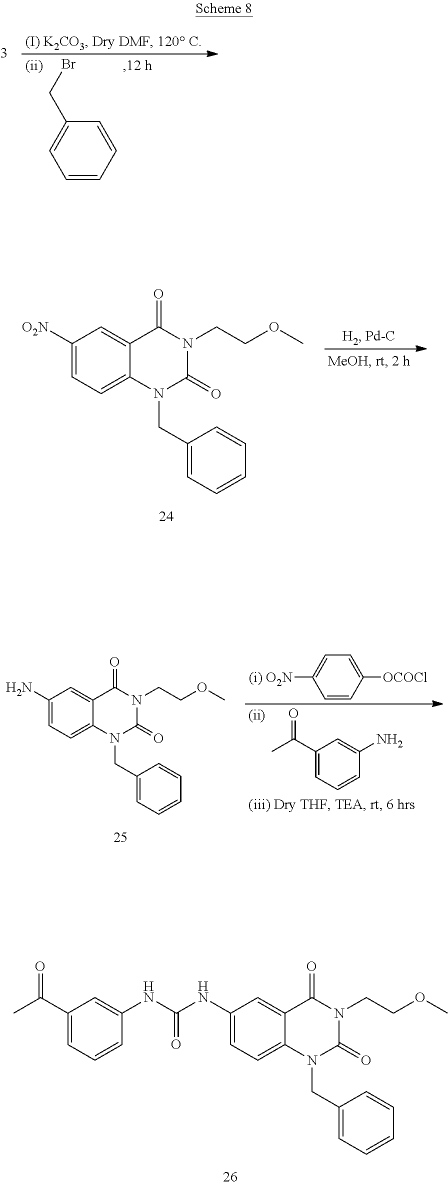

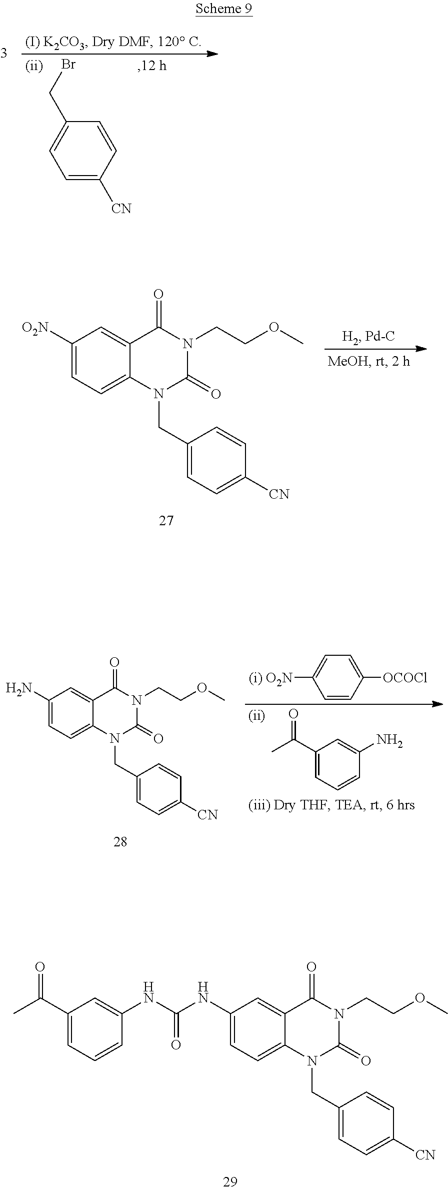

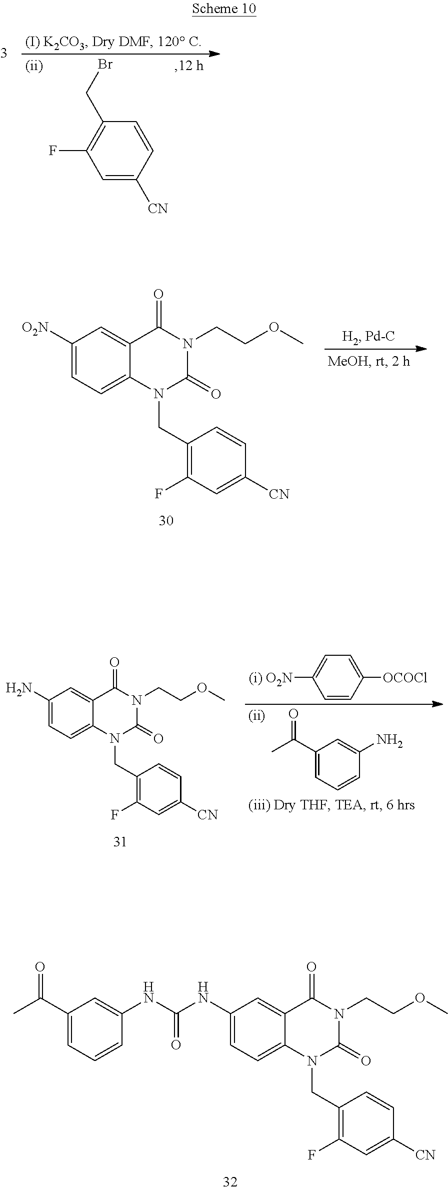

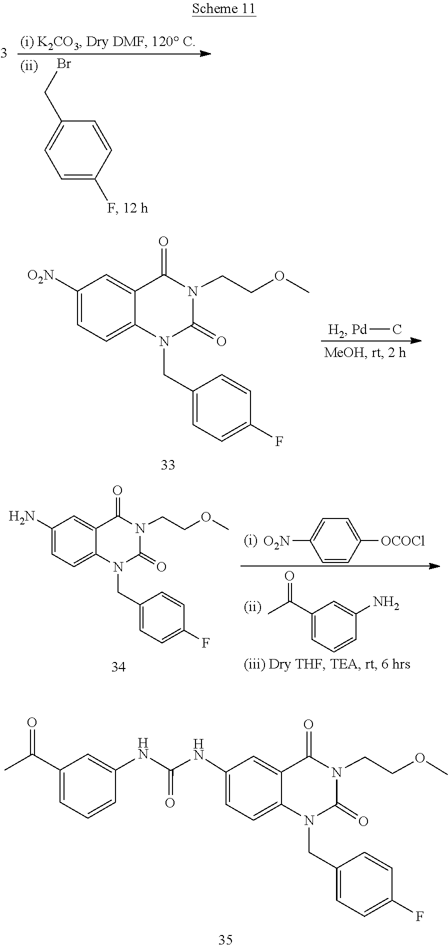

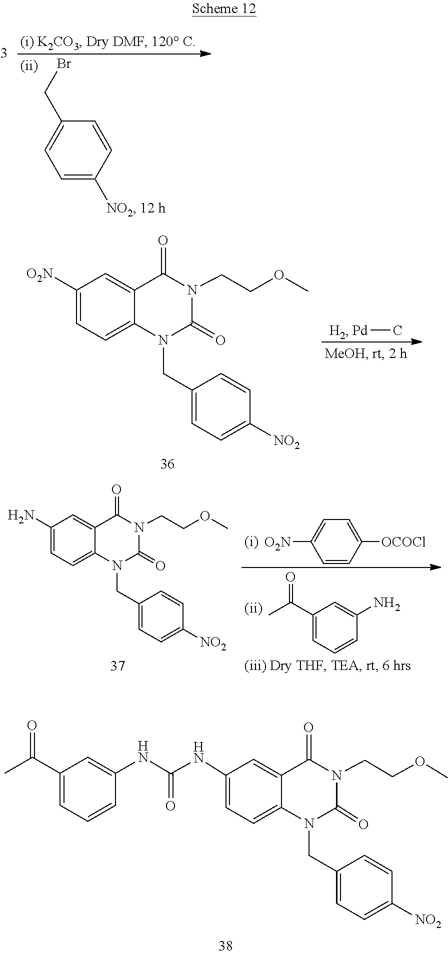

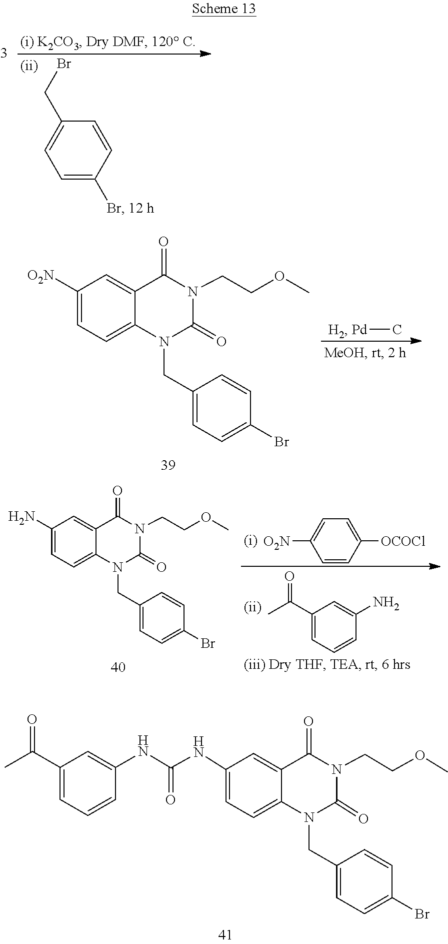

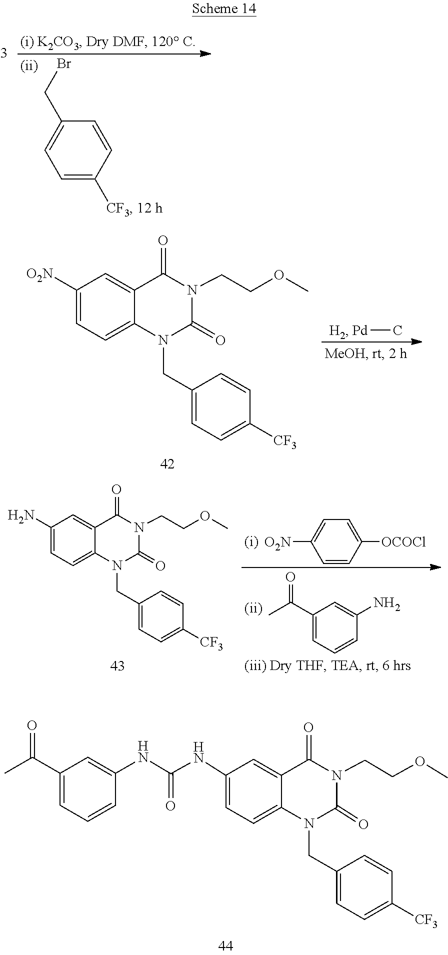

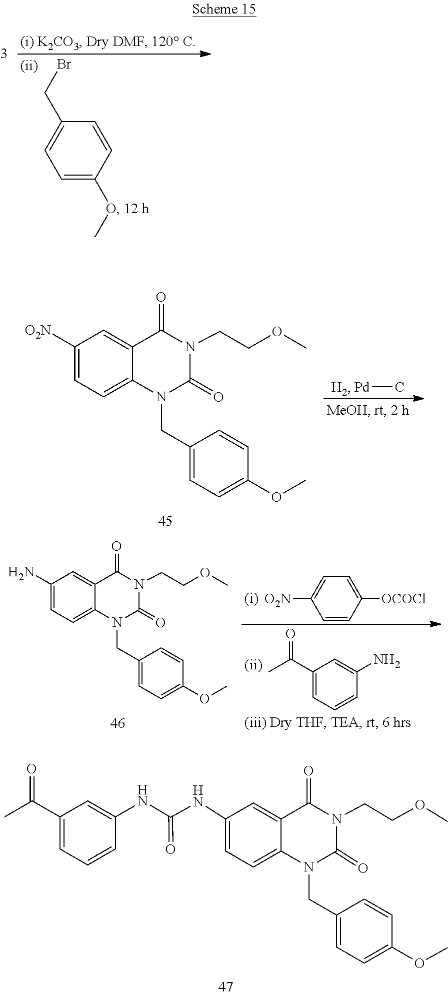

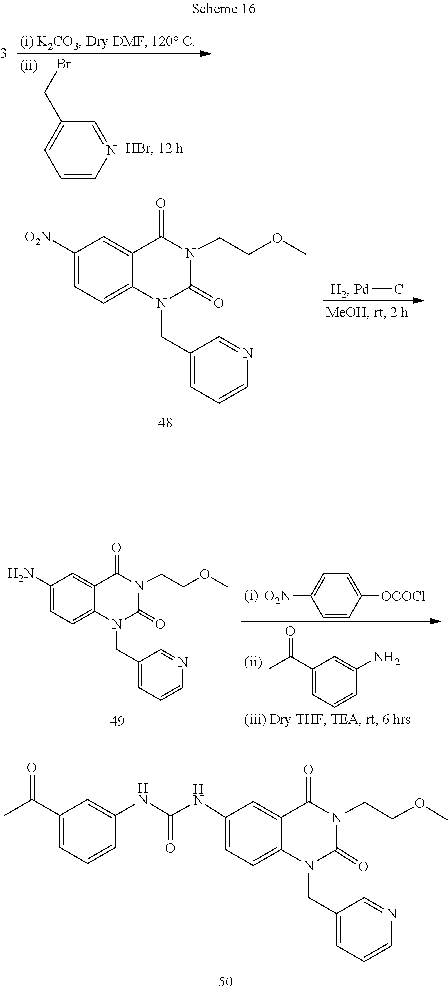

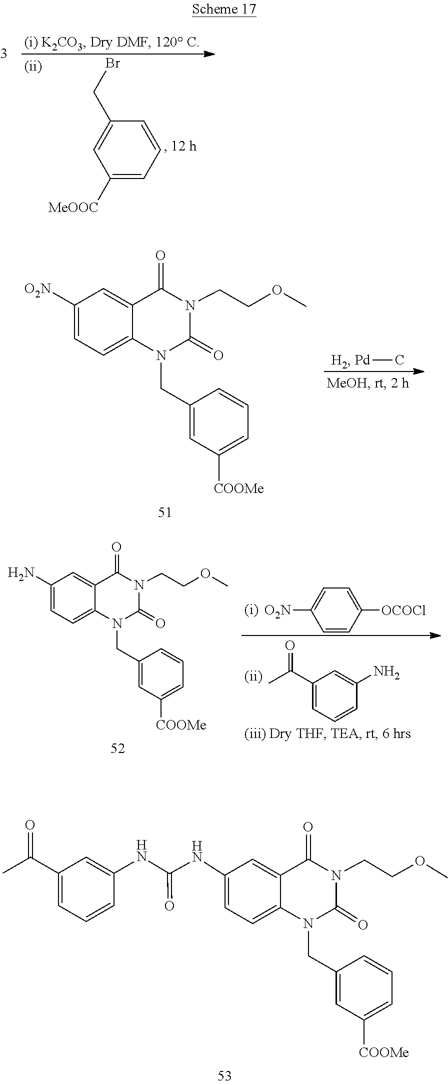

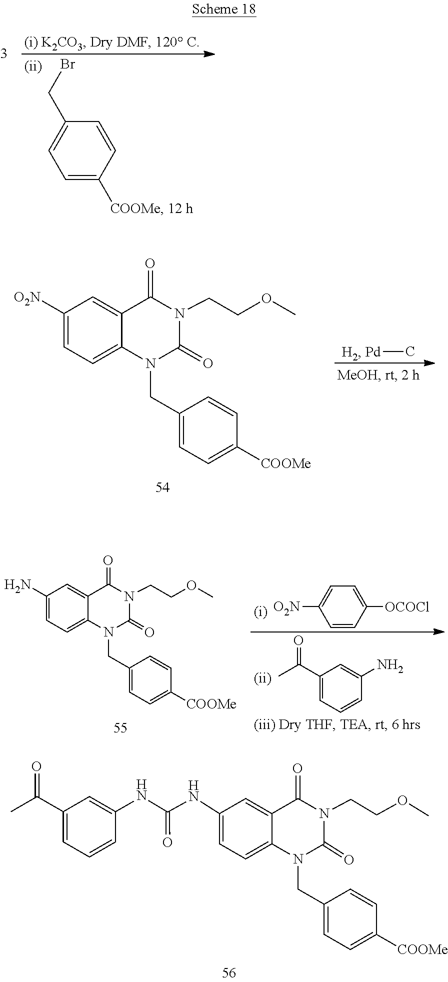

### Example 16

Preparation of compound 59, 62 (Scheme 19, 20)

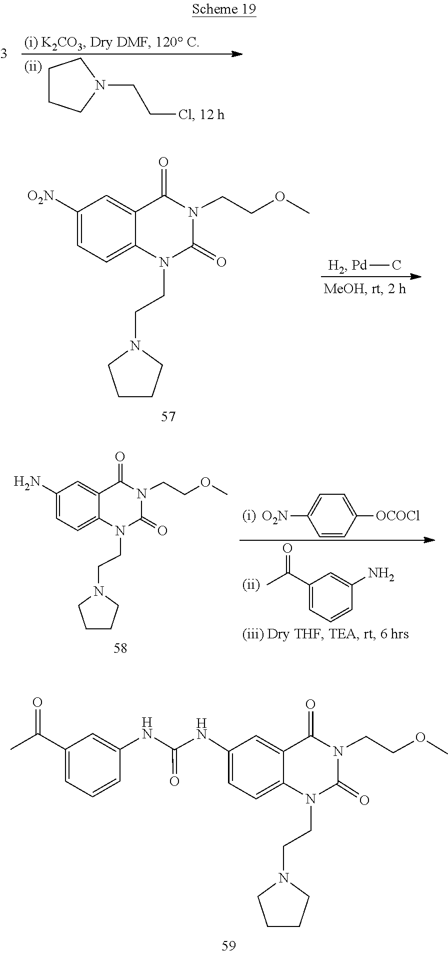

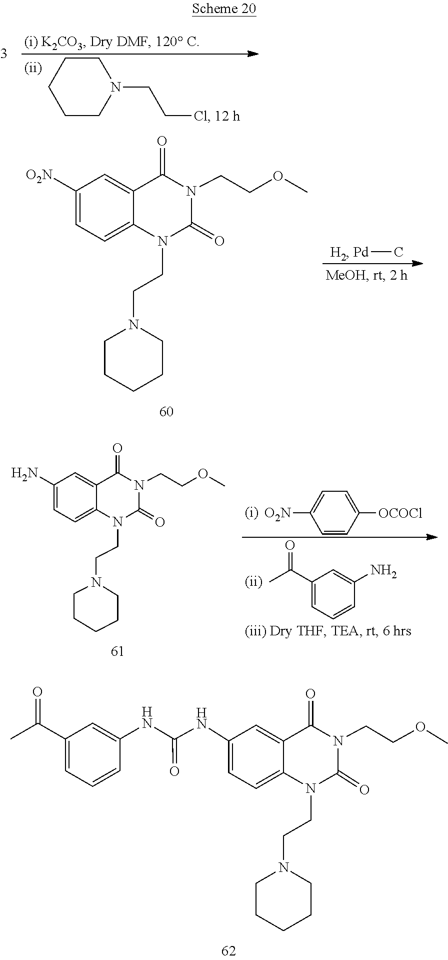

### Synthesis of 3-(2-methoxyethyl)-6-nitro-1-(2-(pyrrolidin-1-yl)ethyl) quinazoline-2,4 (1H,3H)-dione (57) (TSG-03-130)

Compound 3 (0.4 g, 1.50 mmol) was dissolved in dry DMF (5 mL) and then sodium tert-butoxide (0.29 g, 3 mmol) was added and the reaction mass was allowed to stir at room temperature for 1 hr. Then a mixture of 1-(2-Chloroethyl) pyrrolidine hydrochloride (0.403 g, 2.25 mmol) in dry TEA (1 mL) was added and the reaction mass and was stirred at 100° C. for overnight. Upon completion of the reaction, reaction mass was worked up with EtOAc and water, then purified by column chromatography (Silica gel, mesh size 100-200) eluting (3% MeOH/CHCl₃) to get compound 57 (0.409 g, 75%) as off-white solid, depicted in scheme 19.

¹H NMR (400 MHZ, CDCl₃) δ in ppm 9.04 (d, J=2.8 Hz, 1H), 8.46 (dd, J=9.2 Hz, 2.8 Hz, 1H), 7.46 (d, J=9.2 Hz, 1H), 4.36-4.30 (m, 4H), 3.67 (t, J=5.6 Hz, 2H), 3.33 (s, 3H), 2.84-2.80 (m, 2H), 2.70-2.67 (m, 4H), 1.83-1.80 (m, 4H). ESI-HRMS m/z 363.1665 (M+H⁺).

### Example 17

### Synthesis of 6-amino-3-(2-methoxyethyl)-1-(2-(67yrrolidine-1-yl)ethyl) quinazoline-2,4 (1H,3H)-dione (58) (TSG-03-132)

Compound 57 (0.2 g, 0.47 mmol) was dissolved in methanol (5 mL) and pinch of 10% wet Pd-C was added. Reaction mixture was degassed by passing nitrogen and H₂ gas was passed for 2 hours to get fully reduced compound. Reaction was thoroughly monitored by checking TLC. Upon completion of the reaction, Pd-C was filtered through celite bed and methanol was evaporated in vacuum to obtain compound 58 (0.110 g, 60%) as light brown solid, depicted in scheme 19.

¹H NMR (400 MHZ, CDCl₃) δ in ppm 7.62 (d, J=8.8 Hz, 1H), 7.41 (d, J=2.4 Hz, 1H), 7.09 (dd, J=8.8 Hz, 2.8 Hz, 1H), 4.61 (t, J=8.0 Hz, 2H), 4.25 (t, J=6.0 Hz, 2H), 3.62 (t, J=6.0 Hz, 2H), 3.31 (s, 3H), 3.29-3.27 (m, 2H), 2.17-2.11 (m, 4H). ESI-HRMS m/z 333.1929 (M+H⁺).

### Example 18

### Synthesis of 1-(3-acetylphenyl)-3-(3-(2-methoxyethyl)-2,4-dioxo-1-(2-(pyrrolidin-1-yl)ethyl)-1,2,3,4-tetrahydroquinazolin-6-yl)urea (59) (TSG-03-134)

Compound 58 (0.08 g, 0.23 mmol) was dissolved in dry THF (3 mL) and 4-Nitrophenylchloroformate (0.069 g, 0.34 mmol) was added portion wise and reaction mixture was stirred for 2 hours till consume of the amine. Reaction was monitored by checking TLC. 3′-aminoacetophenone (0.046 g, 0.34 mmol) was added followed by TEA (0.06 mL, 0.46 mmol) and reaction mixture was stirred for another 3 hours. Upon completion of the reaction, reaction mass was evaporated in vacuum to remove THF and washed with satd. NaHCO₃ solution and extracted with EtOAc to give yellow coloured crude mass which was then purified by column chromatography (Silica gel, mesh size 100-200) eluting (10% MeOH/CHCl₃) to get compound 59 (0.049 g, 42%) as off-white solid.

¹H NMR (400 MHZ, CDCl₃) δ in ppm 8.74 (s, 1H), 8.50 (s, 1H), 8.02-7.99 (m, 1H), 7.87 (d, J=2.4 Hz, 1H), 7.80-7.77 (m, 1H), 7.66-7.64 (m, 1H), 7.54-7.51 (m, 1H), 7.33-7.29 (m, 1H), 7.12-7.09 (m, 1H), 4.33 (t, J=6.4 Hz, 2H), 4.21 (t, J=5.2 Hz, 2H), 3.61 (t, J=5.2 Hz, 2H), 3.28 (s, 3H), 3.16-3.13 (m, 2H), 3.04-2.98 (m, 4H), 2.55 (s, 3H), 1.94-1.89 (m, 4H). ESI-HRMS m/z 449.2403 (M+H⁺).

### Example 19

### Synthesis of 3-(2-methoxyethyl)-6-nitro-1-(2-(piperidin-1-yl)ethyl) quinazoline-2,4 (1H,3H)-dione (60) (TSG-03-107)

Compound 3 (0.3 g, 1.13 mmol), K₂CO₃ (0.312 g, 2.26 mmol), 1-(2-Chloroethyl) piperidine hydrochloride (0.312 g, 1.69 mmol) were taken in dry DMF in a pressure tube and stirred at 120° C. for overnight. Upon completion of the reaction, reaction mass was worked up with EtOAc and water, then purified by column chromatography (Silica gel, mesh size 100-200) eluting (80% EtOAc/Pet ether) to get compound 60 (0.289 g, 68%) as off white solid.

¹H NMR (400 MHZ, CDCl₃) δ in ppm 9.03 (d, J=2.8 Hz, 1H), 8.47 (dd, J=9.2 Hz, 2.8 Hz, 1H), 7.53 (d, J=9.2 Hz, 1H), 4.35 (t, J=7.2 Hz, 2H), 4.31 (t, J=5.6 Hz, 2H), 3.67 (t, J=5.6 Hz, 2H), 3.33 (s, 3H), 2.69 (t, J=7.6 Hz, 2H), 2.59-2.57 (m, 4H), 1.64-1.58 (m, 4H), 1.48-1.43 (m, 2H). ESI-HRMS m/z 377.1837 (M+H⁺).

### Example 20

### Synthesis of 6-amino-3-(2-methoxyethyl)-1-(2-(piperidin-1-yl)ethyl) quinazoline-2,4 (1H,3H)-dione (61) (TSG-03-116)

Compound 60 (0.2 g, 0.51 mmol) was dissolved in methanol (5 mL) and pinch of 10% wet Pd-C was added. Reaction mixture was degassed by passing nitrogen and H₂ gas was passed for 2 hours to get fully reduced compound. Reaction was thoroughly monitored by checking TLC. Upon completion of the reaction, Pd-C was filtered through celite bed and methanol was evaporated in vacuum to obtain compound 61 (0.118 g, 64%) as light brown solid.

¹H NMR (400 MHZ, DMSO-d6) δ in ppm 7.18 (d, J=2.8 Hz, 2H), 6.98 (dd, J=8.8 Hz, 2.8 Hz, 1H), 5.25 (br.s 2H), 4.18-4.09 (m, 2H), 4.06 (t, J=6 Hz, 2H), 3.47 (t, J=6 Hz, 2H), 3.31-3.26 (m, 4H), 3.19 (s, 3H), 2.55-2.48 (m, 2H), 1.54-1.44 (m, 4H), 1.40-1.30 (m, 2H). ESI-HRMS m/z 347.2078 (M+H⁺).

### Example 21

### Synthesis of 1-(3-acetylphenyl)-3-(3-(2-methoxyethyl)-2,4-dioxo-1-(2-(piperidin-1-yl)ethyl)-1,2,3,4-tetrahydroquinazolin-6-yl)urea (62) (TSG-03-117)

Compound 61(0.08 g, 0.23 mmol) was dissolved in dry THF (3 mL) and 4-Nitrophenylchloroformate (0.071 g, 0.34 mmol) was added portion wise and reaction mixture was stirred for 2 hour till consume of the amine. Reaction was monitored by checking TLC. 3′-aminoacetophenone (0.048 g, 0.34 mmol) was added followed by TEA (0.07 mL, 0.46 mmol) and reaction mixture was stirred for another 3 hours. Upon completion of the reaction, reaction mass was evaporated in vacuum to remove THF and washed with satd. NaHCO₃ solution and extracted with EtOAc to give yellow coloured crude mass which was then purified by column chromatography (Silica gel, mesh size 100-200) eluting (10% MeOH/CHCl₃) to get compound 62 (0.057 g, 48%) as off white solid.

¹H NMR (400 MHZ, DMSO-d6) δ in ppm 9.11-9.05 (m, 2H), 8.18 (d, J=2.4 Hz, 1H), 8.04-8.03 (m, 1H), 7.76 (dd, J=8.8 Hz, 2.4 Hz, 1H), 7.66-7.63 (m, 1H), 7.55 (d, J=7.6 Hz, 1H), 7.41-7.38 (m, 2H), 4.24-4.19 (m, 2H), 4.10 (t, J=6.0 Hz, 2H), 3.50 (t, J=6.0 Hz, 2H), 3.51-3.48 (m, 6H), 3.20 (s, 3H), 2.52 (s, 3H), 1.54-1.42 (m, 4H), 1.39-1.29 (m, 2H). ESI-HRMS m/z 508.2555 (M+H⁺).

### Preparation of Compound 65, 68, 72, 75, 76, 79, 82, 85, and 88 (Schemes 21-29)

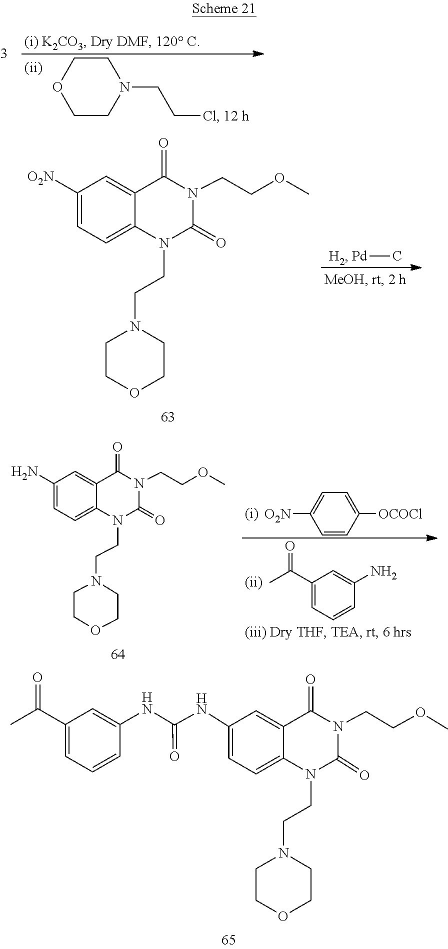

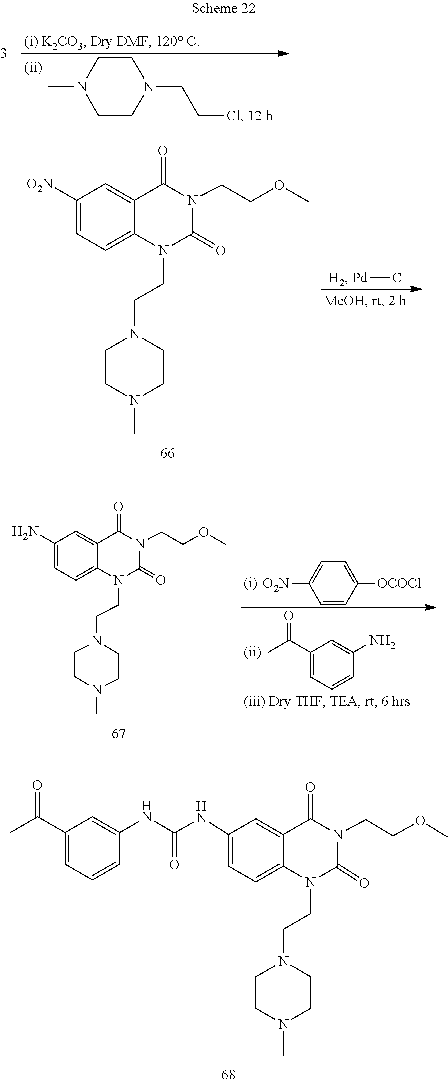

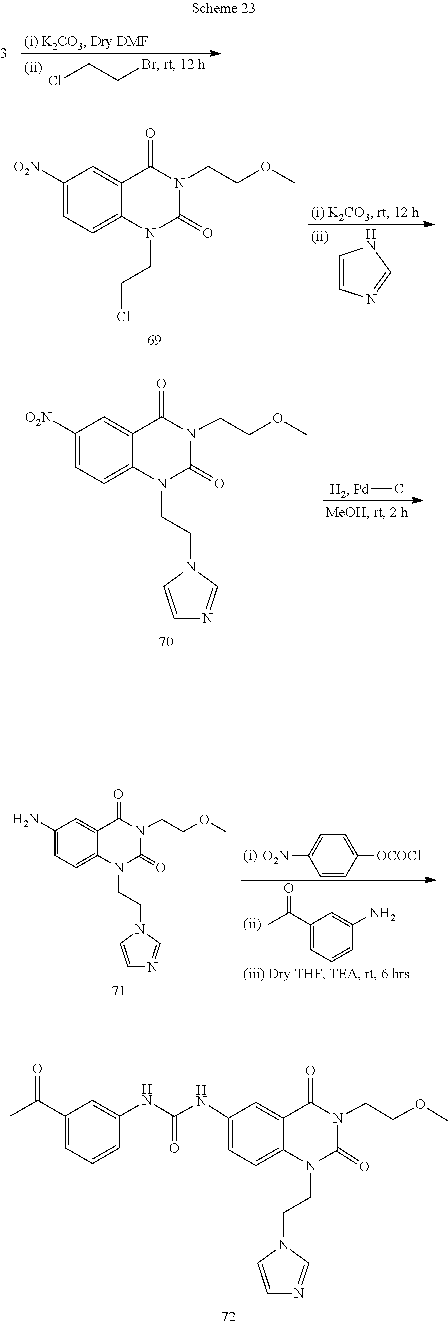

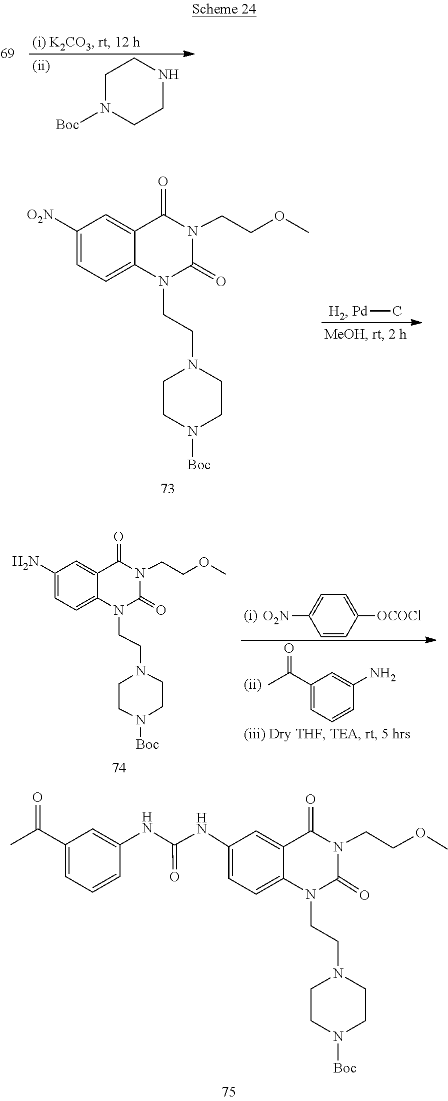

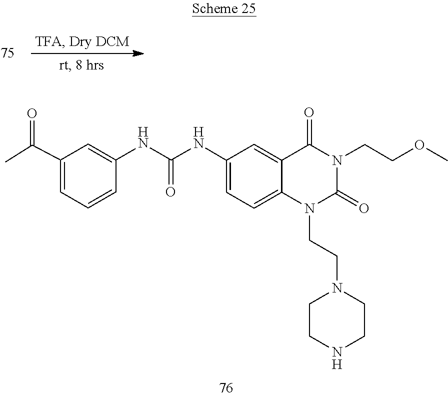 [SMILES: COCCn1c(=O)c2cc(NC(=O)Nc3cccc(C(C)=O)c3)ccc2n(CCN2CCNCC2)c1=O]

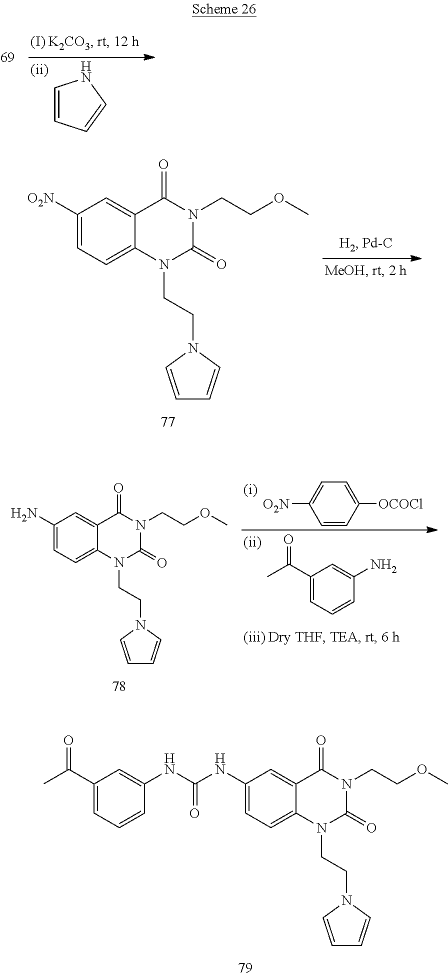

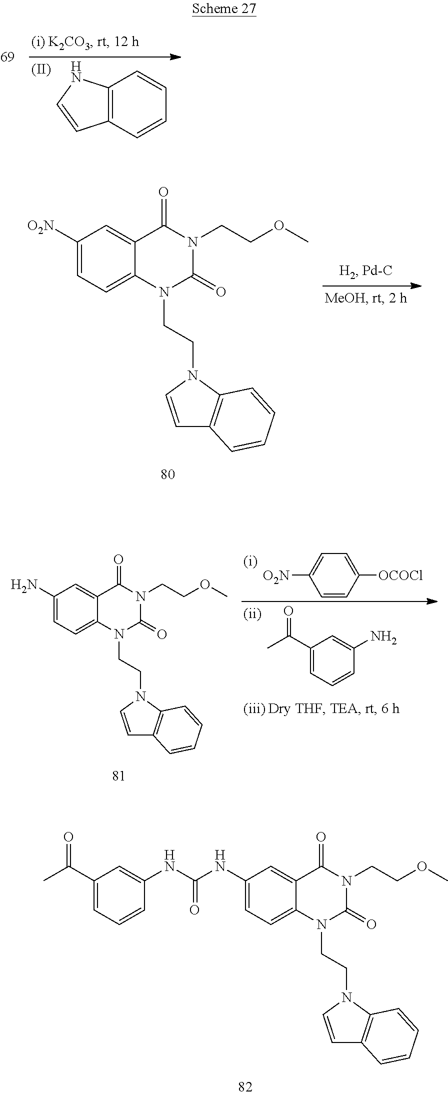

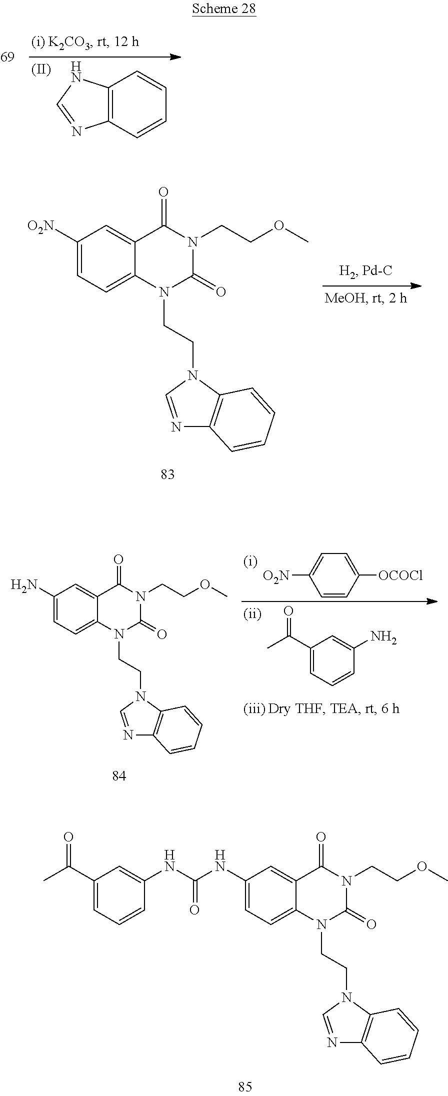

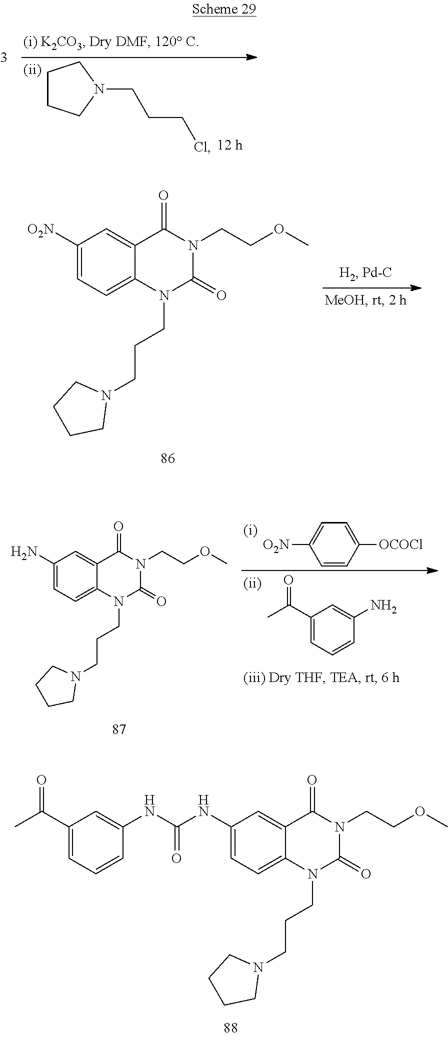

### Example 22

### Synthesis of 3-(2-methoxyethyl)-1-(2-morpholinoethyl)-6-nitroquinazoline-2,4 (1H,3H)-dione (63) (TSG-03-129)

Compound 3 (0.4 g, 1.50 mmol) was dissolved in dry DMF (5 mL) and then sodium tert-butoxide (0.29 g, 3 mmol) was added and the reaction mass was allowed to stir at room temperature for 1 hr. Then a mixture of 4-(2-Chloroethyl) morpholine hydrochloride (0.421 g, 2.25 mmol) in dry TEA (1 mL) was added and the reaction mass and was stirred at 100° C. for overnight. Upon completion of the reaction, reaction mass was worked up with EtOAc and water, then purified by column chromatography (Silica gel, mesh size 100-200) eluting (3% MeOH/CHCl₃) to get compound 63 (0.225 g, 65%) as off-white solid.

¹H NMR (400 MHZ, CDCl₃) δ in ppm 9.06 (d, J=2.8 Hz, 1H), 8.47 (dd, J=9.2 Hz, 2.4 Hz, 1H), 7.35 (d, J=9.2 Hz, 1H), 4.34-4.29 (m, 4H), 3.69-3.65 (m, 6H), 3.34 (s, 3H), 2.66 (t, J=6.8 Hz, 2H), 2.55-2.53 (m, 4H). ESI-HRMS m/z 379.1611 (M+H⁺).

### Example 23

### Synthesis of 6-amino-3-(2-methoxyethyl)-1-(2-morpholinoethyl) quinazoline-2,4 (1H,3H)-dione (64) (TSG-03-131)

Compound 63 (0.180 g, 0.47 mmol) was dissolved in methanol (5 mL) and pinch of 10% wet Pd-C was added. Reaction mixture was degassed by passing nitrogen and H₂ gas was passed for 2 hours to get fully reduced compound. Reaction was thoroughly monitored by checking TLC. Upon completion of the reaction, Pd-C was filtered through celite bed and methanol was evaporated in vacuum to obtain compound 64 (0.107 g, 65%) as light brown solid.

¹H NMR (400 MHZ, CDCl₃) δ in ppm 7.45 (d, J=2.4 Hz, 1H), 7.05-6.95 (m, 2H), 4.29 (t, J=6.0 Hz, 2H), 4.20 (t, J=7.2 Hz, 2H), 3.77 (s, 2H), 3.69-3.64 (m, 6H), 3.34 (s, 3H), 2.62 (t, J=7.2 Hz, 2H), 2.56-2.54 (m, 4H). ESI-HRMS m/z 349.1877 (M+H⁺).

### Example 24

### Synthesis of 1-(3-acetylphenyl)-3-(3-(2-methoxyethyl)-1-(2-morpholinoethyl)-2,4-dioxo-1,2,3,4-tetrahydroquinazolin-6-yl)urea (65) (TSG-03-133)

Compound 64(0.08 g, 0.23 mmol) was dissolved in dry THF (3 mL) and 4-Nitrophenylchloroformate (0.069 g, 0.34 mmol) was added portion wise and reaction mixture was stirred for 2 hour till consume of the amine. Reaction was monitored by checking TLC. 3′-aminoacetophenone (0.046 g, 0.34 mmol) was added followed by TEA (0.06 mL, 0.46 mmol) and reaction mixture was stirred for another 3 hours. Upon completion of the reaction, reaction mass was evaporated in vacuum to remove THF and washed with satd. NaHCO₃ solution and extracted with EtOAc to give yellow coloured crude mass which was then purified by column chromatography (Silica gel, mesh size 100-200) eluting (70% EtOAc/Pet ether) to get compound 65 (0.052 g, 45%) as off white solid. ¹H NMR (400 MHZ, CDCl₃) δ in ppm 8.32 (s, 1H), 8.24 (dd, J=9.2 Hz, 2.4 Hz, 1H), 8.14 (s, 1H), 7.89 (d, J=2.8 Hz, 1H), 7.87-7.84 (m, 2H), 7.61-7.58 (m, 1H), 7.41-7.36 (m, 1H), 7.18 (d, J=9.2 Hz, 1H), 4.32 (t, J=5.6 Hz, 2H), 4.22 (t, J=6.8 Hz, 2H), 3.72-3.66 (m, 6H), 3.30 (s, 3H), 2.65-2.62 (m, 2H), 2.61 (s, 3H), 2.57-2.53 (m, 4H). ESI-HRMS m/z 510.2355 (M+H⁺).

### Preparation of Compounds 91, 94, 97, 102, 107, 112, 117, 122, 127, 132, 137, 142, 147, 152, 157, 162, 167, 172, 177-193 (Schemes 30-59)

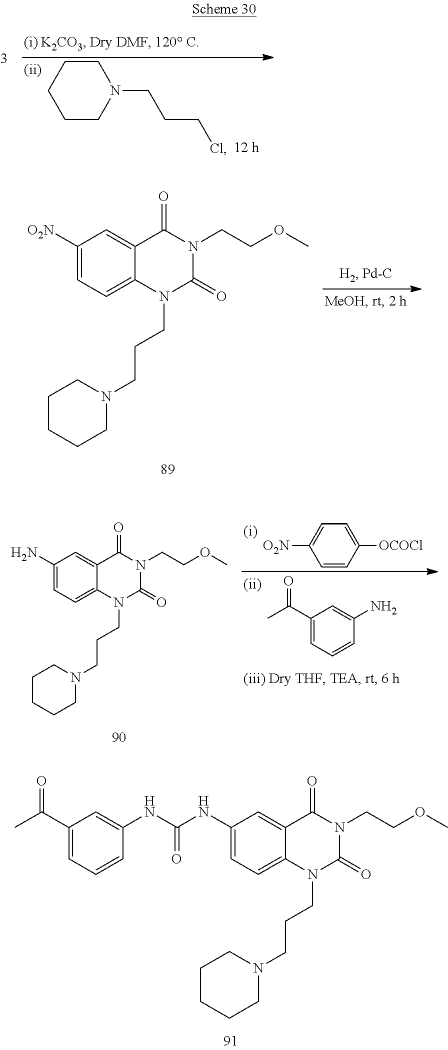

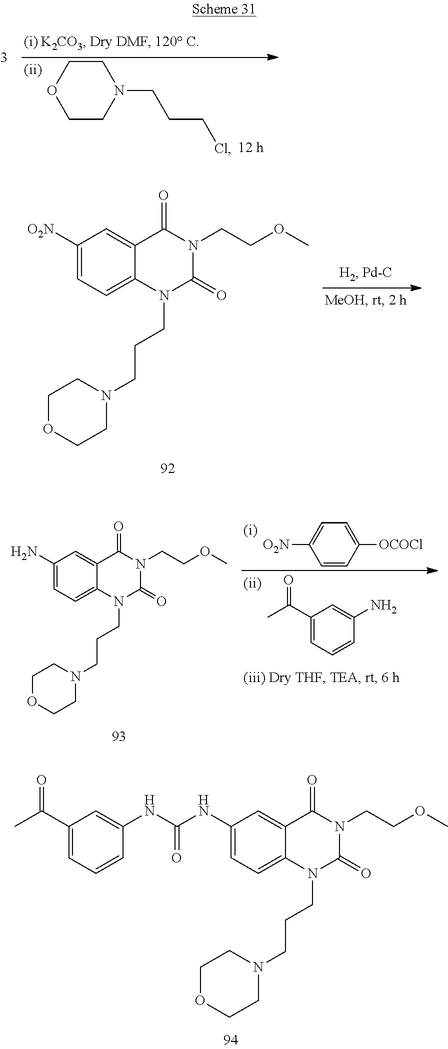

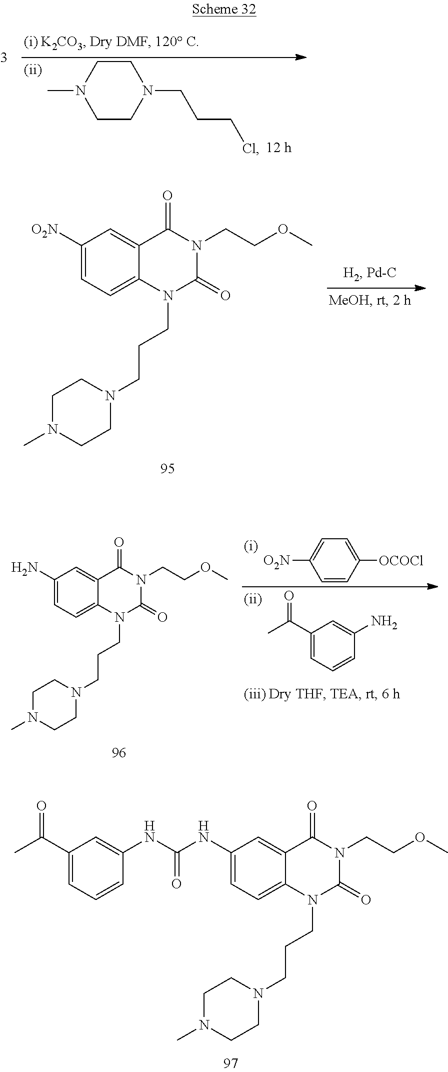

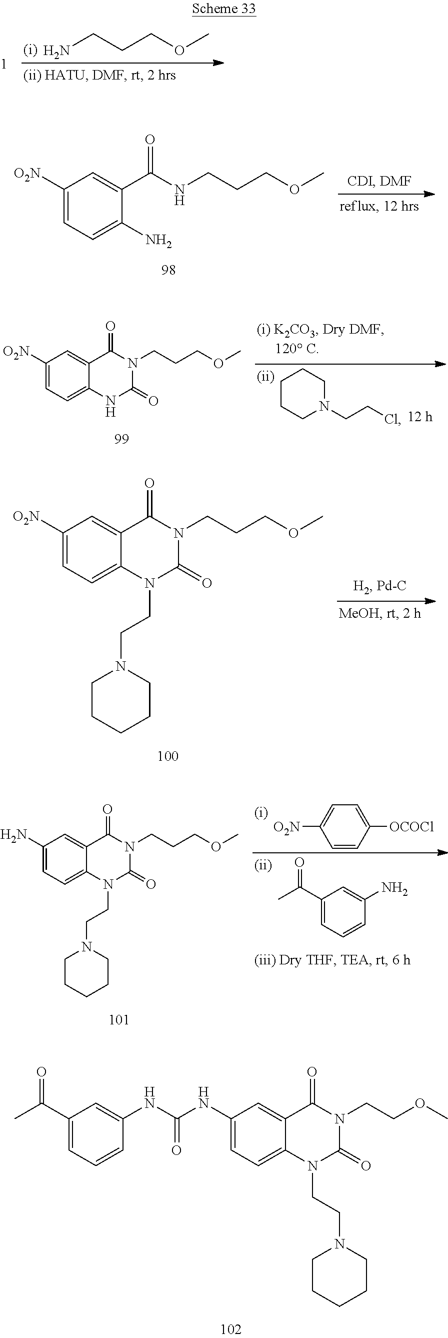

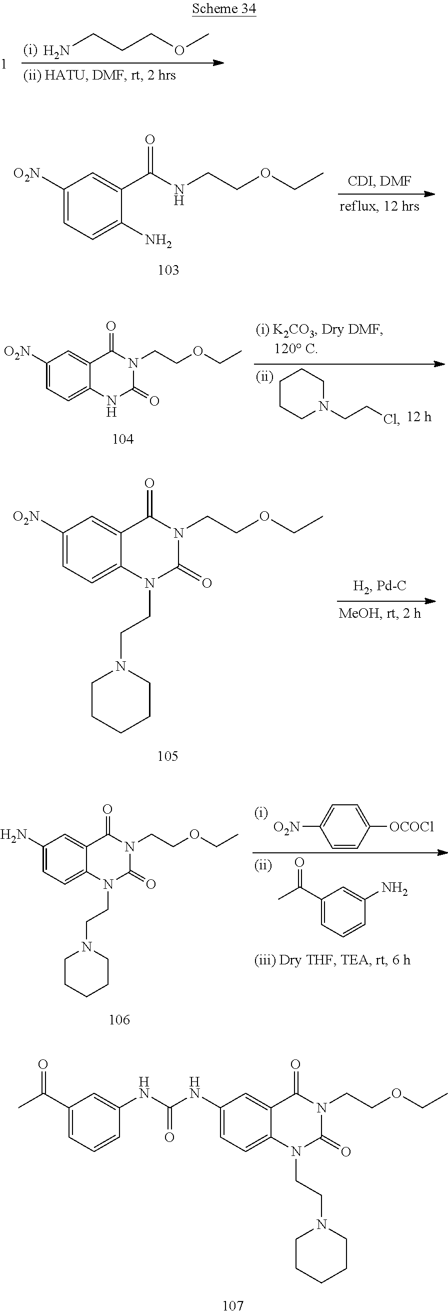

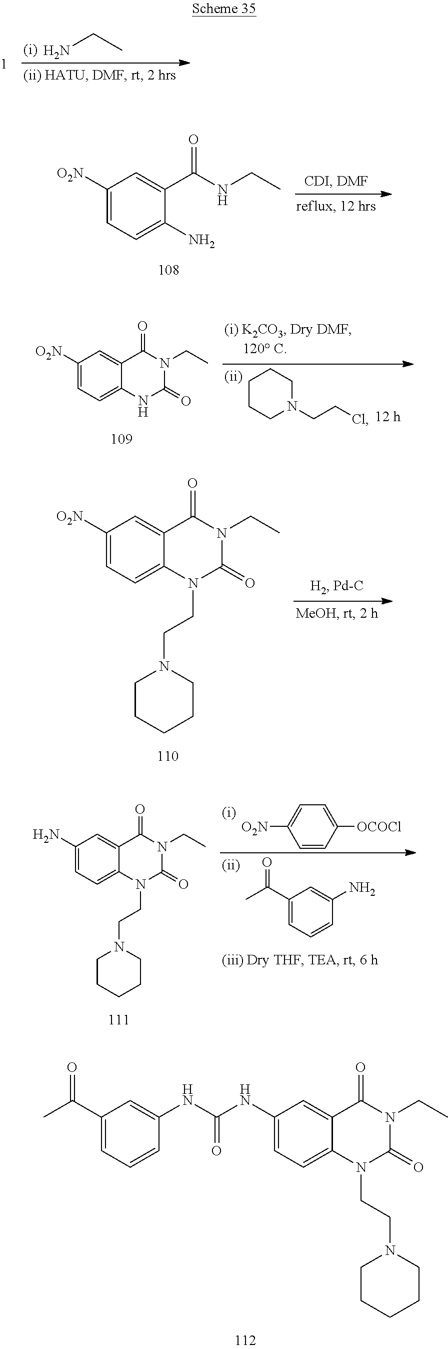

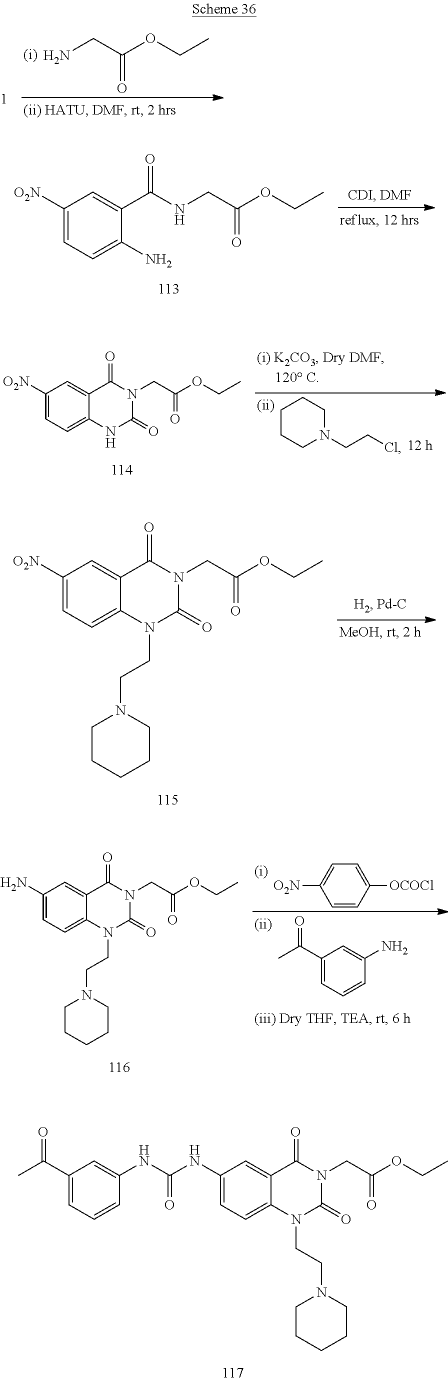

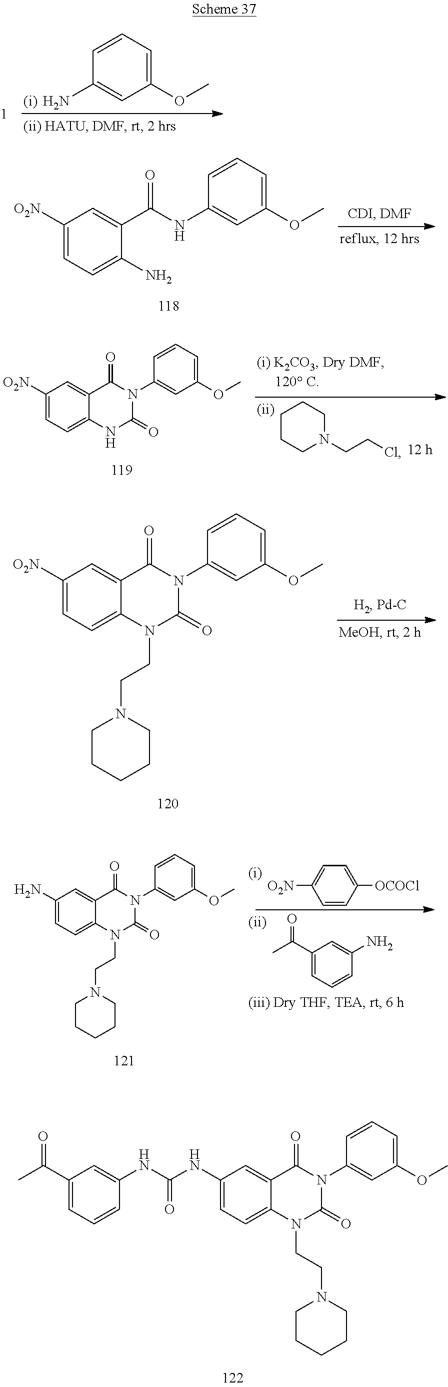

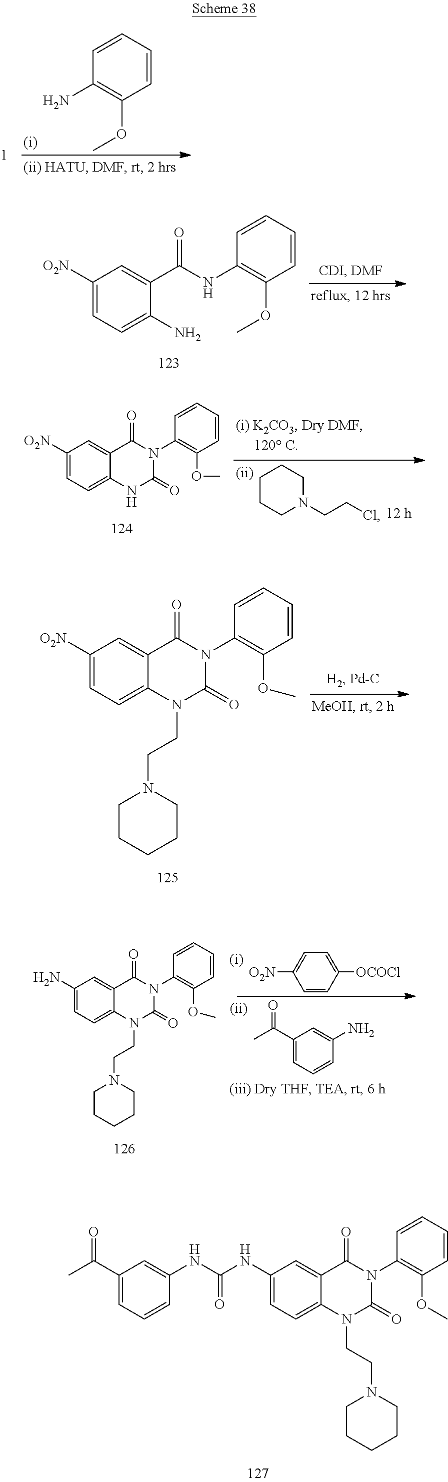

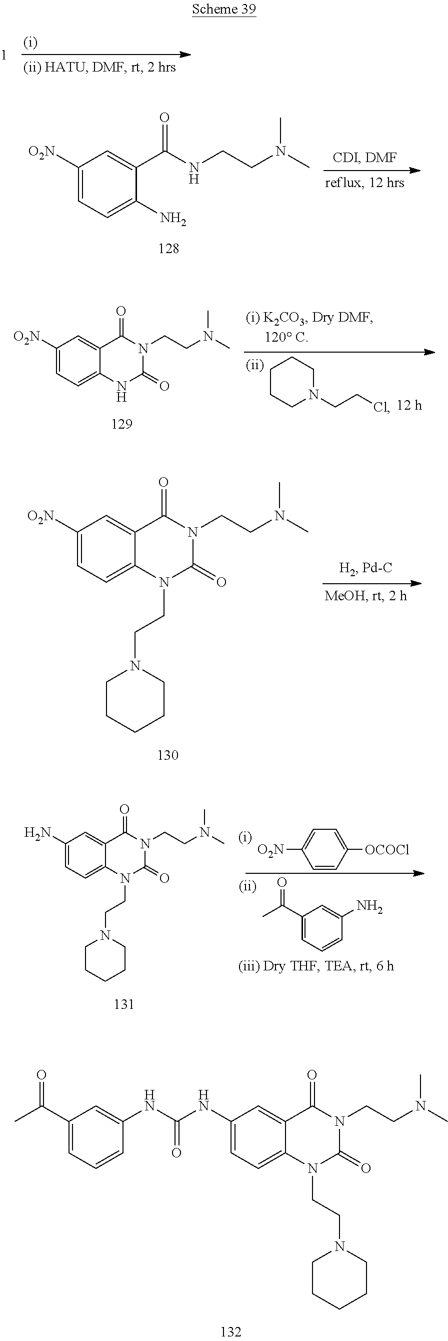

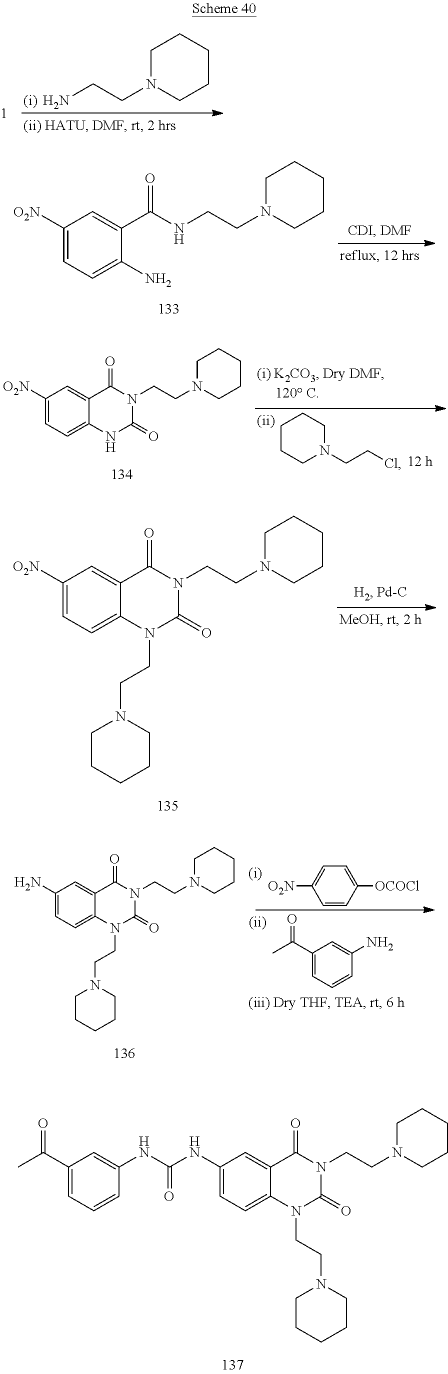

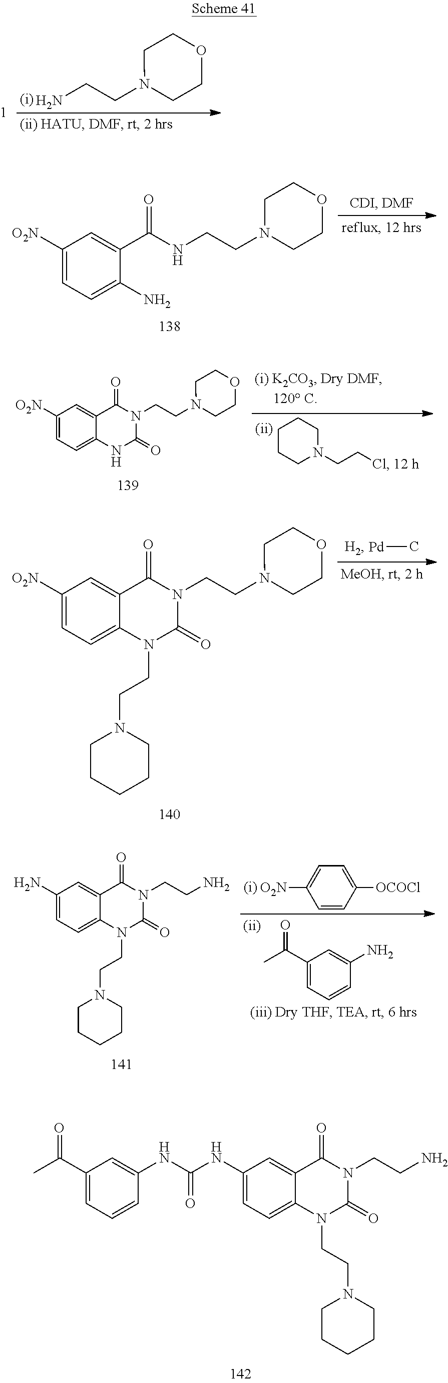

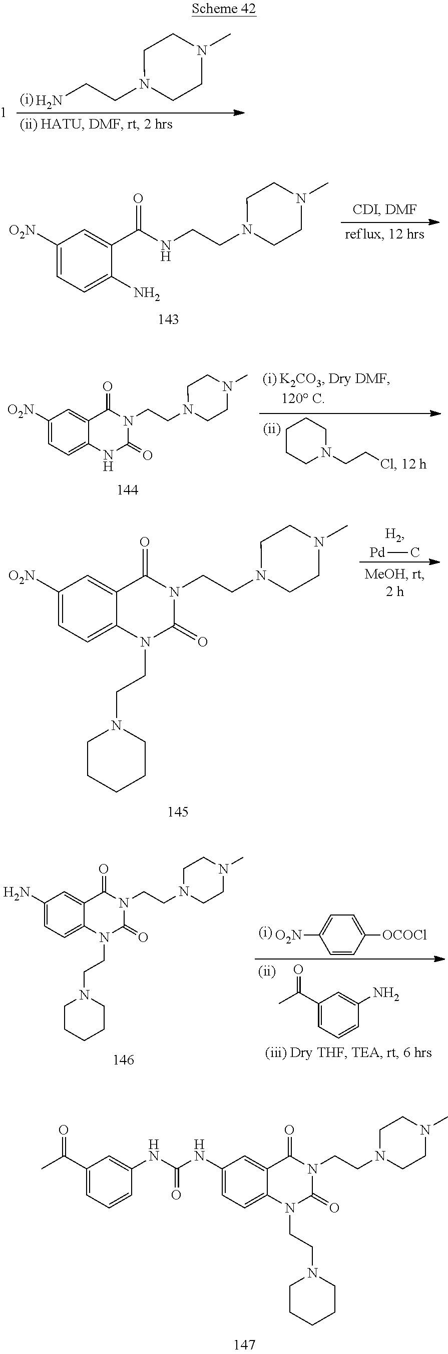

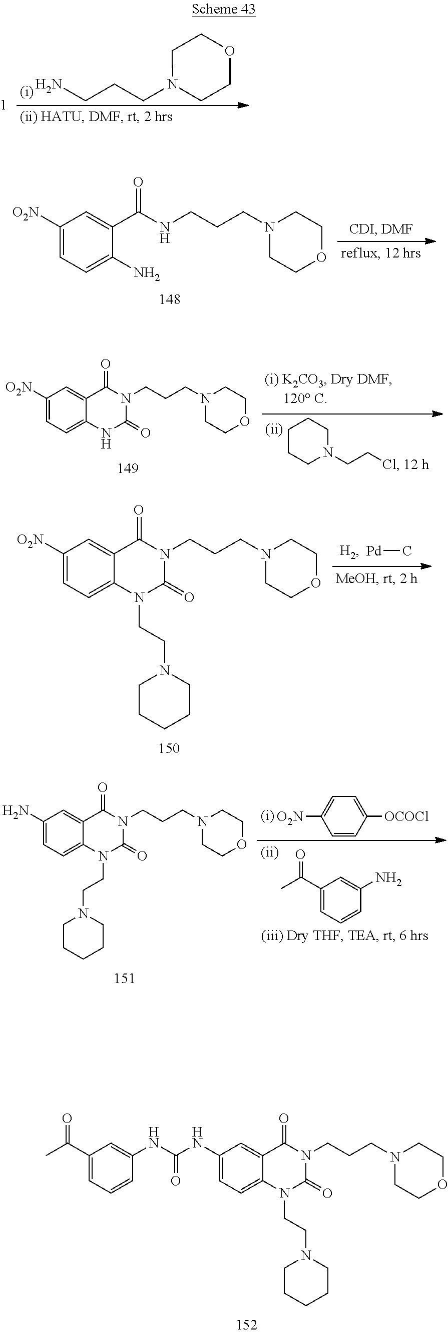

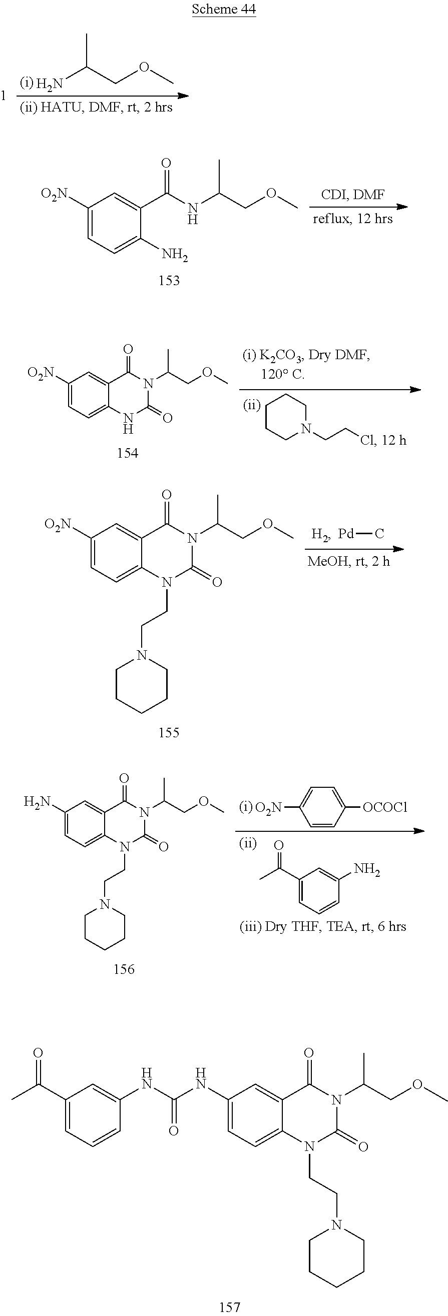

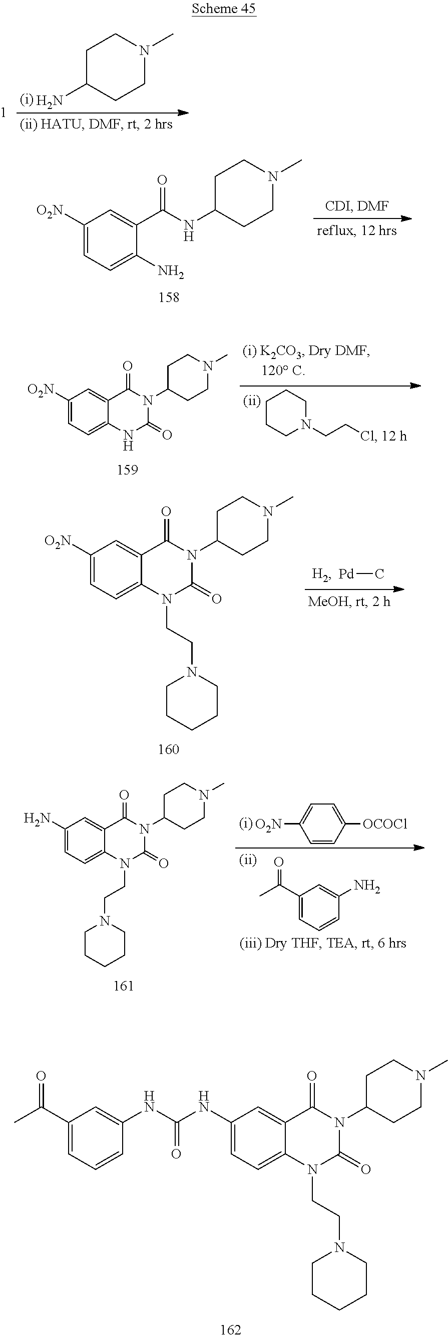

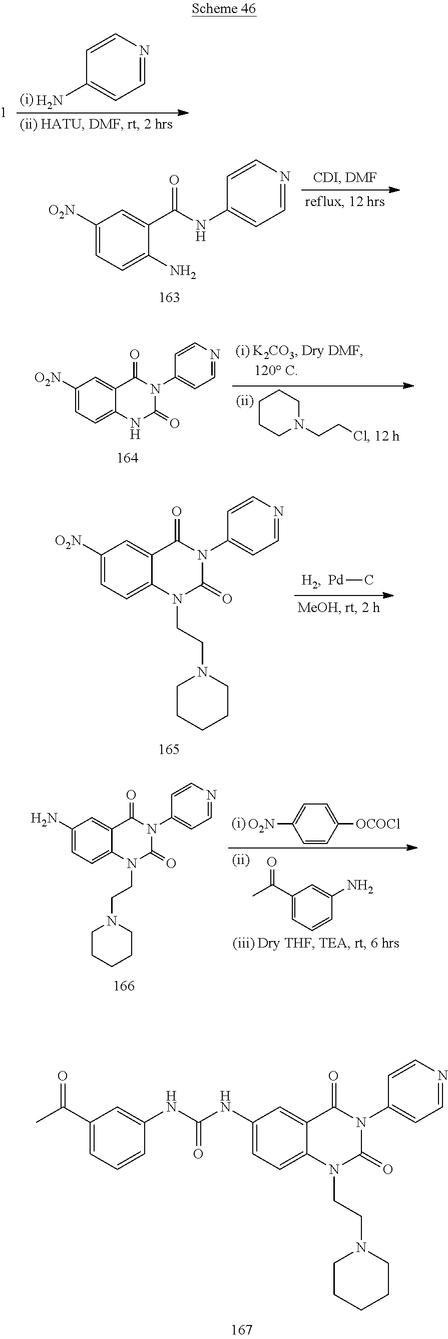

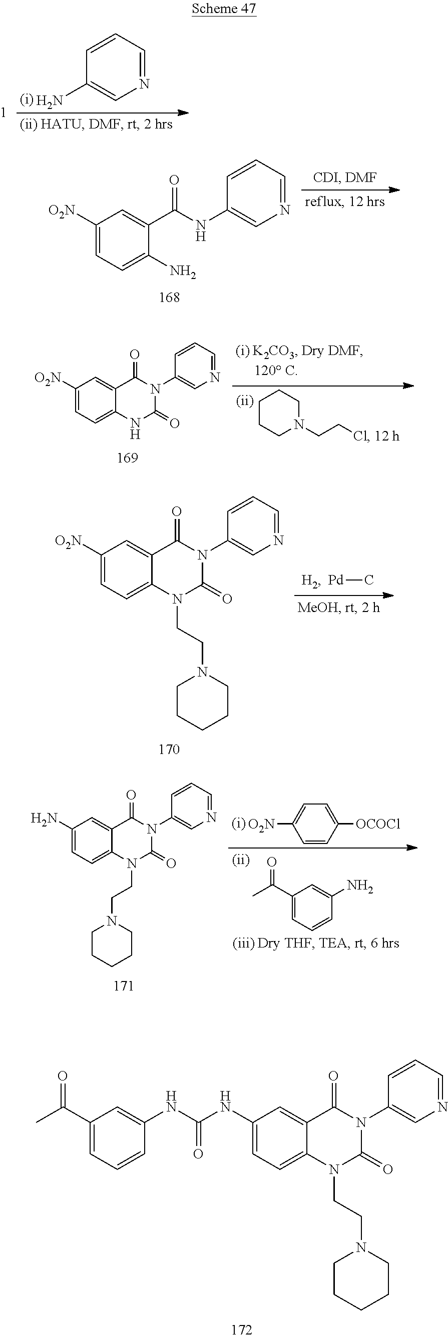

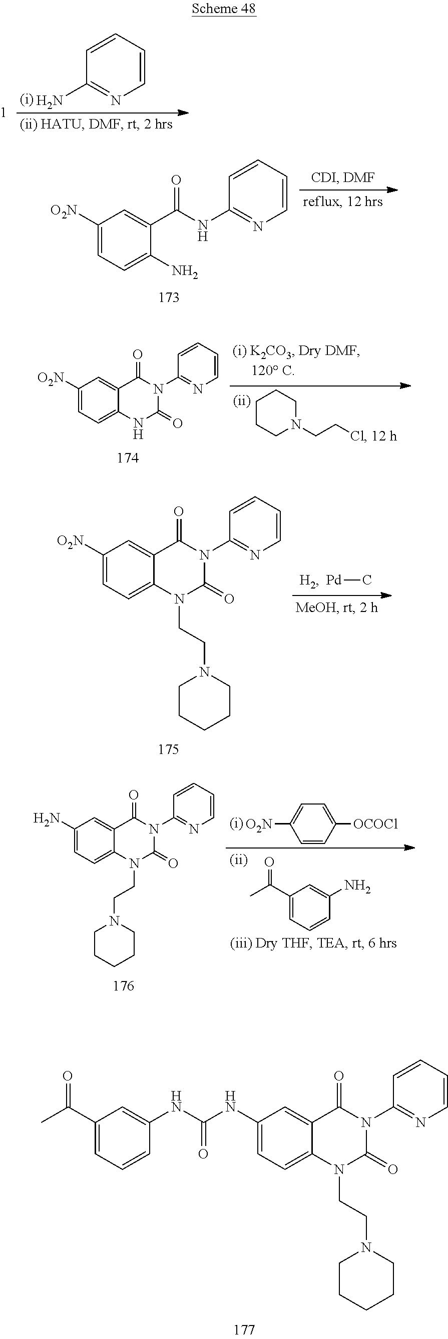

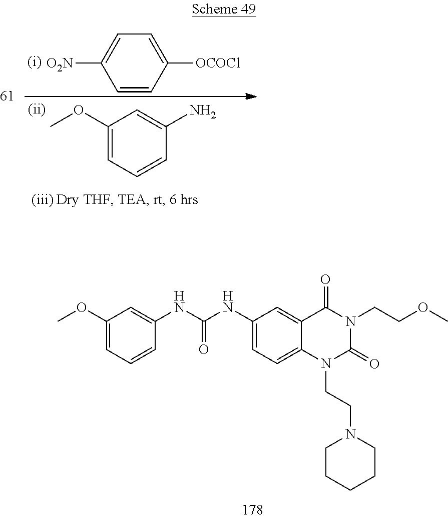

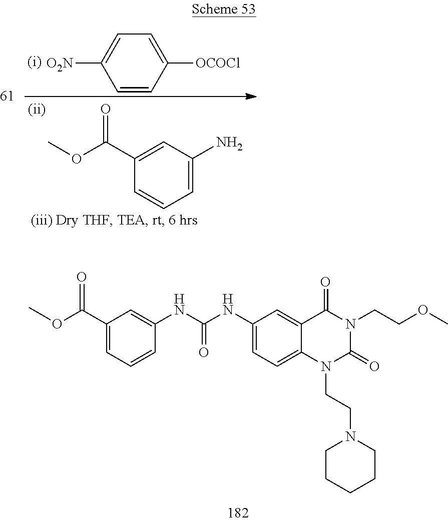

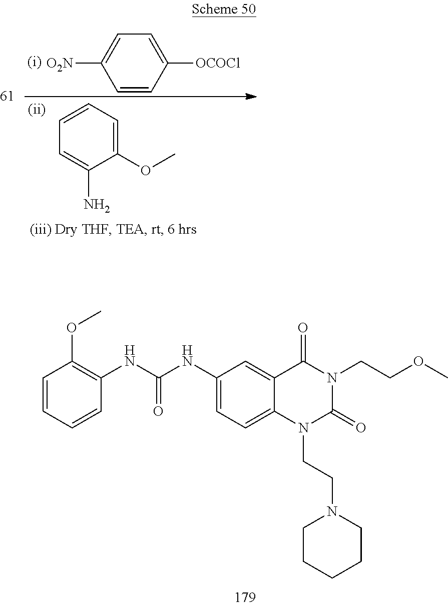

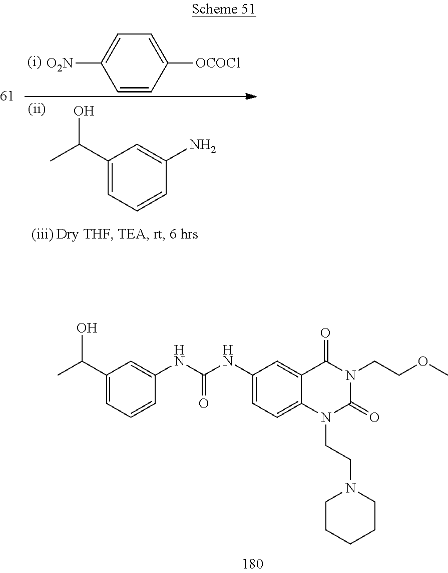

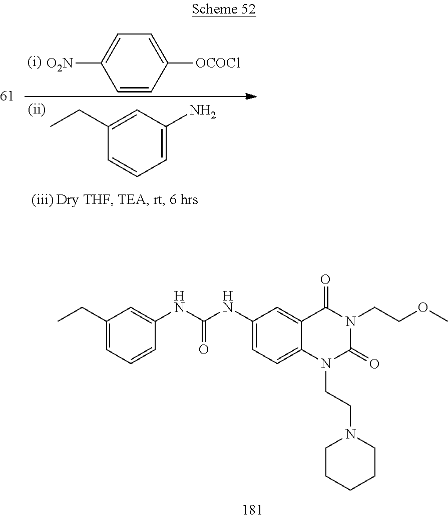

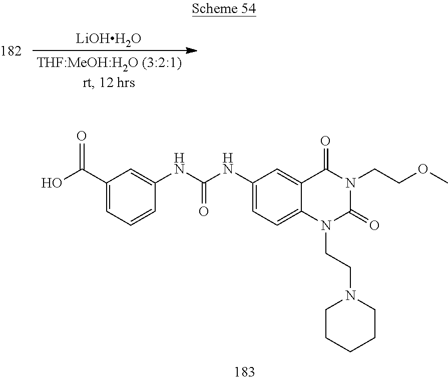 [SMILES: "LiOH·H2O" ; COCCn1c(=O)c2cc(NC(=O)Nc3cccc(C(=O)O)c3)ccc2n(CCN2CCCCC2)c1=O]

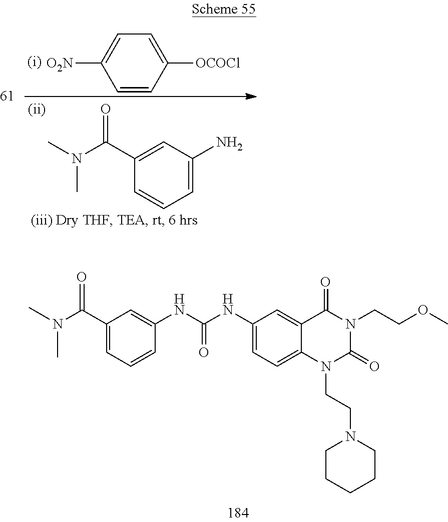

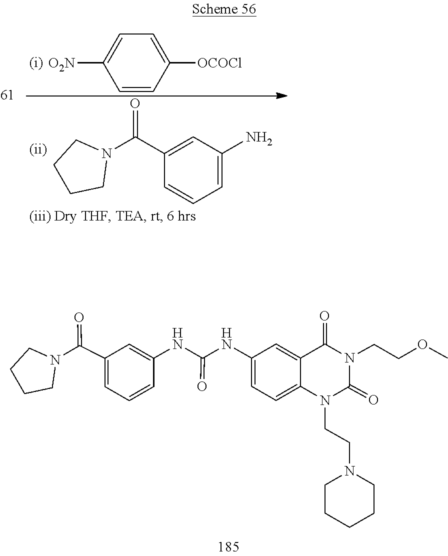

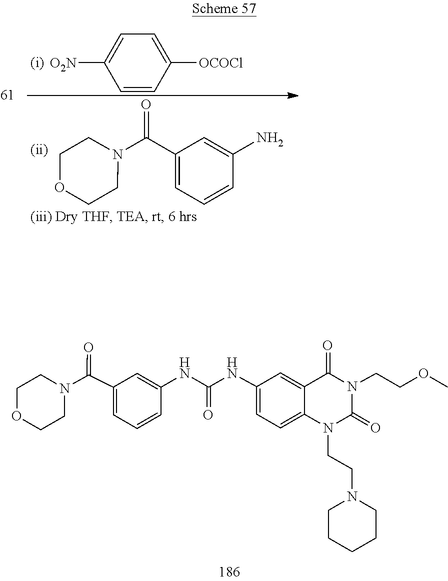

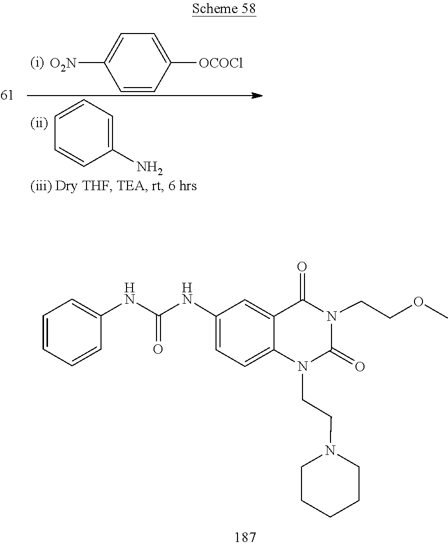

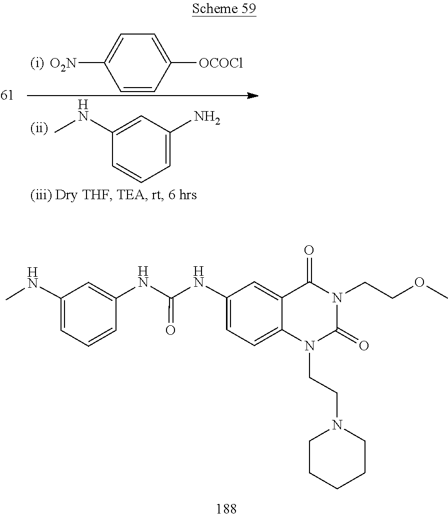

### Example 25

### Synthesis of 3-(2-methoxyethyl)-6-nitro-1-(3-(piperidin-1-yl)propyl) quinazoline-2,4 (1H,3H)-dione (89) (TSG-03-156)

Compound 3 (0.3 g, 1.13 mmol), K₂CO₃ (0.312 g, 2.26 mmol), 1-(3-Chloropropyl) piperidine hydrochloride (0.269 g, 1.69 mmol) were taken in dry DMF in a pressure tube and stirred at 120° C. for overnight. Upon completion of the reaction, reaction mass was worked up with EtOAc and water, then purified by column chromatography (Silica gel, mesh size 100-200) eluting (80% EtOAc/Pet ether) to get compound 89 (0.258 g, 62%) as off white solid.

¹H NMR (400 MHZ, CDCl₃) δ in ppm 9.05 (d, J=2.4 Hz, 1H), 8.44 (dd, J=9.2 Hz, 3.2 Hz, 1H), 7.61 (d, J=9.2 Hz, 1H), 4.32 (t, J=6.0 Hz, 2H), 4.22 (t, J=7.2 Hz, 2H), 3.67 (t, J=6.0 Hz, 2H), 3.34 (s, 3H), 2.42-2.32 (m, 6H), 1.93-1.86 (m, 2H), 1.60-1.53 (m, 4H), 1.48-1.41 (m, 2H).

### Example 26

### Synthesis of 6-amino-3-(2-methoxyethyl)-1-(3-(piperidin-1-yl) propyl) quinazoline-2,4 (1H,3H)-dione (90) (TSG-03-181)

Compound 89(0.2 g, 0.51 mmol) was dissolved in methanol (5 mL) and pinch of 10% wet Pd-C was added. Reaction mixture was degassed by passing nitrogen and H₂ gas was passed for 2 hours to get fully reduced compound. Reaction was thoroughly monitored by checking TLC. Upon completion of the reaction, Pd-C was filtered through celite bed and methanol was evaporated in vacuum to obtain compound 90 (0.122 g, 65%) as light brown solid. ¹H NMR (400 MHZ, CDCl₃) δ in ppm 7.45 (d, J=2.8 Hz, 1H), 7.15 (d, J=8.8 Hz, 1H), 6.97 (dd, J=8.8 Hz, 2.8 Hz, 1H), 4.28 (t, J=6.0 Hz, 2H), 4.09 (t, J=7.2 Hz, 2H), 3.64 (t, J=6.0 Hz, 2H), 3.33 (s, 3H), 2.46-2.35 (m, 4H), 1.46-1.39 (m, 2H). ESI-HRMS m/z 361.2246 (M+H⁺).

### Example 27

### Synthesis of 1-(3-acetylphenyl)-3-(3-(2-methoxyethyl)-2,4-dioxo-1-(3-(piperidin-1-yl)propyl)-1,2,3,4-tetrahydroquinazolin-6-yl)urea (91) (TSG-03-183)

Compound 90(0.08 g, 0.22 mmol) was dissolved in dry THF (3 mL) and 4-Nitrophenylchloroformate (0.072 g, 0.34 mmol) was added portion wise and reaction mixture was stirred for 2 hour till consume of the amine. Reaction was monitored by checking TLC. 3′-aminoacetophenone (0.048 g, 0.34 mmol) was added followed by TEA (0.07 mL, 0.46 mmol) and reaction mixture was stirred for another 3 hours. Upon completion of the reaction, reaction mass was evaporated in vacuum to remove THF and washed with satd. NaHCO₃ solution and extracted with EtOAc to give yellow coloured crude mass which was then purified by column chromatography (Silica gel, mesh size 100-200) eluting (10% MeOH/CHCl₃) to get compound 91 (0.052 g, 46%) as off white solid.

¹H NMR (400 MHZ, CDCl₃) δ in ppm 9.08 (s, 1H), 9.04 (s, 1H), 8.22 (d, J=1.6 Hz, 1H), 8.09-8.07 (m, 1H), 7.82-7.89 (m, 1H), 7.70-7.68 (m, 1H), 7.59 (d, J=5.2 Hz, 1H), 7.51 (d, J=6.0 Hz, 1H), 7.46-7.42 (m, 1H), 4.14 (t, J=4.0 Hz, 2H), 4.11 (t, J=4.4 Hz, 2H), 3.53 (t, J=4.0 Hz, 2H), 3.24 (s, 3H), 2.56 (s, 3H), 2.44-2.24 (m, 6H), 1.81-1.75 (m, 2H), 1.49-1.43 (m, 4H), 1.40-1.33 (m, 2H). ESI-HRMS m/z 522.2722 (M+H⁺).

### Example 28

### Synthesis of 3-(2-methoxyethyl)-1-(3-morpholinopropyl)-6-nitroquinazoline-2,4 (1H,3H)-dione (92) (TSG-03-155)

Compound 3 (0.4 g, 1.50 mmol) was dissolved in dry DMF (5 mL) and then sodium tert-butoxide (0.29 g, 3 mmol) was added and the reaction mass was allowed to stir at room temperature for 1 hr. Then a mixture of 4-(3-Chloropropyl) morpholine (0.296 g, 1.8 mmol) in dry TEA (1 mL) was added and the reaction mass and was stirred at 100° C. for overnight. Upon completion of the reaction, reaction mass was worked up with EtOAc and water, then purified by column chromatography (Silica gel, mesh size 100-200) eluting (3% MeOH/CHCl₃) to get compound 92 (0.366 g, 62%) as off white solid.

¹H NMR (400 MHZ, CDCl₃) δ in ppm 9.05 (d, J=2.4 Hz, 1H), 8.44 (dd, J=9.2 Hz, 2.8 Hz, 1H), 7.49 (d, J=9.2 Hz, 1H), 4.32 (t, J=5.6 Hz, 2H), 4.26 (t, J=7.2 Hz, 2H), 3.70-3.66 (m, 6H), 3.34 (s, 3H), 2.46-2.41 (m, 6H), 1.92-1.87 (m, 2H).

### Example 29

### Synthesis of 6-amino-3-(2-methoxyethyl)-1-(3-morpholinopropyl) quinazoline-2,4 (1H,3H)-dione (93) (TSG-03-158)

Compound 92 (0.2 g, 0.51 mmol) was dissolved in methanol (5 mL) and pinch of 10% wet Pd-C was added. Reaction mixture was degassed by passing nitrogen and H₂ gas was passed for 2 hours to get fully reduced compound. Reaction was thoroughly monitored by checking TLC. Upon completion of the reaction, Pd-C was filtered through celite bed and methanol was evaporated in vacuum to obtain compound 93 (0.118 g, 64%) as light brown solid.

¹H NMR (400 MHz, CDCl₃) δ in ppm 7.39 (d, J=2.8 Hz, 1H), 7.07 (d, J=8.8 Hz, 1H), 6.98-6.95 (m, 1H), 4.23 (t, J=6.0 Hz, 2H), 4.06 (t, J=7.2 Hz, 2H), 3.65-3.63 (m, 4H), 3.59 (t, J=6.0 Hz, 2H), 3.29 (s, 3H), 2.42-2.37 (m, 6H), 1.87-1.79 (m, 2H).

### Example 30

### Synthesis of 1-(3-acetylphenyl)-3-(3-(2-methoxyethyl)-1-(3-morpholinopropyl)-2,4-dioxo-1,2,3,4-tetrahydroquinazolin-6-yl)urea (94) (TSG-03-159)

Compound 93(0.08 g, 0.23 mmol) was dissolved in dry THF (3 mL) and 4-Nitrophenylchloroformate (0.071 g, 0.34 mmol) was added portion wise and reaction mixture was stirred for 2 hour till consume of the amine. Reaction was monitored by checking TLC. 3′-aminoacetophenone (0.048 g, 0.34 mmol) was added followed by TEA (0.07 mL, 0.46 mmol) and reaction mixture was stirred for another 3 hours. Upon completion of the reaction, reaction mass was evaporated in vacuum to remove THF and washed with satd. NaHCO₃ solution and extracted with EtOAc to give yellow coloured crude mass which was then purified by column chromatography (Silica gel, mesh size 100-200) eluting (10% MeOH/CHCl₃) to get compound 94 (0.061 g, 53%) as off white solid.

¹H NMR (400 MHZ, DMSO-d6) δ in ppm 8.94 (s, 1H), 8.91 (s, 1H), 8.17 (d, J=2.8 Hz, 1H), 8.04-8.02 (m, 1H), 7.75 (dd, J=8.8 Hz, 2.4 Hz, 1H), 7.67-7.63 (m, 1H), 7.56-7.53 (m, 1H), 7.45 (d, J=9.2 Hz, 1H), 7.42-7.37 (m, 1H), 4.13-4.05 (m, 4H), 3.52-3.45 (m, 6H), 3.20 (s, 3H), 2.52 (s, 3H), 2.37-2.32 (m, 2H), 2.30-2.22 (m, 4H), 1.78-1.69 (m, 2H).

### Preparation of Compounds 194, 195, 197, 199, 201, 203, 205, 207, 209, 211, 216, 217, 222, 227, 228-231 (Schemes 60-81)

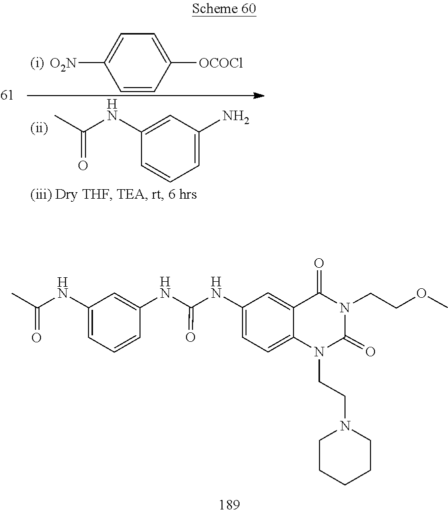

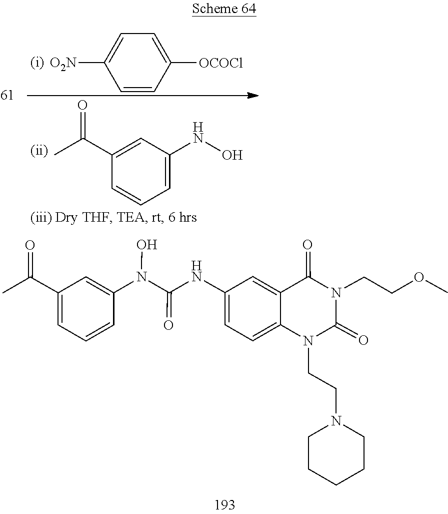

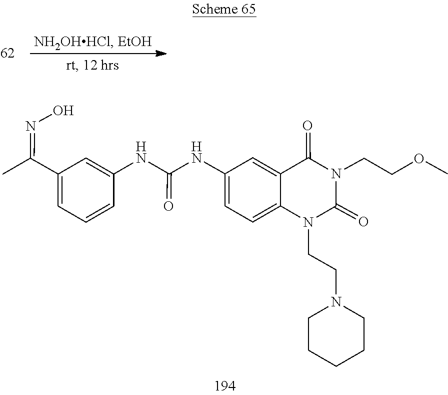 [SMILES: COCCn1c(=O)c2cc(NC(=O)Nc3cccc(/C(C)=N\O)c3)ccc2n(CCN2CCCCC2)c1=O]

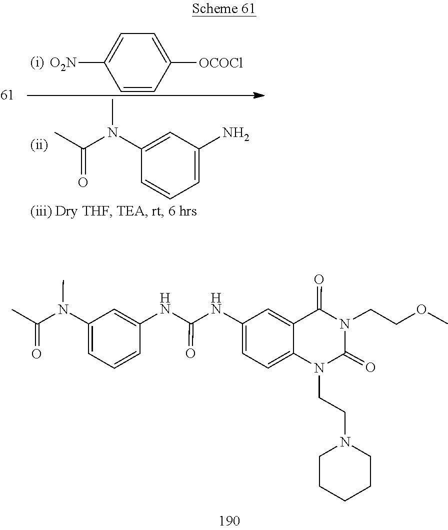

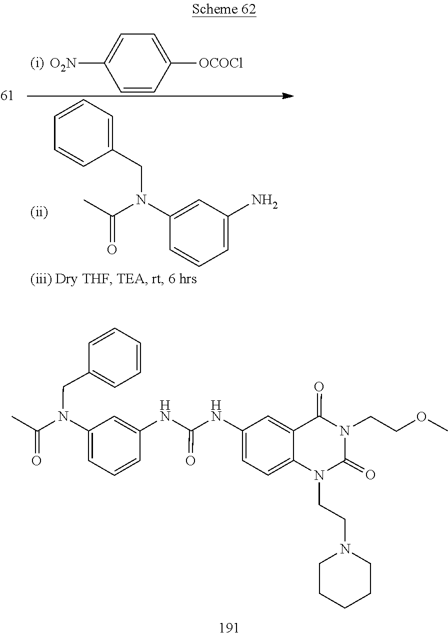

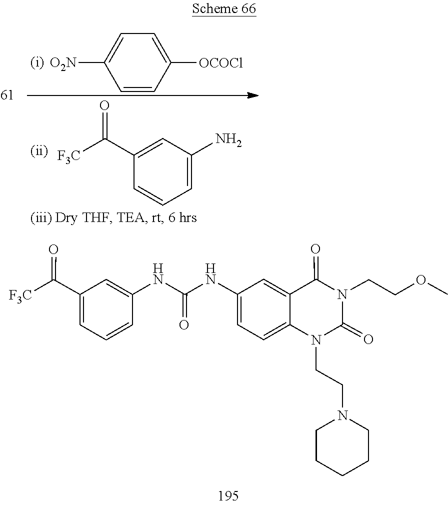

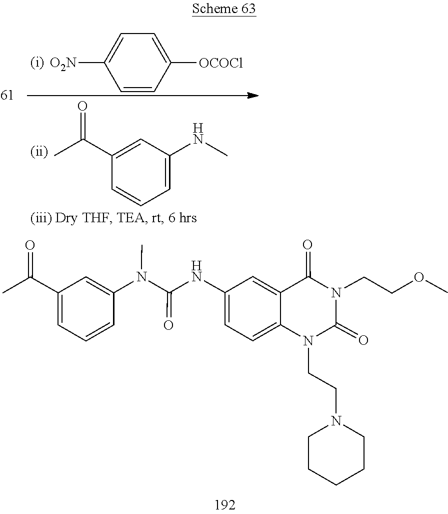

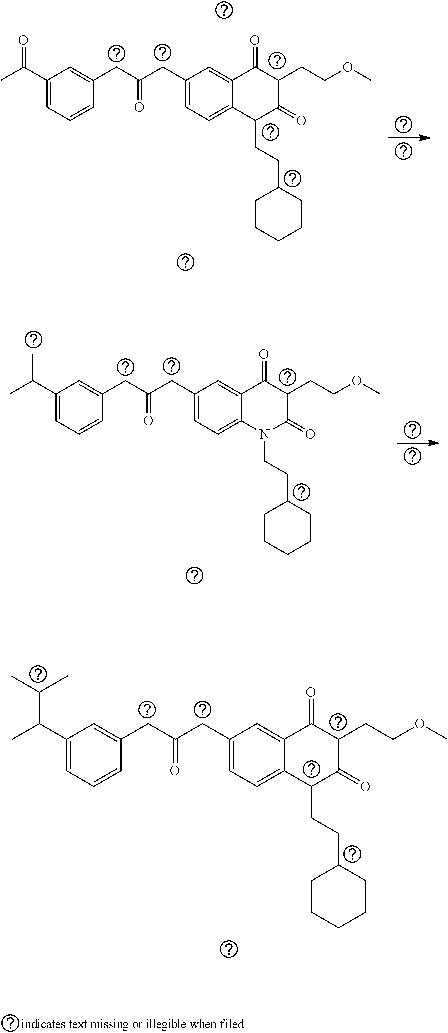

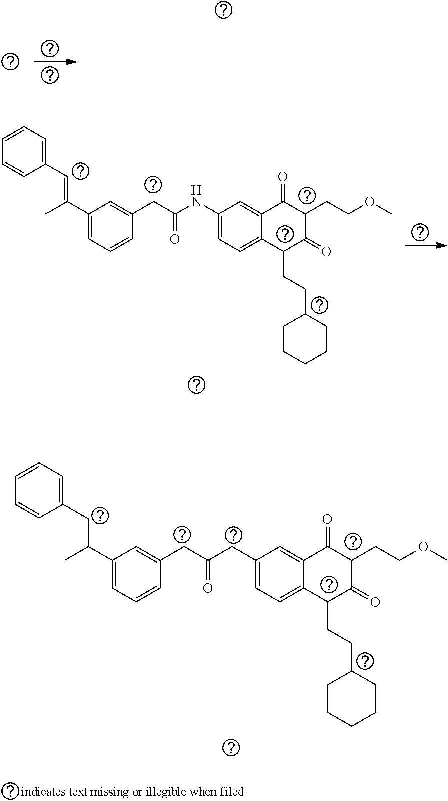 [SMILES: COCCC1C(=O)c2cc(NC(=O)Cc3cccc(/C(C)=C/c4ccccc4)c3)ccc2C(CCC2CCCCC2)C1=O ; COCCC1C(=O)c2cc(CC(=O)Cc3cccc(C(C)Cc4ccccc4)c3)ccc2C(CCC2CCCCC2)C1=O]

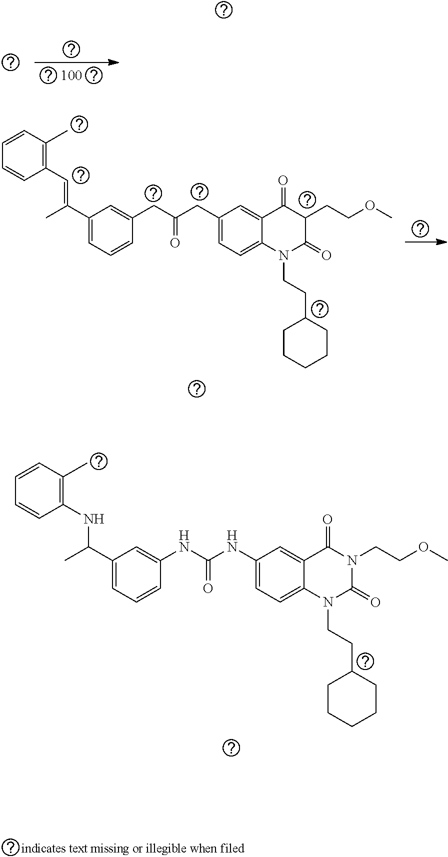 [SMILES: COCCC1C(=O)c2cc(CC(=O)Cc3cccc(/C(C)=C/c4ccccc4C)c3)ccc2N(CCC2CCCCC2)C1=O ; COCCn1c(=O)c2cc(NC(=O)Nc3cccc(C(C)Nc4ccccc4C)c3)ccc2n(CCC2CCCCC2)c1=O]

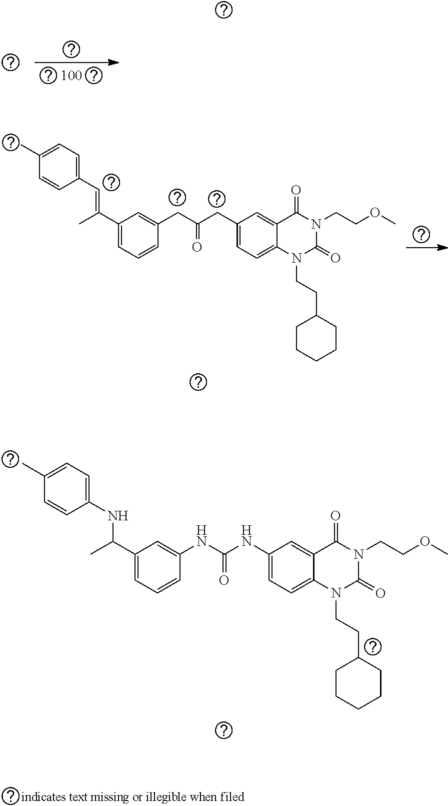

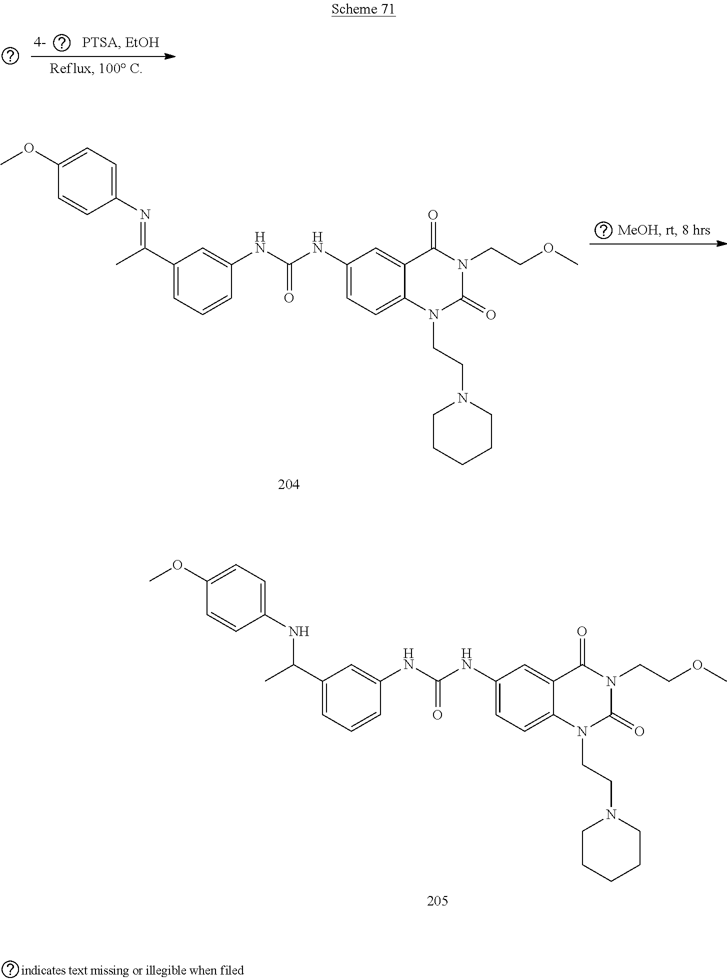

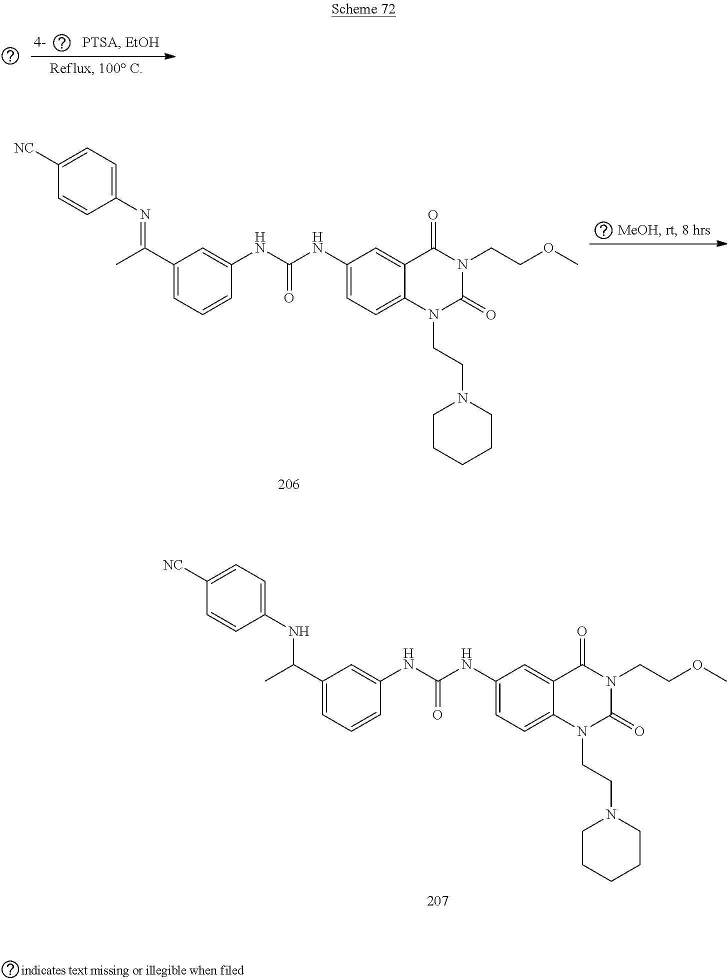 [SMILES: COCCn1c(=O)c2cc(NC(=O)Nc3cccc(/C(C)=N/c4ccc(C#N)cc4)c3)ccc2n(CCN2CCCCC2)c1=O ; COCCn1c(=O)c2cc(NC(=O)Nc3cccc(C(C)Nc4ccc(C#N)cc4)c3)ccc2n(CCN2CCCCC2)c1=O]

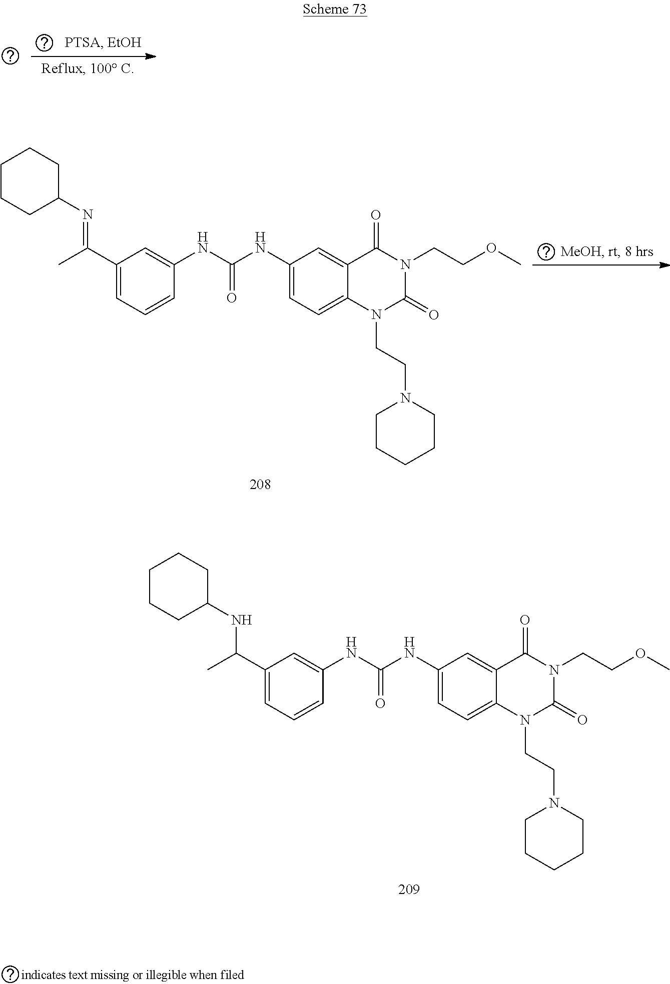 [SMILES: COCCn1c(=O)c2cc(NC(=O)Nc3cccc(/C(C)=N/C4CCCCC4)c3)ccc2n(CCN2CCCCC2)c1=O ; COCCn1c(=O)c2cc(NC(=O)Nc3cccc(C(C)NC4CCCCC4)c3)ccc2n(CCN2CCCCC2)c1=O]

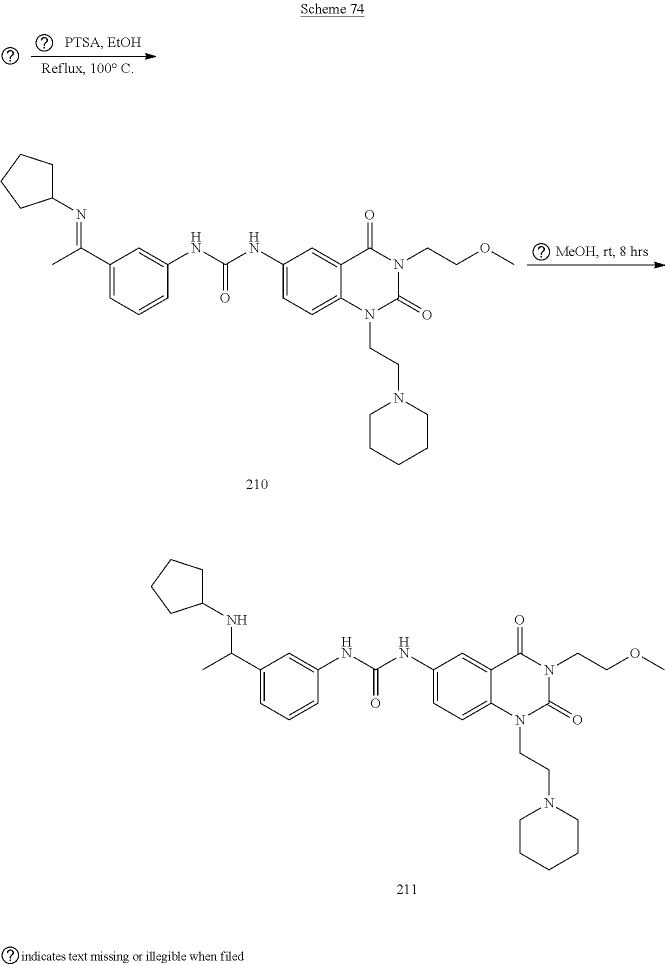 [SMILES: COCCn1c(=O)c2cc(NC(=O)Nc3cccc(/C(C)=N/C4CCCC4)c3)ccc2n(CCN2CCCCC2)c1=O ; COCCn1c(=O)c2cc(NC(=O)Nc3cccc(C(C)NC4CCCC4)c3)ccc2n(CCN2CCCCC2)c1=O]

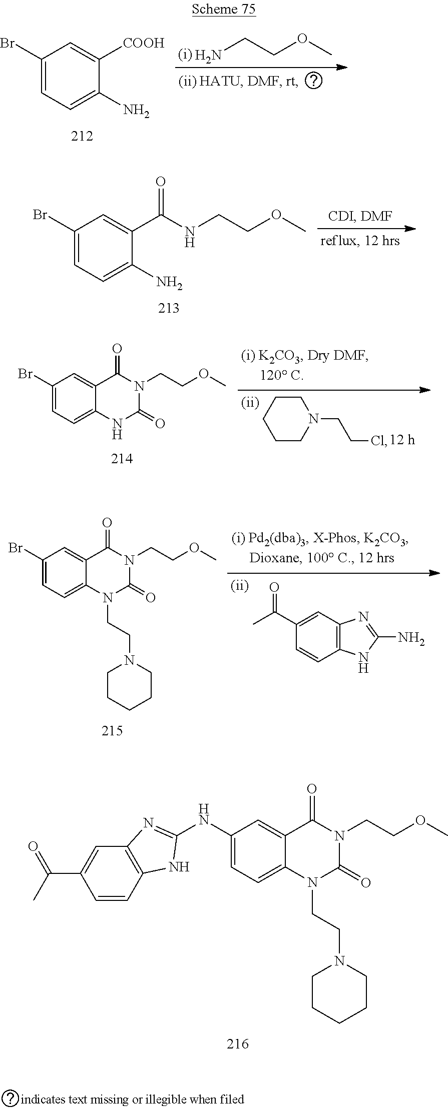

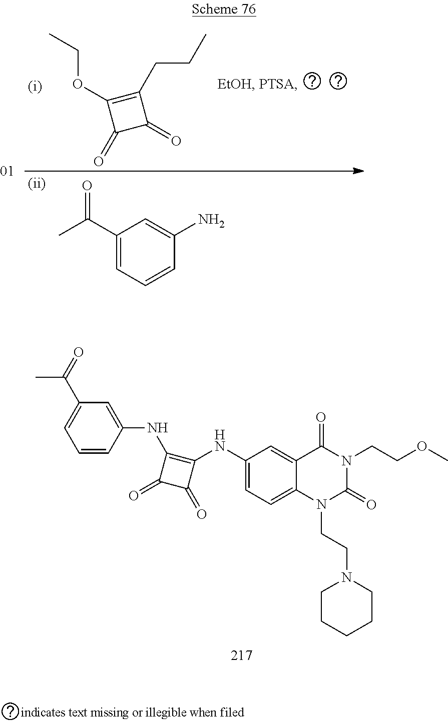

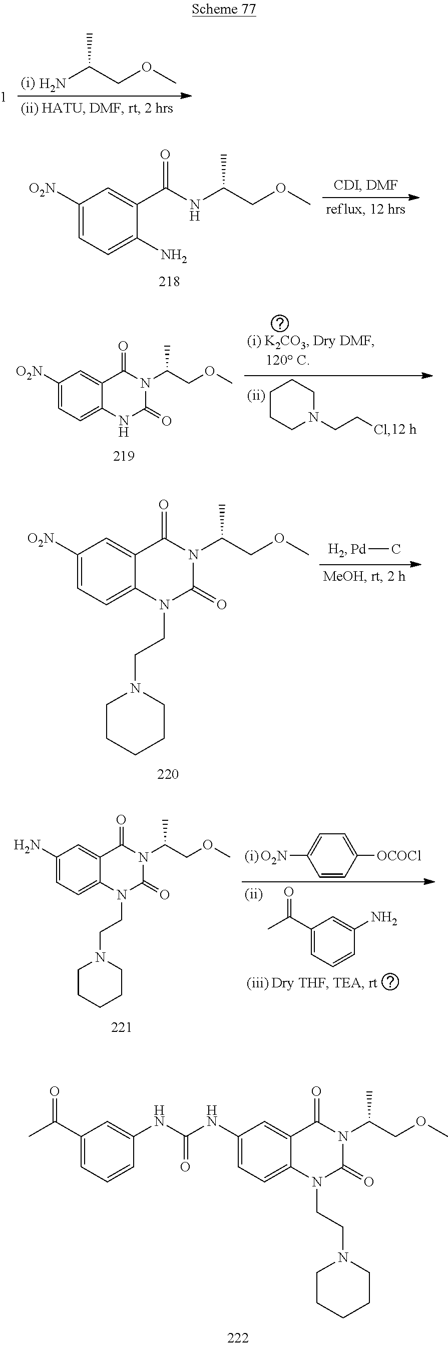

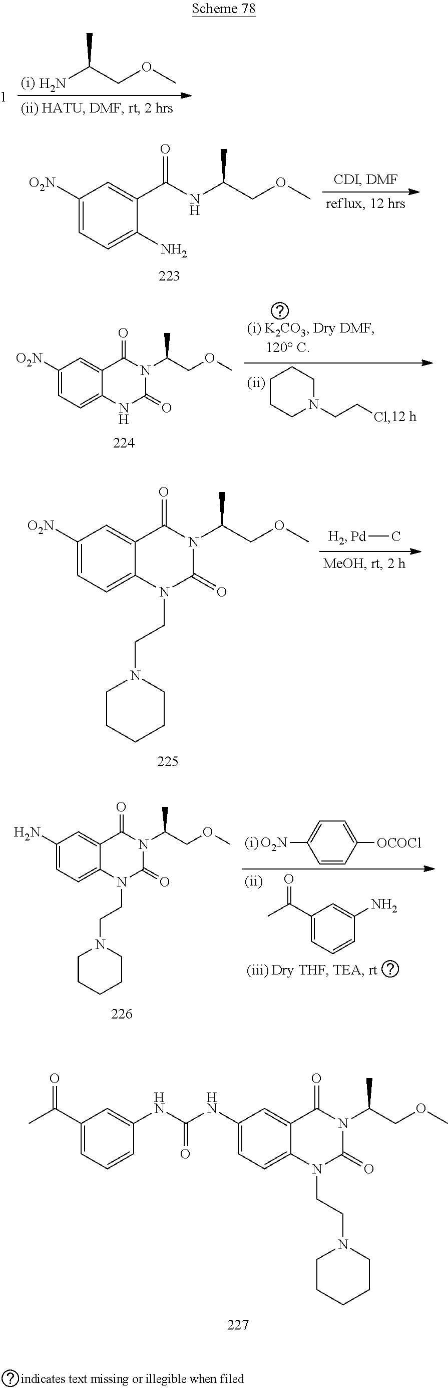

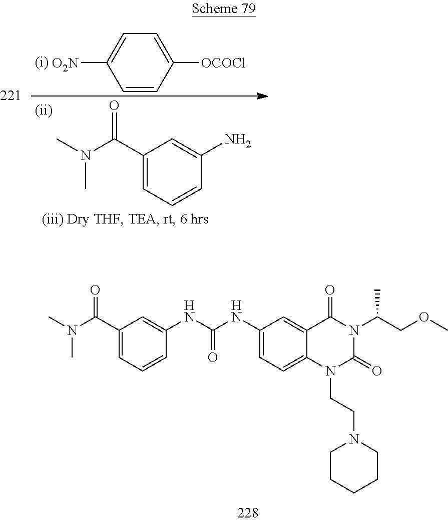

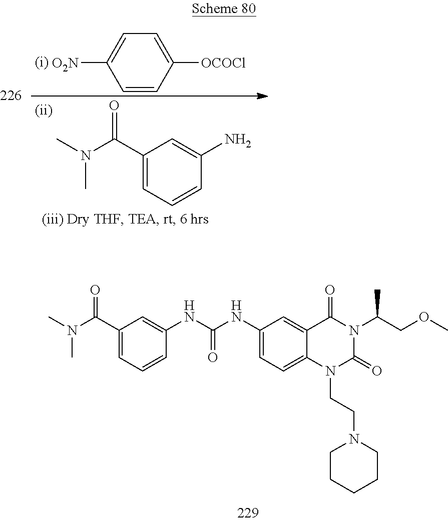

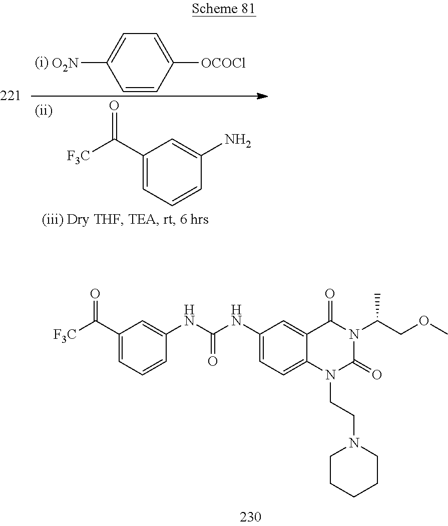

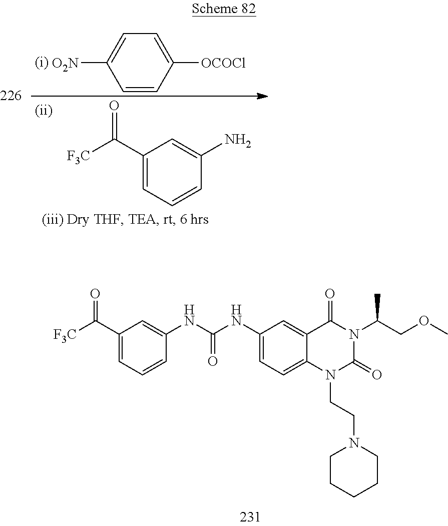

### Example 31

### Synthesis of (E)-1-(3-(1-(hydroxyimino)ethyl)phenyl)-3-(3-(2-methoxyethyl)-2,4-dioxo-1-(2-(piperidin-1-yl)ethyl)-1,2,3,4-tetrahydroquinazolin-6-yl)urea (194) (TSG-03-186)

Compound 62 (0.1 g, 0.19 mmol) was dissolved in Ethanol (3 mL), then Hydroxyl amine hydrochloride (0.028 g, 0.38 mmol) was added and reaction mixture was refluxed for 4 hours. After completion of the reaction ethanol was evaporated out and reaction mass was worked up by EtOAc and water, then purified by column chromatography (Silica gel, mesh size 100-200) eluting (3% MeOH/CHCl₃) to get compound 194 (0.058 g, 59%) as off white solid.

¹H NMR (400 MHZ, DMSO-d6) δ in ppm 11.17 (s, 1H), 8.20 (d, J=2.8 Hz, 1H), 7.80-7.78 (m, 1H), 7.75 (d, J=2.4 Hz, 1H), 7.59 (d, J=9.2 Hz, 1H), 7.47-7.43 (m, 1H), 7.27-7.22 (m, 1H), 7.20-7.16 (m, 1H), 4.45 (t, J=6.8 Hz, 2H), 4.09 (t, J=6.4 Hz, 2H), 3.50 (t, J=6.4 Hz, 2H), 3.40-3.37 (m, 2H), 3.35-3.32 (6H), 3.21 (s, 3H), 2.09 (s, 3H), 1.94-1.84 (m, 4H).

### Biological Assay

To select the compounds capable of inhibiting the ubiquitination of ATGL by COP1 by targeting the VP motif, confocal microscopy was performed with the provided molecules. If the compound was effective in inhibiting the interaction, there would have been a reduction in the number of fat droplets in the cells after treatment. This was because the increased ATGL levels would hydrolyse the accumulated TAG in oleate induced HepG2 cells and bring about the aforementioned reduction. With this rationale in mind, HepG2 cells were induced to accumulate lipid droplets after treatment with 250 μM of oleate and 10 μM of the specific compounds were added. The potential of the compounds to bring about a reduction in the number of fat droplets was then checked by comparison with oleate induced cells by counting number of droplets of approximately 20 cells from each treatment and calculating the average number of lipid droplets of each cell. The selected compounds were then subjected to dose dependent treatments and the ones which could maintain its potency to reduce fat droplets at lower doses were then selected for western blot analysis. The compound which could reduce the number of fat droplets in the cells were expected to raise the levels of ATGL since they were likely to deter COP1 from ubiquitinating ATGL. This increase was visible only in the protein level and gene expression was likely to remain unchanged since ubiquitination is a post transcriptional modification. Thus, western blot was performed to check ATGL levels in the cells with the selected molecules.

### Example 32

### Western Blotting

HepG2 cells were treated with the compounds 5, 8, 11, 59, 62, 91, 94 (50 nM, 100 nM, 200 nM, 500 nM, 1 μM and 5 μM for dose dependent assays) for 24 hours. After removing media from the cells, the wells were washed with 1×PBS twice to remove any remnant media. Cells were then lysed in lysis buffer containing 50 mM Tris-HCl (pH 7.4), 100 mM NaCl, 1 mM EDTA, 1 mM EGTA, 1% Triton X-100 and protease inhibitor cocktail (Millipore, Billierica, MA, USA). Following centrifugation at 20,000 g for 20 minutes, the protein solution was extracted from the cells. Protein was estimated using Bradford assay. Bradford's reagent (BioRad) was diluted in 1:4 ratio in double distilled water. 2 μl of protein sample was added to 100 μl of the reagent and absorbance was measured at 595 nm. 30 μg of protein was diluted in lysis buffer. 1X loading buffer diluted from 5X stock containing 250 mM Tris-HCl (pH 6.8), 10% SDS, 50% glycerol, 0.1% bromophenol Blue and 10% β-mercaptoethanol was added. The protein samples were then heated at 95° C. for 10 minutes, cooled and centrifuged at 12,000 g for 2 minutes prior to loading.

For western blotting, the proteins were resolved in 10% SDS PAGE (discontinuous buffer system). 1X running buffer containing SDS, Tris Base and Glycine was used to run the gel at 80V for approximately 2 hours. Transfer was done using PVDF membrane (Millipore) having pore size of 0.45 μm. 1X transfer buffer containing Tris-Base, Glycine and 20% methanol was used for wet transfer. Transfer was done at 90V for 3 hours. Following transfer, the PVDF membrane containing the proteins were washed in 1×PBST comprising of 1×PBS and 1% Tween 20 (Sigma Aldrich). The membrane was then incubated for 1 hour at room temperature in 5% skim milk powder to block the non-specific sites. Following multiple washes with 1×PBST to wash away any remaining blocking buffer, the required primary antibody (COP1 [BethylLaboraties], ATGL [Cell Signalling Technology] or Actin[Cell Signalling Technology]) prepared with 1×PBST, 1% Bovine Serum Albumin and 0.04% Sodium Azide was added to the membrane and incubated overnight at 4° C. The next day, the membrane was again washed multiple times with 1×PBST to remove any unbound primary antibody. The membrane was then incubated with goat anti-rabbit secondary antibody (Genei) for 1 hour at room temperature and washed again for multiple times with 1×PBST. The membrane was then developed using Clarity™ ECL Western Blotting Substrate (BioRad) and viewed in ChemiDoc (BioRad).

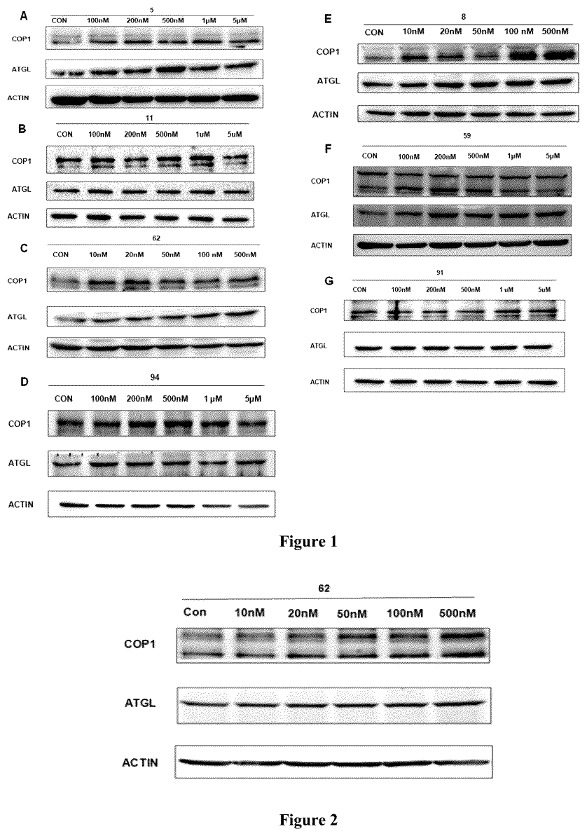

FIG. 1 (A to G) illustrates results of Western Blot Analysis in HepG2 cells after treatment with compounds 5, 8, 11, 59, 62, 91 and 94. The figure also describes the dose dependant Western Blot analysis of following compounds 5, 8, 11, 59, 62, 91 and 94. These compounds showed increased ATGL level irrespective of the treated doses. Increase in intensity of ATGL and COP1 bands with respect to control denotes elevation in the respective protein levels upon compound treatment. Actin was used as a loading control.

### Example 33

### The Effect of the Compounds in Primary Mouse Hepatocytes

To further strengthen the efficiency of the compound 62, primary hepatocytes were isolated from mice and these compounds were treated in a dose dependent manner for 24 hours. Primary hepatocytes and adipose tissue explants were isolated from mice and subjected to compound treatment for 24 hours at the doses of 10 nM, 20 nM, 50 nM, 100 nM and 500 nM. Post cell harvesting, western blot was carried out with the lysate to check the ATGL level. ATGL and COP1 antibodies were used. Actin served the purpose of a loading control.

### Culture of Primary Mouse Hepatocytes

Hepatocytes-2-4 months old chow-fed black male mouse (C57b1/6) was sacrificed using chloroform (SRL) and was cleaned with 70% ethanol. Under aseptic conditions, the ventral side of the mouse was cut open, until the liver, portal vein (PV) and inferior vena cava (IVC) were sufficiently exposed. Blood was drawn from the heart in order to prevent backflow into liver while perfusion. The butterfly cannula was inserted into the PV and 20 ml of HBSS (Hank's Balanced Salt Solution; 5 mM KCl, 0.4 mM KH₂PO₄, 4 mM NaHCO₃, 140 mM NaCl, 0.3 mM Na₂HPO₄, 6 mM Glucose, HEPES, 0.5 mM MgCl₂.6H₂O, 0.4 mM MgSO4·7H₂O, 0.5 mM EDTA; not containing 1 mM CaCl₂)) was allowed to pass through the liver (Perfusion) at a constant flow rate of 3 ml/min, maintained by Masterflex digital peristaltic pump (Cole-Parmer). The IVC was cut as soon as the passage of the buffer through the liver began, so that blood and perfusate from liver was drained through the IVC. The liver blanched and became pale in color upon this treatment.

After the passage of HBSS, 25 ml of Collagenase (Roche) solution (1 mg/ml) in HBSS (containing 1 mM CaCl₂)) was allowed to pass through the liver at a constant flow rate of 2 ml/min. After this digestion, the flow was stopped, the cannula removed and the pale and soggy lobes of the liver were gently excised from the body. The gall bladder was removed from the isolated liver. The pieces of digested liver tissue were then minced on a 10 cm culture plate in HBSS (containing 1 mM CaCl₂)). The resulting suspension was then passed through a 100μ cell strainer (SPL) to allow hepatocytes to pass through to the filtrate and retain cellular clumps and undigested tissue. The filtrate was centrifuged at 50 g for 2 minutes at 4° C. The supernatant was discarded, and the cellular pellet was carefully resuspended in DMEM. The resulting suspension was centrifuged at 50 g for 2 minutes at 4° C. The supernatant was discarded, and the cellular pellet was carefully resuspended in required volume of DMEM for plating.

The hepatocytes were plated according to experimental requirements and were maintained in an incubator at 37° C. with 5% CO₂. Cells were washed once with HBSS and DMEM 6-7 hours after plating and the adhered hepatocytes were maintained and subjected to requisite treatments.

FIG. 2 illustrates ATGL protein status in mouse primary hepatocytes after compound treatment. The level of ATGL was found to be increased in a dose-dependent manner. This provided a more profound and direct evidence of the effectiveness of the compounds. Compound 62 increased ATGL level in primary mouse hepatocytes as evidenced by increase in intensity of the corresponding band with respect to control in Western blot analysis.

### Example 34

### Status of ATGL Ubiquitination Upon Compound Treatment Assessed by Immunoprecipitation Assay

COP1, an E3 ubiquitin ligase and ATGL, one of its targets which got ubiquitinated and ultimately degraded via proteasomal mediated pathway. Thus, the molecules inhibiting COP1 by targeting the VP motif of ATGL were actually expected to bring about a reduction in the ubiquitination levels of ATGL. The compounds of the present invention have shown a reduction in the lipid droplet count with a corresponding increase in ATGL protein levels while gene expression remained unaltered. However, it was of utmost importance to check the changes taking place at the ubiquitination level of ATGL upon treatment with the compounds.

To this end, an immunoprecipitation assay was performed wherein HepG2 cells overexpressing myc-ATGL were transfected with HA-Ubiquitin and treated with 5 μM of the compounds 5, 62, and 94.MG-132, a proteasomal inhibitor, was added4 hours before harvesting the cells. Immune complexes were pulled down with anti-myc antibody and immunoblot was done using anti-HA antibody. The resultant smears on the blot reflect the ubiquitination status of ATGL.

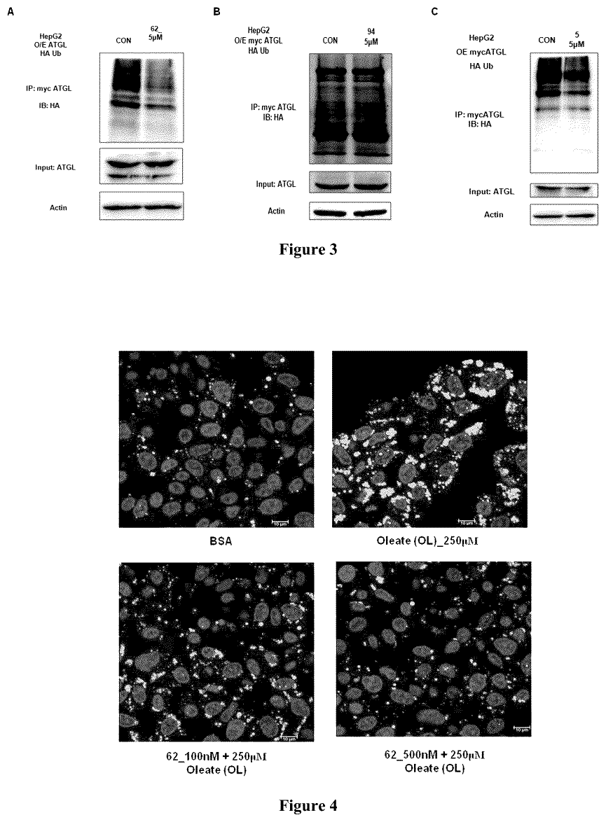

FIG. 3 (A to C) illustrate results of immunoprecipitation assay to check ubiquitination status of ATGL and COP1 after treatment with compounds 62, 94 and 5. For compounds 5 and 62, a stark reduction in the ubiquitination smear upon treatment was observed as compared with control. This reduction, however, could not be seen in cells treated with compound 94 which indicates that compound 5 and 62 might be more potent in inhibiting COP1 by blocking the ubiquitination of ATGL to some extent compared to compound 94. Compounds 62 and 5 were effective in reducing the ATGL ubiquitination by COP1 in HepG2 cells as evidenced by the decrease in the intensity of the poly-Ubiquitin smear whereas compound 94 exercised no such effect.

### Example 35

### Confocal Microscopy

HepG2 cells were plated in confocal dishes (SPL, Genetix Biotech Asia Pvt. Ltd.). The cells were allowed to adhere and divide for 16 hours. 100 nM and 500 nM concentrations were used for dose dependent assays of the compound 62, which was dissolved in DMSO and added to the cells. 250 μM of oleate was used for induction. BSA (Sigma Aldrich) was used as a negative control. Post 24 hours of treatment, media was decanted from the cells and washed with 1×PBS solution to remove any remnant. 200 μl of staining solution containing 200 ng/ml BODIPY (Invitrogen) and 25 μg/ml HOECKST342 (Invitrogen) were added to the cells and incubated at 37° C. for 30 minutes under dark conditions. Cells were washed 3 times with 1×PBS to remove excess stain. FLUOVIEW FV10i (Olympus) was used to visualise the cells.

FIG. 4 illustrates images of compound 62 screening on HepG2 cells using confocal microscopy. The green foci in the cells denote lipid droplets. Increase or decrease in the number of green foci therefore indicated the corresponding status of lipid droplets in the cells. Oleate induction resulted in an increase in lipid droplets whereas treatment with compound 62 caused a decrease in the number of lipid droplets upon oleate induction.

### Example 36

### Western Blotting

HepG2 cells were treated with the compounds 5, 8, 11, 59, 62, 65, 91, 94 at 5 μM for 24 hours. After removing media from the cells, the wells were washed with 1×PBS twice to remove any remnant media. Cells were then lysed in lysis buffer containing 50 mM Tris-HCl (pH 7.4), 100 mM NaCl, 1 mM EDTA, 1 mM EGTA, 1% Triton X-100 and protease inhibitor cocktail 1 (Millipore, Billierica, MA, USA). Following centrifugation at 20,000 g for 20 minutes, the protein solution was extracted from the cells. Protein was estimated using Bradford assay. Bradford's reagent (BioRad) was diluted in 1:4 ratio in double distilled water. 2 μl of protein sample was added to 100 μl of the reagent and absorbance was measured at 595 nm. 30 μg of protein was diluted in lysis buffer. 1X loading buffer diluted from 5X stock containing 250 mM Tris-HCl (pH 6.8), 10% SDS, 50% glycerol, 0.1% bromophenol Blue and 10% β-mercaptoethanol was added. The protein samples were then heated at 95° C. for 10 minutes, cooled and centrifuged at 12,000 g for 2 minutes prior to loading.

For western blotting, the proteins were resolved in 10% SDS PAGE (discontinuous buffer system). 1X running buffer containing SDS, Tris Base and Glycine was used to run the gel at 80V for approximately 2 hours. Transfer was done using PVDF membrane (Millipore) having pore size of 0.45 μm. 1X transfer buffer containing Tris-Base, Glycine and 20% methanol was used for wet transfer. Transfer was done at 90V for 3 hours. Following transfer, the PVDF membrane containing the proteins were washed in 1×PBST comprising of 1×PBS and 1% Tween 20 (Sigma Aldrich). The membrane was then incubated for 1 hour at room temperature in 5% skim milk powder to block the non-specific sites. Following multiple washes with 1×PBST to wash away any remaining blocking buffer, the required primary antibody (COP1 [BethylLaboraties], ATGL [Cell Signalling Technology] or Actin[Cell Signalling Technology]) prepared with 1×PBST, 1% Bovine Serum Albumin and 0.04% Sodium Azide was added to the membrane and incubated overnight at 4° C. The next day, the membrane was again washed multiple times with 1×PBST to remove any unbound primary antibody. The membrane was then incubated with goat anti-rabbit secondary antibody (Genei) for 1 hour at room temperature and washed again for multiple times with 1×PBST. The membrane was then developed using Clarity™ ECL Western Blotting Substrate (BioRad) and viewed in ChemiDoc (BioRad).

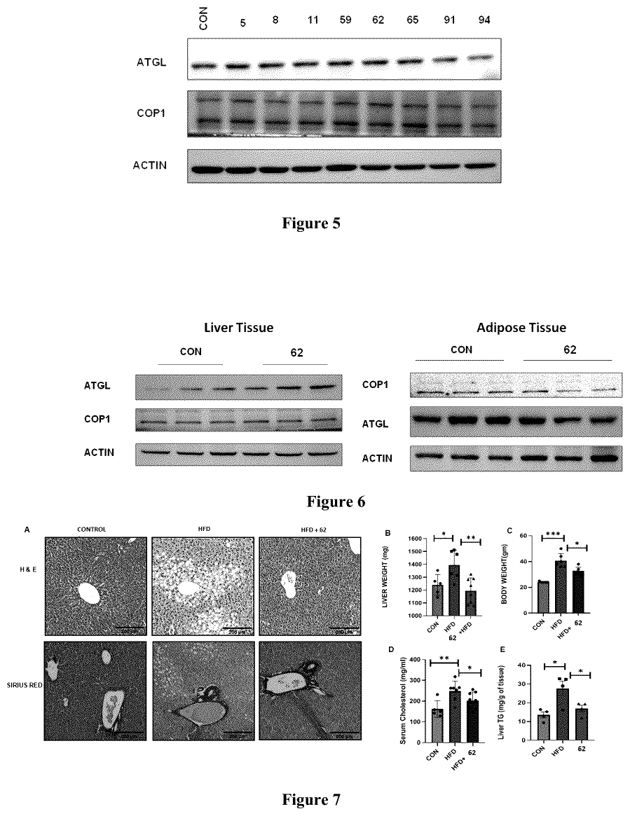

FIG. 5 suggest the comparison of induction of ATGL by the treatment of compounds 5, 8, 11, 59, 62, 65, 91, 94 at 5 μM. All the compounds have ability to induce ATGL and stabilize the COP1 by modulating its activity.

### Example 37

### In Vivo Study of Compound to Check the Expression Level of ATGL and COP1 in Liver

6-8 weeks old healthy male C57BL/6 mice (average weight: 28 grams) were taken for the study. These were then divided into two groups comprising of three mice per group (Control, compound 62). Mice were fed with 80 mg/kg of compound 62 orally. The compound was dissolved in 10% EtOH, 40% PEG, 20% PG, 30% NaCl solution (0.9%). The control group was fed only with the solvent in which the compound was dissolved. Post 9 days of one-time feeding mice were sacrificed and a portion of the excised liver tissue and adipose was homogenized in lysis buffer containing protease inhibitor cocktail. The homogenate was centrifuged at 20,000 g for 30 minutes following which the supernatant containing the protein lysate was collected. The lysate was then diluted accordingly, and protein estimation was carried out by Bradford Assay. This was followed by Western Blotting wherein the levels of ATGL and COP1 were checked. Actin was used as the loading control.

FIG. 6 illustrates results of in vivo study of compounds in mice measuring ATGL and COP1 levels. The western blotting from liver and adipose lysates revealed that the compound 62 was effective in increasing ATGL and COP1 levels.

### Example 38

In Vivo Study of HFD Induced Fatty Liver Disease Model with Compound to Check the Expression Level of ATGL and COP1 in Liver

A preclinical therapeutic model was developed where C57BL/6 mice were fed with 60% kcal high fat diet (HFD) for 12 weeks, and for the last 4 weeks the mice were orally fed with the compound 62 at 80 mg/kg body weight dose.

FIG. 7 illustrates considerable depletion of hepatic lipid droplets (FIG. 7A) and improvement in hepatic fibrogenesis (FIG. 7B). Reductions in mice; total liver weight (FIG. 7B), body weight (FIG. 7C) serum cholesterol level (FIG. 7D) and liver triglyceride (FIG. 7E) content were observed.

### Example 39

### Determination of EC50

TABLE 3

| SI. | Compound | | EC₅₀ (in-vitro) in |
|---|---|---|---|
| No. | No. | Structure | nM |
| 1 | 8 | 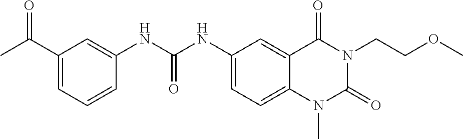 [SMILES: COCCn1c(=O)c2cc(NC(=O)Nc3cccc(C(C)=O)c3)ccc2n(C)c1=O] | 17.78 |
| 2 | 11 | 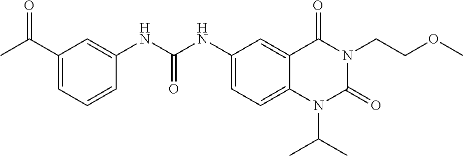 [SMILES: COCCn1c(=O)c2cc(NC(=O)Nc3cccc(C(C)=O)c3)ccc2n(C(C)C)c1=O] | 100.01 |
| 3 | 59 | 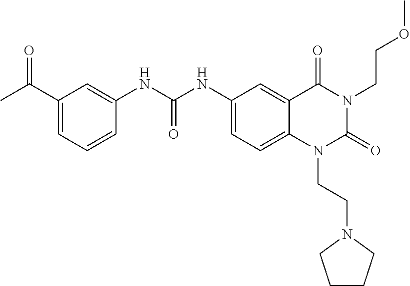 [SMILES: COCCn1c(=O)c2cc(NC(=O)Nc3cccc(C(C)=O)c3)ccc2n(CCN2CCCC2)c1=O] | 208.92 |
| 4 | 62 | 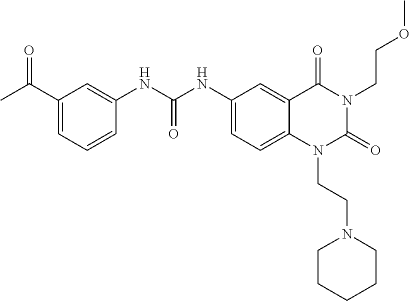 [SMILES: COCCn1c(=O)c2cc(NC(=O)Nc3cccc(C(C)=O)c3)ccc2n(CCN2CCCCC2)c1=O] | 45.70 |
| 5 | 65 | 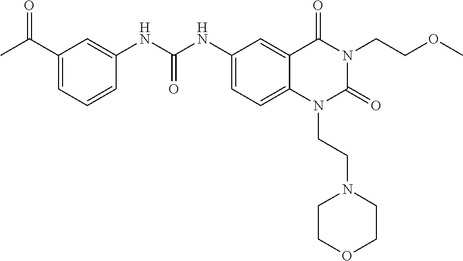 [SMILES: COCCn1c(=O)c2cc(NC(=O)Nc3cccc(C(C)=O)c3)ccc2n(CCN2CCOCC2)c1=O] | 50.01 |

### Example 40

### Fold Change (Induction by the Treatment of Compounds) in ATGL

Densitimetric analysis was performed by image J to calculate the fold change of ATGL with respect to control. The values were normalized with actin.

TABLE 4

| SI. | Compound | | |
|---|---|---|---|
| No. | No. | Structure | Fold change |
| 1 | 5 | 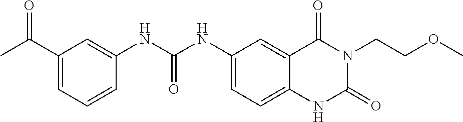 [SMILES: COCCn1c(=O)[nH]c2ccc(NC(=O)Nc3cccc(C(C)=O)c3)cc2c1=O] | 1.44 |
| 1 | 8 | 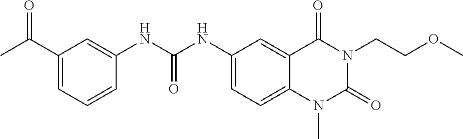 [SMILES: COCCn1c(=O)c2cc(NC(=O)Nc3cccc(C(C)=O)c3)ccc2n(C)c1=O] | 1.34 |
| 2 | 11 | 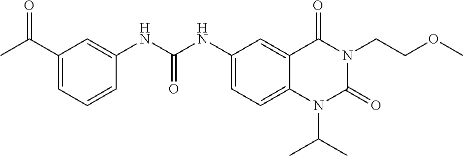 [SMILES: COCCn1c(=O)c2cc(NC(=O)Nc3cccc(C(C)=O)c3)ccc2n(C(C)C)c1=O] | 1.25 |
| 3 | 59 | 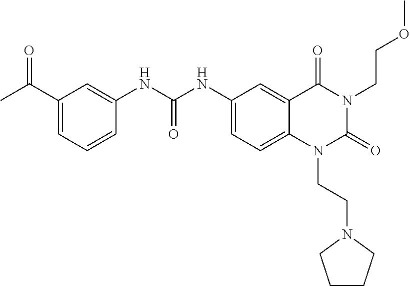 [SMILES: COCCn1c(=O)c2cc(NC(=O)Nc3cccc(C(C)=O)c3)ccc2n(CCN2CCCC2)c1=O] | 1.18 |
| 4 | 62 | 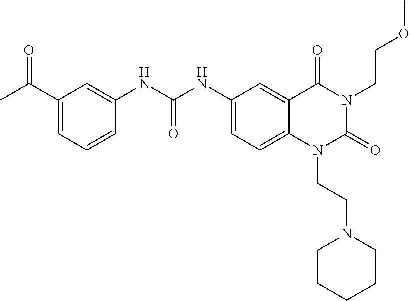 [SMILES: COCCn1c(=O)c2cc(NC(=O)Nc3cccc(C(C)=O)c3)ccc2n(CCN2CCCCC2)c1=O] | 1.55 |
| 5 | 65 | 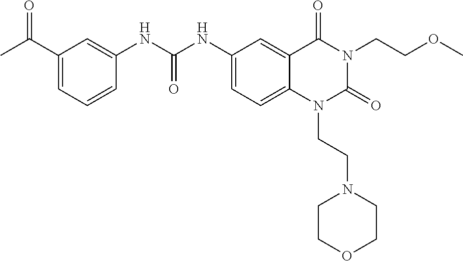 [SMILES: COCCn1c(=O)c2cc(NC(=O)Nc3cccc(C(C)=O)c3)ccc2n(CCN2CCOCC2)c1=O] | 1.44 |
| 6 | 91 | 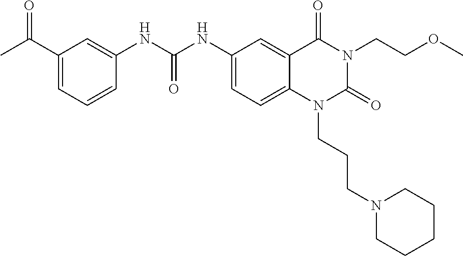 [SMILES: COCCn1c(=O)c2cc(NC(=O)Nc3cccc(C(C)=O)c3)ccc2n(CCCN2CCCCC2)c1=O] | 1.17 |
| 7 | 94 | 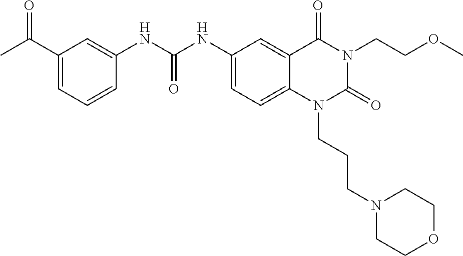 [SMILES: COCCn1c(=O)c2cc(NC(=O)Nc3cccc(C(C)=O)c3)ccc2n(CCCN2CCOCC2)c1=O] | 0.84 |

### Example 41

### Lipophilicity Assay

### Assay Procedure

1.56 g NaH₂PO₄·2H₂O was dissolved in 0.5 L water in a 1 L beaker. After adjusting pH to 7.4 using NaOH solution, the volume was made up to 1 L. Equal volumes of sodium phosphate buffer (10 mM, pH 7.4) and n-octanol were added to a separation funnel and mixed thoroughly by shaking and inverting the funnel several times. The two layers were allowed to separate for overnight and then dispensed in two separate glass bottles. 10 mM stock solution was prepared in 100% DMSO and stored at 4° C. 495 μL of organic phase (1-octanol) was added to each well of a 2 mL deep well plate, followed by 495 μL of buffer and 10 μL of test substance was added. The plate was incubated for 3 hrs at room temperature on a plate shaker at 500 rpm. After incubation, the samples were allowed to equilibrate for 20 min and then centrifuged at 4000 rpm for 30 min for complete phase separation and analysed by LC-UV. Log D=Log (area of octanol/area of buffer)

TABLE 5

| SI. | Compound | | LogD @ pH |
|---|---|---|---|
| No. | No. | Structure | 7.4 |
| 1 | 5 | 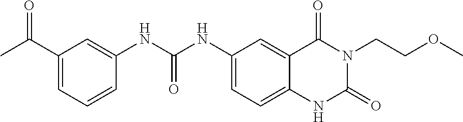 [SMILES: COCCn1c(=O)[nH]c2ccc(NC(=O)Nc3cccc(C(C)=O)c3)cc2c1=O] | 2.17 |
| 2 | 11 | 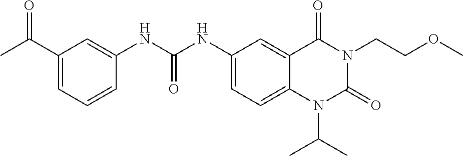 [SMILES: COCCn1c(=O)c2cc(NC(=O)Nc3cccc(C(C)=O)c3)ccc2n(C(C)C)c1=O] | 2.67 |
| 5 | 59 | 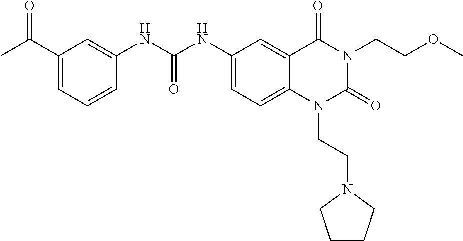 [SMILES: COCCn1c(=O)c2cc(NC(=O)Nc3cccc(C(C)=O)c3)ccc2n(CCN2CCCC2)c1=O] | 2.58 |
| 6 | 62 | 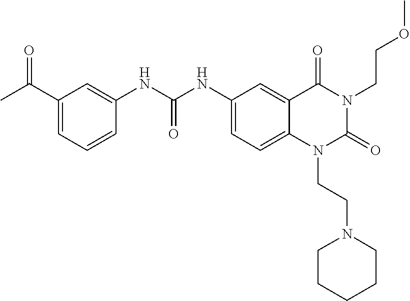 [SMILES: COCCn1c(=O)c2cc(NC(=O)Nc3cccc(C(C)=O)c3)ccc2n(CCN2CCCCC2)c1=O] | 2.78 |
| 3 | 65 | 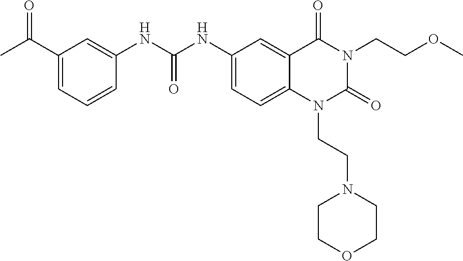 [SMILES: COCCn1c(=O)c2cc(NC(=O)Nc3cccc(C(C)=O)c3)ccc2n(CCN2CCOCC2)c1=O] | 2.08 |
| 4 | 94 | 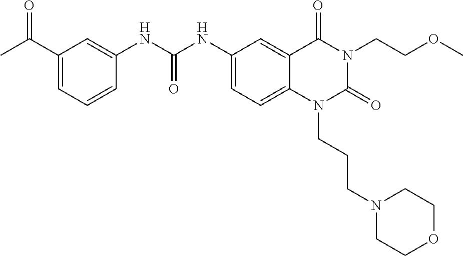 [SMILES: COCCn1c(=O)c2cc(NC(=O)Nc3cccc(C(C)=O)c3)ccc2n(CCCN2CCOCC2)c1=O] | 0.97 |
| 5 | Ketoconazole | | 4.29 |
| | (Control) | | |
| 6 | Metroprolol | | −0.04 |
| | (Control) | | |
| 7 | Propranolol | | 1.25 |
| | (Control) | | |

### Log D Criteria: <1: Hydrophilic; 1-2: Moderate Lipophilic; >2: High Lipophilic

### Example 42

### Plasma Stability Assay:

### Assay Procedure

1 mM Stock of test compound was prepared from 10 mM initial stock solution of compounds by diluting 10 μL of 10 mM stock with 90 μL of DMSO. Then 10 μL of 1 mM stock was diluted with 90 μL of DMSO to give 100 μM concentration. The frozen plasma was thawed at room temperature and centrifuged at 1400 rpm at 4° C., for 15 minutes. Approximately 90% of the clear supernatant fraction was transferred to a separate tube and was used for the assay. Final working stock of 1 μM was prepared by diluting 3 μL of 100 μM with 297 μL of plasma. Plasma containing the test compound was incubated for 120 min at 37° C. in shaker with 500 rpm. 50 μL of aliquot of sample at 0, 15, 30, 60 and 120 minutes were precipitated with 150 μL of acetonitrile containing internal standard and centrifuged at 4000 rpm at 4° C. for 20 minutes. 120 μL of supernatant was diluted with 120 μL of water and analysed by LC-MS/MS.

TABLE 6

| | | | Mean % |
|---|---|---|---|
| | | | remaining at |
| SI. | Compound | | 2 hrs in human |
| No. | No. | Structure | Plasma |
| 1 | 5 | 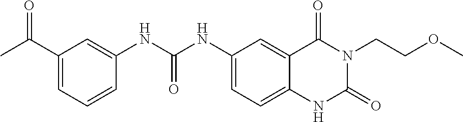 [SMILES: COCCn1c(=O)[nH]c2ccc(NC(=O)Nc3cccc(C(C)=O)c3)cc2c1=O] | 97.40 |
| 2 | 11 | 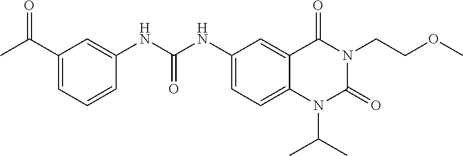 [SMILES: COCCn1c(=O)c2cc(NC(=O)Nc3cccc(C(C)=O)c3)ccc2n(C(C)C)c1=O] | 80.80 |
| 3 | 59 | 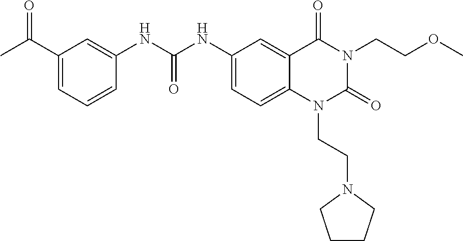 [SMILES: COCCn1c(=O)c2cc(NC(=O)Nc3cccc(C(C)=O)c3)ccc2n(CCN2CCCC2)c1=O] | 32.80 |
| 4 | 62 | 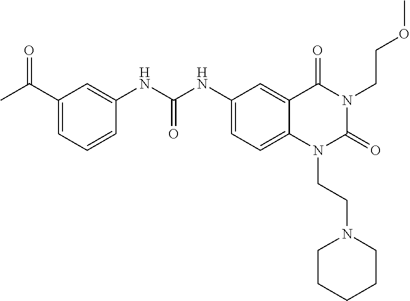 [SMILES: COCCn1c(=O)c2cc(NC(=O)Nc3cccc(C(C)=O)c3)ccc2n(CCN2CCCCC2)c1=O] | 26.18 |
| 5 | 65 | 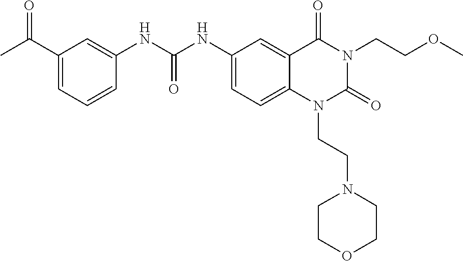 [SMILES: COCCn1c(=O)c2cc(NC(=O)Nc3cccc(C(C)=O)c3)ccc2n(CCN2CCOCC2)c1=O] | 48.20 |
| 6 | 94 | 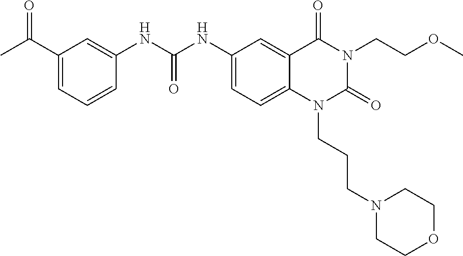 [SMILES: COCCn1c(=O)c2cc(NC(=O)Nc3cccc(C(C)=O)c3)ccc2n(CCCN2CCOCC2)c1=O] | 51.74 |
| 7 | Propantheline | | 1.70 |
| | (Control) | | |

### Example 43

### In-Vitro Evaluation of Metabolic Stability Using Human Liver Microsomes (Human Liver Microsomal Stability-HLM)

### Assay Procedure

1 mM stock solution of test compound was prepared in DMSO and diluted with Acetonitrile:Water (1:1) to get a 100 μM working concentration. 100 mL of Milli Q water was added to K₂HPO₄ (1.398 g) and KH₂PO₄ (0.27 g) to get final pH 7.4 solution of potassium phosphate buffer. 3.333 mg/mL microsomal suspension was prepared by diluting 499.95 μL of 20 mg/mL microsomal stock to 2500.05 μL with buffer. 532.5 μL of 16 mM NADPH stock was added to 2467.5 μL of potassium phosphate buffer to get 2.84 mM working stock. 75 μL of 3.333 mg/mL working stock of liver microsomes and 85 μL of buffer was added to 2.5 μL of test compounds (100 μM). The above mixture was pre incubated for 15 minutes at 37° C. After pre incubation, 32.5 μL of the mixture was added to 17.5 μL of buffer, this was incubated for 60 minutes at 37° C. [60 min Without Cofactor (NADPH)]. 16.25 μL of the pre incubated mixture and 8.75 μL of cofactor was added to 150 μL of acetonitrile containing internal standard [0 min Sample]. 62 μL of cofactor was added to remaining pre incubation mixture [Incubation mixture]. 25 μL of incubation mixture at 0, 5, 15, 30, 60 min and 60 min without cofactor were precipitated with 150 μL of acetonitrile containing internal standard, vortexed and centrifuged at 4000 rpm at 4° C. for 20 minutes. 120 μL of supernatant was diluted with 120 μL of water and analyzed by LC-MS/MS [sample preparation].

TABLE 7

| SI. | Compound | | % QH (Well |
|---|---|---|---|
| No. | No. | Structure | stirred model) |
| 1 | 5 | 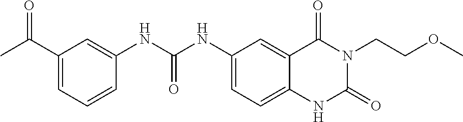 [SMILES: COCCn1c(=O)[nH]c2ccc(NC(=O)Nc3cccc(C(C)=O)c3)cc2c1=O] | 7.60 |
| 2 | 11 | 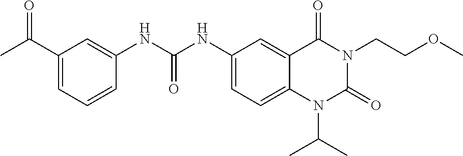 [SMILES: COCCn1c(=O)c2cc(NC(=O)Nc3cccc(C(C)=O)c3)ccc2n(C(C)C)c1=O] | 40.30 |
| 3 | 59 | 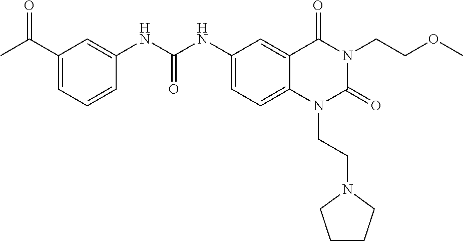 [SMILES: COCCn1c(=O)c2cc(NC(=O)Nc3cccc(C(C)=O)c3)ccc2n(CCN2CCCC2)c1=O] | 17.46 |
| 4 | 62 | 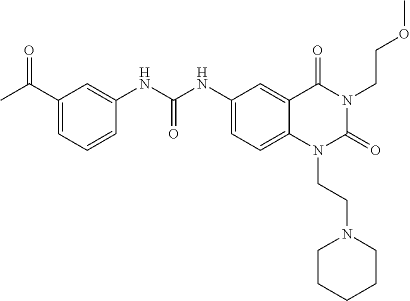 [SMILES: COCCn1c(=O)c2cc(NC(=O)Nc3cccc(C(C)=O)c3)ccc2n(CCN2CCCCC2)c1=O] | 56.74 |
| 5 | 65 | 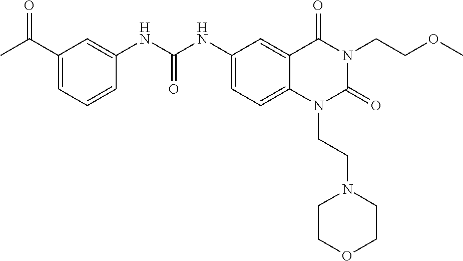 [SMILES: COCCn1c(=O)c2cc(NC(=O)Nc3cccc(C(C)=O)c3)ccc2n(CCN2CCOCC2)c1=O] | 62.0 |
| 6 | 94 | 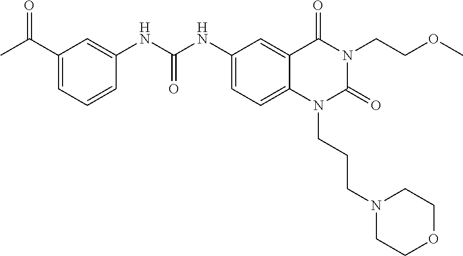 [SMILES: COCCn1c(=O)c2cc(NC(=O)Nc3cccc(C(C)=O)c3)ccc2n(CCCN2CCOCC2)c1=O] | 59.60 |
| 7 | Verapamil | | 81.60 |
| | HCl (Control) | | |

Classifications criteria: % QH; <30: Low Clearance 30-70: Medium Clearance >70: High Clearance

### Example 4

### In-Vitro Evaluation of Kinetic Solubility

PBS sachet was dissolved in 0.9 L of Milli Q water and pH was adjusted to 7.4. Final volume was made up to 1 L with the water and stored at ambient temperature 21-25° C. Stock solution (50 mM) of respective compounds (5, 11, 59, 62, 65, 94) were prepared in DMSO and stored at 4-8° C. 4 μL from the 50 mM stock solution were added to deep well plate containing 396 μL of pH 7.4 phosphate buffer, mixed and incubated for 24 hours at room temperature with constant mixing at 300 rpm. The plate was sealed well during the incubation process. After incubation, the samples were centrifuged for 20 min at 4000 rpm. The supernatant was analysed by HPLC-UV. The DMSO content in the sample was 1.0%. The final concentration of the compound in deep well plate was 500 μM.

TABLE 8

| SI. | Compound | | Kinetic solubility |
|---|---|---|---|
| No. | No. | Structure | (μg/mL) |
| 1 | 5 | 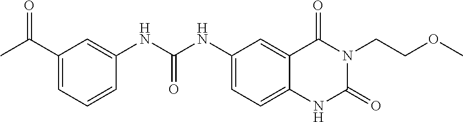 [SMILES: COCCn1c(=O)[nH]c2ccc(NC(=O)Nc3cccc(C(C)=O)c3)cc2c1=O] | 0.8 |
| 2 | 11 | 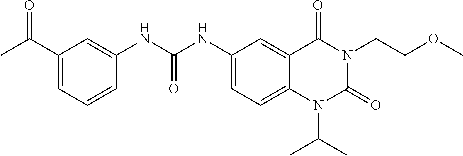 [SMILES: COCCn1c(=O)c2cc(NC(=O)Nc3cccc(C(C)=O)c3)ccc2n(C(C)C)c1=O] | 2.8 |
| 3 | 59 | 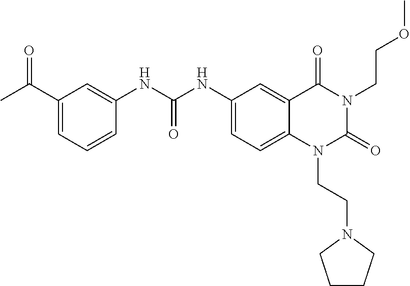 [SMILES: COCCn1c(=O)c2cc(NC(=O)Nc3cccc(C(C)=O)c3)ccc2n(CCN2CCCC2)c1=O] | 124.3 |
| 4 | 62 | 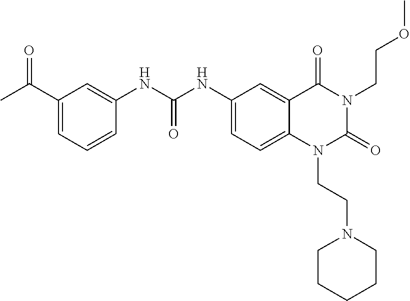 [SMILES: COCCn1c(=O)c2cc(NC(=O)Nc3cccc(C(C)=O)c3)ccc2n(CCN2CCCCC2)c1=O] | 115.5 |
| 5 | 65 | 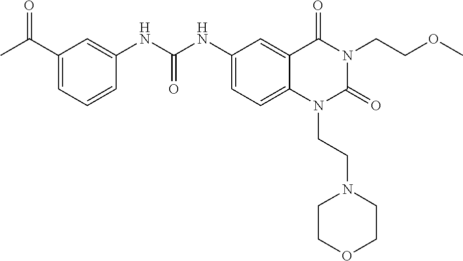 [SMILES: COCCn1c(=O)c2cc(NC(=O)Nc3cccc(C(C)=O)c3)ccc2n(CCN2CCOCC2)c1=O] | 20.8 |
| 6 | 94 | 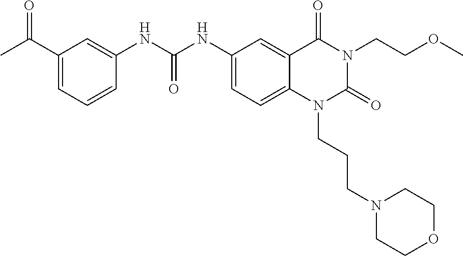 [SMILES: COCCn1c(=O)c2cc(NC(=O)Nc3cccc(C(C)=O)c3)ccc2n(CCCN2CCOCC2)c1=O] | 89.9 |
| 5 | Verapamil | | 81.6 |
| | HCl (Control) | | |

Although the subject matter has been described in considerable detail with reference to certain examples and implementations thereof, other implementations are possible.

### Advantages of the Invention

The compounds of the present invention having structure I have several advantages. The compounds having structure I are capable of modulating COP1 Ubiquitin Ligase enzyme through stabilization in hepatocytes. The compounds having structure I can reduce the level of triglycerides in hepatocytes. Hence, they can be used in a clinical application for treating conditions involving Non-Alcoholic Fatty Liver Disease (NAFLD). The compounds having structure I possess good invitro ADME property (kinetic solubility, Log D and metabolic stability)

The invention claimed is:

1. A compound having structure I or salts thereof, 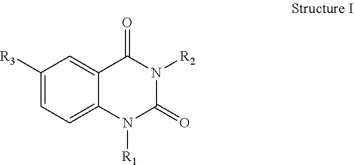
wherein
R₁ is independently selected from the group consisting of: 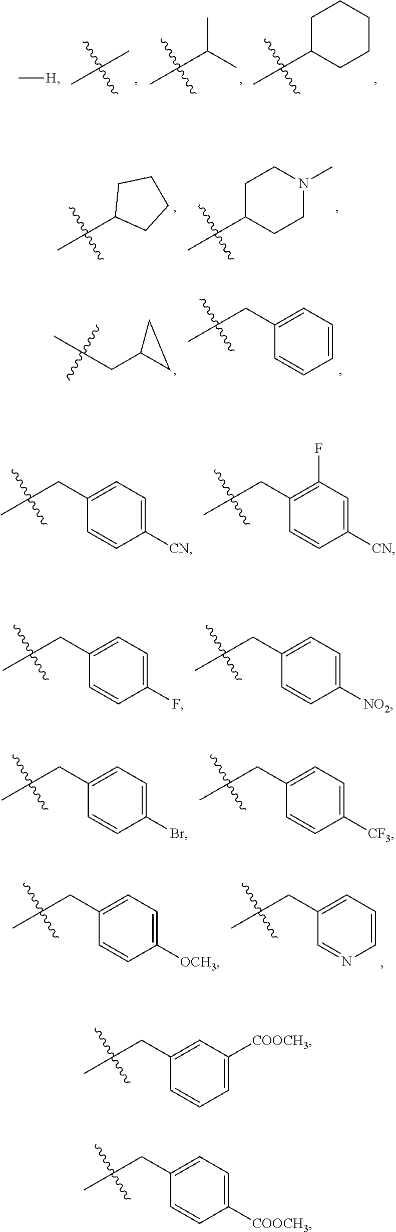 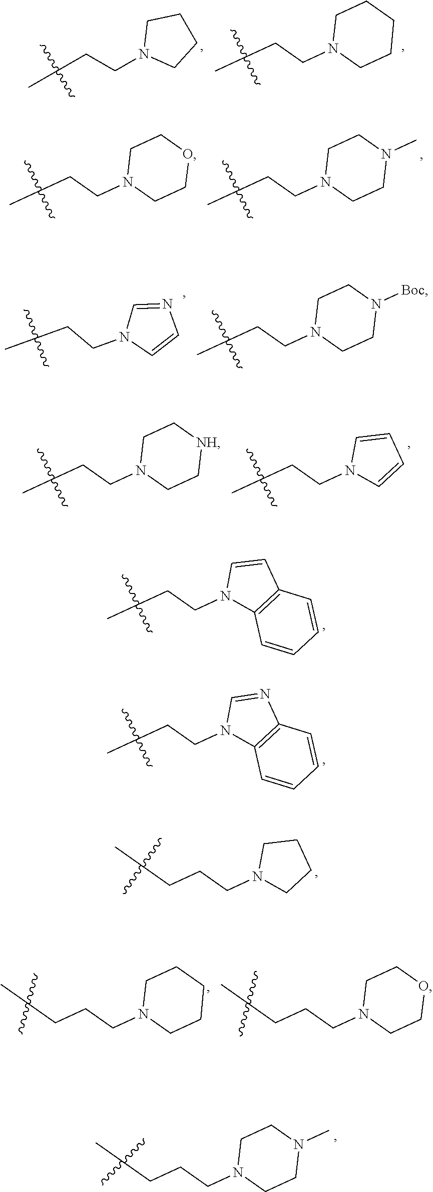
R₂ is independently selected from the group consisting of: 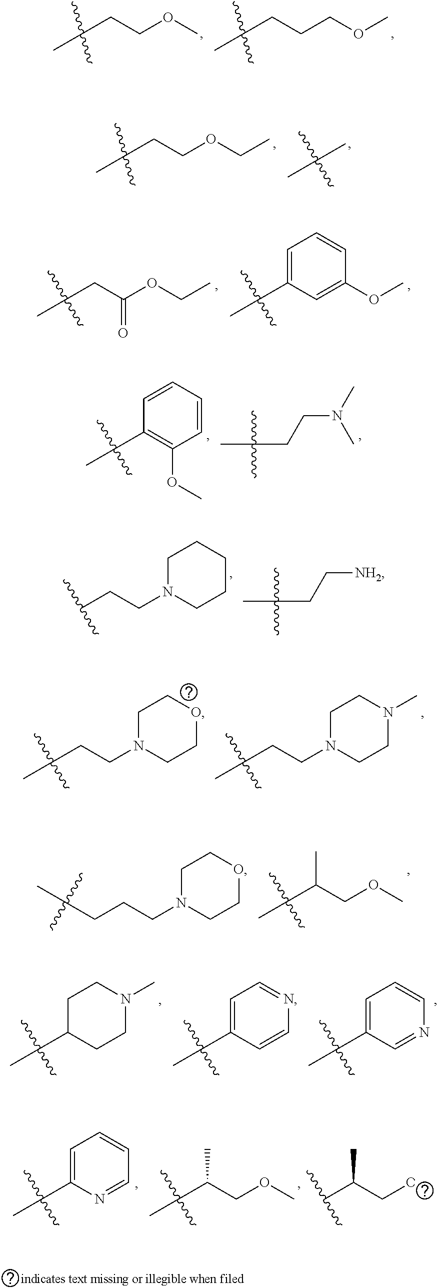
R₃ is independently selected from the group consisting of:

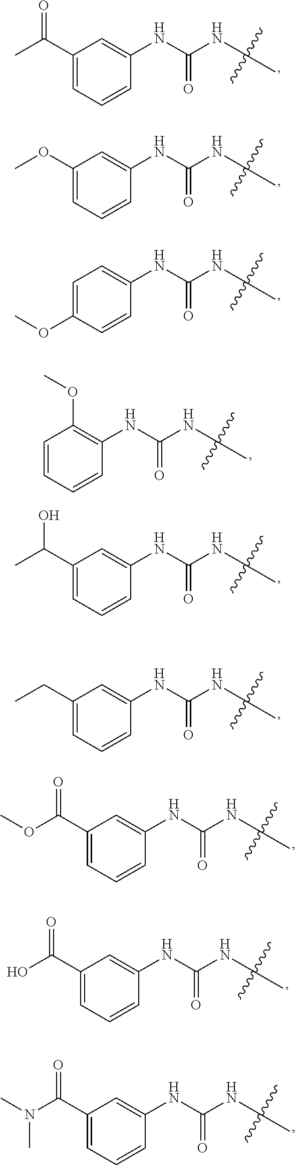

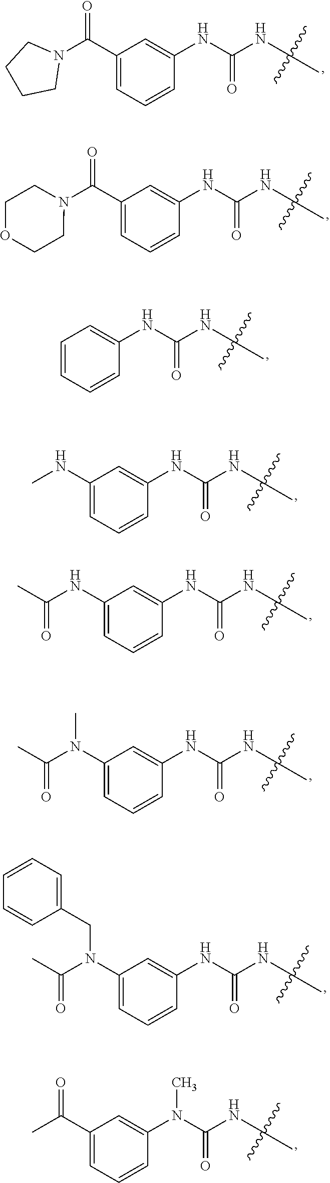

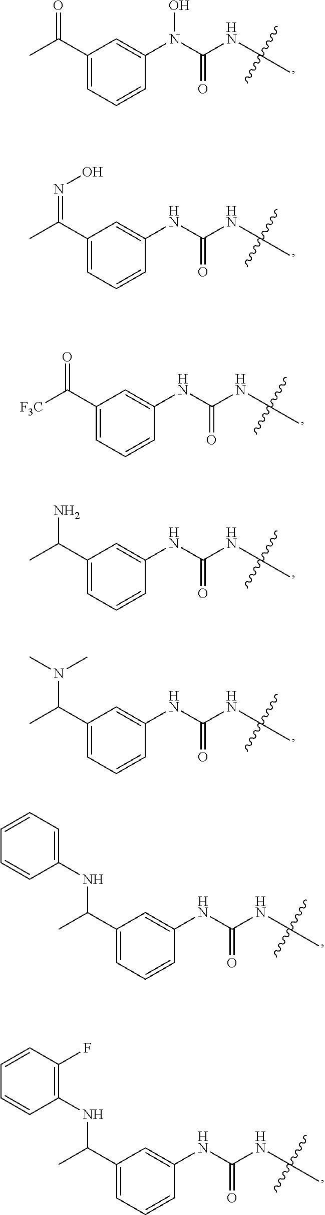

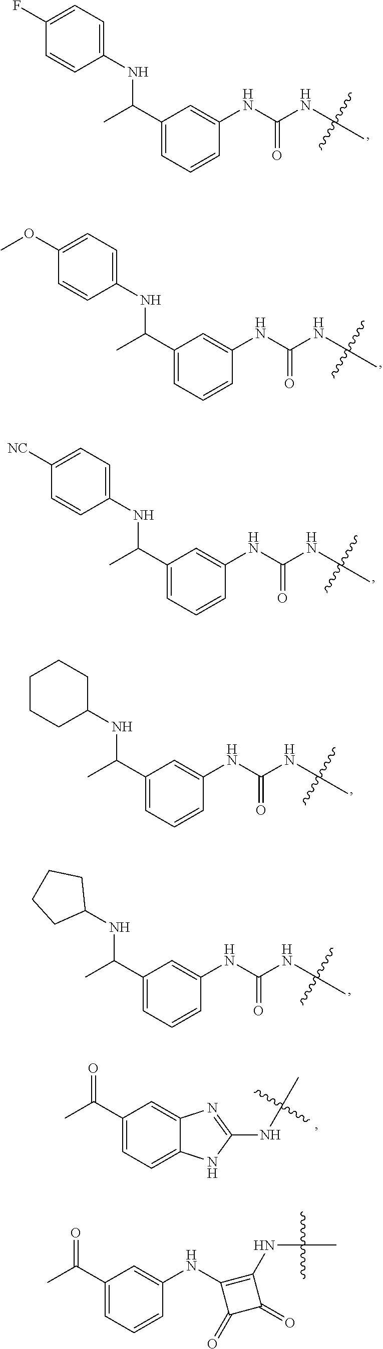

2. The compound having structure 1 as claimed in claim 1 selected from the group consisting of:
1-(3-acetylphenyl)-3-(3-(2-methoxyethyl)-2,4-dioxo-1,2,3,4-tetrahydroquinazolin-6-yl)urea (5),
1-(3-acetylphenyl)-3-(3-(2-methoxyethyl)-1-methyl-2,4-dioxo-1,2,3,4-tetrahydroquinazolin-6-yl)urea (8),
1-(3-acetylphenyl)-3-(1-isopropyl-3-(2-methoxyethyl)-2,4-dioxo-1,2,3,4-tetrahydroquinazolin-6-yl)urea (11),
1-(3-acetylphenyl)-3-(1-cyclohexyl-3-(2-methoxyethyl)-2,4-dioxo-1,2,3,4-tetrahydroquinazolin-6-yl)urea (14),
1-(3-acetylphenyl)-3-(1-cyclopentyl-3-(2-methoxyethyl)-2,4-dioxo-1,2,3,4-tetrahydroquinazolin-6-yl)urea (17),
1-(3-acetylphenyl)-3-(3-(2-methoxyethyl)-1-(1-methylpiperidin-4-yl)-2,4-dioxo-1,2,3,4-tetrahydroquinazolin-6-yl)urea (20),
1-(3-acetylphenyl)-3-(1-(cyclopropylmethyl)-3-(2-methoxyethyl)-2,4-dioxo-1,2,3,4-tetrahydroquinazolin-6-yl)urea (23),
1-(3-acetylphenyl)-3-(1-benzyl-3-(2-methoxyethyl)-2,4-dioxo-1,2,3,4-tetrahydroquinazolin-6-yl)urea (26),
1-(3-acetylphenyl)-3-(1-(4-cyanobenzyl)-3-(2-methoxyethyl)-2,4-dioxo-1,2,3,4-tetrahydroquinazolin-6-yl)urea (29),
1-(3-acetylphenyl)-3-(1-(4-cyano-2-fluorobenzyl)-3-(2-methoxyethyl)-2,4-dioxo-1,2,3,4-tetrahydroquinazolin-6-yl)urea (32),
1-(3-acetylphenyl)-3-(1-(4-fluorobenzyl)-3-(2-methoxyethyl)-2,4-dioxo-1,2,3,4-tetrahydroquinazolin-6-yl)urea (35),
1-(3-acetylphenyl)-3-(3-(2-methoxyethyl)-1-(4-nitrobenzyl)-2,4-dioxo-1,2,3,4-tetrahydroquinazolin-6-yl)urea (38),
1-(3-acetylphenyl)-3-(1-(4-bromobenzyl)-3-(2-methoxyethyl)-2,4-dioxo-1,2,3,4-tetrahydroquinazolin-6-yl)urea (41),
1-(3-acetylphenyl)-3-(3-(2-methoxyethyl)-2,4-dioxo-1-(4-(trifluoromethyl)benzyl)-1,2,3,4-tetrahydroquinazolin-6-yl)urea (44),
1-(3-acetylphenyl)-3-(1-(4-methoxybenzyl)-3-(2-methoxyethyl)-2,4-dioxo-1,2,3,4-tetrahydroquinazolin-6-yl)urea (47),
1-(3-acetylphenyl)-3-(3-(2-methoxyethyl)-2,4-dioxo-1-(pyridin-3-ylmethyl)-1,2,3,4-tetrahydroquinazolin-6-yl)urea (50),
Methyl 3-((6-(3-(3-acetylphenyl)ureido)-3-(2-methoxyethyl)-2,4-dioxo-3,4-dihydroquinazolin-1 (2H)-yl)methyl)benzoate (53),
methyl 4-((6-(3-(3-acetylphenyl)ureido)-3-(2-methoxyethyl)-2,4-dioxo-3,4-dihydroquinazolin-1 (2H)-yl)methyl)benzoate (56),
1-(3-acetylphenyl)-3-(3-(2-methoxyethyl)-2,4-dioxo-1-(2-(pyrrolidin-1-yl)ethyl)-1,2,3,4-tetrahydroquinazolin-6-yl)urea (59),
1-(3-acetylphenyl)-3-(3-(2-methoxyethyl)-2,4-dioxo-1-(2-(piperidin-1-yl)ethyl)-1,2,3,4-tetrahydroquinazolin-6-yl)urea (62),
1-(3-acetylphenyl)-3-(3-(2-methoxyethyl)-1-(2-morpholinoethyl)-2,4-dioxo-1,2,3,4-tetrahydroquinazolin-6-yl)urea (65),
1-(3-acetylphenyl)-3-(3-(2-methoxyethyl)-1-(2-(4-methylpiperazin-1-yl)ethyl)-2,4-dioxo-1,2,3,4-tetrahydroquinazolin-6-yl)urea (68),
1-(1-(2-(1H-imidazol-1-yl)ethyl)-3-(2-methoxyethyl)-2,4-dioxo-1,2,3,4-tetrahydroquinazolin-6-yl)-3-(3-acetylphenyl)urea (72),
tert-butyl 4-(2-(6-(3-(3-acetylphenyl)ureido)-3-(2-methoxyethyl)-2,4-dioxo-3,4-dihydroquinazolin-1 (2H)-yl)ethyl) piperazine-1-carboxylate (75),
1-(3-acetylphenyl)-3-(3-(2-methoxyethyl)-2,4-dioxo-1-(2-(piperazin-1-yl)ethyl)-1,2,3,4-tetrahydroquinazolin-6-yl)urea (76),
1-(1-(2-(1H-pyrrol-1-yl)ethyl)-3-(2-methoxyethyl)-2,4-dioxo-1,2,3,4-tetrahydroquinazolin-6-yl)-3-(3-acetylphenyl)urea (79),
1-(1-(2-(1H-indol-1-yl)ethyl)-3-(2-methoxyethyl)-2,4-dioxo-1,2,3,4-tetrahydroquinazolin-6-yl)-3-(3-acetylphenyl)urea (82),
1-(1-(2-(1H-benzo[d]imidazol-1-yl)ethyl)-3-(2-methoxyethyl)-2,4-dioxo-1,2,3,4-tetrahydroquinazolin-6-yl)-3-(3-acetylphenyl)urea (85),
1-(3-acetylphenyl)-3-(3-(2-methoxyethyl)-2,4-dioxo-1-(3-(pyrrolidin-1-yl)propyl)-1,2,3,4-tetrahydroquinazolin-6-yl)urea (88),
1-(3-acetylphenyl)-3-(3-(2-methoxyethyl)-2,4-dioxo-1-(3-(piperidin-1-yl)propyl)-1,2,3,4-tetrahydroquinazolin-6-yl)urea (91),
1-(3-acetylphenyl)-3-(3-(2-methoxyethyl)-1-(3-morpholinopropyl)-2,4-dioxo-1,2,3,4-tetrahydroquinazolin-6-yl)urea (94),
1-(3-acetylphenyl)-3-(3-(2-methoxyethyl)-1-(3-(4-methylpiperazin-1-yl)propyl)-2,4-dioxo-1,2,3,4-tetrahydroquinazolin-6-yl)urea (97),
1-(3-acetylphenyl)-3-(3-(3-methoxypropyl)-2,4-dioxo-1-(2-(piperidin-1-yl)ethyl)-1,2,3,4-tetrahydroquinazolin-6-yl)urea (102),
1-(3-acetylphenyl)-3-(3-(2-ethoxyethyl)-2,4-dioxo-1-(2-(piperidin-1-yl)ethyl)-1,2,3,4-tetrahydroquinazolin-6-yl)urea (107),
1-(3-acetylphenyl)-3-(3-ethyl-2,4-dioxo-1-(2-(piperidin-1-yl)ethyl)-1,2,3,4-tetrahydroquinazolin-6-yl)urea (112),
ethyl 2-(6-(3-(3-acetylphenyl)ureido)-2,4-dioxo-1-(2-(piperidin-1-yl)ethyl)-1,2-dihydroquinazolin-3 (4H)-yl)acetate (117),
1-(3-acetylphenyl)-3-(3-(3-methoxyphenyl)-2,4-dioxo-1-(2-(piperidin-1-yl)ethyl)-1,2,3,4-tetrahydroquinazolin-6-yl)urea (122),
1-(3-acetylphenyl)-3-(3-(2-methoxyphenyl)-2,4-dioxo-1-(2-(piperidin-1-yl)ethyl)-1,2,3,4-tetrahydroquinazolin-6-yl)urea (127),
1-(3-acetylphenyl)-3-(3-(2-(dimethylamino)ethyl)-2,4-dioxo-1-(2-(piperidin-1-yl)ethyl)-1,2,3,4-tetrahydroquinazolin-6-yl)urea (132),
1-(3-acetylphenyl)-3-(2,4-dioxo-1,3-bis(2-(piperidin-1-yl)ethyl)-1,2,3,4-tetrahydroquinazolin-6-yl)urea (137),
1-(3-acetylphenyl)-3-(3-(2-aminoethyl)-2,4-dioxo-1-(2-(piperidin-1-yl)ethyl)-1,2,3,4-tetrahydroquinazolin-6-yl)urea (142),
1-(3-acetylphenyl)-3-(3-(2-(4-methylpiperazin-1-yl)ethyl)-2,4-dioxo-1-(2-(piperidin-1-yl)ethyl)-1,2,3,4-tetrahydroquinazolin-6-yl)urea (147),
1-(3-acetylphenyl)-3-(3-(3-morpholinopropyl)-2,4-dioxo-1-(2-(piperidin-1-yl)ethyl)-1,2,3,4-tetrahydroquinazolin-6-yl)urea (152),
1-(3-acetylphenyl)-3-(3-(1-methoxypropan-2-yl)-2,4-dioxo-1-(2-(piperidin-1-yl)ethyl)-1,2,3,4-tetrahydroquinazolin-6-yl)urea (157),
1-(3-acetylphenyl)-3-(3-(1-methylpiperidin-4-yl)-2,4-dioxo-1-(2-(piperidin-1-yl)ethyl)-1,2,3,4-tetrahydroquinazolin-6-yl)urea (162),
1-(3-acetylphenyl)-3-(2,4-dioxo-1-(2-(piperidin-1-yl)ethyl)-3-(pyridin-4-yl)-1,2,3,4-tetrahydroquinazolin-6-yl)urea (167),
1-(3-acetylphenyl)-3-(2,4-dioxo-1-(2-(piperidin-1-yl)ethyl)-3-(pyridin-3-yl)-1,2,3,4-tetrahydroquinazolin-6-yl)urea (172),
1-(3-acetylphenyl)-3-(2,4-dioxo-1-(2-(piperidin-1-yl)ethyl)-3-(pyridin-2-yl)-1,2,3,4-tetrahydroquinazolin-6-yl)urea (177),
1-(3-(2-methoxyethyl)-2,4-dioxo-1-(2-(piperidin-1-yl)ethyl)-1,2,3,4-tetrahydroquinazolin-6-yl)-3-(3-methoxyphenyl)urea (178),
1-(3-(2-methoxyethyl)-2,4-dioxo-1-(2-(piperidin-1-yl)ethyl)-1,2,3,4-tetrahydroquinazolin-6-yl)-3-(2-methoxyphenyl)urea (179),
1-(3-(1-hydroxyethyl)phenyl)-3-(3-(2-methoxyethyl)-2,4-dioxo-1-(2-(piperidin-1-yl)ethyl)-1,2,3,4-tetrahydroquinazolin-6-yl)urea (180),
1-(3-ethylphenyl)-3-(3-(2-methoxyethyl)-2,4-dioxo-1-(2-(piperidin-1-yl)ethyl)-1,2,3,4-tetrahydroquinazolin-6-yl)urea (181),
Methyl 3-(3-(3-(2-methoxyethyl)-2,4-dioxo-1-(2-(piperidin-1-yl)ethyl)-1,2,3,4-tetrahydroquinazolin-6-yl) ureido)benzoate (182),
3-(3-(3-(2-methoxyethyl)-2,4-dioxo-1-(2-(piperidin-1-yl)ethyl)-1,2,3,4-tetrahydroquinazolin-6-yl) ureido)benzoic acid (183),
3-(3-(3-(2-methoxyethyl)-2,4-dioxo-1-(2-(piperidin-1-yl)ethyl)-1,2,3,4-tetrahydroquinazolin-6-yl) ureido)-N,N-dimethylbenzamide (184),
1-(3-(2-methoxyethyl)-2,4-dioxo-1-(2-(piperidin-1-yl)ethyl)-1,2,3,4-tetrahydroquinazolin-6-yl)-3-(3-(pyrrolidine-1-carbonyl)phenyl)urea (185),
1-(3-(2-methoxyethyl)-2,4-dioxo-1-(2-(piperidin-1-yl)ethyl)-1,2,3,4-tetrahydroquinazolin-6-yl)-3-(3-(morpholine-4-carbonyl)phenyl)urea (186),
1-(3-(2-methoxyethyl)-2,4-dioxo-1-(2-(piperidin-1-yl)ethyl)-1,2,3,4-tetrahydroquinazolin-6-yl)-3-phenylurea (187),
1-(3-(2-methoxyethyl)-2,4-dioxo-1-(2-(piperidin-1-yl)ethyl)-1,2,3,4-tetrahydroquinazolin-6-yl)-3-(3-(methylamino)phenyl)urea (188),
N-(3-(3-(3-(2-methoxyethyl)-2,4-dioxo-1-(2-(piperidin-1-yl)ethyl)-1,2,3,4-tetrahydroquinazolin-6-yl)ureido)phenyl)acetamide (189),
N-(3-(3-(3-(2-methoxyethyl)-2,4-dioxo-1-(2-(piperidin-1-yl)ethyl)-1,2,3,4-tetrahydroquinazolin-6-yl) ureido)phenyl)-N-methylacetamide (190),
N-benzyl-N-(3-(3-(3-(2-methoxyethyl)-2,4-dioxo-1-(2-(piperidin-1-yl)ethyl)-1,2,3,4-tetrahydroquinazolin-6-yl)ureido)phenyl)acetamide (191),
1-(3-acetylphenyl)-3-(3-(2-methoxyethyl)-2,4-dioxo-1-(2-(piperidin-1-yl)ethyl)-1,2,3,4-tetrahydroquinazolin-6-yl)-1-methylurea (192),
1-(3-acetylphenyl)-1-hydroxy-3-(3-(2-methoxyethyl)-2,4-dioxo-1-(2-(piperidin-1-yl)ethyl)-1,2,3,4-tetrahydroquinazolin-6-yl)urea (193),
(Z)-1-(3-(1-(hydroxyimino)ethyl)phenyl)-3-(3-(2-methoxyethyl)-2,4-dioxo-1-(2-(piperidin-1-yl)ethyl)-1,2,3,4-tetrahydroquinazolin-6-yl)urea (194),
1-(3-(2-methoxyethyl)-2,4-dioxo-1-(2-(piperidin-1-yl)ethyl)-1,2,3,4-tetrahydroquinazolin-6-yl)-3-(3-(2,2,2-trifluoroacetyl)phenyl)urea (195),
1-(3-(1-aminoethyl)phenyl)-3-(3-(2-methoxyethyl)-2,4-dioxo-1-(2-(piperidin-1-yl)ethyl)-1,2,3,4-tetrahydroquinazolin-6-yl)urea (196),
1-(3-(1-(dimethylamino)ethyl)phenyl)-3-(3-(2-methoxyethyl)-2,4-dioxo-1-(2-(piperidin-1-yl)ethyl)-1,2,3,4-tetrahydroquinazolin-6-yl)urea (197),
1-(3-(2-methoxyethyl)-2,4-dioxo-1-(2-(piperidin-1-yl)ethyl)-1,2,3,4-tetrahydroquinazolin-6-yl)-3-(3-(1-(phenylamino)ethyl)phenyl)urea (199),
1-(3-(1-((2-fluorophenyl)amino)ethyl)phenyl)-3-(3-(2-methoxyethyl)-2,4-dioxo-1-(2-(piperidin-1-yl)ethyl)-1,2,3,4-tetrahydroquinazolin-6-yl)urea (201),
1-(3-(1-((4-fluorophenyl)amino)ethyl)phenyl)-3-(3-(2-methoxyethyl)-2,4-dioxo-1-(2-(piperidin-1-yl)ethyl)-1,2,3,4-tetrahydroquinazolin-6-yl)urea (203),
1-(3-(2-methoxyethyl)-2,4-dioxo-1-(2-(piperidin-1-yl)ethyl)-1,2,3,4-tetrahydroquinazolin-6-yl)-3-(3-(1-((4-methoxyphenyl)amino)ethyl)phenyl)urea (205),
1-(3-(1-((4-cyanophenyl)amino)ethyl)phenyl)-3-(3-(2-methoxyethyl)-2,4-dioxo-1-(2-(piperidin-1-yl)ethyl)-1,2,3,4-tetrahydroquinazolin-6-yl)urea (207),
1-(3-(1-(cyclohexylamino)ethyl)phenyl)-3-(3-(2-methoxyethyl)-2,4-dioxo-1-(2-(piperidin-1-yl)ethyl)-1,2,3,4-tetrahydroquinazolin-6-yl)urea (209),
1-(3-(1-(cyclopentylamino)ethyl)phenyl)-3-(3-(2-methoxyethyl)-2,4-dioxo-1-(2-(piperidin-1-yl)ethyl)-1,2,3,4-tetrahydroquinazolin-6-yl)urea (211),
6-((5-acetyl-1H-benzo[d]imidazol-2-yl)amino)-3-(2-methoxyethyl)-1-(2-(piperidin-1-yl)ethyl) quinazoline-2,4 (1H,3H)-dione (216),
6-((2-((3-acetylphenyl)amino)-3,4-dioxocyclobut-1-en-1-yl)amino)-3-(2-methoxyethyl)-1-(2-(piperidin-1-yl)ethyl) quinazoline-2,4 (1H,3H)-dione (217),
(R)-1-(3-acetylphenyl)-3-(3-(1-methoxypropan-2-yl)-2,4-dioxo-1-(2-(piperidin-1-yl)ethyl)-1,2,3,4-tetrahydroquinazolin-6-yl)urea (222),
(S)-1-(3-acetylphenyl)-3-(3-(1-methoxypropan-2-yl)-2,4-dioxo-1-(2-(piperidin-1-yl)ethyl)-1,2,3,4-tetrahydroquinazolin-6-yl)urea (226),
(R)-3-(3-(3-(1-methoxypropan-2-yl)-2,4-dioxo-1-(2-(piperidin-1-yl)ethyl)-1,2,3,4-tetrahydroquinazolin-6-yl) ureido)-N,N-dimethylbenzamide (228),
(S)-3-(3-(3-(1-methoxypropan-2-yl)-2,4-dioxo-1-(2-(piperidin-1-yl)ethyl)-1,2,3,4-tetrahydroquinazolin-6-yl) ureido)-N,N-dimethylbenzamide (229),
(R)-1-(3-(1-methoxypropan-2-yl)-2,4-dioxo-1-(2-(piperidin-1-yl)ethyl)-1,2,3,4-tetrahydroquinazolin-6-yl)-3-(3-(2,2,2-trifluoroacetyl)phenyl)urea (230),
(S)-1-(3-(1-methoxypropan-2-yl)-2,4-dioxo-1-(2-(piperidin-1-yl)ethyl)-1,2,3,4-tetrahydroquinazolin-6-yl)-3-(3-(2,2,2-trifluoroacetyl)phenyl)urea (231).

3. A process for preparation of the compound having structure I as claimed in claim 1, the process steps comprising:
(i) reacting 2-amino-5-nitrobenzoic acid (compound 1) with an aliphatic or an aromatic amine selected from the group consisting of 2-methoxyethylamine, methoxypropylamine, 2-ethoxyethylamine, ethylamine 2M in THF, glycine ethylester hydrochloride, m-anisidine, o-anisidine, N,N-dimethylethylenediamine, 1-(2-aminoethyl) piperidine, 4-(2-aminoethyl) morpholine, 1-(2-Aminoethyl)-4-methylpiperazine, 3-(4-morpholinyl) propylamine, rac-1-methoxy-2-propylamine, 4-amino-1-methylpiperidine, 4-aminopyridine, 3-aminopyridine, 2-aminopyridine, (R)-1-methoxy-2-propylamine, (S)-1-methoxy-2-propylamine in presence of HATU/DMF followed by TEA as a base at room temperature for 1-3 hours to obtain an amide compound selected from the group consisting of 2, 98, 103, 108, 113, 118, 123, 128, 133, 138, 143, 148, 153, 158, 163, 168, 173, 218, 223;
(ii) cyclizing the compound selected from the group consisting of 2, 98, 103, 108, 113, 118, 123, 128, 133, 138, 143, 148, 153, 158, 163, 168, 173, 218, 223 obtained in step (i) using a cyclizing agent CDI in DMF as solvent at 100° C. for 12-16 hours to obtain a compound selected from the group consisting of 3, 99, 104, 109, 114, 119, 124, 129, 134, 139, 144, 149, 154, 159, 164, 169, 174, 219, 224;
(iii) reacting the compound 3 obtained in step (ii) with methyliodide and dry DMF at 0° C. for 12 hours to obtain the compound 6;
(iv) reacting the compound 3, 99, 104, 109, 114, 119, 124, 129, 134, 139, 144, 149, 154, 159, 164, 169, 174, 219, 224 obtained in step (ii) with suitable alkyl chloride selected from the group consisting of2-iodopropane, bromocyclohexane, bromocyclopentane, 4-bromo 1-methyl piperidine, (bromomethyl)cyclopropane, benzyl bromide, 4-(bromomethyl)benzonitrile, 4-(bromomethyl)-3-fluorobenzonitrile, 4-fluorobenzyl bromide, 4-nitrobenzyl bromide, 4-bromobenzyl bromide, 4-Methoxybenzyl bromide, 3-(bromomethyl)pyridin hydrobromide, methyl 3-(bromomethyl)benzoate, methyl 4-(bromomethyl)benzoate, 1-(2-chloroethyl)pyrrolidine, 1-(2-chloroethyl)piperidine, 4-(2-chloroethyl)morpholine, 1-(2-chloroethyl)-4-methylpiperazine, 1-(3-chloropropyl)pyrrolidine, 1-(3-chloropropyl)piperidine, 4-(3-chloropropyl) morpholine, 1-(3-chloropropyl)-4-methylpiperazine with K₂CO₃ and dry DMF at 120° C. for 12 hours to obtain the compound selected from the group consisting of 9, 12, 18, 21, 24, 27, 30, 33, 36, 39, 42, 45, 48, 51, 54, 57, 60, 63, 66, 86, 89, 92, 95, 100, 105, 110, 115, 120, 125, 130, 135, 140, 145, 150, 155, 160, 165, 170, 175, 220, 225;
(v) reacting the compound 3 obtained in step (ii) with chlorobromoethane K₂CO₃ and dry DMF at 120° C. for 12 hours to obtain the compound 69;
(vi) reacting the compound 69 obtained in step (v) with suitable amine from the group consisting of Imidazole, N-Boc piperazine, pyrrole, indole, benzimidazole K₂CO₃ and dry DMF at 120° C. for 12 hours to obtain the compound selected from the group consisting of 70, 73, 77, 80, 83;
(vii) reducing the compound selected from the group consisting of3, 6, 9, 12, 18, 21, 24, 27, 30, 33, 36, 39, 42, 45, 48, 51, 54, 57, 60, 63, 66, 70, 73, 77, 80, 83, 86, 89, 92, 95, 100, 105, 110, 115, 120, 125, 130, 135, 140, 145, 150, 155, 160, 165, 170, 175, 220, 225 obtained in steps (ii), (iii) and (iv) using Palladium-Charcoal (5% or 10% wet) at room temperature for 2-5 hours in presence of H₂ to obtain an amine compound selected from the group consisting of 4, 7, 10, 13, 19, 22, 25, 28, 31, 34, 37, 40, 43, 46, 49, 52, 55, 58, 61, 64, 67, 71, 74, 78, 81, 84, 87, 90, 93, 96, 101, 106, 111, 116, 121, 126, 131, 136, 141, 146, 151, 156, 161, 166, 171, 176, 221, 226;
(viii) treating the compound 4, 7, 10, 13, 19, 22, 25, 28, 31, 34, 37, 40, 43, 46, 49, 52, 55, 58, 61, 64, 67, 71, 74, 78, 81, 84, 87, 90, 93, 96, 101, 106, 111, 116, 121, 126, 131, 136, 141, 146, 151, 156, 161, 166, 171, 176, 221, 226 obtained in step (vii) with 4-nitrophenylchloroformate in presence of TEA as a base followed by reaction with an amine 3′-aminoacetophenone in dry THF at room temperature for 3-8 hours to obtain the compound having structure I selected from the group consisting of 5, 8, 11, 14, 20, 23, 26, 29, 32, 35, 38, 41, 44, 47, 50, 53, 56, 59, 62, 65, 68, 72, 75, 79, 82, 85, 88, 91, 94, 97, 102, 107, 112, 117, 122, 127, 132, 137, 142, 147, 152, 157, 162, 167, 172, 177, 222, 227;
(ix) treating the compound 61, 221, 226 obtained in step (vii) with 4-nitrophenylchloroformate in presence of TEA as a base followed by reaction with an amine selected from the group consisting of m-anisidine, o-anisidine, 1-(3-aminophenyl)ethanol, 3-ethylaniline, methyl 3-aminobenzoate, 3-amino-N,N-dimethylbenzamide, (3-aminophenyl)(pyrrolidin-1-yl)methanone, (3-aminophenyl)(morpholino)methanone, aniline, N1-methylbenzene-1,3-diamine, N-(3-aminophenyl)acetamide, N-(3-aminophenyl)-N-methylacetamide, N-(3-aminophenyl)-N-benzylacetamide, 1-(3-(methylamino)phenyl)ethanone, 1-(3-(hydroxyamino)phenyl)ethanone, 1-(3-aminophenyl)-2,2,2-trifluoroethanone in dry THF at room temperature for 3-8 hours to obtain the compound having structure I selected from the group consisting of 178, 179, 180, 181, 182, 184, 185, 186, 187, 188, 189, 190, 191, 192, 193, 195, 228, 229, 230, 231,
(x) treating the compound 182 obtained in step (ix) with LiOH. H₂O in THF, Methanol and H₂O in proportion of (3:2:1) at room temperature for 12 hours to obtain the compound 183 having structure I;
(xi) treating the compound 62 obtained in step (viii) with hydroxylamine hydrochloride in EtOH at room temperature for 12 hours to obtain the compound 194 having structure I;
(xii) treating the compound 75 obtained in step (viii) with trifluoroacetic acid in DCM at room temperature for 8 hours to obtain the compound 76 having structure I;
(xiii) treating the compound 62 obtained in step (viii) with NH₃/Methanol and followed by NaBH₄ in Methanol at room temperature for 12 hours to obtain the compound 196 having structure I;
(xiv) treating the compound 196 obtained in step (xiii) with HCOOH and formaldehyde in presence of conc. HCl for 8 hours at 100° C. to obtain the compound 197 having structure I;
(xv) treating the compound 62 obtained in step (viii) with various aromatic amines (Aniline, 2-Fluoroaniline, 4-Fluoroaniline, 4-Methoxyaniline, 4-Aminobenzonitrile, cyclohexylamine, cyclopentylamine) in presence of p-Toluenesulfonic acid (PTSA) in EtOH at 100° C. to obtain the compounds having structure I selected from the group consisting of 196, 200, 202, 204, 206, 208, 210;
(xvi) treating the compounds 196, 200, 202, 204, 206, 208, 210 obtained in step (xv) with Sodium cyanoborohydride (NaCNBH₃) in dry methanol at room temperature for 8 hours to obtain the compounds having structure I selected from the group consisting of197, 201, 203, 205, 207, 209, 211;
(xvii) treating the compound which is commercially available 212 with HATU and 2-methoxyethylamine in DMF at room temperature for 2 hours to obtain the compound 213 having structure I;
(xviii) treating the compound obtained in step (xvii) with CDI in DMF for 12 hours at 100° C. to obtain the compound 214 having structure I;
(xix) reacting the compound 214 obtained in step (xviii) with 1-(2-chloroethyl) piperidine and K₂CO₃ and dry DMF at 120° C. for 12 hours to obtain the compound 215;
(xx) reacting the compound 215 obtained in step (xix) with 1-(2-amino-1H-benzo[d]imidazol-5-yl) ethenone and Pd₂(dba)₃ as catalyst and X-Phos as ligand and base K₂CO₃ at 100° C. for 12 hours to obtain the compound 216;
(xxi) reacting the compound 61 obtained in step (vii) with 3,4-Diethoxy-3-cyclobutene-1,2-dione and p-toluenesulfonic acid (PTSA) in EtOH and 3-aminoacetophenone at 80° C. for 12 hours to obtain the compound 217.

4. The compound as claimed in claim 1, for use in treating diseases and disorders related to modulation of COP1 enzyme through its stabilization or modulation of ATGL.

5. The compound as claimed in claim 1 for use in decreasing the level of triglycerides in hepatocytes.

6. The compound as claimed in claim 1, for use in treatment of disease selected from Non-Alcoholic Fatty Liver Disease (NAFLD) or Non-Alcoholic Steatohepatitis (NASH).

7. A composition comprising the compound having structure I as claimed in claim 1 along with pharmaceutically acceptable excipients.

8. A method of modulation COP1 enzyme through its stabilization by compound having structure I as claimed in claim 1.

9. A method of increasing the level of ATGL by compound having structure I as claimed in claim 1.
